(19) United States
(12) Patent Application Publication
(10) Pub. No.: US 2021/0030675 A1
(43) Pub. Date: Feb. 4, 2021

(54) MESOPOROUS SILICA NANOPARTICLES AND SUPPORTED LIPID BI-LAYER NANOPARTICLES FOR BIOMEDICAL APPLICATIONS

(71) Applicants: C. Jeffrey Brinker, Albuquerque, NM (US); Paul N. Durfee, Albuquerque, NM (US); Jason Townson, Albuquerque, NM (US); Yu-Shen Lin, Seattle, WA (US); Stanley Shihyao Chou, Albuquerque, NM (US); Jacob Erstling, Orlando, FL (US)

(72) Inventors: C. Jeffrey Brinker, Albuquerque, NM (US); Paul N. Durfee, Albuquerque, NM (US); Jason Townson, Albuquerque, NM (US); Yu-Shen Lin, Seattle, WA (US); Stanley Shihyao Chou, Albuquerque, NM (US); Jacob Erstling, Orlando, FL (US)

(21) Appl. No.: 16/828,137

(22) Filed: Mar. 24, 2020

Related U.S. Application Data
(63) Continuation of application No. 15/757,269, filed on Mar. 2, 2018, now abandoned.
(60) Provisional application No. 62/214,513, filed on Sep. 4, 2015.
(60) Provisional application No. 62/214,436, filed on Sep. 4, 2015.
(60) Provisional application No. 62/262,991, filed on Dec. 4, 2015.
(60) Provisional application No. 62/358,475, filed on Jul. 5, 2016.

(51) Int. Cl. A61K 9/127; A61K 47/62; A61K 47/69; A61P 31/14; A61P 35/02

(52) U.S. Cl. CPC A61K 9/127; A61K 9/1271; A61K 47/62; B82Y 40/00; A61P 31/14; A61P 35/02; A61K 47/6923

(57) ABSTRACT

The present disclosure is directed to methods of producing monosized protocells from monosized mesoporous silica nanoparticles (mMSNPs) and their use for targeted drug delivery formulations and systems and for biomedical applications. The present disclosure is also directed in part to a multilamellar or unilamellar protocell vaccine to deliver full length viral protein and/or plasmid encoded viral protein to antigen presenting cells (APCs) in order to induce an immunogenic response to a virus.

### CROSS-REFERENCE TO RELATED APPLICATIONS

This application claims the benefit of the filing dates of U.S. application Ser. No. 62-214,513, filed on Sep. 4, 205. U.S. application Ser. No 62/214,436. filed on Sep. 4. 2015. U.S. application Ser. No 62/358,475. filed on Jul. 5, 2016, and U.S. application Ser. No 62/282,991. filed on Dec. 4, 2015, the disclosures of which are incorporated by reference herein.

### STATEMENT OF GOVERNMENT INTEREST

This invention was made with government supped under grant no. PHS 2 PN2 EY016570B awarded by the National Institutes of Health: grant no 1U01CA151792-01 awarded by the National Cancer Institute; grant no. FA 9550-07-1-0054/9550-10-1-0054 awarded by the Air Force Office of Scientific Research; grant no 1U19ES019528-01 awarded by the National Institute of Environmental Health; grant no NSF:EF-0820117 awarded by the National Science Foundation, grant no. DGE-0504276 awarded by the National Science Foundation, grant no. U01 CA1519201 awarded by the National Institutes of Health, and contract no. DE-AC04-94AL85000 awarded by the U.S. Department of Energy to Sandia Corporation. The government has certain rights in the invention.

### BACKGROUND

Targeted nanoparticle-based drug delivery systems hold the promise of precise administration of therapeutic cargos to specific sites sparing collateral damage to non-targeted cells/tissues and potentially overcoming multiple drug resistance mechanisms (Bertrand et al., 2014: Tarn et al., 2013). However, successful development of such targeted nanocarriers has proven to be a complicated task, in some cases because subtle details like charge density distribution vis-à-vis net charge/zeta-potential (Townson et al., 2013) impact the in vivo behavior of nanoparticles (Petros et al., 2010; Hrkach et al., 2012 Crist et al , 2013; Dobrovoiskala and McNeil, 2013).

An effective targeted nanocarrier for in vivo applications would include; 1) uniform and controllable particle size and shape. 2) high colloidal stability under physiological and storage conditions 3) minimal non-specific binding interactions, uptake by the mononuclear phagocyte system (MPS), or removal by excretory systems, allowing extended circulation time; 4) high specifity to abnormal cells or tissues. 5) noninvasive imaging and diagnosis; 6) high capacity for and precise release of diverse therapeutic cargos; and 7) low immunogenicity and cytotoxicity. Dramatic advances have been made in the last 10 years in developing multifunctional nanocarriers via procedures including surface and charge modification (Townson et al., 2013; Wang et al., 2010. Perry et al., 2012; Lin et al., 2011; Zhu et al., 2014; Zhang et al.,. 2014) development of hybrid material chemistries (Lee et al., 2011; Lee et al., 2012); incorporation of functional machines such as stimuli-responsive agents (Li et al., 2012; Roggers et al., 2012) and conjugation with targeting ligands (Stechen et al., 2013). However, few nanocarrier platforms exhibit the combined desirable characteristics enumerated above.

In this context, mesoporous silica nanoparticles (MSNs) and MSN-supported lipid layer nanoparticles (e.g., bi-layer nanoparticles) are unique. In some instances, the MSN-supported lioud layer nanoparticles is called a protocell. Their modular design and combined properties, including controlled size and shape, large internal surface area, tunable pore and surface chemistry, considerable cargo diversity, high specificity and limited toxicity could allow simultaneous attainment and optimization of needed in vivo characteristics (Lin et al., 2012; Ashley et al., 2011; Ashley et al., 2012; Epler et al., 2012; Cauda et al., 2010; Mackowiak et al., 2013; Wang et al., 2013; Zhang et al., 2014). However, the full potential of these platforms has remained unrealized due to difficulty controlling their physicochemical properties and in vivo stability. This is not a unique problem to MSN based carriers, as the confounding effect of nanoparticle aggregation and poor colloidal stability on a broad range of nanoparticles has been attributed as the source of inaccurate and irreproducible results in complex biological systems (Petros et al., 2010; Lin et al., 2012).

In a non-limiting instance, a ‘protocell’ (Ashley et al., 2011; Ashley et al., 2012; Epler et al., 2012) is a supported lipid bi-layer (SLB) shown to have marked efficacy for targeted delivery of anti-cancer drugs, siRNA, and enzymes in vitro ((Ashley et al., 2011; Ashley et al., 2012; Epler et al., 2012). However, preliminary in vivo experiments conducted in an ex ovo chicken embryo model suggested that these ‘first generation’ protocells rapidly became trapped in the capillary bed and engulfed by immune cells. The synthesis of MSN ‘cores’ by evaporation induced self-assembly (EISA) Lu et al., 1999), leads to a wide particle size distribution (about 20 to about 800 nm). Subsequent calcinations resulted in irreversible particle aggregation (large hydrodynamic size, >500 nm), a characteristic that was responsible for the impaired circulation times.

### SUMMARY

The present disclosure provides for the synthesis of protocells with control over size, shape, pore structure, pore size, surface chemistry, and targeting, while maintaining particle size monodispersity and in vivo stability.

In one embodiment, a population of monosized protocells comprising a population of monosized mesoporous silica nanoparticles (mMSNPs or mMSNs) is provided, each of said nanoparticles comprising a lipid layer, e.g., a bi-layer or multi-lamellar, coating (e.g., fused thereto), e.g., completely covering the surface of the mMSNPs, wherein said population of protocells exhibits a polydispersity index (PdI or DPI) of less than about 0.1 to no more than about 0.2. In certain embodiments, the population of protocells exhibits a polydispersity index of less than about 0.1.

In one embodiment, a population of monosized (monodisperse) protocells is provided comprising a population of mMSNPs to each of which is coated with (fused thereto) a lipid bi-layer, said lipid bi-layer completely covering the surface of said mMSNPs, said lipid bi-layer being fused onto said nanoparticles. In one embodiment, at least one lipid in the bilayer at a weight ratio of at least about 200% by weight, e.g., about 200% to about 1000% by weight (e.g., about 2:1 to about 10:1) of said population of nanoparticles, wherein said lipid is at least one cationic, anionic or zwitterionic lipid, e.g., at least one zwitterionic lipid, optionally comprising cholesterol and further optionally comprising a lipid containing a functional group to which may be covalently bonded a targeting or other functional moiety.

Also provided are monosized protocells comprising a population of particle cores comprising monosized mMSNPs and a single lipid bi-layer fused (e.g., a supported lipid bi-layer, SLB) onto the surface of each nanoparticle, said lipid bi-layer comprising at least one lipid and being fused onto said nanoparticle as a monosized liposome in aqueous, e.g., a buffer, solution, wherein said liposome has an internal surface area which is equal to or greater than the external surface area of said nanoparticle. In one embodiment, the lipid bi-layer comprises about 50 to about 99.99 mole percent of at least one anionic, cationic or zwitterionic lipid, e.g., a phospholipid, or at least one zwitterionic phospholipid. In alternative embodiments, the lipid bi-layer comprises 0% to about 50% mole percent, at least about 0.1 up to about 50 mole percent cholesterol (a minor component of cholesterol), for example, about 0.1 to about 10 mole percent, about 0.5 to about 1.5 mole percent, about 1 mole percent cholesterol), about 0.01 to about 25 mole percent, about 0.1 to about 20 mole percent, about 0.25 to about 10 mole percent, or about 0.5 to about 5 to 7.5 mole percent of at least one lipid which contains a functional group to which a targeting moiety (e.g., a peptide, polypeptide such as a monoclonal antibody, etc. or agonist/antagonist of a receptor) or other functional moiety (e.g., a fusogenic peptide or a drug, among numerous others such as toll receptor agonists for immunogenic compositions) may be covalently attached.

In some embodiments, the monosized protocells comprise a SLB which has a lipid transition temperature or Tₘ which is greater than the temperature at which the protocells are stored or used. Accordingly, by utilizing a SLB with a Tₘ which is greater than the temperature at which the protocells are stored or used, the monosized protocells exhibit extended storage stability when stored in an aqueous solution and colloidal stability when these compositions containing these protocells are used to treat patients and subjects.

mMSNPs may range in diameter from about 1 nm to about 500 nm, about 5 nm to about 350 nm, about 10 nm to about 300 nm, about 15 nm to about 250 nm, about 20 nm to about 200 nm, about 25 nm to about 350 nm, or about 20 nm to about 100 nm. In one embodiment, the mMSNPs are about 80 to about 100 nm in diameter. In each instance, in a population of monodisperse MSNPs, each MSNP does not vary more than about 5% from the average diameter of the mMSNPs in the population and exhibits a polydispersity index (PdI or DPI) of less than about 0.1, or less than about 0.2. e.g., less than about 0.1.

Monosized protocells exhibit colloidal and/or storage stability. In particular, monosized protocells exhibit colloidal stability and storage stability in aqueous solution (water, buffer, blood, plasma, etc.) such that the protocells maintain their monodispersity for a period of at least several hours (about 2, 3, 4, 5 or 6 hours), at least about 12 hours, at least about 24 hours, at least about two days, three days, four days, five days, six days, one week, two weeks, four weeks, two months, three months, four months, five months, six months, one year or longer. In one embodiment, the protocells are stored in phosphate buffered saline solutions, saline solution (isotonic saline solution), other aqueous buffer solutions, or water (especially distilled water). The monosized protocells maintain their monodispersity in blood, plasma, serum and/or other body fluids for extended periods of time.

Monosized protocells may further comprise at least one additional component, for example, a cell targeting species (e.g., a peptide, antibody, such as a monoclonal antibody, an affibody or a small molecule moiety which binds to a cell, among others); a fusogenic peptide that promotes endosomal escape of protocells; a cargo, including one or more drugs (e.g., an anti-cancer agent, anti-viral agent, antibiotic, antifungal agent, etc.); a polynucleotide, such as encapsulated DNA, double stranded linear DNA, a plasmid DNA, small interfering RNA, small hairpin RNA, microRNA, a peptide, polypeptide or protein, an imaging agent, or a mixture thereof, among others), wherein one of said cargo components is optionally conjugated further with a nuclear localization sequence.

In certain embodiments, protocells comprise a nanoporous silica core with a supported lipid bi-layer; a cargo comprising at least one therapeutic agent (for example, an anti-viral agent, antibiotic or an anti-cancer agent which optionally facilitates cancer cell death, such as a traditional small molecule, a macromolecular cargo, e.g., siRNA such as S565, S7824 and/or s10234, among others, shRNA or a protein toxin such as a ricin toxin A-chain or diphtheria toxin A-chain) and/or a packaged plasmid DNA (in certain embodiments—histone packaged) disposed within the nanoporous silica core (e.g., supercoiled as otherwise described herein in order to more efficiently package the DNA into protocells as a cargo element) which is optionally modified with a nuclear localization sequence to assist in localizing/presenting the plasmid within the nucleus of the cancer cell and the ability to express peptides involved in therapy (e.g., apoptosis/cell death of the cancer cell) or as a reporter (fluorescent green protein, fluorescent red protein, among others, as otherwise described herein) for diagnostic applications. Protocells may include a targeting peptide which targets cells for therapy (e.g., cancer cells in tissue to be treated, infected cells or other cells requiring therapy) such that binding of the protocell to the targeted cells is specific and enhanced and a fusogenic peptide that promotes endosomal escape of protocells and encapsulated DNA. Protocells may be used in therapy or diagnostics, more specifically to treat cancer and other diseases, including viral infections, including hepatocellular (liver) and other cancers which occur secondary to viral infection. In other aspects, protocells use binding peptides which selectively bind to cancer tissue (MET peptides for example, as disclosed in WD 2012/149376, published Nov. 1, 2012 and CRLF2 peptides, for example, as disclosed in WD 2013/103814, published Jul. 11, 2013, relevant portions of which applications are incorporated by reference herein).

In another embodiment, a storage stable composition is provided comprising a population of monosized protocells in an aqueous solution such as buffered saline, water, or isotonic saline solutions, among others.

In an additional embodiment, pharmaceutical compositions (e.g., storage stable compositions) are provided comprising an effective amount of a population of protocells as described herein, in combination with at least one carrier, additive and/or excipient.

In still another embodiment, a method of producing monosized protocells is provided. The method includes providing a population of mMSNPs and exposing said nanoparticles to a population of monosized liposomes comprising at least one lipid (the lipid mixture may be simple or complex, depending on the ultimate function of the protocell), the liposome to mMSNP mass ratio being at least 2:1 (the liposomes may have an internal surface area which is greater than the external surface area of the nanoparticles), wherein the nanoparticles are exposed to the liposomes in an aqueous solution (e.g., an aqueous buffer solution such as phosphate buffered saline solution, although other solutions, including buffered saline solutions may be used). In one embodiment, the liposomes have a hydrodynamic diameter of than about 100 nm and low PDI value of less than about 0.2, or less than 0.1. In one embodiment, the monosized liposomes and mMSNPs are combined in buffered saline solution, sonicated or otherwise agitated for several seconds up to a minute or more) to allow the liposomal lipid to coat/fuse to the nanoparticles and the non-fused liposomes in solution are removed/separated from the protocells, for instance, by centrifugation. The pelleted protocells are redispersed at least once (e.g., in phosphate buffered saline solution or other solutions in which the protocells are to be stored and/or used) via agitation (e.g., sonication).

In still another embodiment, therapeutic methods comprise administering a pharmaceutical composition comprising a population of monosized protocells to a patient in need in order to treat a disease state or condition from which the patient is suffering. The disease state includes but is not limited to cancer, a viral infection, a bacterial infection, a fungal infection or other infection.

Thus, the disclosure provides therapeutic formulations with increased therapeutic efficacy in vivo. The dramatic therapeutic efficacy of numerous targeted nanoparticle-based delivery platforms observed in vitro has rarely translated into similar performance in vivo. In exceedingly complex living systems, particle polydispersity, sequestration, and instability have limited the delivery of cargos to specific cell types despite the presence of effective targeting agents. Described herein is a process for the synthesis and characterization of monodisperse mesoporous silica-supported lipid bi-layer nanoparticles (e.g., protocells) designed to exhibit in vivo stability and targeted cell binding. Specific aspects of the modular synthesis protocol allows for precise control of size, shape, pore structure, and surface chemistry that can be tailored to achieve colloidal stability and targeted binding for a range of applications. The demonstrated in vitro stability attributed to the supported lipid bi-layer was confirmed in vivo using real-time, high resolution microscopic analysis in a chicken embryo chorioallantoic membrane (CAM) model combined with hydrodynamic size analysis. Moreover, by establishing synthetic protocols that enabled colloidal stability and avoided non-specific binding of non-targeted protocells, antibody conjugation was demonstrated to direct highly selective binding in vivo.

In another embodiment, a multilamellar protocell T cell vaccine is provided that delivers full length viral protein and/or plasmid encoded viral protein to antigen presenting cells (APCs). The multilamellar protocell contains a nanoparticle core and at least an inner lipid bi-layer and an outer lipid bi-layer and, optionally, an inner aqueous layer which separates the core from the inner lipid bi-layer and further optionally, an outer aqueous layer which separates the inner lipid bi-layer from the outer lipid bi-layer. The outer lipid bi-layer of the protocell is functionalized with a Toll-like receptor (TLR) agonist (e.g., monophosphoryl lipid A (MPLA) and/or flagellin) to facilitate and initiate an immunological signaling cascade, said outer bi-layer further including a fusogenic peptide such as octa-arginine (R8) peptide to induce cellular uptake of the protocell. In addition, full length viral proteins may be distributed throughout the outer lipid bi-layer or said optional inner aqueous layer or outer aqueous layer, e.g., the outer aqueous layer, to be processed in the endosome and presented to CD4+ T cells through the MHC Class I pathway. The inner lipid bi-layer is functionalized with an endosomolytic peptide such as H5WYG (or alternatively, INF7, GALA, KALA, or RALA) which enhances endosomal escape. In some embodiments, the protocell includes an internal porous silica core loaded with plasmid DNA encoding viral proteins and/or viral proteins fused to ubiquitin to be processed in the cytoplasm and presented to CD8+ T cells through the MHC Class I pathway. The plasmid is transcribed into a template and further translated into viral proteins, which are labeled with ubiquitin, a regulatory protein that tags and directs proteins to the proteasome for further degradation in preparation for antigen presentation.

In one embodiment, a multilamellar protocell is provided comprising a nanoporous silica or metal oxide core and a multilamellar lipid bi-layer coating, said core comprising an inner lipid bi-layer and an outer lipid bi-layer and optionally, an inner aqueous layer separating said core and said inner lipid bi-layer and an optional outer aqueous layer separating said inner lipid bi-layer and said outer lipid bi-layer, said outer lipid bi-layer of said multilamellar lipid bi-layer comprising: at least one TLR agonist such as MPLA and/or flaggellin to initiate an immunological signaling cascade; a fusogenic peptide (e.g., octa-arginine (R8) peptide) to induce cellular uptake of the protocell; and optionally at least one cell targeting species which selectively binds to a target (peptide, receptor or other target) on APCs; said inner lipid bi-layer of said multilamellar bi-layer comprising an endosomolytic peptide (e.g., H5WYG) to enhance endosomal escape, and said outer lipid bi-layer and/or said inner lipid bi-layer and/or said optional outer aqueous layer and/or said optional inner aqueous layer further comprising at least one viral antigen (e.g., a full length viral protein, which is optionally ubiquitylated as a fusion protein) distributed throughout said outer lipid bi-layer, said inner lipid bi-layer and/or said optional outer aqueous layer and/or said optional inner aqueous layer; said nanoporous core of said protocell being loaded with a pre-ubiquitylated viral protein (e.g., as a single peptide chain that includes ubiquitin or a ubiquitylated viral antigen) or a plasmid DNA encoding viral protein, which is optionally labeled with ubiquitin.

Multilamellar protocells may also comprise a drug (including, for example, an anti-viral agent) or other agent to enhance an immunogenic response such as an adjuvant.

Additional embodiments are directed to compositions comprising at least two different or separate populations of unilamellar protocells (optionally containing an aqueous layer separating a core from the single lipid bi-layer) such that the combined populations of protocells comprise the same elements (in the different/separate populations) as in the multilamellar protocells described above, but the separate populations of protocells, for instance, deliver viral antigen (e.g., as a ubiquitylated viral antigen) and/or plasmid DNA which encodes a viral antigen (e.g., as a viral antigen fused to ubiquitin). In one aspect, a first population of unilamellar protocells delivers viral antigen (often in the absence of ubiquitinylation and the absence of at least one endosomolytic peptide) and the second population of unilamellar protocells delivers viral protein/antigen and/or DNA plasmid expressing viral antigen in the presence of endosomolytic peptide.

In one embodiment, one or more of the populations of protocells (often at least two and in certain embodiments all populations of the protocells) comprise at least one TLR agonist, at least one fusogenic peptide (e.g., R8 octa-arginine to facilitate cellular uptake of the protocells) and at least one targeting species to facilitate binding of the protocells to a target on the antigen presenting cells in the lipid bi-layer of the protocell; one or more populations of protocells in said composition (often at least two and in some embodiment all of the protocells) comprise at least one endosomolytic peptide in the lipid bi-layer. One population of protocells comprises at least one viral antigen (which may be a full length viral protein) in the lipid bi-layer or optional aqueous layer of said protocell. This population may comprise an endosomolytic peptide or may exclude such a peptide and one or more populations of protocells in the composition is loaded in the core of said protocell with a viral protein, such as a full length viral protein which is optionally ubiquitinylated (and presented as a fusion protein) and/or a plasmid DNA encoding at least one viral protein (e.g., a full length viral protein), which is optionally and labeled with ubiquitin (expressed as a fusion protein), this protocell population may comprise an endosomolytic peptide. Optionally, one or more populations of protocells in said composition are loaded with at least one bioactive agent, for instance an anti-viral agent.

Accordingly, in some embodiments, the population of protocells is comprised of multiple components, as described above, either in a multilamellar protocell (e.g., as a single population of protocells) or two or more populations of unilamellar protocells which comprise at least the minimum elements of the multilamellar protocells, but in more than one population of protocells to obtain a similar result. This approach uses a unilamellar fusion of CD4+ stimulating and/or CD8+ stimulating protocells mixed and injected simultaneously or sequentially to provide a similar effect to the multilamellar protocells described herein, but in different/separate populations of protocells. It is noted generally that plasmid DNA encoding at least one viral protein (which is optionally ubiquitinylated) or antigen including a full length viral protein (which is optionally ubiquitinylated) in the presence of an endosolytic peptide generally provides CD8+ stimulation and viral antigen (whether ubiquitinylated or not) in the absence of an endosomolytic peptide generally provides CD4+ stimulation (but can also provide CD8+ stimulation).

Pharmaceutical compositions are provided comprising a population of multilamellar or unilamellar protocells in an immunogenic effective amount in combination with at least one additive, excipient and/or carrier. The pharmaceutical composition may comprise additional bioactive agents and other components such as adjuvants (these may also be incorporated into the protocell. Compositions may be used to induce an immunogenic response and/or protective effective against any number of viral infections.

In another embodiment, methods of instilling immunity and/or an immunogenic response or vaccinating a patient or subject at risk for a disease (e.g., an infection such as a viral infection), are provided. The methods include administering a composition to a patient or subject in need in order to induce an immunogenic response in that patient or subject to a virus in order to reduce the likelihood that said patient or subject wil become infected with said virus and/or to reduce the likelihood that a virus will cause an acute or chronic infection in said patient or subject.

In one embodiment, a hybrid bilayer protocell is provided comprising a mesoporous silica nanoparticle (MSNP or MSN) which is coated on its surface with a hydrocarbon layer, often comprising a silyl hydrocarbon (generally, a C₆-C₄₀ linear, branched or cyclic silylhydrocarbon (e.g., alkylsilane), a C₈-C₃₂ linear, branched or cyclic silylhydrocarbon (e.g., alkyl silane), a C₁₀-C₂₈ linear, branched or cyclic silylhydrocarbon (e.g., alkyl silane or), or a C₁₂-C₂₈ linear, branched or cyclic silyl hydrocarbon (alkyl silane)), the hydrocarbon layer being further coated with a lipid monolayer and a hydrophobic cargo, often a hydrophobic drug loaded into the hybrid bilayer protocell. In alternative embodiments, the hydrocarbon layer comprises a lipid with a primary amine modified headgroup, for example, an amine-containing phospholipid (e.g. DOPE, DMPE, DPPE or DSPE) which is conjugated to the surface of the MSNP through a carboxyl group formed on the surface of the MSNP and a crosslinking agent which crosslinks the surface of the MSNP (through the carboxylic acid moiety) with the amine group of the primary amine containing lipid. The loaded hybrid lipid protocell may be formulated in pharmaceutical dosage form for administering to a patient for the treatment or diagnosis of disease and/or related conditions. In certain embodiments, the hybrid bilayer protocell may contain on the surface of the lipid monolayer PEG groups, targeting peptides and other components which facilitate the administration of the hydrophobic cargo to a particular target, including a cell.

In one embodiment, MSNPs are synthesized utilizing standard methods in the art as described herein. After formation of the MSNP, the MSNP is then reacted with a chlorosilane hydrocarbon to covalently bond (through Si—O—Si) the silyl hydrocarbon to the surface of the MSNP. The step of reacting the chlorosilane hydrocarbon to the MSNP may occur before or after hydrothermal treatment (e.g., between about 12 and 24 hours at elevated temperatures, e.g. 70° C.).

Alternatively, the MSNPs are reacted with a carboxylation agent (e.g., 3-(Triethoxysilyl)propylsuccinic anhydride or other agent to incorporate a carboxyl group on the surface of the MSN) at about 0.1% to about 20% of the molar ratio of TEOS or other silica precursor) for a time sufficient for the carboxylation agent to react with the surface of the MSNP to provide a carboxyl moiety on the surface of the MSNP. The carboxylation step may occur before or after hydrothermal treatment. The carboxylated MSNP is thereafter reacted with a crosslinking agent, e.g., EDC and the crosslinked MSNP is further reacted with an amine containing phospholipid (DOPE, DMPE, DPPE, DSPE or other amine-containing phospholipid to provide a hydrocarbon group on the surface of the MSNP through the crosslinking agent.

The MSNPs which have hydrocarbon surfaces are then mixed with one or more phospholipids, generally, a mixture of a phospholipid containing a PEG group as otherwise described herein and another phospholipid as described herein. The hydrocarbon coated MSNPs and phospholipid are mixed in solvent (often chloroform or methylene chloride) often along with a cargo to be incorporated into the final hybrid bilayer protocell and dried together (evaporation of solvent) to form a film. The film is then hydrated with PBS or other buffer and washed several times to form the final MSNPs containing cargo. The cargo may be loaded into the hybrid bilayer protocells at the time that the phospholipid is coated/fused onto the MSNP or alternatively, the cargo may be added at the time after film formation by incorporating the hydrophobic cargo into the hybrid bilayer protocell when the film is hydrated with buffer.

Hybrid bilayer protocells, in addition to containing at least one hydrophobic cargo, may also include one or more of the following: a targeting species including, for example, targeting peptides including oligopeptides, antibodies, aptamers, and PEG (polyethylene glycol) (including PEG covalently linked to specific targeting species); a cell penetration peptide such as a fusogenic peptide or an endosomolytic peptide as otherwise described herein; a hybrid bilayer protocell comprising a mesoporous silica nanoparticle (MSNP) with a hydrocarbon coating on said MSNP and a lipid monolayer coated onto said hydrocarbon coating, wherein said protocell is loaded with a hydrophobic cargo. In one embodiment, the hydrocarbon coating comprises a C₆-C₄₀ silylhydrocarbon. In one embodiment, the hydrocarbon coating comprises is a C₁₂-C₂₈ alkyl silane. In one embodiment, the hydrocarbon coating is formed by reacting a chlorosilylhydrocarbon with the surface of the MSNP. In one embodiment, the hydrocarbon is formed by reacting carboxylic moieties on the surface of the MSNP with a lipid comprising a primary amine modified headgroup through a crosslinking agent. In one embodiment, the lipid is DOPE. DMPE, DPPE or DSPE. In one embodiment, the crosslinking agent is selected from the group consisting of 1-Ethyl-3-[3-dimethylaminopropyl]carbodiimide hydrochloride (EDC), succinimidyl 4-[N-malelmidomethyl]cyclohexane-1-carboxylate (SMCC), Succinimidyl 6-[ß-Maleimidopropionamido]hexanoate (SMPH), N-[ß-Maleimidopropionic acid] hydrazide (BMPH), NHS-(PEG)ₙ-maleimide, succinimidyl-[(N-maleimidopropionamido)-tetracosaethylenegycol] ester (SM(PEG)₂₄), and succinimidyl 6-[3′-(2-pyridyldithio)-propionamido] hexanoate (LC-SPDP). In one embodiment, the lipid monolayer comprises a pegylated phospholipid. In one embodiment, the lipid monolayer comprises a mixture of a phospholipid and a pegylated phospholipid. In one embodiment, the lipid monolayer comprises DSPE-PEG and/or DOPE-PEG (wherein the PEG average molecular weight is 2000) and optionally one or more of DHPC, DMPC, DOPE, DPPC and cholesterol. In one embodiment, the lipid monolayer includes cholesterol in a minor amount (i.e., less than 50% by weight of the lipid in the lipid monolayer). In one embodiment, the hydrophobic cargo is a drug. In one embodiment, the hydrophobic cargo is a reporter. Also provided is a pharmaceutical composition comprising a population of hybrid protocells in combination with a pharmaceutically acceptable carrier, additive and/or excipent. Further provided is a method of treating a disease state or condition in a patient in need comprising administering to said patient the pharmaceutical composition. In one embodiment, the disease state is cancer.

### BRIEF DESCRIPTION OF THE FIGURES

FIGS. 1A-D. A) Representative TEM image of bare mMSNs with ordered hexagonally arranged mesopores. B) Cryo-TEM image of monosized protocells. White arrows highlight the supported lipid bi-layers. Scale bars=50 nm. C) Hydrodynamic size comparison between bare mMSNs and protocells in different buffer conditions. D) Digital photograph of RITC-labeled mMSNs in DI H₂O and PBS and the corresponding protocells in PBS.

FIGS. 2A-D. A) Hydrodynamic size of particles prepared using different lipid to mMSN mass ratios (w:w)—bottom, and respective calculated surface area ratios—top. Dashed fine indicates optimal protocell size range. B) Hydrodynamic size comparison of synthesized monosized protocells under differing ionic strength fusion conditions. Nanopartile hydrodynamic diameter measurements over 72 hours at 37° C. in C) 1×PBS and D) DMEM+10% FBS. Data represent mean±SD, n=3.

FIG. 3A-E2. Hydrodynamic size measurements of bare mMSNs (left) and corresponding protocells (right) prepared from various mMSN cores: spherical mMSN with 2.5 nm pore, dendritic mMSN with 5 nm pore, dendritic mMSN with 8 nm pore, or rod-shaped mMSNs with 2.8 nm pore. Data represent mean±SD, n=3. Conventional TEM images of (b1) spherical, (c1) dendritic with 5 nm pores, (d1) dendritic with 8 nm pores, and (e1) rod-shaped mMSNs and the cryo-TEM images of (b2, c2, d2, e2) the corresponding protocells. White arrows highlight lipid bi-layer. Scale bars=50 nm.

FIGS. 4A-D. Differential binding/uptake of A) RITC-labeled mMSNs and B) protocells after 4 hours incubation with human endothelial cells (EA.hy928) at 20 μg/mL. (blue—nuclei stained by Hoechst 33342, green—actin stained by Alexa Fluor® 488 phalloidin). Scale bar=20 μm. Flow cytometry measurements of C) RITC-labeled mMSNs and D) protocells. %=percent population shift due to particle fluorescence (white=control, no particle exposure, grey=mMSNs or protocells).

FIG. 5A-D. RITC-labeled mMSN and protocell flow patterns observed in vivo using the CAM model. Representative fluorescent image section insets highlight differential flow characteristics between (a and b) mMSNs with diminished flow and aggregation compared to (c and d) protocells with unobstructed flow and prolonged circulation, captured at 5 minutes and 30 minutes post injection. Scale bar=50 μm.

FIGS. 6A-D. A) RITC-labeled protocells prepared from mMSNs extracted from CAM after 10 minutes circulation and imaged on a glass slide with brightfield and fluorescent overlay. B) DLS measurements of mMSNs, protocells, and protocells after separation from avain blood. C) IV injected RITC-labeled protocells extracted from a Balb/c mouse after 10 minutes circulation and D) corresponding DLS measurement of mMSNs and protocells pre-injection and post-separation from mouse blood. Scale bar=20 μm. Data represent mean±SD, n=3.

FIG. 7A-D. In vitro fluorescent microscopy images which reveal A) minimal EGFR targeted protocells (red) binding observed after 1 hour incubation with non-EGFR expressing Ba/F3 cell line (blue-nuclei, green-cell membrane), while B) targeted protocells (red) exhibit a high degree of specificity for EGFR expressing Ba/F3 cell line. Flow cytometry analysis of anti-EGFR protocells incubated with C) Ba/F3 and D) Ba/F3+EGFR support fluorescent microscopy analysis, %=percent population shift due to particle fluorescence (white=control, no particle exposure, grey=mMSNs or protocells). Scale bar=10 μm.

FIGS. 8A-B. Fluorescent microscopy images acquired in vivo in the CAM model show: A) stable circulation of anti-EGFR targeted protocells (red) and the initial stages binding to Ba/F3+EGFR (green) 10 minutes post injection; B) internalized anti-EGFR targeted protocells (yellow, due to merged green and red) within Ba/F3+EGFR (green) 20 hours post-injection. Scale bar=10 μm.

FIG. 9. The schematic shows liposome fusion to mMSN, formation of a protocell, and targeting chemistry approach. Liposomes containing DSPE-PEG₂₀₀₀-NH₂ are prepared and mixed with mMSNs to form aminated protocells. The primary amine is converted into a thiol group with the addition of Traut's reagent. The thiol group on the protocell reacts with the maleimide modified NeutrAvidin. In the final step, biotinylated antibodies bind to the NeutrAvidin on the protocell surface to form targeted protocells.

FIGS. 10A-B. A) Dynamic light scattering measurements of mMSN and monosized protocells. B) Zeta potentials of protocell component parts in phosphate buffered saline.

FIG. 11. In vivo stable mesoporous silica supported lipid bi-layer nanoparticles, or “protocells,” require monosized, colloidally stable cores. Monosized mesoporous silica nanoparticle support is essential for in vivo stable protocell platform. The lipid bi-layer coating reduces non-specific interactions in vitro, improves circulation time in vivo, and can be modified to enhance target specificity. The monosized protocells are an improvement upon the previous platform design with demonstration of in vitro stability coupled with in vivo performance.

FIGS. 12A-D. Conventional TEM image of MSNs prepared from A) EISA synthesis route. Scale bar=200 nm. B) Histogram of particle size distributions of EISA and mMSN cores. C) Hydrodynamic size comparison of bare EISA particle, EISA protocell, bare mMSN, and monosized protocell. Data represent mean±SD, n=3. D) mMSNs synthesized from solution-based method. Scale bar=200 nm.

FIG. 13. N₂ adsorption-desorption isotherms and pore size distribution (inset) of mMSNs composed of hexagonally arranged pores.

FIG. 14. N₂ adsorption-desorption isotherms and pore size distribution (inset) of dendritic mMSNs with 5 nm or 9 nm pores.

FIGS. 15A-B. A) Percentage of lysed human red blood cells (hRBCs) after exposure to 25, 50, 100, 200, and 400 μg/mL of mMSNs and protocells for 3 hours at 37° C. Data represent mean±SD, n=3. B) Digital photographs of hRBCs after 3 hours incubation with (top) bare mMSNs or (bottom) protocells at different particle concentrations (25, 50, 100, 400 μg/mL). Presence of red hemoglobin in supernatant indicates membrane damaged hRBCs after NP exposure.

FIGS. 16A-D. Flow cytometry measurements of EA.hy926 endothelial cells after incubation with 20 μg/ml of A) EISA MSN, B) EISA protocell, C) mMSN, and D) monosized protocells for 4 hours. Percent population shift due to particle fluorescence (grey=control, no particle exposure, blue outline=mMSNs or protocells).

FIG. 17. Representative in vivo binding and flow patterns of RITC-labeled EISA protocells (red) in CAM 5 minutes post-injection. The white arrows highlight large EISA protocell aggregates rapidly trapped in capillary bed or engulfed by immune cells. Blue-autofluorescence from tissue.

FIG. 18. The composition and hydrodynamic size data of liposomes used for preparation of protocells.

FIG. 19. In vitro targeting of anti-EGFR affibody MSNPs.

FIG. 20A-C. In vitro targeting of GE11 conjugated MSNPs.

FIG. 21A-B. Evidence of affibody binding both in vitro and in vivo. Left=nanoparticles, with nuclei, right=extravascular space, including nanoparticles, and target A431 cells.

FIG. 22. Evidence of peptide crosslinked nanoparticles binding to target Hep3B cells ex ovo. The extravascular space, nanoparticles, and target Hep3B cells are shown.

FIG. 23. Top image shows untargeted protocells do not bind to cells (HeLa cells), but with folate conjugated to the SLB a high degree of specific binding is observed (bottom image). Green=action; blue=DNA (DAPI); red=folate.

FIG. 24. Amine terminated lipid head groups can be modified with copper free click moiety (DBCO) which is then capable of bonding to azide (N3) functional groups on molecules, peptides, antibodies, affibodies, single chain variable fragments (scFvs). DSPE-PEG-DBCO is also commercially available and can be incorporated in the standard SLB formulations. Lipids can be modified before or after liposome preparation, and or fusion to MSNP support.

FIG. 25. Measure of size and stability of protocells modified with copper free click lipid head groups (DPSE-PEG-DBCO). The figure shows protocells fluorescence due to successful click reaction to the SLB surface using Carboxyrhodamine 110. The top image shows no fluorescence because it only contains clickable lipid group, middle image shows major aggregation in the absence of SLB, and the bottom image shows disperse population of green labelled protocells in solution. Data on left show that this targeting strategy does not destabilize the protocell because the hydrodynamic size is slightly larger than the MSNP core and the PdI<0.1.

FIGS. 26A-B. A) Highly specific protocell binding observed 30 minutes post injection using intravital imaging technique, demonstrating that monosized protocell targeting can be achieved in complex biological systems. B) Protocell binding with high affinity and or internalization is observed 21 hours post injection using intravital imaging technique, demonstrating that monosized protocell targeting can be achieved longer term in complex biological systems.

FIG. 27. Folate targeted protocell and cargo release in vivo. A) Targeted HeLa cell, B) Internalized folate conjugated protocells, C) membrane impermeable cargo, and D) merging with vasculature.

FIGS. 28 A-F. A) Flow cytometry analysis of REH+EGFR cells incubated with red fluorescent EGFR targeted protocells at multiple time points. Corresponding fluorescent microscopy analysis of REH+EGFR cells fixed and stained (blue-nuclei, green-cytoskeleton, red-proto cells) (B) untreated, or at (C) 5 min, (D) 15 min, (E) 30 min, and (F) 60 min incubation times. These data illustrate rapid in vitro protocell binding in as little as 5 min in complete medium, and maximal protocell accumulation after 30 min. Scale bar=5 μm.

FIG. 29A-C. Intravital fluorescent microscopy images acquired ex ovo in the CAM model reveal stable circulation of EGFR targeted protocells (red) and binding to REH+EGFR cells (green) in circulation at 1 h (left), 4 h (top right), and 9 h (bottom right) time points. Systemic protocell circulation is diminished after 4 h, however protocells remain associated with target cells for up to 9 h. Scale bar (left)=50 μm, Scale bars (right)=10 μm.

FIG. 30. Decrease in viability of REH+EGFR cells with increasing concentration of GEM loaded EGFR-targeted protocells. REH+EGFR cells incubated with protocells from 0 to 50 ug/ml for 1 hour, then washed to remove unbound protocells. Viability was assessed at 24 hours. Viability data highlights target specific delivery of cytotoxic cargo using monosized protocell platform. Data represents mean±SD, n=3.

FIG. 31. Increasing the concentration of Gemcitabine (GEM) loading does not destabilize protocells or influence the size of targeted protocells

FIGS. 32A-I. Intravital fluorescent microscopy images acquired ex ovo in the CAM model reveal stable circulation of non-targeted protocells but no association with A-C) REH+EGFR cells at 1 hour (A), 4 hours (B) and 9 hours (C); D-F) REH NeutrAvidin cells at 1 hour (D), 4 hours (E) and 9 hours (F); G-I) parental REH cells in circulation at 1 hour (G), 4 hours (H), and 9 hours (I) time points. EGFR targeted protocells circulate but do not associate with parental REH cell in circulation. Scale bar (left)=50 μm, Scale bars (right)=10 μm.

FIGS. 33A-C. Flow cytometry analysis of red fluorescent non-targeted protocells incubated with A) REH+EGFR cells and B) parental REH cells at multiple time points. Flow cytometry data confirm components used with our targeting strategy do not contribute to non-specific binding in vitro. In addition, red fluorescent EGFR-targeted protocells incubated with C) parental REH cells at multiple time points do not bind, demonstrating a high degree of specificity with our targeting strategy.

FIGS. 34A-B. Green fluorescent EGFR expressing cells were injected into chorioallantoic member (CAM) and allowed to circulate and arrest in the capillary bed for 30 minutes. After 30 minutes, monosized anti EGFR targeted protocells were injected and allowed to circulate for 1 hour. These figures show that intravital imaging reveals significant targeted protocell binding with target cells. In addition, flow patterns observed in red fluorescent lines indicate that targeted protocells maintain colloidal stability while circulating in a live animal system.

FIG. 35. A schematic which demonstrates that B cell vaccines produce soluble antibodies that neutralize pathogens outside of the host cell. T cell (purple) vaccines recognize surface expression of pathogen protein components via the T cell receptor and directly kill the pathogen.

FIG. 36. A schematic illustration of one embodiment of a multilamellar protocell modified with various targeting ligands and loaded with viral protein and DNA cargo. Note that the protocell contains both an inner lipid bi-layer and an outer lipid bi-layer and an inner aqueous layer separating the inner lipid bi-layer from the core and an outer aqueous layer separating the inner lipid bi-layer from the outer lipid bi-layer.

FIG. 37. A schematic of protocell uptake and immune signaling cascade initiation through TLR. Once internalized, the outer protocell layer will be broken down to release viral protein cargo, which is further degraded in the endosome. The internal lipid bi-layer is functionalized with an endosomolytic peptide (such as H5WYG) will release the viral protein/or plasmid cargo into the cytoplasm.

FIG. 38. A schematic of MHC Class I Pathway. Endogenous proteins are broken down into peptide fragments that can be expressed on MHC Class II molecules and presented to CD8+ T cells.

FIG. 39. A schematic of MHC Class II Pathway. Exogenous proteins are broken down into peptide fragments that can be expressed on MHC Class II molecules and presented to CD4+ T cells.

FIG. 40. A schematic illustration of engineered unilamellar protocells modified with various targeting ligands and loaded with ubiquitinylated viral protein and DNA cargo expressing viral protein. This protocell is illustrative of a unilamellar protocell adapted to produce CD8+ T cells (cytotoxic) pursuant to the MHC class I pathway. Note that the unilamellar liposomes depicted here may be administered alone or in combination with unilamellar liposomes which are adapted to produce CD4+ T cells (helper) pursuant to the MHC class I pathway.

FIG. 41. A schematic illustration of engineered unilamellar protocells modified with various targeting ligands and loaded with viral antigen as cargo. This protocell is illustrative of a unilamellar protocell adapted to produce CD4+ T cells (helper) pursuant to the MHC class II pathway. Note that the unilamellar liposomes depicted here may be administered alone or in combination (simultaneously or sequentially) with unilamellar liposomes which are adapted to produce CD8+ T cells (cytotoxic) pursuant to the MHC class I pathway.

FIG. 42. Schematic depicting lipid vesicle fusion onto nanoparticles to form mesoporous silica-supported lipid bi-layer nanoparticles (e.g., protocells). Drug (gemcitabine) and/or fluorescent molecular cargo (YO-PRO®-1) loaded protocells were assembled by soaking nanoparticle cores with cargo for 24 hours in aqueous buffer. Liposomes composed of either pre-targeted (DSPC:chol:DSPE-PEG₂₀₀₀-NH₂—49:49:2 mol ratio) or non-targeted (DSPC:chol:DSPE-PEG₂₀₀₀—54:44:2 mol ratio) were then fused to either loaded or unloaded cores. Leukemia cell targeting ability was added to the protocell by successive modifications to the DSPE-PEG₂₀₀₀-NH₂ supported lipid bi-layer component resulting in highly specific EGFR-targeted protocells. Lipid bi-layer and supported lipid bi-layer thickness is nearly identical as shown in cryogenic TEM images.

FIGS. 43A-M. Representative TEM and Cryo-TEM images of MSNs and corresponding protocells of various shape and pore morphology including (A and B) Hexagonal mMSNs and protocells, (C and D), Spherical 2.8 nm pore mMSNs and protocells, (E and F) Spherical 5 nm pore mMSNs and protocells, (G and H) Spherical 8 nm pore mMSNs and protocells, (I and J) Rod-like 2.8 nm pore mMSNs and protocells, (K and L) Aerosol assisted EISA MSNs and protocells. Yellow arrows highlight the SLB (about 4.6 nm) in the Cryo-TEM images. M) Hydrodynamic size analysis by DLS shows an increase in nanoparticle diameter following SLB fusion. DLS data represent mean±SD, n=3. Scale bars=50 nm.

FIGS. 44A-B. A) Comparison of Hexagonal protocells prepared in differing ionic strength conditions using different liposome to mMSN mass ratios (w:w)—bottom, and respective calculated inner liposome to outer mMSN surface area ratios—top. Hydrodynamic size (Left axis) corresponds to bar graph with black dashed line indicating optimal protocell size range. Polydispersity index (Right axis) corresponds to box plots with blue dashed line indicating threshold for monodispersity, values below the dashed line are considered monodisperse (PdI<0.1). Green arrow identifies the optimal ionic strength and liposome:mMSN ratio fusion conditions used for subsequent experiments. B) Fluorescently labelled mMSNs and protocells in cuvettes illustrate the colloidal stability of mMSNs in H₂O and aggregation driven settling of mMSNs in 160 mM PBS, protocells remain suspended in 160 mM PBS.

FIG. 45. Hydrodynamic size characteristics and zeta potential measurements of modular protocell components. Liposome formulation DSPC:chol:DSPE-PEGMO (mol % 54:44:2). Data represent mean±SD, n=3.

FIG. 46. Cryo-TEM image of 18 nm pore structured mMSNs mixed with liposomes under optimized fusion conditions as established in FIG. 46 showing large lipid-associated aggregates. (Inset): conventional TEM of 18 nm pore structured mMSNs. Yellow arrows highlight regions of liposome to silica interactions, red arrows highlight exposed silica surfaces. Scale bar=100 nm. Corresponding hydrodynamic size measurements: mMSNs with 18 nm pore diameter, Z-average diameter=123.0±0.3 nm (Avg PdI=0.056±0.018); lipid associated aggregates Z-average diameter=396.9±13.0 nm (Avg PdI=0.139±0.043). DLS data represent mean±SD, n=3

FIGS. 47A-B. A) Hydrodynamic size of protocells prepared with differing SLB formulations versus incubation time at 37° C. in 160 mM PBS. Trend in size change appears dependent on Tm of SLB components rather than PEGylation. B) Hydrodynamic size of PEGylated protocells prepared with differing SLB formulations versus incubation time at 37° C. in DMEM+10% FBS. All data represent mean±SD, n=3.

FIGS. 48A-D. Fluorescently-labelled nanoparticle flow patterns observed using ex ovo CAM model. Representative sections highlight differential flow characteristics between A) monosized protocells 5 minutes post injection and B) 30 minutes post injection compared to C) EISA protocells 5 minutes post injection and D) 30 minutes post injection. Scale bar=50 μm.

FIGS. 49A-D. A) Fluorescent labeled protocells pulled from CAM 10 minutes post-injection and imaged on glass slide with Zeiss AxioExaminer upright microscope. We observed protocells in motion moving in and out of frame in a Brownian pattern with no apparent direct association with red blood cells. B) Hydrodynamic size and PdI of core Hexagonal mMSNs, protocells, and protocells separated from CAM blood. C) Fluorescent protocells injected and pulled from Balb/c mouse 10 minutes post-injection. D) Hydrodynamic size and PdI of core Hexagonal mMSNs, protocells, and protocells separated from mouse blood. Injected protocells were separated from blood by variable speed centrifugation. Microscopy image scale bars=20 μm and DLS data represent mean±SD, n=3. Data provides evidence of size stability (A and B) ex ovo and (C and D) In vivo as assessed by minimal change in hydrodynamic size and PdI values.

FIGS. 50A-B. Flow cytometry analysis of REH+EGFR A) and parental REH-EGFR B) cells incubated with red fluorescent EGFR targeted protocells at multiple time points. This data illustrates rapid specific in vitro protocell binding to REH+EGFR in as little as 5 minutes in complete medium, and maximal protocell accumulation after 30 minutes A). Red arrows highlight non-EGFR expressing population of the engineered REH+EGFR cell line. There is minimal non-specific binding to parental REH cells B).

FIGS. 51A-C. Intravital fluorescent microscopy images acquired ex ovo in the CAM model reveal stable circulation of EGFR targeted protocells (red) and binding to REH+EGFR cells (green) in circulation at (A) 1 hour, (B) 4 hours, and (C) 9 hours time points. Systemic protocell circulation is diminished after 4 hours, however protocells remain associated with target cells for up to 9 hours. Scale bar (A)=50 μm, Scale bars (B and C)=10 μm.

FIGS. 52A-F. Still frames which capture the targeted protocell binding to green fluorescent labelled REH+EGFR cell in the (A-C) top and (D-E) bottom of the frame from a video with arrows indicating points where red fluorescent protocells appear to bind and remain associated with the cells. The capture of real-time fluorescent nanoparticle binding is made difficult by the exposure of three fluorescent channels in succession at each time point, therefore the motion of an individual nanoparticle binding event cannot be captured using this imaging technique. Scale bar=20 μm.

FIGS. 53A-F. Flow cytometry analysis to assess internalization of A) red fluorescent EGFR-targeted protocells by REH+EGFR cells in vitro at multiple time points and B) delivery of model drug, YO-PRO®-1, a green cell impermeant dye. After each time point, cells were acid washed to strip surface bound protocells then fixed. These data show an increase in the internalization of protocells and release of cargo with increasing incubation time. C) Maintained viability of REH cells and decrease in viability of REH+EGFR cells with increasing concentration of GEM loaded EGFR-targeted protocells. REH and REH+EGFR cells incubated with protocells from 0 to 50 ug/ml for 1 hour, then washed to remove unbound protocells. Viability was assessed at 24 hours. D) Loss in cell viability of REH and REH+EGFR cells with exposure to increasing concentration of free GEM. Both cell lines were incubated with free GEM from 0 to 30 uM for 1 hour, then washed to remove unassociated free drug. Viability was assessed at 24 hour. Viability data highlights target specific delivery of cytotoxic cargo using monosized protocell platform and the non-specific cytotoxicity of free drug under the same conditions. E) Cell viability of parental REH and REH+EGFR cells incubated with increasing concentrations of cargo-free anti-EGFR protocells for 1 hour followed by washing to remove unbound protocells. Viability was assessed at 24 hours. Viability data supports the biocompatibility of the monosized protocell platform. F) Flow cytometry analysis of the EFGR expression of REH+EGFR cells as detected by binding of a PE-conjugated anti-EGFR antibody. Right-shifted histogram (blue) shows a majority of the population to be expressing EGFR. However, a minority population does not shift corresponding probably to REH+EGFR cells that have lost EGFR expression. Viability data represents mean±SD, n=3.

FIGS. 54A-F. Intravital fluorescent microscopy images acquired ex ovo in the CAM model showing green YO-PRO®-1 cell impermeant cargo loaded, red fluorescent labelled EGFR-targeted protocells interacting and releasing cargo into REH+EGFR cells in a live animal model. (A) Fluorescent overlay of (blue) REH+EGFR cell, (red) protocell, (green) YO-PRO®-1 cargo, (lavender) lectin vascular stain at 4 hours post injection. (B) Red channel shows protocell fluorescence, and (C) green channel shows YO-PRO®-1 fluorescence associated with the protocells. However, after 16 hours, (D) fluorescent overlay shows release of YO-PRO®-1 cargo within the cell. (E) Red channel shows 16 h protocell fluorescence and (F) green channel shows YO-PRO®-1 release into the cell. Images acquired at 63× magnification, Scale bar=5 μm.

FIG. 55. Composition and representative hydrodynamic size data of liposomes used for preparation of protocells. Data represent mean±SD, n=3.

FIG. 56. Average hydrodynamic size comparison of mMSNs of various size, shape, and pore morphology before and after SLB fusion, data accompanies images in FIG. 56, data represents mean±SD, n=3. Average mMSN dimensions from TEM images of mMSNs, data represents mean±SD, n=50. Surface area and pore volume measurements calculated from Nitrogen sorption data, * data from the literature.¹ Estimated numbers calculated from equations described later.

FIGS. 57A-D. N2 adsorption-desorption isotherms and pore size distribution (inset) of A) Hexagonal mMSNs with 2.8 nm pores, B) Spherical mMSNs with 2.8 nm pores, C) Spherical mMSNs with 5, 9, or 18 nm pores, and D) Rod-like mMSNs with 2.8 nm pores.

FIG. 58. Analysis of hydrodynamic size and PdI change in protocells prepared under differing PBS ionic strength conditions and transferred to physiological ionic strength (160 mM) PBS. The size change of protocells prepared in the absence of salt suggests that protocells do not form in water, since the size increase is clearly larger than all protocells prepared in increasing ionic strength conditions. Data represent mean±SD, n=3.

FIG. 59. Average lipid bi-layer thickness measured from TEM images. Data represents mean±SD, n=33.

FIG. 60. Comparison of protocells assembled using the methods described in our paper and those assembled using probe sonication conditions described in the literature.^{2,3} Both methods produced protocells of similar size and monodispersity profile. Data represent mean±SD, n=3.

FIG. 61. Hydrodynamic size measurement and polydispersity index values of liposomes, Hexagonal mMSNs, and assembled protocells using technique described in our paper with different liposome formulations described herein. Data represent mean±SD, n=3.

FIG. 62. Analysis of PdI of bare Hexagonal mMSNs and protocells after incubation for 72 hours at 37° C. in either PBS or DMEM+10% FBS. Data corresponds to size data reported in FIGS. 43C and 43D. Data represent mean±SD, n=3.

FIG. 63. Hydrodynamic size characteristics of Hexagonal mMSN and protocells after 6 month storage under static conditions at 25° C. SLB formulation DSPC:chol:DSPE-PEG2000 (mol % 54/44/2). Data represent mean±SD, n=3.

FIGS. 64A-B. Hydrodynamic size of A) DOPC-based protocells or B) DSPC-based protocells stored in either 160 mM standard PBS or deoxygenated PBS at 37° C. for 7 days. The presence of oxygen in solution appears to cause a size increase likely due to the oxidation of the double bonds present in the acyl chains of DOPC. Neither the presence nor absence of oxygen appears to influence the size of DSPC-based protocells, as they do not contain any double bonds in the acyl chains. Data represent mean±SD, n=3.

FIGS. 65A-B. A) Conventional TEM image of Hexagonal MSNs prepared from EISA synthesis route. Scale bar=200 nm. B) Histogram of particle size distributions of EISA and Hexagonal mMSN cores measured from TEM images. Data represent mean±SD, n=220.

FIGS. 66A-D. Flow cytometry measurements of EA.hy926 endothelial cells after incubation with 20 μg/mL of A) EISA MSN, B) EISA protocell, C) Hexagonal mMSN, and D) monosized protocells for 4 hours. Percent population shift due to particle fluorescence (grey=control, no particle exposure, blue outline=mMSNs or protocells).

FIGS. 67A-B. Differential binding of Hexagonal mMSNs and protocells observed after 4 hours incubation in complete medium. A) Bare Hexagonal mMSNs (red) bind non-specifically to EA.hy926 (blue—DAPI stained nuclei, green—phalloidin stained actin), while B) protocells (red) do not interact with cells in culture. Scale bar=50 μm.

FIGS. 68A-B. Fluorescently-labelled nanoparticle flow patterns observed using ex ovo CAM model. Representative sections highlight differential flow characteristics between A) Hexagonal mMSNs 5 minutes post injection and B) 30 minutes post injection. Red: mMSN; Blue: autofluorescence from tissue. Scale bar=50 μm.

FIGS. 69A-B. A) Percentage of lysed human red blood cells (hRBCs) after exposure to 25, 50, 100, 200, and 400 μg/mL of mMSNs and protocells for 2 hours at 37° C. Data represent mean±SD, n=3. B) Digital photographs of hRBCs after 2 hours incubation with (top) mMSNs or (bottom) protocells at different particle concentrations (25 to 400 μg/mL). Presence of red hemoglobin in supernatant indicates membrane damaged hRBCs.

FIG. 70. Hydrodynamic size comparison of pre-injected protocells and protocells separated from CAM blood at different time points. Data provides evidence of size stability ex ovo as assessed by modest change in hydrodynamic size over multiple times up to 240 minutes in circulation. Data represent mean±SD, n=3.

FIGS. 71A-E. Fluorescent microscopy analysis of REH+EGFR cells incubated with EGFR targeted protocells at multiple time points, fixed and stained (blue—nuclei, green—cytoskeleton, red—protocells): A) untreated, B) 5 minutes, C) 15 minutes, D) 30 minutes, and E) 60 minutes. These data illustrate rapid in vitro protocell binding in as little as 5 minutes in complete medium, and maximal protocell accumulation after 30 minutes. Scale bar=5 μm.

FIGS. 72A-C. A) Mean fluorescence intensity graph of REH and REH+EGFR cells incubated with either non-targeted or EGFR-targeted protocells shows targeting specificity of EGFR targeted protocells. B) Flow cytometry analysis of REH+EGFR cells incubated with red fluorescent non-targeted protocells at multiple time points. C) Flow cytometry analysis of parental REH cells incubated with red fluorescent non-targeted protocells at multiple time points. These data demonstrate the high specific binding of EGFR-targeted protocells to REH+EGFR and low non-specific binding of both targeted and non-targeted to protocells.

FIGS. 73A-D. A) Fluorescent microscopy shows minimal EGFR targeted protocell (red) interactions with a non-EGFR expressing BAF cell line after 1 hour incubation (blue—DAPI stained nuclei, green—phalloidin stained actin), while B) targeted protocells (red) exhibit a high degree of binding to an EGFR expressing BAF cell line. Flow cytometry analysis of protocells incubated with C) BAF and D) BAF+EGFR confirm fluorescent microscopy analysis (grey=no protocell control, blue=EGFR targeted protocells). Scale bar=10 μm.

FIGS. 74A-I. Neither EGFR targeted nor non-targeted protocells display non-specific binding to target and non-target cells. Intravital fluorescent microscopy images acquired ex ovo in the CAM model reveal stable circulation of EFGR-targeted protocells (red) but no association with A-C) parental REH cells (green) and non-targeted protocells with D-F) parental REH cells and G-I) REH-EGFR cells in circulation at 1 hour (left), 4 hours (top right), and 9 hours (bottom right) time points. Scale bar (left)=50 μm, Scale bars=(right top and bottom)10 μm.

FIGS. 75A-B. Intravital fluorescent microscopy images acquired in the CAM model show: A) stable circulation of anti-EGFR targeted protocells (red) and the initial stages binding to Ba/F3+EGFR (green) 10 minutes post injection; B) maintained association of anti-EGFR targeted protocells (yellow, due to merged green and red) with Ba/F3+EGFR (green) 20 hours post-injection. Scale bar=10 μm.

FIG. 76. Hydrodynamic size characteristics and zeta potential measurements of loaded and unloaded targeted protocells. Multiple batches were synthesized, superscript (* and **) denotes mMSN cores prepared from the identical batches. Data represent mean±SD, n=3.

FIGS. 77A-E. Fluorescence microscopy analysis to assess delivery of model drug, wYO-PRO®-1 a green cell impermeant dye, via targeted protocells to REH+EGFR cells at multiple time points. After each time point, cells were acid washed to strip surface bound protocells then fixed. REH+EGFR cells (DIC-cell structure, red-protocells, green-YO-PRO®-1) at A) untreated, B) 1 hour, C) 8 hours, D) 16 hours, and E) 24 hours incubation times. These data illustrate internalization of protocells within 1 hour and the release of YO-PRO®-1 cargo which appears to localize in the nucleus of the target cells at later time points. Scale bar=25 μm.

FIG. 78. Comparison of drug release percentage (left axis) from GEM-loaded protocells in extracellular physiological conditions (pH 7.4), PBS and simulated lysosomal conditions (pH 5.0), citrate buffer and protocell size change (right axis) for 72 hours at 37° C. Increased GEM release was observed at pH 5.0 and significant size increase at 48 hours with a 228-fold size increase at 72 hours suggesting protocell destabilization and aggregation due to lower pH conditions. Drug release at pH 5.0 correlates with protocell size increase overtime. Protocells maintain size stability at pH 7.4 for 72 hours at 37° C., however they do appear to release about 14% GEM after 72 hours.

FIG. 79. Components of a protocell loaded with cargo.

FIG. 80. Features of a hybrid bilayer protocell according to one embodiment. The embodiment provides increased loading space and may improve projection of surface moieties.

FIG. 81. Hydrophobic modification in one embodiment (method number 1) involves hydrocarbon chlorosilanes.

FIG. 82. The stability of 100 nm silica with DSPE-PEG 2K over time. The hybrid particle size is shown with respect to three silanes. Control particles remained stable over 8 weeks, remained monodispersed and increased in size by only 13%. Silane 1 and silane 2 aggregated within 2-3 weeks. Silane 3 remained stable over 8 weeks, remained monodispersed and increased in size by only 7%. Silane 3 modification exhibited the greater stability over time. Hydrophobically modified MSNs were stable in chloroform. Hybrid bilayer protocells were stable in DMSO and PBS.

FIG. 83. The stability of 100 nm silica with DSPE-PEG 5K over time. The hybrid particle size is shown with respect to the three silanes. Silane 3 modification exhibited the greater stability over time.

FIG. 84. MSN:Lipid ratios for DSPE-PEG 2K. A ratio of 1:2 forms the smallest hybrid bilayer protocells. Particles with the 1:2 ratio had the smallest PdI and particles with a longer PEG length showed better circulation in a CAM model.

FIGS. 85A-B. Average size of 50 nm MSN hybrid protocells with silane hydrocarbon modification and various lipid formulations.

FIG. 86. A hydrophobic modification method involving carboxyl modification of the MSN surface which can be modified using a number of approaches. Following the reaction of the carboxyl moiety with EDC crosslinker (or other crosslinker) produces a silane having an amine function group on its surface. The reaction of the carboxyl moiety with DPPE lipid forms the hydrophobic moiety (through an amide bond) using an alternative approach as indicated.

FIG. 87. Average particle size using the carboxyl surface modification. All particles were monodispersed. Control particles aggregated within 6 days, while particles in Trials 1-4 remained stable within 6 days.

FIGS. 88A-C. Core particle characterization. A) TEM image of MSNs showing hexagonally ordered anisotropic and uniformly distributed pore structure. B) Increased average diameter of MSNs with hydrophobic modification and hybrid bi-layer protocell formation in aqueous buffer. C) Carboxylic acid modification of MSNs confirmed by Fourier transform infra-red spectrometry (FTIR) as evidenced by carbonyl stretching.

FIG. 89. Schematic of hybrid bilayer protocell synthesis.

FIGS. 90A-B. Nanoparticle stability.

FIGS. 91A-B. Impact of lipid ratio on particle size and circulation.

FIGS. 92A-C. Nanoparticle biocompatibility of several hybrid bilayer protocells with different PEG length coatings. An increase in PEG length shows increased biocompatibility.

FIG. 93. Impact of conjugation method on hybrid bilayer protocell particle size.

FIGS. 94A-C. A) Hexagonal mMSN. B) Spherical mMSN. C) Rod-like mMSN.

### DETAILED DESCRIPTION

These and/or other embodiments of may be readily gleaned from the following description.

### Definitions

Where a range of values is provided, it is understood that each intervening value, to the tenth of the unit of the lower limit unless the context clearly dictates otherwise, between the upper and lower limit of that range and any other stated or intervening value in that stated range is encompassed. The upper and lower limits of these smaller ranges may independently be included in the smaller ranges is also encompassed, subject to any specifically excluded limit in the stated range. Where the stated range includes one or both of the limits, ranges excluding either both of those included limits are also included.

Unless defined otherwise, al technical and scientific terms used herein have the same meaning as commonly understood by one of ordinary skill in the art to which this disclosure belongs. Although any methods and materials similar or equivalent to those described herein can also be used in the practice or testing of the present disclosure, exemplary methods and materials are now described.

It must be noted that as used herein and in the appended claims, the singular forms “a,” “and” and “the” include plural references unless the context clearly dictates otherwise.

Reference to “about” a value or parameter herein includes (and describes) variations that are directed to that value or parameter per se. For example, description referring to “about X” Includes description of “X”.

The term “monodisperse” and “monosized” are used synonymously to describe both mesoporous particles, e.g., nanoparticles (although the particles may range up to about 6 microns in diameter) and protocells (i.e., mesoporous nanoparticles having a fused lipid bi-layer on the surface of the nanoparticles) which are monodisperse.

The term “monosized mesoporous silica nanoparticles” or mMSNPs is used to describe a population of monosized (monodispersed) mesoporous silica nanoparticles. Example particles are produced using a solution-based surfactant directed self-assembly strategy conducted under basic conditions, followed by hydrothermal treatment to provide mMSNPs with tunable core structure, pore sizes and shape. Certain methods for producing silica nanoparticles are described in Lin et al., 2005; Lin et al., 2010; Lin et al., 2011; Chen et al., 2013; Bayu et al., 2009; Wang et al., 2012; Shen et al., 2014; Huang et al., 2011; and Yu et al., 2011, among others. mMSNPs may be provided in various shapes, including spherical, oval, hexagonal, dendritic, cylindrical, rod-shaped, disc-like, tubular and polyhedral pursuant to the above-described methods. Monodispersity can also be described as having a polydispersity index (PdI or DPI) of about 0.1 to about 0.2, less than about 0.2, or less than about 0.1.

The synthetic procedures for providing monodisperse MSNPs may be varied to vary the contents and size of the mMSNPs, as well as the pore size. In typical synthesis, mMSNPs are produced using a solution based surfactant directed self-assembly strategy conducted under basic conditions (e.g., triethylamine or other weak base), followed by a hydrothermal treatment. Size adjustment may be facilitated by increasing the concentration of catalyst (e.g., ammonium hydroxide). Increasing the concentration of the catalyst will increase the size of the resulting mMSNPs, whereas decreasing the concentration of the catalyst will decrease the size of the resulting mMSNPs. Increasing the amount of silica precursor (e.g., TEOS) will also increase the particle size, as will decreasing the temperature during synthesis. Decreasing the amount of silica precursor and/or increasing the temperature during synthesis will decrease the particle size. All of the above parameters may be modified to adjust the sizes of the mesopores within the nanoparticles. To change the nature of the silica particles, amine-containing silanes such as N-(2-aminoethyl)-3-aminopropyltrimethoxysilane (AEPTMS) or 3-aminopropyltriethoxysilane (APTES) may be added to the solution containing TEOS or other silica precursor. The addition of an amine-containing silane will produce a silica particle with a zeta potential (mV) with a less negative to neutral/positive zeta potential, depending on the amount of amine-containing silane including in the reaction mixture to form the nanoparticles. The nanoparticles have a zeta potential (mV) ranging from about −50 mV to about +35 mV depending upon the amount of amine containing silane added to the synthesis (e.g., from about 0.01% up to about 50% by weight, often about 0.1% to about 20% by weight, about 0.25% to about 15% by weight, about 0.5% to about 10% by weight), with a greater amount of amine containing silane increasing the zeta potential and a lesser amount (to none) providing a nanoparticle with a negative zeta potential.

Surfactants which can be used in the synthesis of mMSNPs include for example, octyltrimethylammonium bromide, decyltrimethylammonium bromide, dodecyltrimethylammonium bromide, tetradecyltrimethylammonium bromide, benzyldimethylhexadecylammonium chloride, hexadecyltrimethylammonium bromide, hexadecyltrimethylammonium chloride, ocadecyltrimethylammonium bromide, octadecyltrimethylammonium chloride, dihexadecyldimethylammonium bromide, dimethyldioctadecylammonium bromide, dimethylditetradecylammonium bromide, didodecyldimethylammonium bromide, didecyldimethylammonium bromide and didecyldimethylammonium bromide, among others.

The term “protocell” is used to describe a porous nanoparticle surrounded by a lipid bi-layer. In some embodiments, the porous nanoparticle is made of a material comprising silica, polystyrene, alumina, titania, zirconia, or generally metal oxides, organometallates, organosilicates or mixtures thereof.

The term “lipid” is used to describe the components which are used to form lipid bi-layers on the surface of nanoparticles.

Porous nanoparticulates used in protocells include mesoporous silica nanoparticles and core-shell nanoparticles. The porous nanoparticulates can also be biodegradable polymer nanoparticulates comprising one or more compositions selected from the group consisting of aliphatic polyesters, poly (lactic acid) (PLA), poly (glycolic acid) (PGA), co-polymers of lactic acid and glycolic acid (PLGA), polycaprolactone (PCL), polyanhydrides, poly(ortho)esters, polyurethanes, poly(butyric acid), poly(valeric acid), poly(lactide-co-caprolactone), alginate and other polysaccharides, collagen, and chemical derivatives thereof, albumin, a hydrophilic protein, zein, a prolamine, a hydrophobic protein, and copolymers and mixtures thereof.

A porous spherical silica nanoparticle may be used for the protocells and is surrounded by a supported lipid or polymer bi-layer or multi-layer. Various embodiments provide nanostructures and methods for constructing and using the nanostructures and providing protocells. Many of the protocells in their most elemental form are known in the art. Porous silica particles of varying sizes ranging in size (diameter) from less than 5 nm to 200 nm or 500 nm or more are readily available in the art or can be readily prepared using methods known in the art (see the examples section) or alternatively, can be purchased from SkySpring Nanomaterials, Inc., Houston, Tex., USA or from Discovery Scientific, Inc., Vancouver, British Columbia. Multimodal silica nanoparticles may be readily prepared using the procedure of Carroll, at al., Langmuir, 25, 13540-13544 (2009). Protocells can be readily obtained using methodologies known in the art. The examples section of the present application provides certain methodology for obtaining protocells. Protocells may be readily prepared, including protocells comprising lipids which are fused to the surface of the silica nanoparticle. See, for example, Liu et al., 2009; Liu et al., 2009: Liu et al., 2009; Lu et al., 1999, Protocells may be prepared according to the procedures which are presented in Ashley et al., 2011; Lu et al., 1999; Caroll et al., 2009, and as otherwise presented in the experimental section which follows.

The terms “nanoparticulate” and “porous nanoparticulate” are used interchangeably herein and such particles may exist in a crystalline phase, an amorphous phase, a semi-crystalline phase, a semi amorphous phase, or a mixture thereof.

A nanoparticle may have a variety of shapes and cross-sectional geometries that may depend, in part, upon the process used to produce the particles. In one embodiment, a nanoparticle may have a shape that is a sphere, a rod, a tube, a flake, a fiber, a plate, a wire, a cube, a prism or a whisker. A nanoparticle may include particles having two or more of the aforementioned shapes. In one embodiment, a cross-sectional geometry of the particle may be one or more of circular, ellipsoidal, triangular, toroidal, rectangular or polygonal. In one embodiment, a nanoparticle may consist essentially of non-spherical particles, especially prisms. For example, such particles may have the form of ellipsoids, which may have all three principal axes of differing lengths, or may be oblate or prelate ellipsoids of revolution. Non-spherical nanoparticles alternatively may be laminar in form, wherein laminar refers to particles in which the maximum dimension along one axis is substantially less than the maximum dimension along each of the other two axes. Non-spherical nanoparticles may also have the shape of frusta of pyramids or cones, or of elongated rods. In one embodiment, the nanoparticles may be irregular in shape. In one embodiment, a plurality of nanoparticles may consist essentially of spherical nanoparticles. In one embodiment, a plurality of nanoparticles may consist essentially of hexagonal prism nanoparticles.

The term ‘monosized protocells’ is used to describe a population of monosized (monodisperse) protocells comprising a lipid bi-layer fused onto a mMSNPs as otherwise described herein. In some embodiments, monosized protocells are prepared by fusing the lipids in monosized unilamellar liposomes onto the mMSNPs in aqueous buffer (e.g., phosphate buffered solution) or other solution at about room temperature, although slightly higher and lower temperatures may be used. The unlamellar liposomes which are fused onto the mMSNPs are prepared by sonication and extrusion according to the method of Akbarzadeh et al., 2013 and are monodisperse with hydrodynamic diameters of less than about 100 nm, often about 65-95 nm, most often about 90-95 nm, although unilamellar liposomes which can be used may faN outside this range depending on the size of the mMSNPs to which lipids are to be fused and low PDI values (generally, less than about 0.5, e.g., less than 0.2). The mass ratio of liposomes to mMSNPs used to create monosized protocells which have a single lipid bi-layer completely surrounding the mMSNPs is that amount sufficient to provide a liposome interior surface area which equals or exceeds the exterior surface area of the mMSNPs to which the lipid is to be fused. This often is provided in a mass ratio of liposomes to mMSNPs of at least about 2:1, often up to about 10:1 or more, with a range often used being about 2:1 to about 5:1. The resulting protocells are monosized (monodisperse). Monosized protocells may exhibit extended storage stability in aqueous solution, e.g., providing a SLB on the protocell which has a transition temperature Tmwhich is greater than the storage, experimental or administration/therapeutic conditions under which the protocells are stored and/or used. Often the protocell is at least about 25-30 nm in diameter larger than the diameter of the mMSNPs.

The phrase “effective average particle size” as used herein to describe a muliparticulate (e.g., a porous nanoparticulate) means that al particles therein are of an average diameter or within +5% of the average diameter. In certain embodiments, nanoparticulates have an effective average particle size (diameter) of less than about 2,000 nm (i.e., 2 microns), less than about 1,900 nm, less than about 1,800 nm, less than about 1,700 nm, less than about 1,600 nm, less than about 1,500 nm, less than about 1,400 nm, less than about 1,300 nm, less than about 1,200 nm, less than about 1,100 nm, less than about 1,000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, less than about 50 nm, less than about 35 nm, less than about 25 nm, as measured by light-scattering methods, microscopy, or other appropriate methods. In exemplary aspects, the average diameter of mMSNPs ranges from about 75 nm to about 150 nm, often about 75 to about 130 nm, often about 75 nm to about 100 nm.

The term “patient” or “subject” is used throughout the specification within context to describe an animal, generally a mammal, especially including a domesticated animal and for example a human, to whom treatment, including prophylactic treatment (prophylaxis), with the compounds or compositions is provided. For treatment of those infections, conditions or disease states which are specific for a specific animal such as a human patient, the term patient refers to that specific animal. In most instances, the patient or subject is a human patient of either or both genders.

The term “effective” is used herein, unless otherwise indicated, to describe an amount of a compound or component which, when used within the context of its use, produces or effects an intended result, whether that result relates to the prophylaxis and/or therapy of an infection and/or disease state or as otherwise described herein. The term effective subsumes all other effective amount or effective concentration terms (including the term “therapeutically effective”) which are otherwise described or used in the present application.

The term ‘compound’ is used herein to describe any specific compound or bioactive agent disclosed herein, including any and all stereoisomers (including diastereomers), individual optical isomers (enantiomers) or racemic mixtures, pharmaceutically acceptable salts and prodrug forms. The term compound herein refers to stable compounds. Within its use in context, the term compound may refer to a single compound or a mixture of compounds as otherwise described herein.

The term “bioactive agent” refers to any biologically active compound or drug which may be formulated for use in an embodiment. Exemplary bioactive agents include the compounds which are used to treat cancer or a disease state or condition which occurs secondary to cancer and may include anti-viral agents, especially anti-HIV, anti-HBV and/or anti-HCV agents (especially where hepatocellular cancer is to be treated) as well as other compounds or agents which are otherwise described herein.

The terms “treat”, “treating”, and “treatment”, are used synonymously to refer to any action providing a benefit to a patient at risk for or afflicted with a disease state or condition, including improvement in the disease state or condition through lessening, inhibition, suppression or elimination of at least one symptom, delay in progression of the disease, prevention, delay in or inhibition of the likelihood of the onset of the disease state and/or condition, etc. In the case of microbial infections, these terms also apply to microbial (e.g., viral or bacterial) infections and may include, in certain particularly favorable embodiments the eradication or elimination (as provided by limits of diagnostics) of the microbe (e.g., a virus or a bacterium) which is the causative agent of the infection.

Treatment, as used herein, encompasses both prophylactic and therapeutic treatment, e.g., of cancer (including inhibiting metastasis or recurrence of a cancer in remission), but also of other disease states, including microbial infections such as bacterial, fungal, protest, aechaea, and viral infections, especially including HBV and/or HCV. Compounds can, for example, be administered prophylactically to a mammal in advance of the occurrence of disease to reduce the likelihood of that disease. Prophylactic administration, e.g., a vaccine, is effective to reduce or decrease the likelihood of the subsequent occurrence of disease in the mammal, or decrease the severity of disease (inhibition) that subsequently occurs, especially including metastasis of cancer. Alternatively, compounds can, for example, be administered therapeutically to a mammal that is already afflicted by disease. In one embodiment of therapeutic administration, administration of the present compounds is effective to eliminate the disease and produce a remission or substantially eliminate the likelihood of metastasis of a cancer. Administration of the compounds is effective to decrease the severity of the disease or lengthen the lifespan of the mammal so afflicted, as in the case of cancer, or inhibit or even eliminate the causative agent of the disease, as in the case of hepatitis B virus (HBV) and/or hepatitis C virus infections (HCV) infections. In another embodiment of therapeutic administration, administration of the present compounds is effective to decrease the likelihood of infection or re-infection by a microbe and/or to decrease the symptom(s) or severity of an infection.

The term “prophylactic administration” refers to any action in advance of the occurrence of disease to reduce the likelihood of that disease or any action to reduce the likelihood of the subsequent occurrence of disease in the subject. Compositions can, for example, be administered prophylactically to a mammal in advance of the occurrence of disease to enhance an immunogenic effect and/or reduce the likelihood of that disease, generally a viral disease. Prophylactic administration is effective to reduce or decrease the likelihood of the subsequent occurrence of disease in the mammal, or decrease the severity of disease (inhibition) that subsequently occurs, especially including a microbial (e.g., a viral or bacterial) infection and/or cancer, its metastasis or recurrence.

The term “antihepatocellular cancer agent” is used throughout the specification to describe an anti-cancer agent which may be used to inhibit, treat or reduce the likelihood of hepatocellular cancer, or the metastasis of that cancer, especially secondary to a viral infection such as HBV and/or HCV Anti-cancer agents which may find use include for example nexavar (sorafenib), sunitinib, bevacizumab, tarceva (erlotinib), tykerb (lapatinib), and mixtures thereof. In addition, other anti-cancer agents may also be used, where such agents are found to inhibit metastasis of cancer, in particular, hepatocellular cancer.

The term “targeting active species” is used to describe a compound or moiety which is complexed or covalently bonded to the surface of a protocell which binds to a moiety on the surface of a cell to be targeted so that the protocell may selectively bind to the surface of the targeted cell and deposit its contents into the cell. In one embodiment, the targeting active species is a “targeting peptide” including a polypeptide including an antibody or antibody fragment, an aptamer, or a carbohydrate, among other species which bind to a targeted cell. A targeting active species may be peptide of a particular sequence which binds to a receptor or other polypeptide in cancer cells and allows the targeting of protocells to particular cells which express a peptide (be it a receptor or other functional polypeptide) to which the targeting peptide binds. Exemplary targeting peptides include, for example, SP94 free peptide (H2N-SFSIILTPILPL-COOH, SEQ ID NO: 3), SP94 peptide modified with a C-terminal cysteine for conjugation with a crosslinking agent (H₂N-GLFHAIAHFIHGGWHGLIHGWYGGC-COOH (SEQ ID. NO:4) or an 8 mer polyarginine (H₂N—RRRRRRRR-COOH, SEQ ID NO:5), a modified SP94 peptide (H₂N-SFSIILTPILPLEEEGGC-COOH, SEQ ID NO:6) or a MET binding peptide or CRLF2 binding peptide as disclosed in WO 2012/149376, published Nov. 1, 2012 and CRLF2 peptides, for example as disclosed in WO 2013/103614, published Jul. 11, 2013, relevant portions of which applications are incorporated by reference herein. Other targeting peptides are known in the art. Targeting peptides may be complexed or covalently linked to the lipid bi-layer through use of a crosslinking agent as otherwise described herein.

The term “MET binding peptide” or “MET receptor binding peptide” is used to describe any peptide that binds the MET receptor. MET binding peptides include at least five (5) 7-mer peptides which have been shown to bind MET receptors on the surface of cancer cells with enhanced binding efficiency. Several small peptides with varying amino acid sequences were identified which bind the MET receptor (a.k.a. hepatocyte growth factor receptor, expressed by gene c-MET) with varying levels of specificity and with varying ability to activate MET receptor signaling pathways. 7-mer peptides were identified using phage display biopanning, with examples of resulting sequences which evidence enhanced binding to MET receptor and consequently to cells such as cancer cells (e.g., hepatocellular, ovarian and cervical) which express high levels of MET receptors, which appear below. Binding data for several of the most commonly observed sequences during the biopanning process is also presented in the examples section of the present application. These peptides are particularly useful as targeting ligands for cell-specific therapeutics. However, peptides with the ability to activate the receptor pathway may have additional therapeutic value themselves or in combination with other therapies. Many of the peptides have been found bind not only hepatocellular carcinoma, which was the original intended target, but also to bind a wide variety of other carcinomas including ovarian and cervical cancer. These peptides are believed to have wide-ranging applicability for targeting or treating a variety of cancers and other physiological problems associated with expression of MET and associated receptors.

The following five 7 mer peptide sequences show substantial binding to MET receptor and may be useful as targeting peptides for use on protocells.

(SEQ ID NO: 7)
ASVHFPP (Ala-Ser-Val-His-Phe-Pro-Pro)
(SEQ ID NO: 8)
TATFWFQ (Thr-Ala-Thr-Phe-Trp-Phe-Gln)
(SEQ ID NO: 9)
TSPVALL (Thr-Ser-Pro-Val-Ala-Leu-Leu)
(SEQ ID NO: 10)
IPLKVHP (Ile-Pro-Leu-Lys-Val-His-Pro)
(SEQ ID NO: 11)
WPRLTNM (Trp-Pro-Arg-Leu-Thr-Asn-Met)

Each of these peptides may be used alone or in combination with other MET peptides within the above group or with other targeting peptides which may assist in binding protocells n to cancer cells, including hepatocellular cancer cells, ovarian cancer cells and cervical cancer cells, among numerous others. These binding peptides may also be used in pharmaceutical compounds alone as MET binding peptides to treat cancer and otherwise inhibit hepatocyte growth factor binding.

The terms “fusogenic peptide” and “endosomolytic peptide” are used synonymously to describe a peptide which is optionally crosslinked onto the lipid bi-layer surface of the protocells. Fusogenic peptides are incorporated onto protocells in order to facilitate or assist escape from endosomal bodies and to facilitate the introduction of protocells into targeted cells to effect an intended result (therapeutic and/or diagnostic as otherwise described herein). Representative fusogenic peptides for use in protocells include but are not limited to H5WYG peptide, H2N-GLFHAIAHFIHGGWHGLIHGWYGGC-COOH (SEQ ID. NO:12) or an 8 mer polyarginine (H₂N—RRRRRRRR-COOH, SEQ ID NO:13), among others known in the art. Additional fusogenic peptides include RALA peptide (NH₂-WEARLARALARALARHLARALARALRAGEA-COOH, SEQ ID NO: 14), KALA peptide (NH₂-WEAKLAKALAKALAKHLAKALAKALKAGEA-COOH), SEQ ID. NO:15), GALA (NH2-WEAALAEALAEALAEHLAEALAEALEALAA-COOH, SEQ ID NO:16) and INF7 (NH2-GLFEAIEGFIENGWEGMIDGWYG-COOH, SEQ ID. NO:17), among others.

Thus, the terms “cell penetration peptide,” “fusogenic peptide” and “endosomolytic peptide” are used to describe a peptide which aids protocell translocation across a lipid bi-layer, such as a cellular membrane or endosome lipid bi-layer and is optionally crosslinked onto a lipid bi-layer surface of the protocells. Endosomolytic peptides are a sub-species of fusogenic peptides as described herein. In both the multilamellar and single layer protocell embodiments, the non-endosomolytic fusogenic peptides (e.g., electrostatic cell penetrating peptide such as R8 octaarginine) are incorporated onto the protocells at the surface of the protocell in order to facilitate the introduction of protocells into targeted cells (APCs) to effect an intended result (to instill an immunogenic and/or therapeutic response as described herein). The endosomolytic peptides (often referred to in the art as a subset of fusogenic peptides) may be incorporated in the surface lipid bi-layer of the protocell or in a lipid sublayer of the multilamellar protocell in order to facilitate or assist in the escape of the protocell from endosomal bodies. Representative electrostatic cell penetration (fusogenic) peptides for use in protocells include an 8 mer polyarginine (H₂N—RRRRRRRR-COOH, SEQ ID NO:1), among others known in the art, which are included in protocells in order to enhance the penetration of the protocell into cells. Representative endosomolytic fusogenic peptides (‘endosomolytic peptides) include H5WYG peptide, H₂N-GLFHAIAHFIHGGWHGLIHGWYGGC-COOH (SEQ ID. NO: 2), RALA peptide (NH₂-WEARLARALARALARHLARALARALRAGEA-COOH, SEQ ID NO: 18), KALA peptide (NH₂-WEAKLAKALAKALAKHLAKALAKALKAGEA-COOH), SEQ ID. NO:19), GALA (NH2-WEAALAEALAEALAEHLAEALAEALEALAA-COOH, SEQ ID NO:20) and INF7 (NH2-GLFEAIEGFIENGWEGMIDGWYG-COOH, SEQ ID. NO:21), among others. At least one endosomolytic peptide is included in protocells in combination with a viral antigen (often pre-ubiquitinylated) and/or a viral plasmid (which expresses viral protein or antigen) In order to produce CD8+ cytotoxic T cells pursuant to a MHC class I pathway.

The term “crosslinking agent” is used to describe a bifunctional compound of varying length containing two different functional groups which may be used to covalently link various components to each other. Crosslinking agents may contain two electrophilic groups (to react with nucleophilic groups on peptides of oligonucleotides, one electrophilic group and one nucleophilic group or two nucleophilic groups). The crosslinking agents may vary in length depending upon the components to be inked and the relative flexibility required. Crosslinking agents are used to anchor targeting and/or fusogenic peptides and other functional moieties (for example toll receptor agonists for immunogenic) to the phospholipid bi-layer, to link nuclear localization sequences to histone proteins for packaging supercoiled plasmid DNA and in certain instances, to crosslink lipids in the lipid bi-layer of the protocells. There are a large number of crosslinking agents which may be used in many commercially available or available in the literature. Exemplary crosslinking agents for use, for example, 1-Ethyl-3-[3-dimethylaminopropyl]carbodiimide hydrochloride (EDC), succinimidyl 4-[N-maleimidomethyl]cyclohexane-1-carboxylate (SMCC), N-[ß-Maleimidopropionic acid]hydrazide (BMPH), NHS-(PEG)ₙ-maleimide, succinimidyl-[(N-maleimidopropionamido)-tetracosaethyleneglycol] ester (SM(PEG)₂₄), and succinimidyl 6-[3′-(2-pyridyldithio)-propionamido] hexanoate (LC-SPDP), among others.

The term “antigen presenting cell” “APC” or “accessory cell” is a cell in the body that displays foreign antigens complexed with major histocompatibility complexes (MHCs) on their surfaces through antigen presentation. These cells include dendritic cells (DCs), macrophages, B-cells which express a B cell receptor (BCR) and specific antibody which binds to the BCR, certain activated epithelial cells (any cell which expresses MHC class I molecules) and any nucleated cell which expresses MHC class I molecules). T cells often recognize these complexes through T-cell receptors. APCs process antigens and present them to T-cells.

The term “crosslinking agent” is used to describe a bifunctional compound of varying length containing two different functional groups which may be used to covalently link various components to each other. Crosslinking agents may contain two electrophilic groups (to react with nucleophilic groups on peptides of oligonucleotides, one electrophilic group and one nucleophilic group or two nucleophilic groups). The crosslinking agents may vary in length depending upon the components to be inked and the relative flexibility required. Crosslinking agents are used to anchor targeting and/or fusogenic peptides to the phospholipid bi-layer, to link nuclear localization sequences to histone proteins for packaging supercoiled plasmid DNA and in certain instances, to crosslink lipids in the lipid bi-layer of the protocells. There are a large number of crosslinking agents which may be used, many commercially available or available in the literature. Exemplary crosslinking agents for use include, for example, 1-Ethyl-3-[3-dimethylaminopropyl]carbodiimide hydrochloride (EDC), succinimidyl 4-[N-maleimidomethyl]cyclohexane-1-carboxylate (SMCC), Succinimidyl 6-[ß-Maleimidopropionamido]hexanoate (SMPH), N-[ß-Maleimidopropionic acid] hydrazide (BMPH), NHS-(PEG)ₙ-maleimide, succinimidyl-[(N-maleimidoproponamido)-tetracosaethyleneglycol] ester (SM(PEG)₂₄), and succinimidyl 6-[3′-(2-pyridyldithio)-propionamido] hexanoate (LC-SPDP), among others.

The term “anti-viral agent” is used to describe a bioactive agent/drug which inhibits the growth and/or elaboration of a virus, including mutant strains such as drug resistant viral strains. Preferred anti-viral agents include anti-HIV agents, anti-HBV agents and anti-HCV agents. In certain aspects of the invention, especially where the treatment of hepatocellular cancer is an object of cotherapy, the inclusion of an anti-hepatitis C agent or anti-hepatitis B agent may be combined with other traditional anticancer agents to effect therapy, given that hepatitis B virus (HBV) and/or hepatitis C virus (HCV) is often found as a primary or secondary infection or disease state associated with hepatocellular cancer. Anti-HBV agents which may be used in the present invention, either as a cargo component in the protocell or as an additional bioactive agent in a pharmaceutical composition which includes a population of protocells includes such agents as Hepsera (adefovir dipivoxil), lamivudine, entecavir, telbivudine, tenofovir, emtricitabine, clevudine, valtoricitabine, amdoxovir, pradefovir, racivir, BAM 205, nitazoxanide, UT 231-B, Bay 41-4109, EHT899, zadaxin (thymosin alpha-1) and mixtures thereof. Typical anti-HCV agents for use in the invention include such agents as boceprevir, daclatasvir, asunapavir, INX-189, FV-100, NM 283, VX-950 (telaprevir), SCH 50304, TMC435, VX-500, BX-813, SCH503034, R1626, ITMN-191 (R7227), R7128, PF-868554, TT033, CGH-759, GI 5005, MK-7009, SIRNA-034, MK-0608, A-837093, GS 9190, GS 9256, GS 9451, GS 5885, GS 6620, GS 9620, GS9669, ACH-1095, ACH-2928, GSK625433, TG4040 (MVA-HCV), A-831, F351, NS5A, NS4B, ANA598, A-689, GNI-104, IDX102, ADX184, ALS-2200, ALS-2158, BI 201335, BI 207127, BIT-225, BIT-8020, GL59728, GL60667, PSI-938, PSI-7977, PSI-7851, SCY-635, ribavirin, pegylated interferon, PHX1766, SP-30 and mixtures thereof.

The term “targeting active species” is used to describe a compound or moiety which binds to a moiety on the surface of a targeted cell so that the protocell may selectively bind to the surface of the targeted cell and deposit its contents into the cell. The targeting active species for use may be a targeting peptide as otherwise described herein, a polypeptide including an antibody or antibody fragment, an aptamer, or a carbohydrate, among other species which bind to a targeted cell, especially an antigen presenting cell.

The term “toll-like receptor (TLR) agonist” or “TLR agonist” refers to a moiety on the surface of the protocells which are provided to bind to toll-like receptors on cells containing these receptors and initiate an immunological signaling cascade in providing an immunogenic response to protocells. These agonists enhance or otherwise favorably influence the engagement of T-cell subsets to both stimulate immune responses and make certain cells better targets for immune-mediated destruction TLR agonists which can be used in protocells include a number of compounds/compositions which have shown activity as agonists for toll-like receptors 1 through 9 (TLR 1, TLR 2, TLR 3, TLR 4, TLR 5, TLR 6, TLR 7, TLR 8 and TLR 9). These compounds/compositions include Pam3Cys, HMGB1, Porins, HSP, GLP (agonists for TLR1/2); BCG-CWS, HP-NAP, Zymosan, MALP2, PSK (agonists for TLR 2/6); dsRNA, Poly AU, Poly ICLC, Poly I:C (agonists for TLR 3); LPS, EDA, HSP, Fibrinogen, Monophosphoryl Lipid A (MPLA) (agonists for TLR 4); Flagellin (agonist for TLR 5); Imiquimod (agonist for TLR 7); and ssRNA, PolyG10 and CpG (agonists for TLR 8), as described by Kaczanowka et al., 2013, TLR agonists are covalently linked to components of the lipid bi-layer using conventional chemistry as described herein above for the fusogenic peptides.

The term “ubiquitin” or “ubiquitnylation” is used throughout the present specification to refer to the use of a ubiquitin protein in combination with a viral antigen (e.g., a full length viral protein) as a fusion protein or conjugated via an isopeptide bond. Ubiquitylation of viral proteins generally speeds the development of immunogenicity. Ubiquitin, also referred to as ubiquitous immunopoietic polypeptide, is a protein involved in ubiquitination in the cell and, facilitates the immunogenic response raised after the protocells are introduced into antigen presenting cells (APCs) by facilitating/regulating the degradation of proteins (via the proteasome and lysosome), coordinating the cellular localization of proteins, activating and inactivating proteins and modulating protein-protein interactions, resulting in an enhancement in antigen processing in both professional and non-professional APCs through exogenous and endogenous pathways.

The term “pharmaceutically acceptable” as used herein means that the compound or composition is suitable for administration to a subject, Including a human patient, to achieve the treatments described herein, without unduly deleterious side effects in light of the severity of the disease and necessity of the treatment.

The term “inhibit” as used herein refers to the partial or complete elimination of a potential effect, while inhibitors are compounds/compositions that have the ability to inhibit.

The term “prevention” when used in context shall mean “reducing the likelihood” or preventing a disease, condition or disease state from occurring as a consequence of administration or concurrent administration of one or more compounds or compositions, alone or in combination with another agent. It is noted that prophylaxis will rarely be 100% effective; consequently the terms prevention and reducing the likelihood are used to denote the fact that within a given population of patients or subjects, administration with compounds will reduce the likelihood or inhibit a particular condition or disease state (in particular, the worsening of a disease state such as the growth or metastasis of cancer) or other accepted indicators of disease progression from occurring.

“Amine-containing silanes” include, but are not limited to, a primary amine, a secondary amine or a tertiary amine functionalized with a silicon atom, and may be a monoamine or a polyamine such as diamine. For example, the amine-containing silane is N-(2-aminoethyl)-3-aminopropyltrimethoxysilane (AEPTMS). Non-limiting examples of amine-containing silanes also include 3-aminopropyltrimethoxysilane (APTMS) and 3-aminopropyltriethoxysilane (APTS), as well as an amino-functional trialkoxysilane. Protonated secondary amines, protonated tertiary alkyl amines, protonated amidines, protonated guanidines, protonated pyridines, protonated pyrimidines, protonated pyrazines, protonated purines, protonated imidazoles, protonated pyrroles, quaternary alkyl amines, or combinations thereof, can also be used to modify the mMSNPs.

The term “reporter” is used to describe an imaging agent or moiety which is incorporated into the phospholipid bi-layer or cargo of protocells according to an embodiment and provides a signal which can be measured. The moiety may provide a fluorescent signal or may be a radioisotope which allows radiation detection, among others. Exemplary fluorescent labels for use in protocells (e.g., via conjugation or adsorption to the lipid bi-layer or silica core, although these labels may also be incorporated into cargo elements such as DNA, RNA, polypeptides and small molecules which are delivered to cells by the protocells, include Hoechst 33342 (350/461), 4′,6-diamidino-2-phenylindole (DAPI, 356/451), Alexa Fluor® 405 carboxylic acid, succinimidyl ester (401/421), CellTracker™ Violet BMQC (415/516), CellTracker™ Green CMFDA (492/517), calcein (495/515), Alexa Fluor® 488 conjugate of annexin V (495/519), Alexa Fluor® 488 goat anti-mouse IgG (H+L) (495/519), Click-iT® AHA Alexa Fluor® 488 Protein Synthesis HCS Assay (495/519), LIVE/DEAD® Fixable Green Dead Cell Stain Kit (495/519), SYTOX® Green nucleic acid stain (504/523), MitoSOX™ Red mitochondrial superoxide indicator (510/580). Alexa Fluor® 532 carboxylic acid, succinimidyl ester(532/554), pHrodo™ succinimidyl ester (558/576), CellTracker™ Red CMTPX (577/602), Texas Red® 1,2-dihexadecanoyl-sn-glycero-3-phosphoethanolamine (Texas Red® DHPE, 583/608), Alexa Fluor® 647 hydrazide (649/666), Alexa Fluor® 647 carboxylic acid, succinimidyl ester (650/668), Ulysis™ Alexa Fluor® 647 Nucleic Acid Labeling Kit (650/670) and Alexa Fluor® 647 conjugate of annexin V (650/665). Moieties which enhance the fluorescent signal or slow the fluorescent fading may also be incorporated and include SlowFade® Gold antifade reagent (with and without DAPI) and Image-iT® FX signal enhancer. All of these are well known in the art. Additional reporters include polypeptide reporters which may be expressed by plasmids (such as histone-packaged supercoiled DNA plasmids) and include polypeptide reporters such as fluorescent green protein and fluorescent red protein. Reporters are utilized principally in diagnostic applications including diagnosing the existence or progression of cancer (cancer tissue) in a patient and or the progress of therapy in a patient or subject.

The term “histone-packaged supercoiled plasmid DNA” is used to describe an exemplary component of protocells, which utilize an exemplary plasmid DNA which has been “supercoiled” (i.e., folded in on itself using a supersaturated salt solution or other ionic solution which causes the plasmid to fold in on itself and “supercoil” in order to become more dense for efficient packaging into the protocells). The plasmid may be virtually any plasmid which expresses any number of polypeptides or encode RNA, including small hairpin RNA/shRNA or small interfering RNA/siRNA, as otherwise described herein. Once supercoiled (using the concentrated salt or other anionic solution), the supercoiled plasmid DNA is then complexed with histone proteins to produce a histone-packaged “complexed” supercoiled plasmid DNA.

“Packaged” DNA herein refers to DNA that is loaded into protocells (either adsorbed into the pores or confined directly within the nanoporous silica core itself). To minimize the DNA spatially, it is often packaged, which can be accomplished in several different ways, from adjusting the charge of the surrounding medium to creation of small complexes of the DNA with, for example, lipids, proteins, or other nanoparticles (usually, although not exclusively cationic). Packaged DNA is often achieved via lipoplexes (i.e. complexing DNA with cationic lipid mixtures). In addition, DNA has also been packaged with cationic proteins (including proteins other than histones), as well as gold nanoparticles (e.g., NanoFlares—an engineered DNA and metal complex in which the core of the nanoparticle is gold).

The term “cancer” is used to describe a proliferation of tumor cells (neoplasms) having the unique trait of loss of normal controls, resulting in unregulated growth, lack of differentiation, local tissue invasion, and/or metastasis. As used herein, neoplasms include, without imitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms are distinguished from benign neoplasms in that the former show a greater degree of dysplasia, or loss of differentiation and orientation of cells, and have the properties of invasion and metastasis. The term cancer also within context, includes drug resistant cancers, including multiple drug resistant cancers. Examples of neoplasms or neoplasias from which the target cell may be derived include, without imitation, carcinomas (e.g., squamous-cell carcinomas, adenocarcinomas, hepatocellular carcinomas, and renal cell carcinomas), particularly those of the bladder, bone, bowel, breast, cervix, colon (colorectal), esophagus, head, kidney, liver (hepatocellular), lung, nasopharyngeal, neck, ovary, pancreas, prostate, and stomach; leukemias, such as acute myelogenous leukemia, acute lymphocytic leukemia, acute promyelocytic leukemia (APL), acute T-cell lymphoblastic leukemia, adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, particularly Burkitt's lymphoma, Non-Hodgkin's lymphoma and B-cell lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer (e.g., small cell lung cancer, mixed small cell and non-small cell cancer, pleural mesothelioma, including metastatic pleural mesothelioma small cell lung cancer and non-small cell lung cancer), ovarian cancer, testicular cancer, thyroid cancer, astrocytoma, esophageal cancer, pancreatic cancer, stomach cancer, liver cancer, colon cancer, and melanoma: mixed types of neoplasias, particularly carcinosarcoma and Hodgkin's disease; and tumors of mixed origin, such as Wilms' tumor and teratocarcinomas, among others. It is noted that certain tumors including hepatocellular and cervical cancer, among others, are shown to exhibit increased levels of MET receptors specifically on cancer cells and are a principal target for compositions and therapies according to embodiments which include a MET binding peptide complexed to the protocell.

The terms “coadminister” and “coadministration” are used synonymously to describe the administration of at least one of the protocell compositions in combination with at least one other agent, often at least one additional anti-cancer agent (as otherwise described herein), which are specifically disclosed herein in amounts or at concentrations which would be considered to be effective amounts at or about the same time. While it is envisioned that coadministered compositions/agents be administered at the same time, agents may be administered at times such that effective concentrations of both (or more) compositions/agents appear in the patient at the same time for at least a brief period of time. Alternatively, in certain aspects, it may be possible to have each coadministered composition/agent exhibit its inhibitory effect at different times in the patient, with the ultimate result being the inhibition and treatment of cancer, especially including hepatocellular or cellular cancer as well as the reduction or inhibition of other disease states, conditions or complications. Of course, when more than disease state, infection or other condition is present, the present compounds may be combined with other agents to treat that other infection or disease or condition as required.

The term “anti-cancer agent” is used to describe a compound which can be formulated in combination with one or more compositions comprising protocells and optionally, to treat any type of cancer, in particular hepatocellular or cervical cancer, among numerous others. Anti-cancer compounds which can be formulated with compounds include, for example, Exemplary anti-cancer agents which may be used include, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, eriotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespen, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-8244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrozole, exemestane, letrozole, DES (diethylstilbestrol), estradiol, estrogen, conjugated estrogen, bevacizumab, IMC-1C11, CHIR-258, 3-[5-(methylsulfonylpiperadinemethyl)-indolyl]-quinolone, vatalanib, AG-013736, AVE-0005, the acetate sail of [D-Ser(But)6, Azgly10] (pyro-Glu-His-Trp-Ser-Tyr-D-Ser(But)-Leu-Arg-Pro-Azgly-NH₂ acetate [C₅₉H₈₄N₁₈O₁₄—(C₂H₄O₂)_{X} where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714, TAK-165, HKI-272, erlotinib, lapatinib, canertinib, ABX-EGF antibody, erbitux, EKB-569, PKI-166, GW-572016, lonafarnib, BMS-214662, tipifarnib, amifostine, NVP-LAQ824, suberoyl anilide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, Idarubicin, Ifosfamide, imatinib, leuprolide, levamisole, lomustine, mechlorethamine, melphalan, 8-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, atretamine, floxuridine, 5-deoxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxifene, spironolactone, finasteride, cimetidine, trastuzumab, denileukin diftitox, gefitinib, bortezomib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, Interferon alfa-2b, azacitidine, PEG-L-asparaginase, lenalidomide, gemtuzumab, hydrocortisone, Interleukin-11, dexrazoxane, alemtuzumab, all-transretinoic acid, ketoconazole, interleukin-2, megestrol, immune globulin, nitrogen mustard, methylprednisolone, ibritumomab tiuxetan, androgens, decitabine, hexamethylmelamine, bexarotene, tositumomab, arsenic trioxide, cortisone, etidronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

The term “antihepatocellular cancer agent” is used throughout the specification to describe an anti-cancer agent which may be used to inhibit, treat or reduce the likelihood of hepatocellular cancer, or the metastasis of that cancer. Anti-cancer agents which may find use include for example, nexavar (sorafenib), sunitinib, bevacizumab, tarceva (erlotinib), tykerb (lapatinib) and mixtures thereof. In addition, other anticancer agents may also be used, where such agents are found to inhibit metastasis of cancer, in particular, hepatocellular cancer.

The term “anti(HCV)-viral agent” is used to describe a bioactive agent/drug which inhibits the growth and/or elaboration of a virus, Including mutant strains such as drug resistant viral strains. Exemplary anti-viral agents include anti-HIV agents, anti-HBV agents and anti-HCV agents. In certain aspects, especially where the treatment of hepatocellular cancer is the object of therapy, the inclusion of an anti-hepatitis C agent or anti-hepatitis B agent may be combined with other traditional anti-cancer agents to effect therapy, given that hepatitis B virus (HBV) and/or hepatitis C virus (HCV) is often found as a primary or secondary infection or disease state associated with hepatocellular cancer. Anti-HBV agents which may be used, either as a cargo component in the protocell or as an additional bioactive agent in a pharmaceutical composition which includes a population of protocells includes such agents as Hepsera (adefovir dipivoxil), lamivudine, entecavir, telbivudine, tenofovir, emtricitabine, clevudine, valtorcitabine, amdoxovir, pradefovir, racivir, BAM 205, nitazoxanide, UT 231-B, Bay 41-4109, EHT899, zadaxin (thymosin alpha-1) and mixtures thereof. Typical anti-HCV agents for use in include such agents as boceprevir, daclatasvir, asunaprevir, INX-189, FV-100, NM 283, VX-950 (telaprevir), SCH 50304, TMC435, VX-500, BX-813, SCH503034, R1626, ITMN-191 (R7227), R7128, PF-868554, TT033, CGH-759, GI 5005, MK-7009, SIRNA-034, MK-0608, A-837093, GS 9190, GS 9256, GS 9451, GS 5885, GS 6620, GS 9620, GS9669, ACH-1095, ACH-2928, GSK625433, TG4040 (MVA-HCV), A-831, F351, NS5A, NS4B, ANA598, A-689, GNI-104, IDX102, ADX184, ALS-2200, ALS-2158, BI 201335, BI 207127, BIT-225, BIT-8020, GL59728, GL60667, PSI-938, PSI-7977, PSI-7851, SCY-635, ribavirin, pegylated interferon, PHX1766, SP-30 and mixtures thereof.

The term “anti-HIV agent” refers to a compound which inhibits the growth and/or elaboration of HIV virus (I and/or II) or a mutant strain thereof. Exemplary anti-HIV agents for use which can be included as cargo in protocells include, for example, including nucleoside reverse transcriptase inhibitors (NRTI), other non-nucleoside reverse transcriptase inhibitors (i.e., those which are not representative), protease inhibitors, fusion inhibitors, among others, exemplary compounds of which may include, for example, 3TC (Lamivudine), AZT (Zidovudine), (−)-FTC, ddI (Didanosine), ddC (zalcitabine), abacavir (ABC), tenofovir (PMPA), D-D4FC (Reverset), D4T (Stavudine), Racivir, L-FddC, L-FD4C, NVP (Nevirapine), DLV (Delavirdine), EFV (Efavirenz), SQVM (Saquinavir mesylate), RTV (Ritonavir, IDV (Indinavir), SQV (Saquinavir), NFV (Nelfinavir), APV (Amprenavir), LPV (Lopinavir), fusion inhibitors such as T20, among others, fuseon and mixtures thereof

### Exemplary Monosized Nanostructures

In an embodiment, the nanostructures include a mesoporous silica core-shell structure which comprises a porous particle core surrounded by a shell of lipid such as a bi-layer, but possibly a monolayer or multi-layer. The porous silica particle core include, for example, a porous nanoparticle surrounded by a lipid bi-layer. In some non-limiting instances, these lipid bi-layer surrounded nanostructures are referred to as “protocells” or “functional protocells” and have a supported lipid bi-layer membrane structure. However, the porous nanoparticle may be surrounded by other naturally occurring or synthetic polymers and those may also be referred to as “protocells.” In some embodiments, the porous particle core of the protocells can be loaded with various desired species (“cargo”), including small molecules (e.g., anti-cancer agents as otherwise described herein), large molecules (e.g., Including macromolecules such as RNA, including small interfering RNA or siRNA or small hairpin RNA or shRNA or a polypeptide which may include a polypeptide toxin such as a ricin toxin A-chain or other toxic polypeptide such as diphtheria toxin A-chain DTx, among others) or a reporter polypeptide (e.g., fluorescent green protein, among others) or semiconductor quantum dots or combinations thereof. In certain exemplary aspects, the protocells are loaded with super-coiled plasmid DNA, which can be used to deliver a therapeutic and/or diagnostic peptide(s) or a small hairpin RNA/shRNA or small interfering RNA/siRNA which can be used to inhibit expression of proteins (such as, for example growth factor receptors or other receptors which are responsible for or assist in the growth of a cell especially a cancer cell, including epithelial growth factor/EGFR, vascular endothelial growth factor receptor/VEGFR-2 or platelet derived growth factor receptor/PDGFR-α, among numerous others, and induce growth arrest and apoptosis of cancer cells).

In certain embodiments, the cargo components can include, but are not limited to, chemical small molecules (especially anti-cancer agents, anti-viral agents and antibiotics, including anti-HIV, anti-HBV and/or anti-HCV agents, nucleic acids (DNA and RNA, including siRNA and shRNA and plasmids which, after delivery to a cell, express one or more polypeptides or RNA molecules), such as for a particular purpose, such as a therapeutic application or a diagnostic application as otherwise disclosed herein.

In some embodiments, the lipid bi-layer of the protocells can provide biocompatibility and can be modified to possess targeting species including, for example, targeting peptides including antibodies, aptamers, and PEG (polyethylene glycol) to allow, for example, further stability of the protocells and/or a targeted delivery into a bioactive cell.

In some embodiments, the protocells particle size distribution is monodisperse. In certain embodiments, protocells generally range in size from greater than about 8-10 nm to about 5 μm in diameter, e.g., about 20-nm-3 μm in diameter, about 10 nm to about 500 nm, about 20-200-nm (including about 150 nm, which may be a mean or median diameter), about 50 nm to about 150 nm, about 75 to about 130 nm, or about 75 to about 100 nm. As discussed above, the protocell population is considered monodisperse based upon the mean or median diameter of the population of protocells. Size is very important to therapeutic and diagnostic aspects as particles smaller than about 8-nm diameter are excreted through kidneys, and those particles larger than about 200 nm are often trapped by the liver and spleen. Thus, an embodiment on smaller monosized protocells are provided of less than about 150 nm for drug delivery and diagnostics in the patient or subject.

In certain embodiments, protocells are characterized by containing mesopores, e.g., pores which are found in the nanostructure material. These pores (at least one, but often a large plurality) may be found intersecting the surface of the nanoparticle (by having one or both ends of the pore appearing on the surface of the nanoparticle) or internal to the nanostructure with at least one or more mesopore interconnecting with the surface mesopores of the nanoparticle. Interconnecting pores of smaller size are often found internal to the surface mesopores. The overall range of pore size of the mesopores can be 0.03-50-nm in diameter. Exemplary pore sizes of mesopores range from about 2-30 nm; they can be monosized or bimodal or graded—they can be ordered or disordered (essentially randomly disposed or worm-like).

Mesopores (IUPAC definition 2-50-nm in diameter) are ‘molded’ by templating agents including surfactants, block copolymers, molecules, macromolecules, emulsions, latex beads, or nanoparticles. In addition, processes could also lead to micropores (IUPAC definition less than 2-nm in diameter) all the way down to about 0.03-nm e.g., if a templating moiety in the aerosol process is not used. They could also be enlarged to macropores, i.e., 50-nm in diameter.

Pore surface chemistry of the nanoparticle material can be very diverse—all organosilanes yielding cationic, anionic, hydrophilic, hydrophobic, reactive groups—pore surface chemistry, especially charge and hydrophobicity, affect loading capacity. Attractive electrostatic interactions or hydrophobic interactions control/enhance loading capacity and control release rates. Higher surface areas can lead to higher loadings of drugs/cargos through these attractive interactions.

In certain embodiments, the surface area of nanoparticles, as measured by the N2 BET method, ranges from about 100 m²/g to >about 1200 m²/g. In general, the larger the pore size, the smaller the surface area. The surface area theoretically could be reduced to essentially zero, if one does not remove the templating agent or if the pores are sub-0.5-nm and therefore not measurable by N₂ sorption at 77K due to kinetic effects. However, in this case, they could be measured by CO₂ or water sorption, but would probably be considered non-porous. This would apply if biomolecules are encapsulated directly in the silica cores prepared without templates, in which case particles (internal cargo) would be released by dissolution of the silica matrix after delivery to the cell.

Typically the protocells are loaded with cargo to a capacity up to over 100 weight %: defined as (cargo weight/weight of protocell)×100. The optimal loading of cargo is often about 0.01 to 30% but this depends on the drug or drug combination which is incorporated as cargo into the protocell. This is generally expressed in μM of cargo per 10¹⁰ particles where values often ranging from 2000-100 μM per 10¹⁰ particles are used. Exemplary protocells exhibit release of cargo at pH about 5.5, which is that of the endosome, but are stable at physiological pH of 7 or higher (7.4).

The surface area of the internal space for loading is the pore volume whose optimal value ranges from about 1.1 to 0.5 cubic centimeters per gram (cc/g). Note that in the protocells according to one embodiment, the surface area is mainly internal as opposed to the external geometric surface area of the nanoparticle.

The lipid bi-layer supported on the porous particle according to one embodiment has a lower melting transition temperature, e.g., is more fluid than a pd bi-layer supported on a non-porous support or the lipid bi-layer in a liposome. This is sometimes important in achieving high affinity binding of targeting ligands at low peptide densities, as it is the bi-layer fluidity that allows lateral diffusion and recruitment of peptides by target cell surface receptors. One embodiment provides for peptides to cluster, which facilitates binding to a complementary target.

The lipid bi-layer may vary significantly in composition. Ordinarily, any lipid or polymer which is may be used in liposomes may also be used in protocells. Exemplary lipids are as otherwise described herein. Particular lipid bi-layers for use in protocells comprise a mixtures of lipids (as otherwise described herein) at a weight ratio of 5% DOPE, 5% PEG, 30% cholesterol, 60% DOPC or DPPC (by weight).

The charge of the mesoporous silica NP core as measured by the Zeta potential may be varied monotonically from −50 to +50 mV by modification with the amine silane, 2-(aminoethyl) propyltrimethoxy-silane (AEPTMS) or other organosilanes. This charge modification, in turn, varies the loading of the drug within the cargo of the protocell. Generally, after fusion of the supported lipid bi-layer, the zeta-potential is reduced to between about −10 mV and +5 mV, which is important for maximizing circulation time in the blood and avoiding non-specific interactions.

Depending on how the surfactant template is removed, e.g., calcination at high temperature (500° C.) versus extraction in acidic ethanol, and on the amount of AEPTMS incorporated in the silica framework, the silica dissolution rates can be varied widely. This in turn controls the release rate of the internal cargo. This occurs because molecules that are strongly attracted to the internal surface area of the pores diffuse slowly out of the particle cores, so dissolution of the particle cores controls in part the release rate.

Further characteristics of protocells according to an embodiment are that they are stable at pH 7, i.e., they don't leak their cargo, but at pH 5.5, which is that of the endosome lipid or polymer coating becomes destabilized initiating cargo release. This pH-triggered release is important for maintaining stability of the protocell up until the point that it is internalized in the cell by endocytosis, whereupon several pH triggered events cause release into the endosome and consequently, the cytosol of the cell. The protocell core particle and surface can also be modified to provide non-specific release of cargo over a specified, prolonged period of time, as well as be reformulated to release cargo upon other biophysical changes, such as the increased presence of reactive oxygen species and other factors in locally inflamed areas. Quantitative experimental evidence has shown that targeted protocells illicit only a weak immune response, because they do not support T-Cell help required for higher affinity IgG, a favorable result.

Protocells may exhibit at least one or more a number of characteristics (depending upon the embodiment) which distinguish them from prior art protocells: 1) In contrast to the prior art, an embodiment specifies monosized nanoparticles whose average size (diameter) is less than about 200-nm—this size is engineered to enable efficient cellular uptake by receptor mediated endocytosis and to minimize binding and uptake by non-target cells and organs; 2) Monodisperse sizes to enable control of biodistribution of the protocells; 3) To targeted nanoparticles that bind selected to cells based upon the inclusion of a targeting species on the protocell; 4) To targeted nanoparticles that induce receptor mediated endocytosis; 5) Induces dispersion of cargo into cytoplasm of targeted cells through the inclusion of fusogenic or endosomolytic peptides; 8) Provides particles with pH triggered release of cargo; 7) Exhibits controlled time dependent release of cargo (via extent of thermally induced crosslinking of silica nanoparticle matrix); 8) Exhibit time dependent pH triggered release; 9) Contain and provide cellular delivery of complex multiple cargoes; 10) Cytotoxicity of target cancer cells; 11) Diagnosis of target cancer cells; 12) Selective entry of target cells; 13) Selective exclusion from off-target cells (selectivity); 14) Enhanced fluidity of the supported lipid bi-layer; 15) Sub-nanomolar and controlled binding affinity to target cells; 16) Sub-nanomolar binding affinity with targeting ligand densities; and/or 17) Colloidal and storage stability of compositions comprising protocells.

Various embodiments provide nanostructures which are constructed from nanoparticles which support a lipid bi-layer(s). In some embodiments, the nanostructures include, for example, a core-shell structure including a porous particle core surrounded by a shell of lipid bi-layer(s). The nanostructure, e.g., a porous silica nanostructure as described above, supports the lipid bi-layer membrane structure.

In some embodiments, the lipid bi-layer of the protocells can provide biocompatibility and can be modified to possess targeting species including, for example, targeting peptides, fusogenic peptides, antibodies, aptamers, and PEG (polyethylene glycol) to allow, for example, further stability of the protocells and/or a targeted delivery into a bioactive cell, in particular a cancer cell. PEG, when included in lipid bi-layers, can vary widely in molecular weight (although PEG ranging from about 10 to about 100 units of ethylene glycol, about 15 to about 50 units, about 40 to 50 units, about 15 to about 20 units, about 15 to about 25 units, about 16 to about 18 units, etc., may be used and the PEG component which is generally conjugated to phospholipid through an amine group comprises about 1% to about 20%, about 5% to about 15%, or about 10% by weight of the lipids which are included in the lipid bi-layer.

Numerous lipids which are used in liposome delivery systems may be used to form the lipid bi-layer on nanoparticles to provide protocells. Virtually any lipid or polymer which is used to form a liposome or polymersome may be used in the lipid bi-layer which surrounds the nanoparticles to form protocells according to an embodiment. Exemplary lipids for use include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine](DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanoamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol, not technically a lipid, but presented as a lipid for purposes of an embodiment of the given the fact that cholesterol may be an important component of the lipid bi-layer of protocells according to an embodiment. Often cholesterol is incorporated into lipid bi-layers of protocells in order to enhance structural integrity of the bi-layer. These lipids are all readily available commercially from Avanti Polar Lipids, Inc. (Alabaster, Ala., USA). DOPE and DPPE are particularly useful for conjugating (through an appropriate crosslinker) peptides, polypeptides, including antibodies, RNA and DNA through the amine group on the lipid.

In certain embodiments, the porous nanoparticulates can also be biodegradable polymer nanoparticulates comprising one or more compositions selected from the group consisting of aliphatic polyesters, poly (lactic acid) (PLA), poly (glycolic acid) (PGA), co-polymers of lactic acid and glycolic acid (PLGA), polycaprolactone (PCL), polyanhydrides, poly(ortho)esters, polyurethanes, poly(butyric acid), poly(valeric acid), poly(lactide-co-caprolactone), alginate and other polysaccharides, collagen, and chemical derivatives thereof, albumin a hydrophilic protein, zein, a prolamine, a hydrophobic protein, and copolymers and mixtures thereof.

In still other embodiments, the porous nanoparticles each comprise a core having a core surface that is essentially free of silica, and a shell attached to the core surface, wherein the core comprises a transition metal compound selected from the group consisting of oxides, carbides, sulfides, nitrides, phosphides, borides, halides, selenides, tellurides, tantalum oxide, iron oxide or combinations thereof.

The silica nanoparticles can be, for example, mesoporous silica nanoparticles and core-shell nanoparticles. The nanoparticles may incorporate an absorbing molecule, e.g., an absorbing dye. Under appropriate conditions, the nanoparticles emit electromagnetic radiation resulting from chemiluminescence. Additional contrast agents may be included to facilitate contrast in MRI, CT, PET, and/or ultrasound imaging.

Mesoporous silica nanoparticles can be, e.g., from around 5 nm to around 500 nm in size, including all integers and ranges there between. The size is measured as the longest axis of the particle. In various embodiments, the particles are from around nm to around 500 nm and from around 10 nm to around 100 nm in size. The mesoporous silica nanoparticles have a porous structure. The pores can be from around 1 to around 20 nm in diameter, including all integers and ranges there between. In one embodiment, the pores are from around 1 to around 10 nm in diameter. In one embodiment, around 90% of the pores are from around 1 to around 20 nm in diameter. In another embodiment, around 95% of the pores are around 1 to around 20 nm in diameter.

The mesoporous nanoparticles can be synthesized according to methods known in the art. In one embodiment, the nanoparticles are synthesized using sol-gel methodology where a silica precursor or silica precursors and a silica precursor or silica precursors conjugated (i.e., covalently bound) to absorber molecules are hydrolyzed in the presence of templates in the form of micelles. The templates are formed using a surfactant such as, for example, hexadecyltrimethylammonium bromide (CTAB). It is expected that any surfactant which can form micelles can be used.

Core-shell nanoparticles comprise a core and shell. The core, in one embodiment, comprises silica and an absorber molecule. The absorber molecule is incorporated in to the silica network via a covalent bond or bonds between the molecule and silica network. The shell comprises silica.

In one embodiment, the core is independently synthesized using known sol-gel chemistry, e.g., by hydrolysis of a silica precursor or precursors. The silica precursors are present as a mixture of a silica precursor and a silica precursor conjugated, e.g., linked by a covalent bond, to an absorber molecule (referred to herein as a “conjugated silica precursor”). Hydrolysis can be carried out under alkaline (basic) conditions to form a silica core and/or silica shell. For example, the hydrolysis can be carried out by addition of ammonium hydroxide to the mixture comprising silica precursor(s) and conjugated silica precursor(s).

Silica precursors are compounds which under hydrolysis conditions can form silica. Examples of silica precursors include, but are not limited to, organosilanes such as, for example, tetraethoxysilane (TEOS), tetramethoxysilane (TMOS) and the like.

The silica precursor used to form the conjugated silica precursor has a functional group or groups which can react with the absorbing molecule or molecules to form a covalent bond or bonds. Examples of such silica precursors include, but are not limited to, isocyanatopropyltriethoxysilane (ICPTS), aminopropyltrimethoxysilane (APTS), mercaptopropyltrimethoxysilane (MPTS), and the like.

In one embodiment, an organosilane (conjugatable silica precursor used for forming the core has the general formula R₄ₙSiXₙ, where X is a hydrolyzable group such as ethoxy, methoxy, or 2-methoxy-ethoxy; R can be a monovalent organic group of from 1 to 12 carbon atoms which can optionally contain, but is not limited to, a functional organic group such as mercapto, epoxy, acrylyl, methacrylyl, or amino; and n is an integer of from 0 to 4. The conjugatable silica precursor is conjugated to an absorber molecule and subsequently co-condensed for forming the core with silica precursors such as, for example, TEOS and TMOS. A silane used for forming the silica shell has n equal to 4. The use of functional mono-, bis- and tris-alkoxysilanes for coupling and modification of co-reactive functional groups or hydroxy-functional surfaces, Including glass surfaces, is also known, see Kirk-Othmer; see also Pluedemann, 1982. The organo-silane can cause gels, so it may be desirable to employ an alcohol or other known stabilizers. Processes to synthesize core-shell nanoparticles using modified Stoeber processes can be found in U.S. patent application Ser. Nos. 10/306,614 and 10/536,569, the disclosures of which are incorporated herein by reference.

In certain embodiments of a protocell, the lipid bi-layer is comprised of one or more lipids selected from the group consisting of phosphatidyl-cholines (PCs) and cholesterol.

In certain embodiments, the lipid bi-layer is comprised of one or more phosphatidyl-cholines (PCs) selected from the group consisting of 1,2-distearoyl-sn-glycero-3-phosphocholine (DSPC) [18:01, 1,2-dioleoyl-sn-glycero-3-phosphocholine (DOPC) 118:1 (Δ9-Cis)], 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), egg PC, and a lipid mixture comprising of one or more unsaturated phosphatidyl-cholines, DMPC [14:0] having a carbon length of 14 and no unsaturated bonds, 1,2-dipalmitoyl-sn-glycero-3-phosphocholine (DPPC) [16:0], POPC [16:0-18:1], and DOTAP [18:1]. The use of DSPC and/or DOPC as well as other zwitterionic phospholipids as a principal component (often in combination with a minor amount of cholesterol) is employed in certain embodiments in order to provide a protocell with a surface zeta potential which is neutral or close to neutral in character.

In other embodiments: (a) the lipid bi-layer is comprised of a mixture of (1) DSPC, DOPC and optionally one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), a lipid mixture comprising (in molar percent) between about 50% to about 70% or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-choline, DMPC [14:0] having a carbon length of 14 and no unsaturated bonds, 1,2-dipalmitoyl-sn-glycero-3-phosphocholine (DPPC) [16:0], POPC [16:0-18:1] and DOTAP [18:1]; and wherein (b) the molar concentration of DSPC and DOPC in the mixture is between about 10% to about 99% or about 50% to about 99%, or about 12% to about 98%, or about 13% to about 97%, or about 14% to about 96%, or about 55% to about 95%, or about 56% to about 94%, or about 57% to about 93%, or about 58% to about 42%, or about 59% to about 91%, or about 50% to about 90%, or about 51% to about 89%.

In certain embodiments, the lipid bi-layer is comprised of one or more compositions selected from the group consisting of a phospholipid, a phosphatidyl-choline, a phosphatidyl-serine, a phosphatidyl-diethanolamine, a phosphatidylinosite, a sphingolipid, and an ethoxylated sterol, or mixtures thereof. In illustrative examples of such embodiments, the phospholipid can be a lecithin; the phosphatidylinosite can be derived from soy, rape, cotton seed, egg and mixtures thereof; the sphingolipid can be ceramide, a cerebroside, a sphingosine, and a sphingomyelin, and a mixture thereof; the ethoxylated sterol can be phytosterol, PEG-(polyethyleneglycol)-5-soy bean sterol, and PEG-(polyethyleneglycol)-5 rapeseed sterol. In certain embodiments, the phytosterol comprises a mixture of at least two of the following compositions: sitosterol, campesterol and stigmasterol.

In still other illustrative embodiments, the lipid bi-layer is comprised of one or more phosphatidyl groups selected from the group consisting of phosphatidyl choline, phosphatidyl-ethanolamine, phosphatidyl-serine, phosphatidyl-inositol, lyso-phosphatidyl-choline, lyso-phosphatidyl-ethanolamine, lyso-phosphatidyl-inositol and lyso-phosphatidyl-inositol.

In still other illustrative embodiments, the lipid bi-layer is comprised of phospholipid selected from a monoacyl or diacylphosphoglyceride.

In still other illustrative embodiments, the lipid bi-layer is comprised of one or more phosphoinositides selected from the group consisting of phosphatidyl-inositol-3-phosphate (PI-3-P), phosphatidyl-inositol-4-phosphate (PI-4-P), phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-Inositol-3,4,5-triphosphate (PI-3,4,5-P3), lysophosphatidyl-Inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidyl-inositol-3,4,5-triphosphate (LPI-3,4,5-P3), and phosphatidyl-Inositol (PI), and lysophosphatidyl-inositol (LPI).

In still other illustrative embodiments, the lipid bi-layer is comprised of one or more phospholipids selected from the group consisting of PEG-poly(ethylene glycol)-derivatized distearoylphosphatidylethanolamine (PEG-DSPE), PEG-poly(ethylene glycol)-derivatized dioleoylphosphatidylethanolamine (PEG-DOPE), poly(ethylene glycol)-derivatized ceramides (PEG-CER), hydrogenated soy phosphatidylcholine (HSPC), egg phosphatidylcholine (EPC), phosphatidyl ethanolamine (PE), phosphatidyl glycerol (PG), phosphatidyl inositol (PI), monosialoganglioside, sphingomyelin (SPM), distearoylphosphatidylcholine (DSPC), dimyristoylphosphatidylcholine (DMPC), and dimyristoylphosphatidylglycerol (DMPG).

In still other embodiments, the lipid bi-layer comprises one or more PEG-containing phospholipids, for example 1,2-dioleoyl-sn-glycero-3-phosphoethanolamine-N-[methoxy(polyethylene glycol)] (ammonium salt) (DOPE-PEG), 1,2-distearoyl-sn-glycero-3-phosphoethanolamine-N-[methoxy(polyethylene glycol)] (ammonium salt) (DSPE-PEG), 1,2-distearoyl-sn-glycero-3-phosphoethanolamine-N-[amino(polyethylene glycol)] (DSPE-PEG-NH₂) (DSPE-PEG). In the PEG-containing phospholipid, the PEG group ranges from about 2 to about 250 ethylene glycol units, about 5 to about 100, about 10 to 75, or about 40-50 ethylene glycol units. In certain exemplary embodiments, the PEG-phospholipid is 1,2-dioleoyl-sn-glycero-3-phosphoethanolamine-N-[methoxy(polyethylene glycol)-2000] (ammonium salt) (DOPE-PEG₂₀₀₀), 1,2-distearoyl-sn-glycero-3-phosphoethanolamine-N-[methoxy(polyethylene glycol)-2000] (ammonium salt) (DSPE-PEG₂₀₀₀), 1,2-distearoyl-sn-glycero-3-phosphoethanolamine-N-[amino(polyethylene glycol)-2000] (DSPE-PEG₂₀₀₀-NH₂) which can be used to covalent bind a functional moiety to the lipid bi-layer.

In one illustrative embodiment of a protocell: (a) the one or more pharmaceutically-active agents include at least one anti-cancer agent; (b) less than around 10% to around 20% of the anti-cancer agent is released from the porous nanoparticulates in the absence of a reactive oxygen species: and (c) upon disruption of the lipid bi-layer as a result of contact with a reactive oxygen species, the porous nanoparticulates release an amount of anti-cancer agent that is approximately equal to around 60% to around 80%, or around 61% to around 79%, or around 62% to around 78%, or around 63% to around 77%, or around 64% to around 77%, or around 65% to around 76%, or around 66% to around 75%, or around 67% to around 74%, or around 68% to around 73%, or around 69% to around 72%, or around 70% to around 71%, or around 70% of the amount of anti-cancer agent that would have been released had the lipid bi-layer been lysed with 5% (w/v) Triton X-100.

One illustrative embodiment of a protocell comprises a plurality of negatively-charged, nanoporous, nanoparticulate silica cores that: (a) are modified with an amine-containing silane selected from the group consisting of (1) a primary amine, a secondary amine a tertiary amine, each of which is functionalized with a silicon atom (2) a monoamine or a polyamine (3) N-(2-aminoethyl)-3-aminopropyltrimethoxysilane (AEPTMS) (4) 3-aminopropyltrimethoxysilane (APTMS) (5) 3-aminopropyltriethoxysilane (APTS) (6) an amino-functional trialkoxysilane, and (7) protonated secondary amines, protonated tertiary alkyl amines, protonated amidines, protonated guanidines, protonated pyridines, protonated pyrimidines, protonated pyrazines, protonated purines, protonated imidazoles, protonated pyrroles, and quaternary alkyl amines, or combinations thereof; (b) are loaded with a siRNA or ricin toxin A-chain; and (c) that are encapsulated by and that support a lipid bi-layer comprising one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof, and wherein the lipid bi-layer comprises a cationic lipid and one or more zwitterionic phospholipids.

Monosized protocells can comprise a wide variety of pharmaceutically-active ingredients such as nucleic acid, e.g., DNA.

Any number of histone proteins, as well as other means to package the DNA into a smaller volume such as normally cationic nanoparticles, lipids, or proteins, may be used to package the supercoiled plasmid DNA “histone-packaged supercoiled plasmid DNA”, but in therapeutic aspects which relate to treating human patients, the use of human histone proteins is envisioned. In certain aspects, a combination of human histone proteins H1, H2A, H2B, H3 and H4 in an exemplary ratio of 1:2:2:2:2, although other histone proteins may be used in other, similar ratios, as is known in the art or may be readily practiced pursuant to the teachings herein. The DNA may also be double stranded linear DNA, instead of plasmid DNA, which also may be optionally supercoiled and/or packaged with histones or other packaging components.

Other histone proteins which may be used in this aspect include, for example, H1F, H1F0, H1FNT, H1FOO, H1FX, H1H1, HIST1H1A, HIST1H1B, HIST1H1C, HIST1H1D, HIST1H1E, HIST1H1T, H2AF, H2AFB1, H2AFB2, H2AFB3, H2AFJ, H2AFV, H2AFX, H2AFY, H2AFY2, H2AFZ, H2A1, HIST1H2AA, HIST1H2AB, HIST1H2AC, HIST1H2AD, HIST1H2AE, HIST1H2AG, HIST1H2AI, HIST1H2AJ, HIST1H2AK, HIST1H2AL, HIST1H2AM, H2A2, HIST2H2AA3, HIST2H2AC, H2BF, H2BFM, HSBFS, HSBFWT, H2B1, HIST1H2BA, HIST1HSBB, HIST1HSBC, HIST1HSBD, HIST1H2BE, HIST1H2BF, HIST1H2BG, HIST1H2BH, HIST1H2BI, HIST1H2BJ, HIST1H2BK, HIST1H2BL, HIST1H2BM, HIST1H2BN, HIST1H2BO, H2B2, HIST2H2BE, H3A1, HIST1H3A, HIST1H3B, HIST1H3C, HIST1H3D, HIST1H3E, HIST1H3F, HIST1H3G, HIST1H3H, HIST1H3I, HIST1H3J, H3A2, HIST2H3C, H3A3, HIST3H3, H41, HIST1H4A, HIST1H4B, HIST1H4C, HIST1H4D, HIST1H4E, HIST1H4F, HIST1H4G, HIST1H4H, HIST1H41, HIST1H4J, HIST1H4K, HIST1H4L, H44 and HIST4H4.

The term “nuclear localization sequence” refers to a peptide sequence incorporated or otherwise crosslinked into histone proteins which comprise the histone-packaged supercoiled plasmid DNA. In certain embodiments, protocells may further comprise a plasmid (often a histone-packaged supercoiled plasmid DNA) which is modified (crosslinked) with a nuclear localization sequence (note that the histone proteins may be crosslinked with the nuclear localization sequence or the plasmid itself can be modified to express a nuclear localization sequence) which enhances the ability of the histone-packaged plasmid to penetrate the nucleus of a cell and deposit its contents there (to facilitate expression and ultimately cell death. These peptide sequences assist in carrying the histone-packaged plasmid DNA and the associated histones into the nucleus of a targeted cell whereupon the plasmid will express peptides and/or nucleotides as desired to deliver therapeutic and/or diagnostic molecules (polypeptide and/or nucleotide) into the nucleus of the targeted cell. Any number of crosslinking agents, well known in the art, may be used to covalently link a nuclear localization sequence to a histone protein (often at a lysine group or other group which has a nucleophilic or electrophilic group in the side chain of the amino acid exposed pendant to the polypeptide) which can be used to introduce the histone packaged plasmid into the nucleus of a cell. Alternatively, a nucleotide sequence which expresses the nuclear localization sequence can be positioned in a plasmid in proximity to that which expresses histone protein such that the expression of the histone protein conjugated to the nuclear localization sequence will occur thus facilitating transfer of a plasmid into the nucleus of a targeted cell.

Proteins gain entry into the nucleus through the nuclear envelope. The nuclear envelope consists of concentric membranes, the outer and the inner membrane. These are the gateways to the nucleus. The envelope consists of pores or large nuclear complexes. A protein translated with a NLS will bind strongly to importin (aka karyopherin), and together, the complex will move through the nuclear pore. Any number of nuclear localization sequences may be used to introduce histone-packaged plasmid DNA into the nucleus of a cell. Exemplary nuclear localization sequences include H₂N-GNQSSNFGPMKGGNFGGRSSGPYGGGGQYFAKPRNQGGYGGC-COOH (SEQ ID NO: 22), RRMKWKK (SEQ ID NO:23), PKKKRKV (SEQ ID NO:24), and KR[PAATKKAGQA]KKKK (SEQ ID NO:25), the NLS of nucleoplasmin, a prototypical bipartite signal comprising two clusters of basic amino acids, separated by a spacer of about 10 amino acids. Numerous other nuclear localization sequences are well known in the art. See, for example, LaCasse et al., 1995; Weis, 1998, TIBS, 23, 185-9 (1998); and Murat Cokol et al., “Finding nuclear localization signals”, at the website ubic.bloc.columbia.edu/papers/2000 nls/paper.htm#ab2.

In general, protocells are biocompatible. Drugs and other cargo components are often loaded by adsorption and/or capillary filling of the pores of the particle core up to approximately 50% by weight of the final protocell (containing all components). In certain embodiments, the loaded cargo can be released from the porous surface of the particle core (mesopores), wherein the release profile can be determined or adjusted by, for example, the pore size, the surface chemistry of the porous particle core, the pH value of the system, and/or the interaction of the porous particle core with the surrounding lipid bi-layer(s) as generally described herein.

The porous nanoparticle core used to prepare the protocells can be tuned in to be hydrophilic or progressively more hydrophobic as otherwise described herein and can be further treated to provide a more hydrophilic surface. For example, mesoporous silica particles can be further treated with ammonium hydroxide and hydrogen peroxide to provide a higher hydrophilicity. In some aspects, the lipid bi-layer is fused onto the porous particle core to form the monosized protocells. Protocells can include various lipids in various weight ratios, including 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine](DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (18:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof.

The lipid bi-layer which is used to prepare protocells are monosized liposomes which can be prepared, for example, by extrusion of liposomes prepared by bath sonication through a filter with pore size of, for example, about 100 nm, using standard protocols known in the art or as otherwise described herein. Alternatively, the monosized liposomes are prepared from lipids using bath and probe sonication without extrusion. While the majority of the monosized liposomes are unilamellar when prepared using extrusion, in the absence of extrusion, the monosized liposomes will have an appreciable percent of multilamellar liposomes. The monosized liposomes can then be fused with the porous particle cores, for example, by sonicating (e.g., bath sonication, other) a mixtures of monosized liposomes and mMSNPs in buffered saline solution (e.g., PBS), followed by separation (centrifugation) and redispersing the pelleted protocells via sonication in a saline or other solution. In exemplary embodiments, excess amount of liposome (e.g., at least twice the amount of liposome to mMSNP) is used. To improve the protocell colloidal and/or storage stability of the protocell composition, the transition melting temperature (Tₘ) of the lipid bi-layer should be greater than the temperature at which the protocells are to be stored and/or used. For storage stable liposomes, the inclusion of appreciable amounts of saturated phospholipids in the lipid bi-layer is often used to increase the Tₘ of the lipid bi-layer.

In certain diagnostic embodiments, various dyes or fluorescent (reporter) molecules can be included in the protocell cargo (as expressed by as plasmid DNA) or attached to the porous particle core and/or the lipid bi-layer for diagnostic purposes. For example, the porous particle core can be a silica core or the lipid bi-layer and can be covalently labeled with FITC (green fluorescence), while the lipid bi-layer or the particle core can be covalently labeled with FITC Texas red (red fluorescence). The porous particle core, the lipid bi-layer and the formed protocell can then be observed by, for example, confocal fluorescence for use in diagnostic applications. In addition, as discussed herein, plasmid DNA can be used as cargo in protocells, such that the plasmid may express one or more fluorescent proteins such as fluorescent green protein or fluorescent red protein which may be used in diagnostic applications.

In various embodiments, the protocell is used in a synergistic system where the lipid bi-layer fusion or liposome fusion (i.e., on the porous particle core) is loaded and sealed with various cargo components with the pores (e.g., mesopores) of the particle core, thus creating a loaded protocell useful for cargo delivery across the cell membrane of the lipid bi-layer or through dissolution of the porous nanoparticle, if applicable. In certain embodiments, in addition to fusing a single lipid (e.g., phospholipids) bi-layer, multiple bi-layers with opposite charges can be successively fused onto the porous particle core to further influence cargo loading and/or sealing as well as the release characteristics of the final protocell

A fusion and synergistic loading mechanism can be included for cargo delivery. For example, cargo can be loaded, encapsulated, or sealed, synergistically through liposome fusion on the porous particles. The cargo can include, for example, small molecule drugs (e.g., especially including anti-cancer drugs and/or anti-viral drugs such as anti-HBV or anti-HCV drugs), peptides, proteins, antibodies, DNA (especially plasmid DNA, Including the exemplary histone-packaged super coiled plasmid DNA), RNAs (including shRNA and siRNA (which may also be expressed by the plasmid DNA incorporated as cargo within the protocells) fluorescent dyes, including fluorescent dye peptides which may be expressed by the plasmid DNA incorporated within the protocell.

In some embodiments, the cargo can be loaded into the pores (mesopores) of the porous particle cores to form the loaded protocell. In various embodiments, any conventional technology that is developed for liposome-based drug delivery, for example, targeted delivery using PEGylation, can be transferred and applied to the protocells.

As discussed above, electrostatics and pore size can play a role in cargo loading. For example, porous silica nanoparticles can carry a negative charge and the pore size can be tunable from about 2 nm to about 10 nm or more. Negatively charged nanoparticles can have a natural tendency to adsorb positively charged molecules and positively charged nanoparticles can have a natural tendency to adsorb negatively charged molecules. In various embodiments, other properties such as surface wettability (e.g., hydrophobicity) can also affect loading cargo with different hydrophobicity.

In various embodiments, the cargo loading can be a synergistic lipid-assisted loading by tuning the lipid composition. For example, if the cargo component is a negatively charged molecule, the cargo loading into a negatively charged silica can be achieved by the lipid-assisted loading. In certain embodiments, for example, a negatively species can be loaded as cargo into the pores of a negatively charged silica particle when the lipid bi-layer is fused onto the silica surface showing a fusion and synergistic loading mechanism. In this manner, fusion of a non-negatively charged (i.e., positively charged or neutral) lipid bi-layer or liposome on a negatively charged mesoporous particle can serve to load the particle core with negatively charged cargo components. The negatively charged cargo components can be concentrated in the loaded protocell having a concentration exceed about 100 times as compared with the charged cargo components in a solution. In other embodiments, by varying the charge of the mesoporous particle and the lipid bi-layer, positively charged cargo components can be readily loaded into protocells.

Once produced, the loaded protocells can have a cellular uptake for cargo delivery into a desirable site after administration. For example, the cargo-loaded protocells can be administered to a patient or subject and the protocell comprising a targeting peptide can bind to a target cell and be internalized or uptaken by the target cell, for example, a cancer cell in a subject or patient. Due to the internalization of the cargo-loaded protocells in the target cell, cargo components can then be delivered into the target cells. In certain embodiments the cargo is a small molecule, which can be delivered directly into the target cell for therapy. In other embodiments, negatively charged DNA or RNA (including shRNA or siRNA), especially including a DNA plasmid which may be formulated as histone-packaged supercoiled plasmid DNA for example modified with a nuclear localization sequence can be directly delivered or internalized by the targeted cells. Thus, the DNA or RNA can be loaded first into a protocell and then into then through the target cells through the internalization of the loaded protocells.

As discussed, the cargo loaded into and delivered by the protocell to targeted cells includes small molecules or drugs (especially anti-cancer or anti-HBV and/or anti-HCV agents), bioactive macromolecules (bioactive polypeptides such as ricin toxin A-chain or diphtheria toxin A-chain or RNA molecules such as shRNA and/or siRNA as otherwise described herein) or histone-packaged supercoiled plasmid DNA which can express a therapeutic or diagnostic peptide or a therapeutic RNA molecule such as shRNA or siRNA, wherein the histone-packaged supercoiled plasmid DNA is optionally modified with a nuclear localization sequence which can localize and concentrate the delivered plasmid DNA into the nucleus of the target cell. As such, loaded protocells can deliver their cargo into targeted cells for therapy or diagnostics.

In various embodiments, the protocells and/or the loaded protocells can provide a targeted delivery methodology for selectively delivering the protocells or the cargo components to targeted cells (e.g., cancer cells). For example, a surface of the lipid bi-layer can be modified by a targeting active species that corresponds to the targeted cell. The targeting active species may be a targeting peptide as otherwise described herein, a polypeptide including an antibody or antibody fragment, an aptamer, a carbohydrate or other moiety which binds to a targeted cell. In exemplary aspects, the targeting active species is a targeting peptide as otherwise described herein. In certain embodiments, exemplary peptide targeting species include a MET binding peptide as otherwise described herein.

For example, by providing a targeting active species (e.g., a targeting peptide) on the surface of the loaded protocell, the protocell selectively binds to the targeted cell in accordance with the present teachings. In one embodiment, by conjugating an exemplary targeting peptide SP94 or analog or a MET binding peptide as otherwise described herein that targets cancer cells, including cancer liver cells to the lipid bi-layer, a large number of the cargo-loaded protocells can be recognized and internalized by this specific cancer cells due to the specific targeting of the exemplary SP94 or a MET or a CRLF2 binding peptide with the cancer (including liver) cells. In most instances, if the protocells are conjugated with the targeting peptide, the protocells will selectively bind to the cancer cells and no appreciable binding to the non-cancerous cells occurs.

Once bound and taken up by the target cells, the loaded protocells can release cargo components from the porous particle and transport the released cargo components into the target cell. For example, sealed within the protocell by the liposome fused bi-layer on the porous particle core, the cargo components can be released from the pores of the lipid bi-layer, transported across the protocell membrane of the lipid bi-layer and delivered within the targeted cell. In embodiments, the release profile of cargo components in protocells can be more controllable as compared with when only using liposomes as known in the prior art. The cargo release can be determined by, for example, interactions between the porous core and the lipid bi-layer and/or other parameters such as pH value of the system. For example, the release of cargo can be achieved through the lipid bi-layer, through dissolution of the porous silica; while the release of the cargo from the protocells can be pH-dependent.

In certain embodiments, the pH value for cargo is often less than 7, or about 4.5 to about 6.0, but can be about pH 14 or less. Lower pHs tend to facilitate the release of the cargo components significantly more than compared with high pHs. Lower pHs tend to be advantageous because the endosomal compartments inside most cells are at low pHs (about 5.5), but the rate of delivery of cargo at the cell can be influenced by the pH of the cargo. Depending upon the cargo and the pH at which the cargo is released from the protocell, the release of cargo can be relative short (a few hours to a day or so) or span for several days to about 20-30 days or longer. Thus, the protocell compositions may accommodate immediate release and/or sustained release applications from the protocells themselves.

In certain embodiments, the inclusion of surfactants can be provided to rapidly rupture the lipid bi-layer, transporting the cargo components across the lipid bi-layer of the protocell as well as the targeted cell. In certain embodiments, the phospholipid bi-layer of the protocells can be ruptured by the application/release of a surfactant such as sodium dodecyl sulfate (SDS), among others to facilitate a rapid release of cargo from the protocell into the targeted cell. Other than surfactants, other materials can be included to rapidly rupture the bi-layer. One example would be gold or magnetic nanoparticles that could use light or heat to generate heat thereby rupturing the bi-layer. Additionally, the bi-layer can be tuned to rupture in the presence of discrete biophysical phenomena, such as during inflammation in response to increased reactive oxygen species production. In certain embodiments, the rupture of the lipid bi-layer can in turn induce immediate and complete release of the cargo components from the pores of the particle core of the protocells. In this manner, the protocell platform can provide an increasingly versatile delivery system as compared with other delivery systems in the art. For example, when compared to delivery systems using nanoparticles only, the disclosed protocell platform can provide a simple system and can take advantage of the low toxicity and immunogenicity of liposomes or lipid bi-layers along with their ability to be PEGylated or to be conjugated to extend circulation time and effect targeting. In another example, when compared to delivery systems using liposome only, the protocell platform can provide a more stable system and can take advantage of the mesoporous core to control the loading and/or release profile and provide increased cargo capacity.

In addition, the lipid bi-layer and its fusion on porous particle core can be fine-tuned to control the loading, release, and targeting profiles and can further comprise fusogenic peptides and related peptides to facilitate delivery of the protocells for greater therapeutic and/or diagnostic effect. Further, the lipid bi-layer of the protocells can provide a fluidic interface for ligand display and multivalent targeting, which allows specific targeting with relatively low surface ligand density due to the capability of ligand reorganization on the fluidic lipid interface. Furthermore, the disclosed protocells can readily enter targeted cells while empty liposomes without the support of porous particles cannot be internalized by the cells.

Pharmaceutical compositions may comprise an effective population of protocells as otherwise described herein formulated to effect an intended result (e.g., therapeutic result and/or diagnostic analysis, Including the monitoring of therapy) formulated in combination with a pharmaceutically acceptable carrier, additive or excipient. The protocells within the population of the composition may be the same or different depending upon the desired result to be obtained. Pharmaceutical compositions may also comprise an addition bioactive agent or drug, such as an anti-cancer agent or an anti-viral agent, for example, an anti-HIV, anti-HBV or an anti-HCV agent.

Generally, dosages and routes of administration of the compound are determined according to the size and condition of the subject, according to standard pharmaceutical practices. Dose levels employed can vary widely, and can readily be determined by those of skill in the art. Typically, amounts in the milligram up to gram quantities are employed. The composition may be administered to a subject by various routes, e.g., orally, transdermally, perineural or parenterally, that is, by intravenous, subcutaneous, Intraperitoneal, intrathecal or intramuscular injection, among others, including buccal, rectal and transdermal administration. Subjects contemplated for treatment according to the method include humans, companion animals, laboratory animals, and the like. The disclosure contemplates immediate and/or sustained/controlled release compositions, including compositions which comprise both immediate and sustained release formulations. This is particularly true when different populations of protocells are used in the pharmaceutical compositions or when additional bioactive agent(s) are used in combination with one or more populations of protocells as otherwise described herein.

Formulations containing the compounds may take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like, e.g., in unit dosage forms suitable for simple administration of precise dosages.

Pharmaceutical compositions typically include a conventional pharmaceutical carrier or excipient and may additionally include other medicinal agents, carriers, adjuvants, additives and the like. In one embodiment, the composition is about 0.1% to about 95%, about 0.25% to about 85%, about 0.5% to about 75% by weight of a compound/composition or compounds/compositions, with the remainder consisting essentially of suitable pharmaceutical excipients.

An injectable composition for parenteral administration (e.g., intravenous, intramuscular or intrathecal) will typically contain the compound in a suitable i.v. solution, such as sterile physiological salt solution. The composition may also be formulated as a suspension in an aqueous emulsion.

Liquid compositions can be prepared by dissolving or dispersing the population of protocells (about 0.5% to about 20% by weight or more), and optional pharmaceutical adjuvants, in a carrier, such as, for example, aqueous saline, aqueous dextrose, glycerol, or ethanol, to form a solution or suspension. For use in an oral liquid preparation, the composition may be prepared as a solution, suspension, emulsion, or syrup, being supplied either in liquid form or a dried form suitable for hydration in water or normal saline.

For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

When the composition is employed in the form of solid preparations for oral administration, the preparations may be tablets, granules, powders, capsules or the like. In a tablet formulation, the composition is typically formulated with additives, e.g., an excipient such as a saccharide or cellulose preparation, a binder such as starch paste or methyl cellulose, a filler, a disintegrator, and other additives typically used in the manufacture of medical preparations.

Methods for preparing such dosage forms are known or would be apparent to those skilled in the art: for example, see Remington's Pharmaceutical Sciences (17th Ed., Mack Pub. Co., 1985). The composition to be administered will contain a quantity of the selected compound in a pharmaceutically effective amount for therapeutic use in a biological system, including a patient or subject.

Methods of treating patients or subjects in need for a particular disease state or infection (especially including cancer and/or a HBV, HCV or HIV infection) comprise administration an effective amount of a pharmaceutical composition comprising therapeutic protocells and optionally at least one additional bioactive (e.g., anti-viral) agent.

Diagnostic methods may comprise administering to a patient in need (a patient suspected of having cancer) an effective amount of a population of diagnostic protocells (e.g., protocells which comprise a target species, such as a targeting peptide which binds selectively to cancer cells and a reporter component to indicate the binding of the protocells to cancer cells if the cancer cells are present) whereupon the binding of protocells to cancer cells as evidenced by the reporter component (moiety) will enable a diagnosis of the existence of cancer in the patient.

An alternative of the diagnostic method can be used to monitor the therapy of cancer or other disease state in a patient, the method comprising administering an effective population of diagnostic protocells (e.g., protocells which comprise a target species, such as a targeting peptide which binds selectively to cancer cells or other target cells and a reporter component to indicate the binding of the protocells to cancer cells if the cancer cells are present) to a patient or subject prior to treatment, determining the level of binding of diagnostic protocells to target cells in said patient and during and/or after therapy, determining the level of binding of diagnostic protocells to target cells in said patient, whereupon the difference in binding before the start of therapy in the patient and during and/or after therapy will evidence the effectiveness of therapy in the patient, including whether the patient has completed therapy or whether the disease state has been inhibited or eliminated (including remission of a cancer).

### Vaccine Embodiments

Historically, vaccines have worked by eliciting long lived soluble antibody production. These B cell vaccines are capable of neutralizing or blocking the spread of pathogens in the body. This long lived antibody response primarily targets and neutralizes pathogens as they are spreading from cell to cell, however, they are less effective at eliminating the pathogen once i has entered the host cell. On the other hand, T cell vaccines generate a population of immune cells capable of identifying infected cells and, through affinity dependent mechanisms, kill the cell; thereby eliminating pathogen production at its source. The CD4+ T cells activate innate immune cells, promote B cell antibody production, and provide growth factors and signals for CD8+ T cell maintenance and proliferation. The CD8+ T cells directly recognize and kill virally infected host cells. The ultimate goal of a T cell vaccine is to develop long lived CD8+ memory T cells capable of rapid expansion to combat microbial, e.g., viral, infection.

In some embodiments of a vaccine, a protocell includes a porous nanoparticle core which is made of a material comprising silica, polystyrene, alumina, titania, zirconia, or generally metal oxides, organometallates, organosilicates or mixtures thereof. A porous spherical silica nanoparticle core is used for the exemplary protocells and is surrounded by a supported lipid or polymer bi-layer or multi-layer (multilamellar). Various embodiments provide nanostructures and methods for constructing and using the nanostructures and providing protocells. Porous silica particles are often used and are of varying sizes ranging in size (diameter) from less than 5 nm to 200 nm or 500 nm or more are readily available in the art or can be readily prepared using methods known in the art or alternatively, can be purchased from Melorium Technologies, Rochester, N.Y. SkySpring Nanomaterials, Inc., Houston, Tex., USA or from Discovery Scientific, Inc., Vancouver, British Columbia. Multimodal silica nanoparticles may be readily prepared using the procedure of Carroll et al., 2009. Protocells can be readily obtained using methodologies known in the art. Protocells may be readily prepared, Including protocells comprising lipids which are fused to the surface of the silica nanoparticle. See, for example, Liu et al. (2009), Liu et al. (2009), Liu et al. (2009), Lu et al. (1999). Other protocells for use are prepared according to the procedures which are presented in Ashley et al. (2010), Lu et al., (1999), Caroll et al., (2009), and as otherwise presented in the experimental section which follows. Multilamellar protocells may be prepared according to the procedures which are set forth in Moon et al., (2011), among others well known in the art. Another approach would be to hydrate lipid films and bath sonicate (without extrusion) and use polydisperse liposome fusion onto monodisperse cores loaded with cargo.

In some embodiments of the vaccine, the protocells include a core-shell structure which comprises a porous particle core surrounded by a shell of lipid which is often a mull-layer (multilamellar), but may include a single bi-layer (unilamellar), (see Lu et al., 2009). The porous particle core can include, for example, a porous nanoparticle made of an inorganic and/or organic material as set forth above surrounded by a lipid bi-layer. In some embodiments of the vaccine, the porous particle core of the protocells can be loaded with various desired species (“cargo”), especially including plasmid DNA which encodes for a microbial protein such as a bacterial protein, e.g., for a vaccine for tetanus, anthrax, haemophilus, pertussis, diphtheria, cholera, lyme disease, bacterial meningitis, Streptococcus pneumoniae, and typhoid, fungal protein, protist protein, archaea protein or a viral protein (fused to ubiquitin or not) or other microbial antigen (each of which may be ubiquitinylated) and additionally, depending upon the ultimate therapeutic goal, small molecules bioactive agents (e.g., antibiotics and/or anti-cancer agents as otherwise such as adjuvants as described herein), large molecules (e.g., especially including plasmid DNA, other macromolecules such as RNA, including small interfering RNA or siRNA or small hairpin RNA or shRNA or a polypeptide. In certain aspects, the protocells are loaded with super-coiled plasmid DNA, which can be used to deliver the microbial protein or optionally, other macromolecules such as a small hairpin RNA/shRNA or small interfering RNA/siRNA which can be used to inhibit expression of proteins (such as, for example growth factor receptors or other receptors which are responsible for or assist in the growth of a cell especially a cancer cell, including epithelial growth factor/EGFR, vascular endothelial growth factor receptor/VEGFR-2 or platelet derived growth factor receptor/PDGFR-α, among numerous others, and induce growth arrest and apoptosis of cancer cells).

In certain embodiments, the cargo components can include, but are not limited to, chemical small molecules (especially anti-microbial agents and/or anti-cancer agents, nucleic acids (DNA and RNA, including siRNA and shRNA and plasmids which, after delivery to a cell, express one or more polypeptides, especially a full length microbial protein, e.g., fused to ubiquitin as a fusion protein or RNA molecules), such as for a particular purpose, as an immunogenic material which may optionally include a further therapeutic application or a diagnostic application.

In some embodiments, the lipid bi-layer of the protocells can provide biocompatibility and can be modified to possess targeting species including, for example, targeting peptides including oligopeptides, antibodies, aptamers, and PEG (polyethylene glycol) (including PEG covalently linked to specific targeting species), among others, to allow, for example, further stability of the protocells and/or a targeted delivery into an antigen presenting cell (APC).

The protocell particle size distribution, according to the vaccine embodiment, depending on the application and biological effect, may be monodisperse or polydisperse. The silica cores can be rather monodisperse (i.e., a uniform sized population varying no more than about 5% In diameter e.g., ±10-nm for a 200 nm diameter protocell especially if they are prepared using solution techniques) or rather polydisperse (i.e., a polydisperse population can vary widely from a mean or medium diameter, e.g., up to ±200-nm or more if prepared by aerosol. Polydisperse populations can be sized into monodisperse populations. All of these are suitable for protocell formation. Protocells may be no more than about 500 nm in diameter, e.g., no more than about 200 nm in diameter in order to afford delivery to a patient or subject and produce an intended therapeutic effect. The pores of the protocells may vary in order to load plasmid DNA and/or other macromolecules into the core of the protocell. These may be varied pursuant to methods which are well known in the art.

Protocells according to the vaccine embodiment generally range in size from greater than about 8-10 nm to about 5 μm in diameter, about 20-nm-3 μm in diameter, about 10 nm to about 500 nm, or about 20-200-nm (including about 150 nm, which may be a mean or median diameter). As discussed above, the protocell population may be considered monodisperse or polydisperse based upon the mean or median diameter of the population of protocells. Size is very important to immunogenic aspects as particles smaller than about 8-nm diameter are excreted through kidneys, and those particles larger than about 200 nm are often trapped by the liver and spleen. Thus, an embodiment focuses in smaller sized protocells for drug delivery and diagnostics in the patient or subject.

Protocells according the vaccine embodiment are characterized by containing mesopores, e.g., pores which are found in the nanostructure material. These pores (at least one, but often a large plurality) may be found intersecting the surface of the nanoparticle (by having one or both ends of the pore appearing on the surface of the nanoparticle) or internal to the nanostructure with at least one or more mesopore interconnecting with the surface mesopores of the nanoparticle. Interconnecting pores of smaller size are often found internal to the surface mesopores. The overall range of pore size of the mesopores can be 0.03-50-nm in diameter. Pore sizes of mesopores range from about 2-30 nm: they can be monosized or bimodal or graded—they can be ordered or disordered (essentially randomly disposed or worm-like). As noted, larger pores are usually used for loading plasmid DNA and/or full length microbial protein which optionally comprises ubiquitin presented as a fusion protein.

Mesopores (IUPAC definition 2-50-nm in diameter) are ‘molded’ by templating agents including surfactants, block copolymers, molecules, macromolecules, emulsions, latex beads, or nanoparticles. In addition, processes could also lead to micropores (IUPAC definition less than 2-nm in diameter) all the way down to about 0.03-nm, e.g., If a templating moiety in the aerosol process is not used. They could also be enlarged to macropores, i.e., 50-nm in diameter.

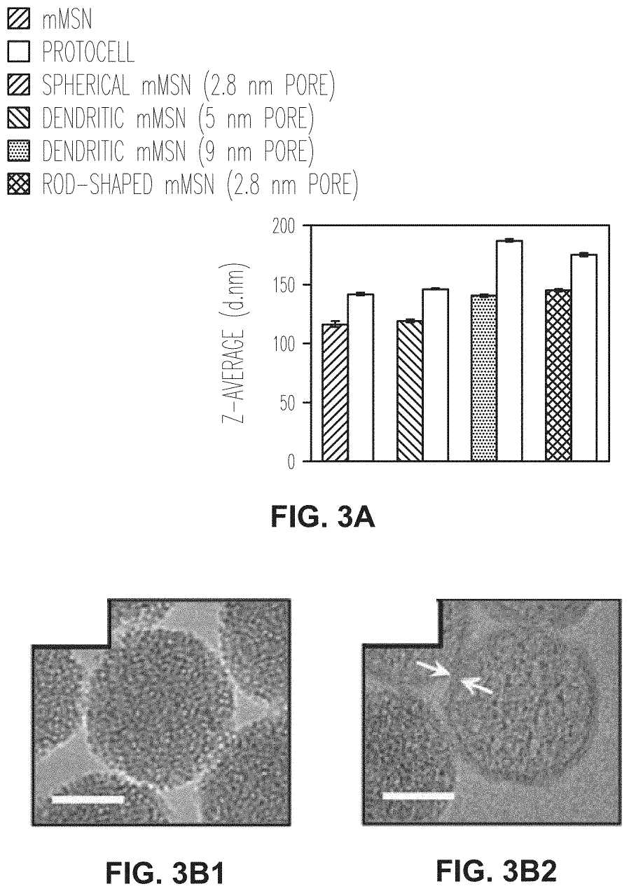

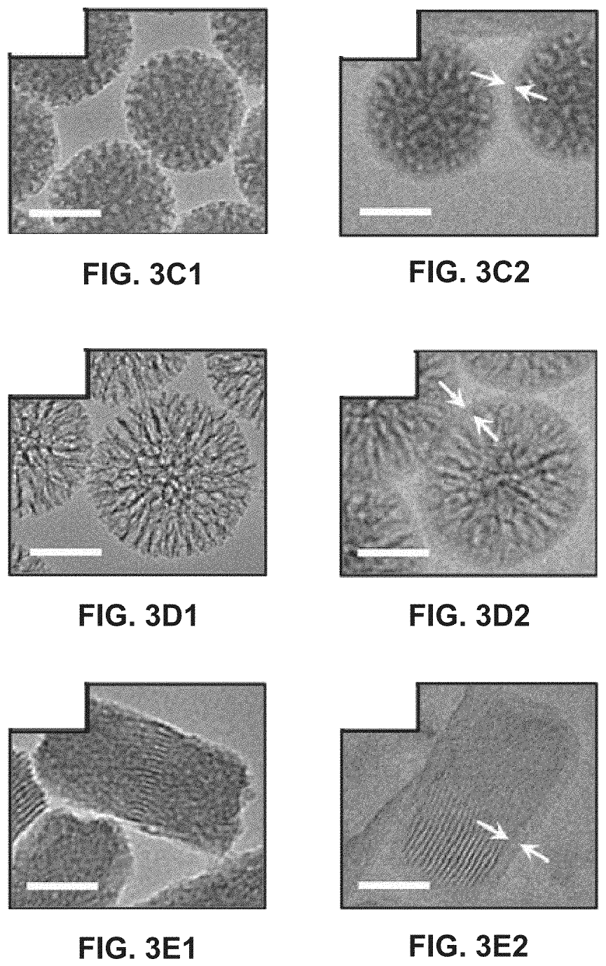

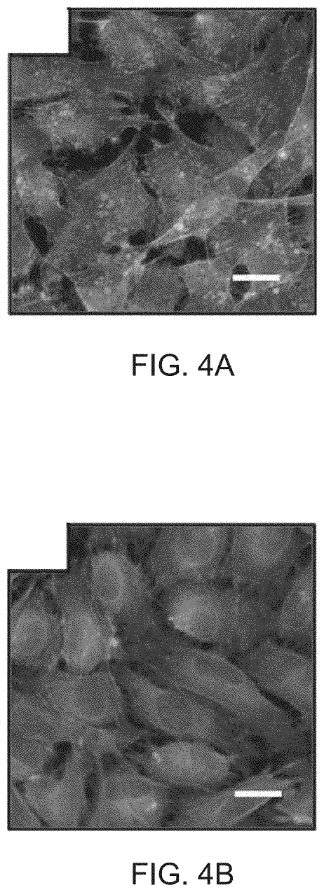

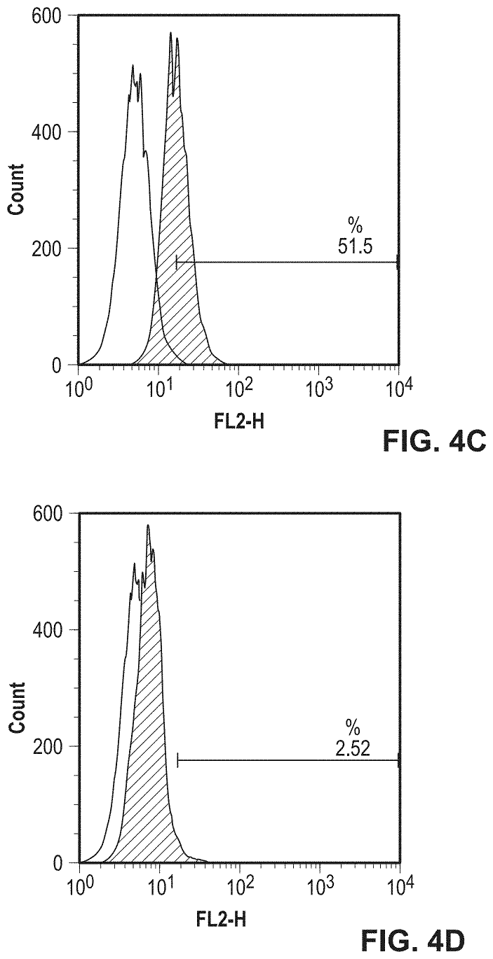

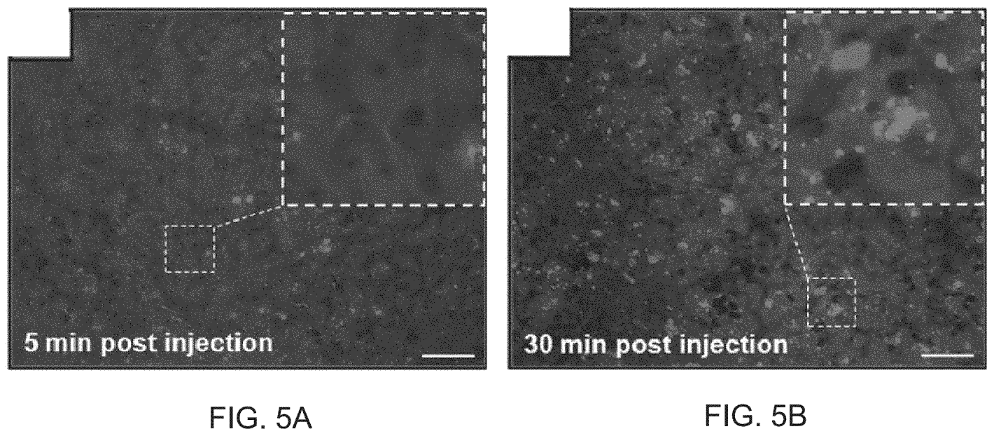

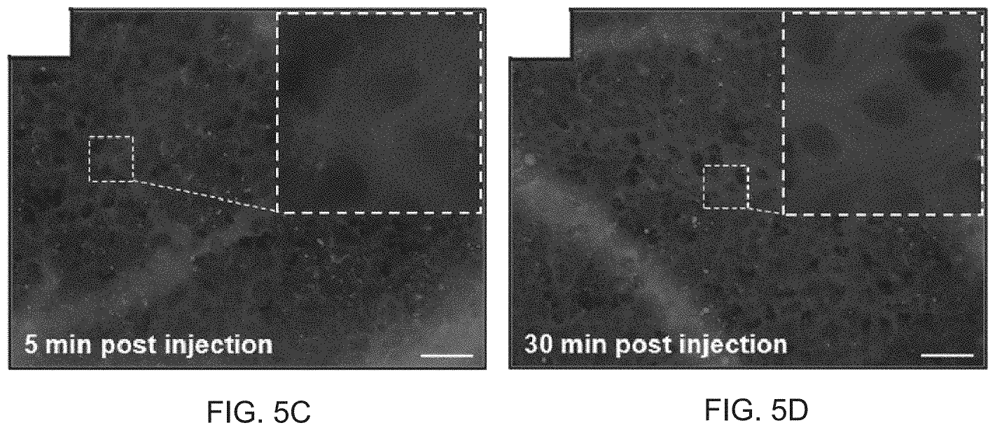

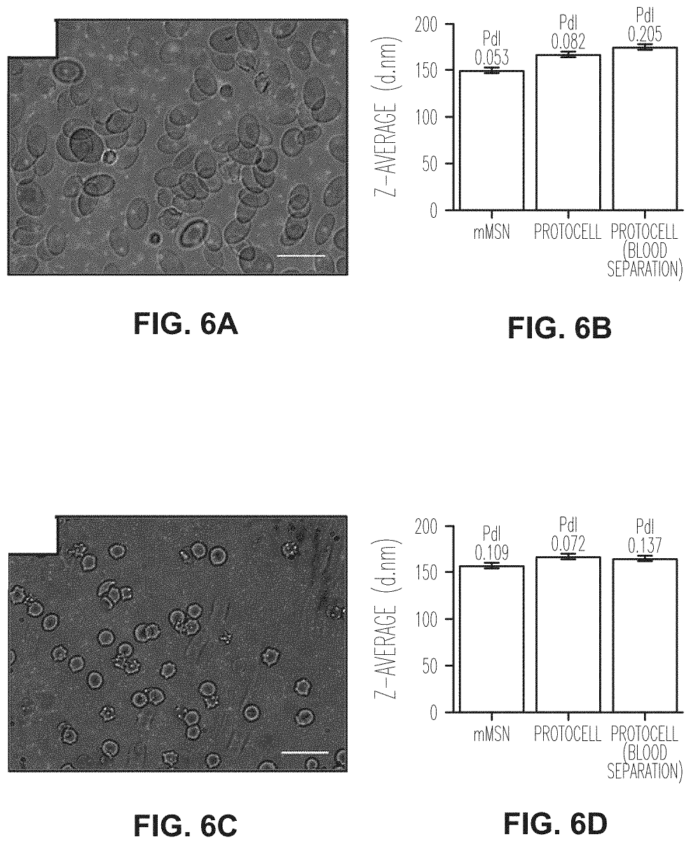

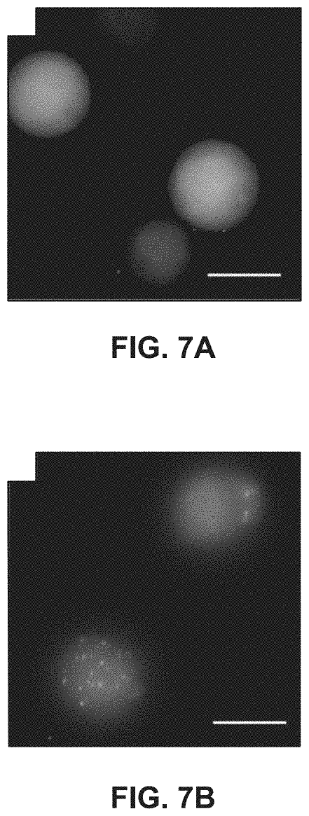

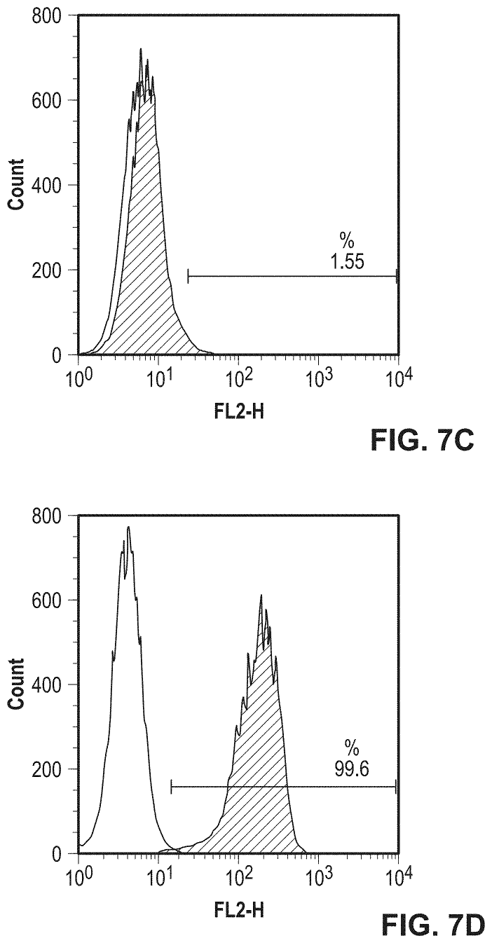

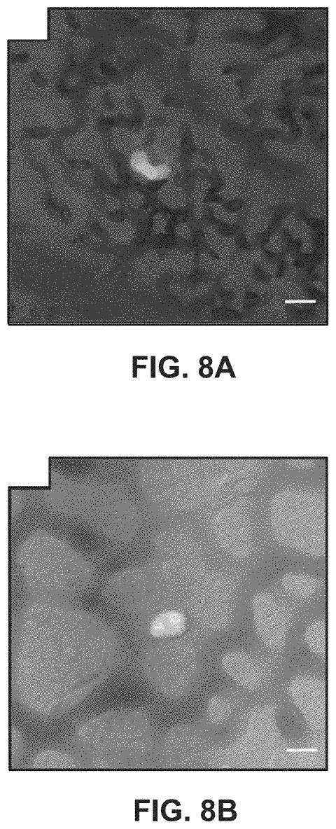

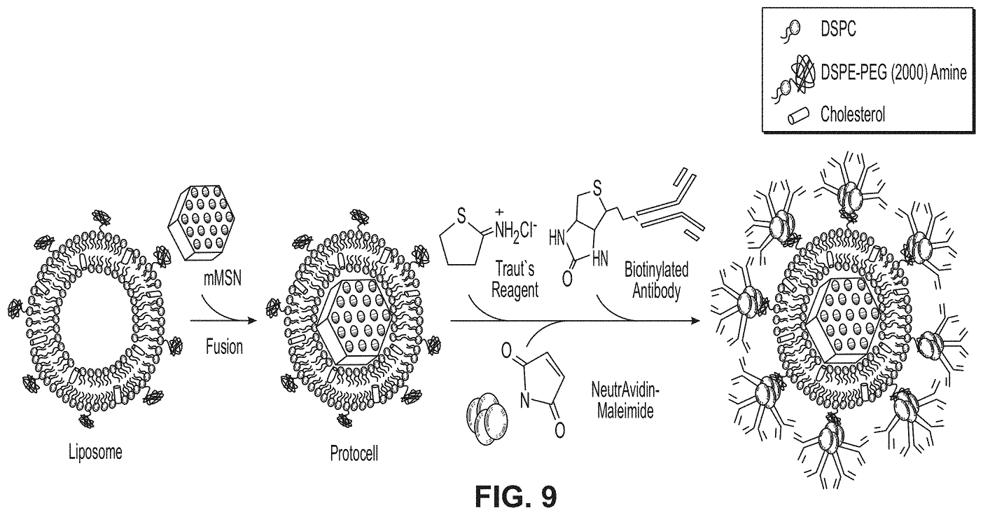

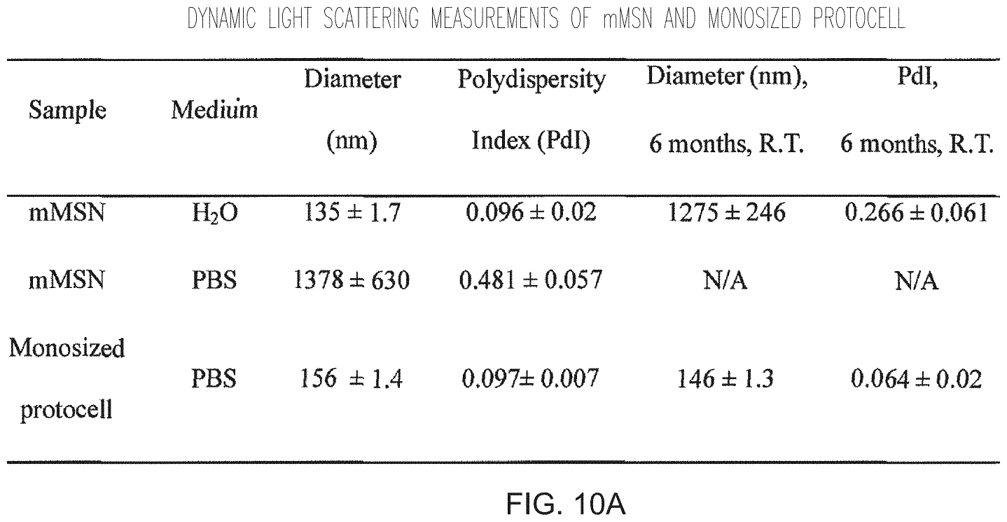

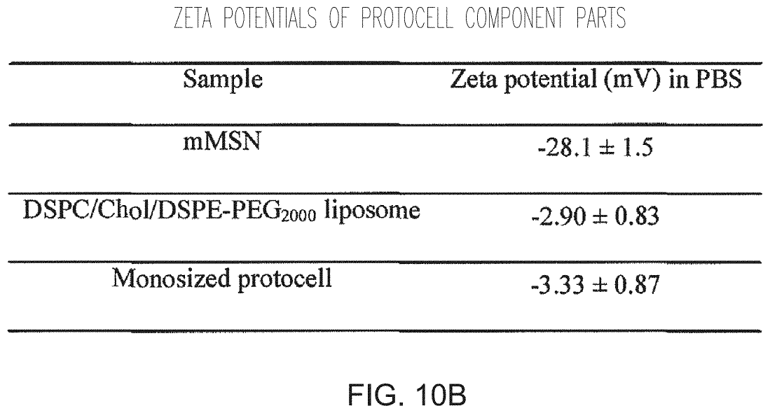

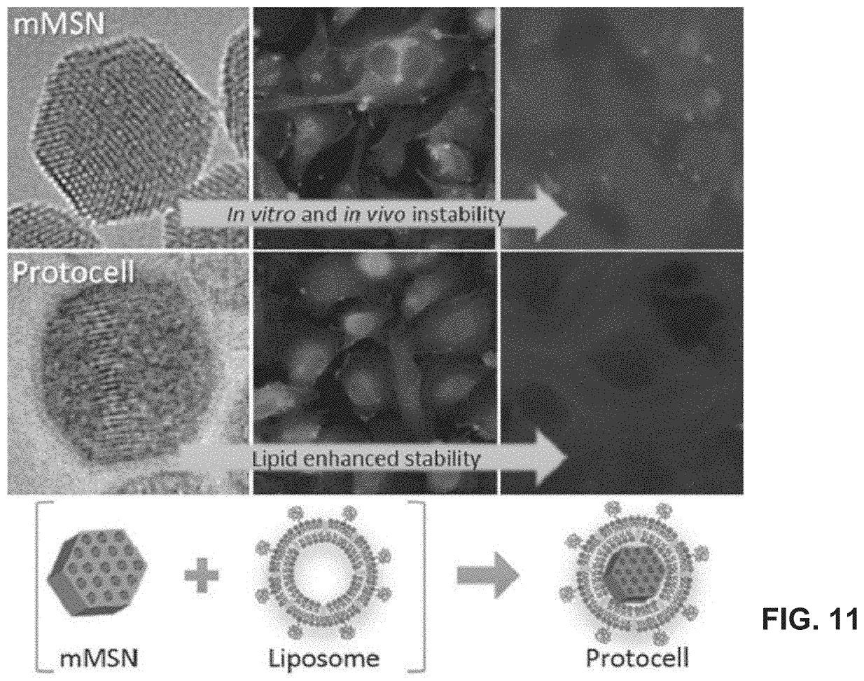

Pore surface chemistry of the nanoparticle material can be very diverse—all organosilanes yielding cationic, anionic, hydrophilic, hydrophobic, reactive groups—pore surface chemistry, especially charge and hydrophobicity, affect loading capacity. See FIG. 3, attached. Attractive electrostatic interactions or hydrophobic interactions control/enhance loading capacity and control release rates. Higher surface areas can lead to higher loadings of drugs/cargos through these attractive interactions, as further explained below.

The surface area of nanoparticles, as measured by the N₂ BET method, ranges from about 100 m²/g to >about 1200 m²/g. In general, the larger the pore size, the smaller the surface area. The surface area theoretically could be reduced to essentially zero, if one does not remove the templating agent or if the pores are sub-0.5-nm and therefore not measurable by N₂ sorption at 77K due to kinetic effects. However, in this case, they could be measured by CO₂ or water sorption, but would probably be considered non-porous. This would apply if biomolecules are encapsulated directly in the silica cores prepared without templates, in which case particles (Internal cargo) would be released by dissolution of the silica matrix after delivery to the cell.

Typically the protocells are loaded with cargo to a capacity up to about 50 weight %: defined as (cargo weight/weight of loaded protocell)×100. The optimal loading of cargo is often about 0.01 to 10% but this depends on the drug or drug combination which is incorporated as cargo into the protocell. This is generally expressed in μM of cargo per 10¹⁰ protocell particles with values ranging, for example, from 2000-100 μM per 10¹⁰ particles. Exemplary protocells exhibit release of cargo at pH about 5.5, which is that of the endosome, but are stable at physiological pH of 7 or higher (7.4).

The surface area of the internal space for loading is the pore volume whose value ranges from about 1.1 to 0.5 cubic centimeters per gram (cc/g). Note that in the protocells according to one embodiment, the surface area is mainly internal as opposed to the external geometric surface area of the nanoparticle.

The lipid bi-layer supported on the porous particle according to one embodiment has a lower melting transition temperature, i.e. is more fluid than a lipid bl-layer supported on a non-porous support or the lipid bi-layer in a liposome. This is sometimes important in achieving high affinity binding of targeting ligands at low peptide densities, as it is the bi-layer fluidity that allows lateral diffusion and recruitment of peptides by target cell surface receptors. One embodiment provides for peptides to cluster, which facilitates binding to a complementary target.

In some embodiments, the lipid bi-layer may vary significantly in composition. Ordinarily, any lipid or polymer which is may be used in liposomes may also be used in protocells. Exemplary lipids are as otherwise described herein. Particular lipid bi-layers for use in protocells comprise mixtures of lipids (as otherwise described herein).

The charge of the mesoporous silica NP core as measured by the Zeta potential may be varied monotonically from −50 to +50 mV by modification with the amine silane, 2-(aminoethyl) propyltrimethoxy-silane (AEPTMS) or other organosilanes. This charge modification, in turn, varies the loading of the drug within the cargo of the protocell. Generally, after fusion of the supported lipid bi-layer, the zeta-potential is reduced to between about −10 mV and +5 mV, which is important for maximizing circulation time in the blood and avoiding non-specific interactions.

Depending on how the surfactant template is removed, e.g., calcination at high temperature (500° C.) versus extraction in acidic ethanol, and on the amount of AEPTMS or other silica amine incorporated into the silica framework, the silica dissolution rates can be varied widely. This in turn controls the release rate of the internal cargo. This occurs because molecules that are strongly attracted to the internal surface area of the pores diffuse slowly out of the particle cores, so dissolution of the particle cores controls in part the release rate.

Further characteristics of protocells according to the vaccine are that they are stable at pH 7, i.e., they don't leak their cargo, but at pH 5.5, which is that of the endosome lipid or polymer coating becomes destabilized initiating cargo release. This pH-triggered release is important for maintaining stability of the protocell up until the point that it is internalized in the cell by endocytosis, whereupon several pH triggered events cause release into the endosome and consequently, the cytosol of the cell. Quantitative experimental evidence has shown that targeted protocells illicit only a weak immune response in the absence of the components which are incorporated into protocells, because they do not support T-Cell help required for higher affinity IgG, a favorable result.

Various embodiments provide nanostructures which are constructed from nanoparticles which support a lipid bi-layer(s). In embodiments according to the vaccine, the nanostructures may include, for example, a core-shell structure including a porous particle core surrounded by a shell of lipid bi-layer(s). The nanostructure, e.g., a porous silica nanostructure as described above, supports the lipid bi-layer membrane structure.

In some embodiments according to the vaccine, the lipid bi-layer of the protocells can provide biocompatibility and can be modified to possess targeting species including, for example, targeting peptides, antibodies, aptamers, and PEG (polyethylene glycol) linked to targeting species to allow, for example, further stability of the protocells and/or a targeted delivery into a bioactive cell, in particular an APC. PEG, when included in lipid bi-layers, can vary widely in molecular weight (although PEG ranging from about 10 to about 100 units of ethylene glycol, about 15 to about 50 units, about 15 to about 20 units, about 15 to about 25 units, about 16 to about 18 units, etc., may be used and the PEG component which is generally conjugated to phospholipid through an amine group comprises about 1% to about 20%, about 5% to about 15%, or about 10% by weight of the lipids which are included in the lipid bi-layer.

Numerous lipids which are used in liposome delivery systems may be used to form the lipid bi-layer on nanoparticles to provide protocells. Virtually any lipid or polymer which is used to form a liposome or polymersome may be used in the lipid bi-layer which surrounds the nanoparticles to form protocells according to an embodiment. Exemplary lipids for use include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol is included as a lipid. Often cholesterol is incorporated into lipid bi-layers of protocells in order to enhance structural integrity of the bi-layer. These lipids are all readily available commercially from Avanti Polar Lipids, Inc. (Alabaster, Ala., USA). DOPE and DPPE are particularly useful for conjugating (through an appropriate crosslinker) peptides, polypeptides, including antibodies, RNA and DNA through the amine group on the lipid.

In certain embodiments, the nanoparticulate cores can also be biodegradable polymer nanoparticulates comprising one or more compositions selected from the group consisting of aliphatic polyesters, poly (lactic acid) (PLA), poly (glycolic acid) (PGA), co-polymers of lactic acid and glycolic acid (PLGA), polycaprolactone (PCL), polyanhydrides, poly(ortho)esters, polyurethanes, poly(butyric acid), poly(valeric acid), poly(lactide-co-caprolactone), alginate and other polysaccharides, collagen, and chemical derivatives thereof, albumin, a hydrophilic protein, zein, a prolamine, a hydrophobic protein, and copolymers and mixtures thereof.

In still other embodiments, the protocells each comprise a core having a core surface that is essentially free of silica, and a shell attached to the core surface, wherein the core comprises a transition metal compound selected from the group consisting of oxides, carbides, sulfides, nitrides, phosphides, borides, halides, selenides, tellurides, tantalum oxide, iron oxide or combinations thereof.

The silica nanoparticles used in the protocells according to the vaccine can be, for example, mesoporous silica nanoparticles and core-shell nanoparticles. The nanoparticles may incorporate an absorbing molecule, e.g., an absorbing dye. Under appropriate conditions, the nanoparticles emit electromagnetic radiation resulting from chemiluminescence. Additional contrast agents may be included to facilitate contrast in MRI, CT, PET, and/or ultrasound imaging.

The cores can be, e.g., from around 5 nm to around 500 nm in size, including all integers and ranges there between. The size is measured as the longest axis of the particle. In various embodiments, the particles are from around 10 nm to around 500 nm and from around 10 nm to around 100 nm in size. In some embodiments, the cores have a porous structure. The pores can be from around 1 to around 20 nm in diameter, including all integers and ranges there between. In one embodiment, the pores are from around 1 to around 10 nm in diameter. In one embodiment, around 90% of the pores are from around 1 to around 20 nm in diameter. In another embodiment, around 95% of the pores are around 1 to around 20 nm in diameter.

In one embodiment, the cores are synthesized using so-gel methodology where a silica precursor or silica precursors and a silica precursor or silica precursors conjugated (i.e., covalently bound) to absorber molecules are hydrolyzed in the presence of templates in the form of micelles. The templates are formed using a surfactant such as, for example, hexadecyltrimethylammonium bromide (CTAB). It is expected that any surfactant which can form micelles can be used.

In certain embodiments, the core-shell nanoparticles comprise a core and shell. The core comprises silica and an optional absorber molecule. The absorber molecule is incorporated in to the silica network via a covalent bond or bonds between the molecule and silica network. The shell comprises silica.

In one embodiment, the core is independently synthesized using known sol-gel chemistry, e.g., by hydrolysis of a silica precursor or precursors. The silica precursors are present as a mixture of a silica precursor and a silica precursor conjugated, e.g., linked by a covalent bond, to an absorber molecule (referred to herein as a “conjugated silica precursor”). Hydrolysis can be carried out under alkaline (basic) conditions to form a silica core and/or silica shell. For example, the hydrolysis can be carried out by addition of ammonium hydroxide to the mixture comprising silica precursor(s) and conjugated silica precursor(s).

Silica precursors are compounds which under hydrolysis conditions can form silica. Examples of silica precursors include, but are not limited to, organosilanes such as, for example, tetraethoxysilane (TEOS), tetramethoxysilane (TMOS) and the like.

The silica precursor used to form the conjugated silica precursor has a functional group or groups which can react with the absorbing molecule or molecules to form a covalent bond or bonds. Examples of such silica precursors include, but are not limited to, Isocyanatopropyltriethoxysilane (ICPTS), aminopropyltrimethoxysilane (APTS), mercaptopropyltrimethoxysilane (MPTS), and the like.

In one embodiment, an organosilane (conjugatable silica precursor) used for forming the core has the general formula R₄ₙSiXₙ, where X is a hydrolyzable group such as ethoxy, methoxy, or 2-methoxy-ethoxy; R can be a monovalent organic group of from 1 to 12 carbon atoms which can optionally contain, but is not limited to, a functional organic group such as mercapto, epoxy, acrylyl, methacrylyl, or amino; and n is an integer of from 0 to 4. The conjugatable silica precursor is conjugated to an absorber molecule and subsequently co-condensed for forming the core with silica precursors such as, for example, TEOS and TMOS. A silane used for forming the silica shell has n equal to 4. The use of functional mono-, bis- and tris-alkoxysilanes for coupling and modification of co-reactive functional groups or hydroxy-functional surfaces, including glass surfaces, is also known, see Kirk-Othmer, Encyclopedia of Chemical Technology, Vol. 20, 3rd Ed., J. Wiley, N.Y.; see also E. Pluedemann, Silane Coupling Agents, Plenum Press, N.Y. 1982. The organo-silane can cause gels, so it may be desirable to employ an alcohol or other known stabilizers. Processes to synthesize core-shell nanoparticles using modified Stoeber processes can be found in U.S. patent application Ser. Nos. 10/306,614 and 10/536,569, the disclosure of such processes therein are incorporated herein by reference.

In certain embodiments of the vaccine, the lipid bi-layer is comprised of one or more lipids selected from the group consisting of phosphatidyl-cholines (PCs) and cholesterol.

In certain embodiments, the lipid bi-layer is comprised of one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), egg PC, and a lipid mixture comprising between about 50% to about 70%, or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-cholines, DMPC [14:0] having a carbon length of 14 and no unsaturated bonds, 1,2-dipalmitoyl-sn-glycero-3-phosphocholine (DPPC) [16:0], 1,2-distearoyl-sn-glycero-3-phosphocholine (DSPC) [18:0], 1,2-dioleoyl-sn-glycero-3-phosphocholine (DOPC) [18:1 (Δ9-Cis)], POPC [16:0-18:1], and DOTAP [18:1].

In other embodiments: (a) the lipid bi-layer is comprised of a mixture of (1) egg PC, and (2) one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), a lipid mixture comprising between about 50% to about 70% or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-choline, DMPC [14:0] having a carbon length of 14 and no unsaturated bonds, 1,2-dipalmitoyl-sn-glycero-3-phosphocholine (DPPC) [16:0], 1,2-distearoyl-sn-glycero-3-phosphocholine (DSPC) [18:0], 1,2-dioleoyl-sn-glycero-3-phosphocholine (DOPC) [18:1 (Δ9-Cis)], POPC [16:0-18:1] and DOTAP [18:1]; and wherein (b) the molar concentration of egg PC in the mixture is between about 10% to about 50% or about 11% to about 49%, or about 12% to about 48%, or about 13% to about 47%, or about 14% to about 46%, or about 15% to about 45%, or about 16% to about 44%, or about 17% to about 43%, or about 18% to about 42%, or about 19% to about 41%, or about 20% to about 40%, or about 21% to about 39%, or about 22% to about 38%, or about 23% to about 37%, or about 24% to about 36%, or about 25% to about 35%, or about 26% to about 34%, or about 27% to about 33%, or about 28% to about 32%, or about 29% to about 31%, or about 30%.

In certain embodiments, the lipid bi-layer is comprised of one or more compositions selected from the group consisting of a phospholipid, a phosphatidyl-choline, a phosphatidyl-serine, a phosphatidyl-diethanolamine, a phosphatidylinosite, a sphingolipid, and an ethoxylated sterol, or mixtures thereof. In illustrative examples of such embodiments, the phospholipid can be a lecithin; the phosphatidylinosite can be derived from soy, rape, cotton seed, egg and mixtures thereof; the sphingolipid can be ceramide, a cerebroside, a sphingosine, and a sphingomyelin, and a mixture thereof; the ethoxylated sterol can be phytosterol, PEG-(polyethyleneglycol)-5-soy bean sterol, and PEG-(polyethyleneglycol)-5 rapeseed sterol. In certain embodiments, the phytosterol comprises a mixture of at least two of the following compositions: sitosterol, campesterol and stigmasterol.

In still other illustrative embodiments, the lipid bi-layer is comprised of one or more phosphatidyl groups selected from the group consisting of phosphatidyl choline, phosphatidyl-ethanolamine, phosphatidyl-serine, phosphatidyl-inositol, lyso-phosphatidyl-choline, lyso-phosphatidyl-ethanolamine, lyso-phosphatidyl-inositol and lyso-phosphatidyl-inositol.

In still other illustrative embodiments, the lipid bi-layer is comprised of phospholipid selected from a monoacyl or diacylphosphoglyceride.

In still other illustrative embodiments, the lipid bi-layer is comprised of one or more phosphoinositides selected from the group consisting of phosphatidyl-inositol-3-phosphate (PI-3-P), phosphatidyl-inositol-4-phosphate (PI-4-P), phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-Inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidyl-inositol-3,4,5-triphosphate (LPI-3,4,5-P3), and phosphatidyl-inositol (PI), and lysophosphatidyl-inositol (LPI).

In still other illustrative embodiments, the lipid bi-layer is comprised of one or more phospholipids selected from the group consisting of PEG-poly(ethylene glycol)-derivatized distearoylphosphatidylethanolamine (PEG-DSPE), poly(ethylene glycol)-derivatized ceramides (PEG-CER), hydrogenated soy phosphatidylcholine (HSPC), egg phosphatidylcholine (EPC), phosphatidyl ethanolamine (PE), phosphatidyl glycerol (PG), phosphatidyl inositol (PI), monosialoganglioside, sphingomyelin (SPM), distearoylphosphatidylcholine (DSPC), dimyristoylphosphatidylcholine (DMPC), and dimyristoylphosphatidylglycerol (DMPG).

In one embodiment of the vaccine a protocell which is included in compositions may include at least one anti-cancer agent, especially an anti-cancer agent which treats a cancer which occurs secondary to a viral infection.

One illustrative embodiment of a protocell of the vaccine comprises a plurality of negatively-charged, nanoporous, nanoparticulate silica cores that: (a) are modified with an amine-containing silane selected from the group consisting of (1) a primary amine, a secondary amine a tertiary amine, each of which is functionalized with a silicon atom (2) a monoamine or a polyamine (3) N-(2-aminoethyl)-3-aminopropyltrimethoxysilane (AEPTMS) (4) 3-aminopropyltrimethoxysilane (APTMS) (5) 3-aminopropyltriethoxysilane (APTS) (6) an amino-functional trialkoxysilane, and (7) protonated secondary amines, protonated tertiary alkyl amines, protonated amidines, protonated guanidines, protonated pyridines, protonated pyrimidines, protonated pyrazines, protonated purines, protonated imidazoles, protonated pyrroles, and quaternary alkyl amines, or combinations thereof; and (b) are encapsulated by and that support a lipid bi-layer comprising one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycero) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-s-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-y)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof, and wherein the lipid bi-layer comprises a cationic lipid and one or more zwitterionic phospholipids.

Protocells can comprise a wide variety of pharmaceutically-active ingredients.

In certain embodiments, the protocells according to the vaccine may include a reporter for diagnosing a disease state or condition. The term “reporter” is used to describe an imaging agent or moiety which is incorporated into the phospholipid bi-layer or cargo of protocells according to an embodiment and provides a signal which can be measured. The moiety may provide a fluorescent signal or may be a radioisotope which allows radiation detection, among others. Exemplary fluorescent labels for use in protocells (e.g., via conjugation or adsorption to the lipid bi-layer or silica core, although these labels may also be incorporated into cargo elements such as DNA, RNA, polypeptides and small molecules which are delivered to cells by the protocells, include Hoechst 33342 (350/461), 4′,6-diamidino-2-phenylindole (DAPI, 356/451), Alexa Fluor® 405 carboxylic acid, succinimidyl ester (401/421), CellTracker™ Violet BMQC (415/516), CellTracker™ Green CMFDA (492/517), calcein (495/515), Alexa Fluor® 488 conjugate of annexin V (495/519), Alexa Fluor® 488 goat anti-mouse IgG (H+L) (495/519), Click-iT® AHA Alexa Fluor® 488 Protein Synthesis HCS Assay (495/519), LIVE/DEAD® Fixable Green Dead Cell Stain Ki (495/519), SYTOX® Green nucleic acid stain (504/523), MitoSOX™ Red mitochondrial superoxide indicator (510/580). Alexa Fluor® 532 carboxylic acid, succinimidyl ester(532/554), pHrodo™ succinimidyl ester (558/576), CellTracker™ Red CMTPX (577/602), Texas Red® 1,2-dihexadecanoyl-sn-glycero-3-phosphoethanolamine (Texas Red® DHPE, 583/608), Alexa Fluor® 647 hydrazide (649/666), Alexa Fluor® 647 carboxylic acid, succinimidyl ester (650/668), Ulysis™ Alexa Fluor® 647 Nucleic Acid Labeling Kit (650/670) and Alexa Fluor® 647 conjugate of annexin V (650/665). Moieties which enhance the fluorescent signal or slow the fluorescent fading may also be incorporated and include SlowFade® Gold antifade reagent (with and without DAPI) and Image-iT® FX signal enhancer. All of these are well known in the art. Additional reporters include polypeptide reporters which may be expressed by plasmids (such as histone-packaged supercoiled DNA plasmids) and include polypeptide reporters such as fluorescent green protein and fluorescent red protein. Reporters are utilized principally in diagnostic applications including diagnosing the existence or progression of a disease state in a patient and or the progress of therapy in a patient or subject.

The term “histone-packaged supercoiled plasmid DNA” is used to describe a y component of protocells which utilize an exemplary plasmid DNA which has been “supercoiled” (i.e., folded in on itself using a supersaturated salt solution or other ionic solution which causes the plasmid to fold in on itself and “supercoil” in order to become more dense for efficient packaging into the protocells). The plasmid may be virtually any plasmid which expresses any number of polypeptides or encode RNA, including small hairpin RNA/shRNA or small interfering RNA/siRNA, as otherwise described herein. Once supercoiled (using the concentrated salt or other anionic solution), the supercoiled plasmid DNA is then complexed with histone proteins to produce a histone-packaged “complexed” supercoiled plasmid DNA.

“Packaged” DNA herein refers to DNA that is loaded into protocells (either adsorbed into the pores or confined directly within the nanoporous silica core itself). To minimize the DNA spatially, it is often packaged, which can be accomplished in several different ways, from adjusting the charge of the surrounding medium to creation of small complexes of the DNA with, for example, lipids, proteins, or other nanoparticles (usually, although not exclusively cationic). Packaged DNA is often achieved via lipoplexes (i.e., complexing DNA with cationic lipid mixtures). In addition, DNA has also been packaged with cationic proteins (including proteins other than histones), as well as gold nanoparticles (e.g., NanoFlares—an engineered DNA and metal complex in which the core of the nanoparticle is gold).

Any number of histone proteins, as well as other means to package the DNA into a smaller volume such as normally cationic nanoparticles, lipids, or proteins, may be used to package the supercoiled plasmid DNA “histone-packaged supercoiled plasmid DNA”, but in therapeutic aspects which relate to treating human patients, the use of human histone proteins is envisioned. In certain aspects, a combination of human histone proteins H1, H2A, H2B, H3 and H4 in an exemplary ratio of 1:2:2:2:2, although other histone proteins may be used in other, similar ratios, as is known in the art or may be readily practiced. The DNA may also be double stranded linear DNA, instead of plasmid DNA, which also may be optionally supercoiled and/or packaged with histones or other packaging components.

Other histone proteins which may be used in this aspect include, for example, H1F, H1F0, H1FNT, H1FOO, H1FX, H1H1, HIST1HA, HIST1H1B, HIST1H1C, HIST1HID, HIST1H1E, HIST1H1T, H2AF, H2AFB1, H2AFB2, H2AFB3, H2AFJ, H2AFV, H2AFX, H2AFY, H2AFY2, H2AFZ, H2A1, HIST1H2AA, HIST1H2AB, HIST1H2AC, HIST1H2AD, HIST1H2AE, HIST1H2AG, HIST1H2AI, HIST1H2AJ, HIST1H2AK, HIST1H2AL, HIST1H2AM, H2A2, HIST2H2AA3, HIST2H2AC, H2BF, H2BFM, HSBFS, HSBFWT, H2B1, HIST1H2BA, HIST1HSBB, HIST1HSBC, HIST1HSBD, HIST1H2BE, HIST1H2BF, HIST1H2BG, HIST1H2BH, HIST1H2BI, HIST1H2BJ, HIST1H2BK, HIST1H2BL, HIST1H2BM, HIST1H2BN, HIST1H2BO, H2B2, HIST2H2BE, H3A1, HIST1H3A, HIST1H3B, HIST11H3C, HIST1H3D, HIST1H3E, HIST1H3F, HIST1H3G, HIST1H3H, HIST1H3I, HIST1H3J, H3A2, HIST2H3C, H3A3, HIST3H3, H41, HIST1H4A, HIST1H4B, HIST1H4C, HIST1H4D, HIST1H4E, HIST1H4F, HIST1H4G, HIST1H4H, HIST1H41, HIST1H4J, HIST1H4K, HIST1H4L, H44 and HIST4H4.

In certain embodiments, protocells comprise a plasmid (which may be a histone-packaged supercoiled plasmid DNA) which encodes a microbial protein, e.g., viral protein, antigen often complexed with ubiquitin protein (e.g., as a fusion protein). The plasmid, including a histone-packaged supercoiled plasmid DNA, may be modified (crosslinked) with a nuclear localization sequence (note that the histone proteins may be crosslinked with the nuclear localization sequence or the plasmid itself can be modified to express a nuclear localization sequence) in order to enhance the ability of the histone-packaged plasmid to penetrate the nucleus of a cell and deposit its contents there (to facilitate expression and ultimately cell death). These peptide sequences assist in carrying the histone-packaged plasmid DNA and the associated histones into the nucleus of a cell to facilitate expression and antigen presentation. Any number of crosslinking agents, well known in the art and as otherwise described herein, may be used to covalently link a nuclear localization sequence to a histone protein (often at a lysine group or other group which has a nucleophilic or electrophilic group in the side chain of the amino acid exposed pendant to the polypeptide) which can be used to introduce the histone packaged plasmid into the nucleus of a cell. Alternatively, a nucleotide sequence which expresses the nuclear localization sequence can be positioned in a plasmid in proximity to that which expresses histone protein such that the expression of the histone protein conjugated to the nuclear localization sequence win occur thus facilitating transfer of a plasmid into the nucleus of a targeted cell. In alternative embodiments, the DNA plasmid is included in the absence of histone packaging and/or a nuclear localization sequence and the plasmid expresses a microbial protein (e.g., full length viral protein) in the cytosol of the cell (APC) to which the protocell is delivered.

Proteins gain entry into the nucleus through the nuclear envelope. The nuclear envelope consists of concentric membranes, the outer and the inner membrane. These are the gateways to the nucleus. The envelope consists of pores or large nuclear complexes. A protein translated with a NLS will bind strongly to importin (aka karyopherin), and together, the complex will move through the nuclear pore. Any number of nuclear localization sequences may be used to introduce histone-packaged plasmid DNA into the nucleus of a cell. Exemplary nuclear localization sequences include H2N-GNQSSNFGPMKGGNFGGRSSGPYGGGGQYFAKPRNQGGYGGC-COOH (SEQ ID NO: 22), RRMKWKK (SEQ ID NO:23), PKKKRKV (SEQ ID NO:24), and KR[PAATKKAGQA]KKKK (SEQ ID NO:25), the NLS of nucleoplasmin, a prototypical bipartite signal comprising two clusters of basic amino acids, separated by a spacer of about 10 amino acids. Numerous other nuclear localization sequences are well known in the art. See, for example, LaCasse et al., 1995; Weis, 1998 and Murat Cokol et al., “Finding nuclear localization signals”, at the website ubic.bloc.columbia.edu/papers/2000 nls/paper.html#tab2.

Viruses that may raise an immunogenic response include any viral bloagent which is an animal virus. Viruses which affect animals, include, for example. Papovaviruses, e.g., polyoma virus and SV40: Poxviruses, e.g., vaccinia virus and variola (smallpox); Adenoviruses, e.g., human adenovirus; Herpesviruses, e.g., Human Herpes Simplex types I and II; Parvoviruses, e.g., adeno associated virus (AAV); Reoviruses, e.g., rotavirus and reovirus of humans; Picomaviruses, e.g., poliovirus; Togaviruses, including the alpha viruses (group A), e.g., Sindbis virus and Semliki forest virus (SFV) and the flaviviruses (group B), e.g., dengue virus, yellow fever virus and the St. Louis encephalitis virus; Retroviruses, e.g., HIV I and II, Rous sarcoma virus (RSV), and mouse leukemia viruses; Rhabdoviruses, e.g., vesicular stomatitis virus (VSV) and rabies virus; Paramyxoviruses, e.g., mumps virus, measles virus and Sendai virus; Arena viruses, e.g., lassa virus: Bunyaviruses, e.g., bunyamwera (encephalitis); Coronaviruses, e.g., common cold, GI distress viruses, Orthomyxovirus, e.g., influenza; Calicviruses, e.g., Norwalk virus, Hepatitis E virus; Filoviruses, e.g., ebola virus and Marburg virus; and Astroviruses, e.g., astrovirus, among others.

Virus such as Sin Nombre virus, Nipah virus, Influenza (especially H5N1 influenza), Herpes Simplex Virus (HSV1 and HSV-2), Coxsackie virus, Human immunodeficiency virus (I and II), Andes virus, Dengue virus, Papilloma, Epstein-Barr virus (mononucleosis). Variola (smallpox) and other pox viruses and West Nile virus, among numerous others viruses.

A short list of animal viruses which are particularly relevant includes the following viruses: Reovirus, Rotavirus, Enterovirus, Rhinovirus, Hepatovirus, Cardiovirus, Aphthovirus, Parechovirus, Erbovirus, Kobuvirus, Teschovirus, Norwalk virus, Hepatitis E virus, Rubella virus, Lymphocytic choriomeningitis virus, HIV-1, HIV-2, HTLV (especially HTLV-1), Herpes Simplex Virus 1 and 2, Sin Nombre virus, Nipah virus, Coxsackie Virus, Dengue virus, Yellow fever virus, Hepatitis A virus, Hepatitis B virus, Hepatitis C virus, Influenza virus A, B and C, Isavirus, Thogotovirus, Measles virus, Mumps virus, Respiratory syncytial virus, California encephalitis virus, Hantavirus, Rabies virus, Ebola virus, Marburg virus, Corona virus. Astrovirus, Borna disease virus, and Variola (smallpox virus).

In certain embodiments, compositions may include protocells which contain an anti-cancer agent as a co-therapy, but principally as a separate distinguishable population from immunogenic protocells otherwise described herein. In such an embodiment, protocells which target cancer cells and which contain an anti-cancer agent may be co-administered with immunogenic protocells.

APCs fall into two categories: professional and non-professional. T cells cannot recognize or respond to “free” antigen. Recognition by T cells occurs when an antigen has been processed and presented by APCs via carrier molecules like MHC and CD1 molecules. Most cells in the body can present antigen to CD8⁺ T cells via MHC class I molecules and, thus, act as “APCs”; however, the term is often limited to specialized cells that can prime T cells (i.e., activate a T cell that has not been exposed to antigen), termed a naive T cell. These professional APCs, in general, express MHC class II as well as MHC class I molecules, and can stimulate CD4+ “helper” T-cells as well as CD8+ “cytotoxic” T cells respectively. The cells that express MHC class II molecules are often referred to as professional antigen-presenting cells an include dendritic cells (DCs), macrophages, B-cells which express a B cell receptor (BCR) and specific antibody which binds to the BCR and certain activated epithelial cells. Professional APCs internalize antigens, generally by phagocytosis or by receptor-mediated endocytosis and then display a fragment of the antigen on the membrane surface of the cell through its binding to a class II MHC molecule. Non-professional APCs do not express the Major Histocompatibility Complex class II (MHC class II) proteins required for interaction with naive T cells: these are only expressed upon stimulation of the non-professional APC by cytokines such as IFN-γ. All nucleated cells express MHC class I molecules and consequently all are considered non-professional APCs. Erythrocytes do not have a nucleus; consequently, they are one of the few cells in the body that cannot display antigens.

Compositions provide their principal immunological reaction through interaction with either professional APCs or non-professional APCs. Non-professional antigen presenting cells include virally infected cells and cancer cells.

In order to covalently link any of the fusogenic peptides or endosomolytic peptides to components of the lipid bi-layer, various approaches, well known in the art may be used. For example, the peptides listed above could have a C-terminal poly-His tag, which would be amenable to Ni-NTA conjugation (lipids commercially available from Avanti). In addition, these peptides could be terminated with a C-terminal cysteine for which heterobifunctional crosslinker chemistry (EDC, SMPH, etc.) to link to aminated lipids would be useful. Another approach is to modify lipid constituents with thiol or carboxylic acid to use the same crosslinking strategy. All known crosslinking approaches to crosslinking peptides to lipids or other components of a lipid layer could be used. In addition we could use click chemistry to modify the peptides with azide or alkyne for cu-catalyzed crosslinking, and we could also use a cu-free click chemistry reaction.

The plasmids described herein are used to express a microbial antigen (e.g., a viral protein). Optionally the antigen is in combination with ubiquitin as a fusion protein. In some embodiments, the plasmid vectors are adenoviral, lentiviral and/or retroviral vectors many, of which may readily accommodate the viral protein. Exemplary recombinant adenovirus vectors include those commercialized as the AdEasy™ System by many companies including Stratagene® (stratagene.com), QBiogene® (qbiogene.com), and the ATCC® (atcc.org). AdEasy™ vectors include pShuttle, pShuttle-CMV, and pAdEasy-1. The pAdEasy-1 vector is devoid of E1 and E3 regions so that the recombinant virus will not replicate in cells other than complementing cells, such as human embryonic kidney 293 (HEK293). These methods are described by He et al., Proc. Nat. Acad. Sci., USA, 95, pp. 2509-2514 (1998). An exemplary lentiviral expression system is the The ViraPower™ Lentiviral Expression System (Invitrogen, Carlsbad, Calif. 92008, invitrogen.com) is loosely based on the HIV-1 strain NL4-3. Other commercial adenoviral, lentiviral and retroviral vectors are well known in the art.

The crystal structure of ubiquitin evidences two accessible lysine groups which are used with the crosslinker chemistry described above to anchor the ubiquitin to a component (e.g., viral protein or peptide or a lipid, phospholipid, other) of a lipid bi-layer of the protocell. Ubiquitination does not have to occur in any specific part of the target peptide, it only acts as a marker to signal degradation. This is only intended to speed up the process; the cell would ubiquitinate a foreign peptide naturally delivering ubiquitinated microbial antigens potentially skip this step and speed up the process. Accordingly, ubiquitin is an optional element of the protocells.

As discussed in detail above, the porous nanoparticle core of the vaccine can include porous nanoparticles having at least one dimension, for example, a width or a diameter of about 3000 nm or less, about 1000 nm or less, about 500 nm or less, about 200 nm or less. In one embodiment, the nanoparticle core is spherical with an exemplary diameter of about 500 nm or less, e.g., about 8-10 nm to about 200 nm. In embodiments, the porous particle core can have various cross-sectional shapes including a circular, rectangular, square, or any other shape. In certain embodiments, the porous particle core can have pores with a mean pore size ranging from about 2 nm to about 30 nm, although the mean pore size and other properties (e.g., porosity of the porous particle core) are not limited in accordance with various embodiments of the present teachings.

In general, protocells according to the vaccine are biocompatible. Drugs and other cargo components are often loaded by adsorption and/or capillary filing of the pores of the particle core up to approximately 50% by weight of the final protocell (containing all components). In certain embodiments, the loaded cargo can be released from the porous surface of the particle core (mesopores), wherein the release profile can be determined or adjusted by, for example, the pore size, the surface chemistry of the porous particle core, the pH value of the system, and/or the interaction of the porous particle core with the surrounding lipid bi-layer(s) as generally described herein.

In the vaccine, the porous nanoparticle core used to prepare the protocells can be tuned in to be hydrophilic or progressively more hydrophobic as otherwise described herein and can be further treated to provide a more hydrophilic surface. For example, mesoporous silica particles can be further treated with ammonium hydroxide and hydrogen peroxide to provide a higher hydrophilicity. In exemplary aspects, the lipid bi-layer is fused onto the porous particle core to form the protocell. Protocells can include various lipids in various weight ratios, including 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-yl)amino]lauroyl]-sn-glycero-3-phosphocholine (18:1-12:0 NBD PC), 1-palmitoyl-2-{12-[(7-nitro-2-1,3-benzoxadiazol-4-y)amino]lauroyl}-sn-glycero-3-phosphocholine (16:0-12:0 NBD PC), cholesterol and mixtures/combinations thereof.

The lipid bi-layer which is used to prepare protocells can be prepared, for example, by extrusion of hydrated lipid films containing other components through a filter with pore size of, for example, about 100 nm, using standard protocols known in the art or as otherwise described herein. The filtered lipid bi-layer films can then be fused with the porous particle cores, for example, by pipette mixing. In certain embodiments, excess amount of lipid bi-layer or lipid bi-layer films can be used to form the protocell in order to improve the protocell colloidal stability.

In various embodiments, the protocell is used in a synergistic system where the lipid bi-layer fusion or liposome fusion (i.e., on the porous particle core) is loaded and sealed with various cargo components with the pores (mesopores) of the particle core, thus creating a loaded protocell useful for cargo delivery across the cell membrane of the lipid bi-layer or through dissolution of the porous nanoparticle, if applicable. In certain embodiments, in addition to fusing a single lipid (e.g., phospholipids) bi-layer, multiple bi-layers with opposite charges can be successively fused onto the porous particle core to further influence cargo loading and/or sealing as well as the release characteristics of the final protocell.

A fusion and synergistic loading mechanism can be included for cargo delivery. For example, cargo can be loaded, encapsulated, or sealed, synergistically through liposome fusion on the porous particles. In addition to microbial proteins, fusion proteins (e.g., viral proteins, including full length viral proteins and fusion proteins based upon viral proteins and ubiquitin) and/or plasmid vectors which can express microbial protein or microbial protein fused with ubiquitin. The cargo can also include, for example, small molecule drugs (e.g., especially including anti-cancer drugs and/or anti-viral drugs such as anti-HBV or anti-HCV drugs), peptides, proteins, antibodies, DNA (other plasmid DNA, RNAs (including shRNA and siRNA (which may also be expressed by plasmid DNA incorporated as cargo within the protocells), fluorescent dyes, including fluorescent dye peptides which may be expressed by the plasmid DNA incorporated within the protocell as reporters for diagnostic methods associated with establishing the mechanism of immunogenicity of protocells.

Loading of plasmid within the porous core may be difficult to achieve. One approach is to synthesize large pore particles; however, it is somewhat likely that the plasmid will interact with the exterior of the MSNP core regardless of pore size. Therefore, modification of the MSNP framework to incorporate cationic amine groups to form the core as described above will enhance the plasmid/MSNP association due to electrostatic attraction (plasmid carries a net negative charge). Another approach would be to incorporate a small amount of cationic lipids (DOPE, DPPE, DSPE, DOTAP, etc.) into the bi-layer formulation to encourage plasmid/MSNP association.

Protein cargo loading can be electrostatically driven, cationic cores/net negative protein or anionic cores/net positive protein. It is possible to conjugate the protein to the MSNP core using the previously mentioned conjugation strategies by modifying the core with amine, carboxylic acid, thiol, click chemistry, etc. We can also make better use of the pores since protein should be much smaller and more compact than the plasmid constructs. Another approach is to digest the protein into smaller pieces and load the particle with fragments of the protein.

In some embodiments, the cargo can be loaded into the pores (mesopores) of the porous particle cores to form the loaded protocell. In various embodiments, any conventional technology that is developed for liposome-based drug delivery, for example, targeted delivery using PEGylation, can be transferred and applied to the protocells.

As discussed above, electrostatics and pore size can play a role in cargo loading. For example, porous silica nanoparticles can carry a negative charge and the pore size can be tunable from about 2 nm to about 10 nm or more. Negatively charged nanoparticles can have a natural tendency to adsorb positively charged molecules and positively charged nanoparticles can have a natural tendency to adsorb negatively charged molecules. In various embodiments, other properties such as surface wettability (e.g., hydrophobicity) can also affect loading cargo with different hydrophobicity.

In various embodiments, the cargo loading can be a synergistic lipid-assisted loading by tuning the lipid composition. For example, if the cargo component is a negatively charged molecule, the cargo loading into a negatively charged silica can be achieved by the lipid-assisted loading. In certain embodiments, for example, a negatively charged species can be loaded as cargo into the pores of a negatively charged silica particle when the lipid bi-layer is fused onto the silica surface showing a fusion and synergistic loading mechanism. In this manner, fusion of a non-negatively charged (i.e., positively charged or neutral) lipid bi-layer or liposome on a negatively charged mesoporous particle can serve to load the particle core with negatively charged cargo components. The negatively charged cargo components can be concentrated in the loaded protocell having a concentration exceed about 100 times as compared with the charged cargo components in a solution. In other embodiments, by varying the charge of the mesoporous particle and the lipid bi-layer, positively charged cargo components can be readily loaded into protocells.

Once produced, the loaded protocells can have a cellular uptake for cargo delivery into a desirable site after administration. For example, the cargo-loaded protocells can be administered to a patient or subject and the protocell comprising a targeting peptide can bind to a target cell and be internalized by the target cell, for example, an APC in a subject or patient. Due to the internalization of the cargo-loaded protocells in the target cell, cargo components can then be delivered into the target cells. In certain embodiments the cargo is a small molecule, which can be delivered directly into the target cell for therapy. In other embodiments, negatively charged DNA or RNA (including shRNA or siRNA), especially including a DNA plasmid which may be formulated as histone-packaged supercoiled plasmid DNA, e.g., modified with a nuclear localization sequence, can be directly delivered or internalized by the targeted cells. Thus, the DNA or RNA can be loaded first into a protocell and then into then through the target cells through the internalization of the loaded protocells.

As discussed, the cargo loaded into and delivered by the protocell to targeted cells includes small molecules or drugs (especially anti-cancer or anti-HBV and/or anti-HCV agents), bioactive macromolecules (bioactive polypeptides such as ricin toxin A-chain or diphtheria toxin A-chain or RNA molecules such as shRNA and/or siRNA as otherwise described herein) or histone-packaged supercoiled plasmid DNA which can express a therapeutic or diagnostic peptide or a therapeutic RNA molecule such as shRNA or siRNA, wherein the histone-packaged supercoiled plasmid DNA is optionally modified with a nuclear localization sequence which can localize and concentrate the delivered plasmid DNA into the nucleus of the target cell. As such, loaded protocells can deliver their cargo into targeted cells for therapy or diagnostics.

In various embodiments, the protocells and/or the loaded protocells can provide a targeted delivery methodology for selectively delivering the protocells or the cargo components to targeted cells (e.g., cancer cells). For example, a surface of the lipid bi-layer can be modified by a targeting active species that corresponds to the targeted cell. The targeting active species may be a targeting peptide as otherwise described herein, a polypeptide including an antibody or antibody fragment, an aptamer, a carbohydrate or other moiety which binds to a targeted cell. In exemplary aspects, the targeting active species is a targeting peptide as otherwise described herein. In certain embodiments, exemplary peptide targeting species include a peptide which targets APC or other cells as otherwise described herein.

For example, by providing a targeting active species (for example, a targeting peptide) on the surface of the loaded protocell, the protocell selectively binds to the targeted cell in accordance with the present teachings. In most instances, if the protocells are conjugated with the targeting peptide, the protocells will selectively bind to the cancer cells and no appreciable binding to the non-cancerous cells occurs.

Once bound and taken up by the target cells, the loaded protocells can release cargo components from the porous particle and transport the released cargo components into the target cell. For example, sealed within the protocell by the liposome fused bi-layer on the porous particle core, the cargo components can be released from the pores of the lipid bi-layer, transported across the protocell membrane of the lipid bi-layer and delivered within the targeted cell. In embodiments, the release profile of cargo components in protocells can be more controllable as compared with when only using liposomes as known in the prior art. The cargo release can be determined by, for example, Interactions between the porous core and the lipid bi-layer and/or other parameters such as pH value of the system. For example, the release of cargo can be achieved through the lipid bi-layer, through dissolution of the porous silica; while the release of the cargo from the protocells can be pH-dependent.

In certain embodiments, the pKa for the cargo is often less than 7, or about 4.5 to about 6.0, but can be about pH 14 or less. Lower pHs tend to facilitate the release of the cargo components significantly more than compared with high pHs. Lower pHs tend to be advantageous because the endosomal compartments inside most cells are at low pHs (about 5.5), but the rate of delivery of cargo at the cell can be influenced by the pH of the cargo. Depending upon the cargo and the pH at which the cargo is released from the protocell, the release of cargo can be relative short (a few hours to a day or so) or span for several days to about 20-30 days or longer. Thus, the protocell compositions may accommodate immediate release and/or sustained release applications from the protocells themselves.

In certain embodiments, the inclusion of surfactants can be provided to rapidly rupture the lipid bi-layer, transporting the cargo components across the lipid bi-layer of the protocell as well as the targeted cell. In certain embodiments, the phospholipid bi-layer of the protocells can be ruptured by the application/release of a surfactant such as sodium dodecyl sulfate (SDS), among others to facilitate a rapid release of cargo from the protocell into the targeted cell. Other than surfactants, other materials can be included to rapidly rupture the bi-layer. One example would be gold or magnetic nanoparticles that could use light or heat to generate heat thereby rupturing the bi-layer. Additionally, the bi-layer can be tuned to rupture in the presence of discrete biophysical phenomena, such as during inflammation in response to increased reactive oxygen species production. In certain embodiments, the rupture of the lipid bi-layer can in turn induce immediate and complete release of the cargo components from the pores of the particle core of the protocells. In this manner, the protocell platform can provide an increasingly versatile delivery system as compared with other delivery systems in the art. For example, when compared to delivery systems using nanoparticles only, the disclosed protocell platform can provide a simple system and can take advantage of the low toxicity and immunogenicity of liposomes or lipid bi-layers along with their ability to be PEGylated or to be conjugated to extend circulation time and effect targeting. In another example, when compared to delivery systems using liposome only, the protocell platform can provide a more stable system and can take advantage of the mesoporous core to control the loading and/or release profile and provide increased cargo capacity.

In addition, the lipid bi-layer and its fusion on porous particle core can be fine-tuned to control the loading, release, and targeting profiles and can further comprise fusogenic peptides and related peptides to facilitate delivery of the protocells for greater therapeutic and/or diagnostic effect. Further, the lipid bi-layer of the protocells can provide a fluidic interface for ligand display and multivalent targeting, which allows specific targeting with relatively low surface ligand density due to the capability of ligand reorganization on the fluidic lipid interface. Furthermore, the disclosed protocells can readily enter targeted cells while empty liposomes without the support of porous particles cannot be internalized by the cells.

Exemplary multilamellar liposomes can be produced by the method of Moon, et al., “Interbi-layer-crosslinked multilamellar vesicles as synthetic vaccines for potent humoral and cellular immune responses”, Nature Materials, 2011, 10, pp. 243-251 through crosslinking by divalent cation crosslinking with dithiol chemistry. Another approach would be to hydrate lipid films and bath sonicate (without extrusion) and use polydisperse liposome fusion onto monodisperse cores loaded with cargo.

Pharmaceutical compositions comprise an effective population of protocells as otherwise described herein formulated to effect an intended result (e.g., therapeutic result and/or diagnostic analysis, including the monitoring of therapy) formulated in combination with a pharmaceutically acceptable carrier, additive or excipient. The protocells within the population of the composition may be the same or different depending upon the desired result to be obtained. Pharmaceutical compositions may also comprise an addition bioactive agent or drug, such as an anti-cancer agent or an anti-microbial agent, for example, an anti-HIV, anti-HBV or an anti-HCV agent.

Generally, dosages and routes of administration of the compound are determined according to the size and condition of the subject, according to standard pharmaceutical practices. Dose levels employed can vary widely, and can readily be determined by those of skill in the art. Typically, amounts in the milligram up to gram quantiles are employed. The composition may be administered to a subject by various mutes, e.g., orally, transdermally, perineurally or parenterally, that is, by intravenous, subcutaneous, intraperitoneal, intrathecal or intramuscular injection, among others, including buccal, rectal and transdermal administration. Subjects contemplated for treatment according to the method include humans, companion animals, laboratory animals, and the like. The present disclosure contemplates immediate and/or sustained/controlled release compositions, including compositions which comprise both immediate and sustained release formulations. This is particularly true when different populations of protocells are used in the pharmaceutical compositions or when additional bioactive agent(s) are used in combination with one or more populations of protocells as otherwise described herein.

Formulations containing the compounds may take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like, e.g., in unit dosage forms suitable for simple administration of precise dosages.

Pharmaceutical compositions typically include a conventional pharmaceutical carrier or excipient and may additionally include other medicinal agents, carriers, adjuvants, additives and the like. In one embodiment, the composition is about 0.1% to about 85%, about 0.5% to about 75% by weight of a compound or compounds, with the remainder consisting essentially of suitable pharmaceutical excipients.

An injectable composition for parenteral administration (e.g., intravenous, intramuscular or intrathecal) will typically contain the compound in a suitable i.v. solution, such as sterile physiological salt solution. The composition may also be formulated as a suspension in an aqueous emulsion.

Liquid compositions can be prepared by dissolving or dispersing the population of protocells (about 0.5% to about 20% by weight or more), and optional pharmaceutical adjuvants, in a carrier, such as, for example, aqueous saline, aqueous dextrose, glycerol, or ethanol, to form a solution or suspension. For use in an oral liquid preparation, the composition may be prepared as a solution, suspension, emulsion, or syrup, being supplied either in liquid form or a dried form suitable for hydration in water or normal saline.

For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

When the composition is employed in the form of solid preparations for oral administration, the preparations may be tablets, granules, powders, capsules or the like. In a tablet formulation, the composition is typically formulated with additives, e.g., an excipient such as a saccharide or cellulose preparation, a binder such as starch paste or methyl cellulose, a filler, a disintegrator, and other additives typically used in the manufacture of medical preparations.

The composition to be administered will contain a quantity of the selected compound in a pharmaceutically effective amount for therapeutic use in a biological system, including a patient or subject.

Methods of treating patients or subjects in need for a particular disease state or infection (especially including cancer and/or a HBV, HCV or HIV infection) comprise administration an effective amount of a pharmaceutical composition comprising therapeutic protocells and optionally at least one additional bioactive (e.g., anti-viral) agent.

Diagnostic methods comprise administering to a patient in need (a patient suspected of having cancer) an effective amount of a population of diagnostic protocells (e.g., protocells which comprise a target species, such as a targeting peptide which binds selectively to APC cells or virus infected cells and a reporter component to indicate the binding of the protocells to APC or virus infected cells if the infection is present) whereupon the binding of protocells to cancer cells as evidenced by the reporter component (moiety) will enable a diagnosis of the existence of cancer in the patient.

An alternative of the diagnostic method can be used to monitor the therapy of cancer or other disease state in a patient, the method comprising administering an effective population of diagnostic protocells (e.g., protocells which comprise a target species, such as a targeting peptide which binds selectively to APC cells or other target cells and a reporter component to indicate the binding of the protocells to the target cells) to a patient or subject prior to treatment, determining the level of binding of diagnostic protocells to target cells in said patient and during and/or after therapy, determining the level of binding of diagnostic protocells to target cells in said patient, whereupon the difference in binding before the start of therapy in the patient and during and/or after therapy win evidence the effectiveness of therapy in the patient, including whether the patient has completed therapy or whether the disease state has been inhibited or eliminated (including remission of a cancer).

### Exemplary Particle Modifications for Hydrophobic Cargo

Porous nanoparticulates used in protocells include mesoporous silica nanoparticles and core-shell nanoparticles. The porous nanoparticulates can also be biodegradable polymer nanoparticulates comprising one or more compositions selected from the group consisting of aliphatic polyesters, poly (lactic acid) (PLA), poly (glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), poly(lactide-co-caprolactone), alginate and other polysaccharides, collagen, and chemical derivatives thereof, albumin a hydrophilic protein, zein, a prolamine, a hydrophobic protein, and copolymers and mixtures thereof.

A porous spherical silica nanoparticle may be surrounded by a supported lipid or polymer bilayer or multilayer. Various embodiments provide nanostructures and methods for constructing and using the nanostructures and providing protocells. Many of the protocells in their most elemental form are known in the art. Porous silica particles of varying sizes ranging in size (diameter) from less than 5 nm to 200 nm or 500 nm or more are readily available in the art or can be readily prepared using methods known in the art (see the examples section) or alternatively, can be purchased from SkySpring Nanomaterials, Inc., Houston. Tex., USA or from Discovery Scientific, Inc., Vancouver, British Columbia. Multimodal silica nanoparticles may be readily prepared using the procedure of Carroll et al., (2009). Protocells can be readily obtained using methodologies known in the art. Protocells may be readily prepared, including protocells comprising lipids which are fused to the surface of the silica nanoparticle. See, for example, Liu et al. (2009), Liu et al. (2009) Lu et al., (1999). In one embodiment, protocells are prepared according to the procedures which are presented in Ashley et al. (2011), Lu et al. (1999), Caroll et al. (2009), and as otherwise presented herein.

One method of making MSNPs is described by Lin et al. (2010) and Lin et al. (2011). In this method, the MSNPs are first produced by standard methods described in the references set forth above by reacting TEOS, TMOS or any other appropriate silane precursor in a surfactant (e.g., CTAB, BDHAC) to produce the MSNPs, which can then be modified with silylhydrocarbon to fully coat the MSNP to form the hydrocarbon coated MSNP. The hydrocarbon coating of the MSNP may be provided prior to a hydrothermal step or after a hydrothermal step by reacting a hydrocarbon silyl chloride (e.g., a mono-, di- or trichloridesilylhydrocarbon) with the MSNP in an appropriate solvent or solvent mixture (e.g., ethanol/chloroform 1:1, cyclohexane, acetonitrile, etc.) at slightly elevated temperature (about 40° C. to about 60° C. until the reaction is complete and the hydrocarbon completely coats the MSMPs (typically about 12 hours or more)). The chlorosilylhydrocarbon is generally used at a molar ratio of at least about 0.5% to about 20%, often about 1% to about 10% (e.g. about 7.5%) to the silica precursor used to form the MSNP in order to ensure that the entire surface of the MSNP is fully coated with the silyl hydrocarbon. Either before or after the coating step, the MSNPs are treated with hydrothermal heating (about 60° C. to about 120° C. in a sealed container for about 12 hours or more). The final MSNPs are fully coated with hydrocarbon by the reaction of SiO groups on the surface of the MSNP with the chlorosilyl groups of the chlorosilylhydrocarbon in order to coat the MSNPs with hydrocarbon through the S—O—Si bonds which occur at the surface of the MSNP with the silyl groups of the silyl hydrocarbon.

In an alternative embodiment, the MSN after formation (about a 12 hour synthesis using standard methods of preparation, as described above) may be first carboxylated (using a silyl carboxyl agent such as 3-(triethoxysilyl)propylsuccinic anhydride at approximately 0.5% to about 20%, often about 1% to about 15%, often about 1% to about 5%, about 1-1.5% of the TEOS utilized) to form a carboxylic acid group on the surface of the MSN inked to the MSN through Si—O—Si bonds formed when the 3-(triethoxysilyl)propylsuccinic acid and the SiOH groups on the surface of the MSN react. This takes about an hour or so. The carboxylated MSN is then subjected to a hydrothermal step (generally about 12-36 hours. e.g., about 24 hours at an elevated temperature ranging from about 60° C. to about 120° C.) to form a final carboxylated MSN which can be reacted with a crosslinker such as EDC or other crosslinker (the amine portion of the crosslinker forms an amide or other stable bond with the carboxyl group) and the carboxylic/electrophilic end of the linker is reacted with an amine containing phospholipid such as DOPE, DMPE, DPPE or DSPE to form the hydrocarbon coated MSN.

The hydrocarbon coated MSN may then be coated with a phospholipid as described herein to produce hybrid bilayer protocells. In this approach, the hydrocarbon coated MSN is then mixed with a phospholipid which can include a PEGylated phospholipid as otherwise described herein in solvent (chloroform, etc.) and a hydrocarbon/lipophilic cargo and dried together into a film (evaporation, etc.). The film is then hydrated in PBS and washed several times by centrifugation providing hybrid bilayer protocells which have been loaded with a hydrophobic cargo. The hydrocarbon cargo can be a drug, especially an anti-cancer drug, or a hydrophobic reporter for diagnostics.

In some embodiments, the lipid bilayer of the protocells can provide biocompatibility and can be modified to possess targeting species including, for example, targeting peptides including oligopeptides, antibodies, aptamers, and PEG (polyethylene glycol) (including PEG covalently linked to specific targeting species), among others, to allow, for example, further stability of the protocells and/or a targeted delivery into an antigen presenting cell (APC).

The protocell particle size distribution depending on the application and biological effect, may be monodisperse or polydisperse. The silica cores can be rather monodisperse (i.e., a uniform sized population varying no more than about 5% in diameter e.g., ±10-nm for a 200 nm diameter protocell especially if they are prepared using solution techniques) or rather polydisperse (e.g., a polydisperse population can vary widely from a mean or medium diameter, e.g., up to ±200-nm or more if prepared by aerosol). Polydisperse populations can be sized into monodisperse populations. All of these are suitable for protocell formation. In some embodiments, protocells are no more than about 500 nm in diameter, or no more than about 200 nm in diameter in order to afford delivery to a patient or subject and produce an intended therapeutic effect. The pores of the protocells may vary in order to load plasmid DNA and/or other macromolecules into the core of the protocell. These may be varied pursuant to methods which are well known in the art.

Hybrid protocells generally range in size from greater than about 8-10 nm to about 5 μm in diameter, about 20-nm-3 μm in diameter, about 10 nm to about 500 nm, or about 20-200-nm (including about 150 nm, which may be a mean or median diameter). In one embodiment, hybrid protocells range in size from about 25 nm up to about 250 nm, e.g., hybrid protocells being less than 200 nm in diameter, less than 150 nm in diameter, or less than about 100 nm in diameter. As discussed above, the protocell population may be considered monodisperse or polydisperse based upon the mean or median diameter of the population of protocells. Size can impact immunogenic aspects as particles smaller than about 8-nm diameter are excreted through kidneys, and those particles larger than about 200 nm are often trapped by the liver and spleen. Thus, an embodiment focuses in smaller sized protocells for drug delivery and diagnostics in the patient or subject.

Protocells are characterized by containing mesopores, e.g., pores which are found in the nanostructure material. These pores (at least one, but often a large plurality) may be found intersecting the surface of the nanoparticle (by having one or both ends of the pore appearing on the surface of the nanoparticle) or internal to the nanostructure with at least one or more mesopore interconnecting with the surface mesopores of the nanoparticle. Interconnecting pores of smaller size are often found internal to the surface mesopores. The overall range of pore size of the mesopores can be 0.03-50-nm in diameter. In one embodiment, pore sizes of mesopores range from about 2-30 nm; they can be monosized or bimodal or graded—they can be ordered or disordered (essentially randomly disposed or worm-like). As noted, larger pores are usually used for loading plasmid DNA and/or full length microbial protein which optionally comprises ubiquitin presented as a fusion protein.

Mesopores (IUPAC definition 2-50-nm in diameter) are ‘molded’ by templating agents including surfactants, block copolymers, molecules, macromolecules, emulsions, latex beads, or nanoparticles. In addition, processes could also lead to micropores (IUPAC definition less than 2-nm in diameter) all the way down to about 0.03-nm e.g. if a templating moiety in the aerosol process is not used. They could also be enlarged to macropores, i.e., 50-nm in diameter.

In an embodiment, the nanostructures include a core-shell structure which comprises a porous particle core surrounded by a shell of lipid such as a bilayer, but possibly a monolayer or multilayer (see Liu et al. (2009)). The porous particle core can include, for example, a porous nanoparticle made of an inorganic and/or organic material as set forth above surrounded by a lipid bilayer. In one embodiment, these lipid bilayer surrounded nanostructures are referred to as “protocells” or “functional protocells,” since they have a supported lipid bilayer membrane structure. In some embodiments, the porous particle core of the protocells can be loaded with various desired species (“cargo”), including small hydrophobic molecules (e.g., anti-cancer agents as otherwise described herein), hydrophobic large molecules, hydrophobic reporters.

In certain embodiments, the cargo components can include, but are not limited to, chemical small molecules (especially anti-cancer agents and antiviral agents, including anti-HIV, anti-HBV and/or anti-HCV agents, such as a therapeutic application or a diagnostic application as otherwise disclosed herein.

In some embodiments, the lipid bilayer of the protocells can provide biocompatibility and can be modified to possess targeting species including, for example, targeting peptides including antibodies, aptamers, and PEG (polyethylene glycol) to allow, for example, further stability of the protocells and/or a targeted delivery into a bioactive cell.

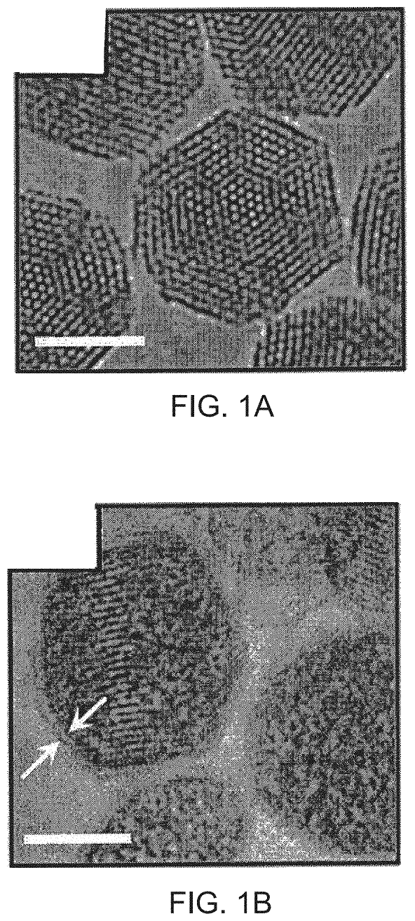

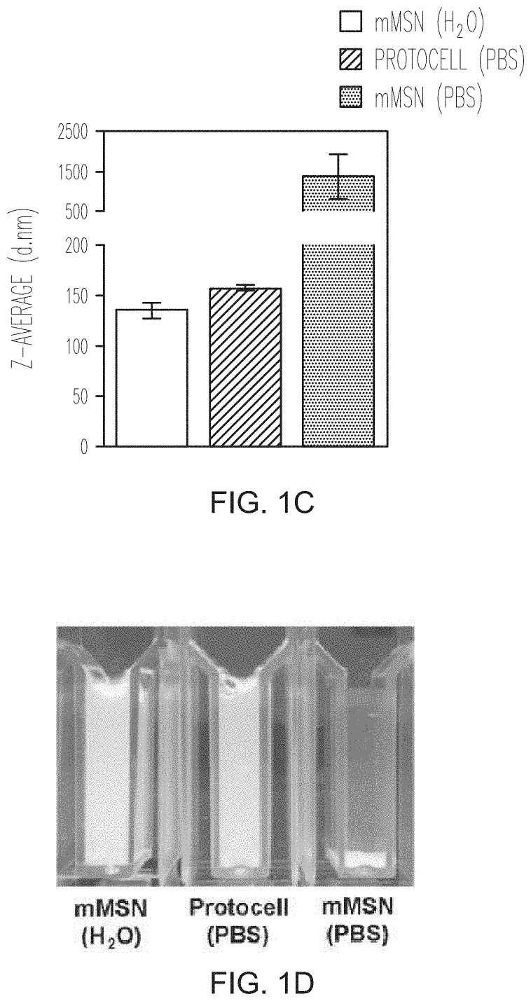

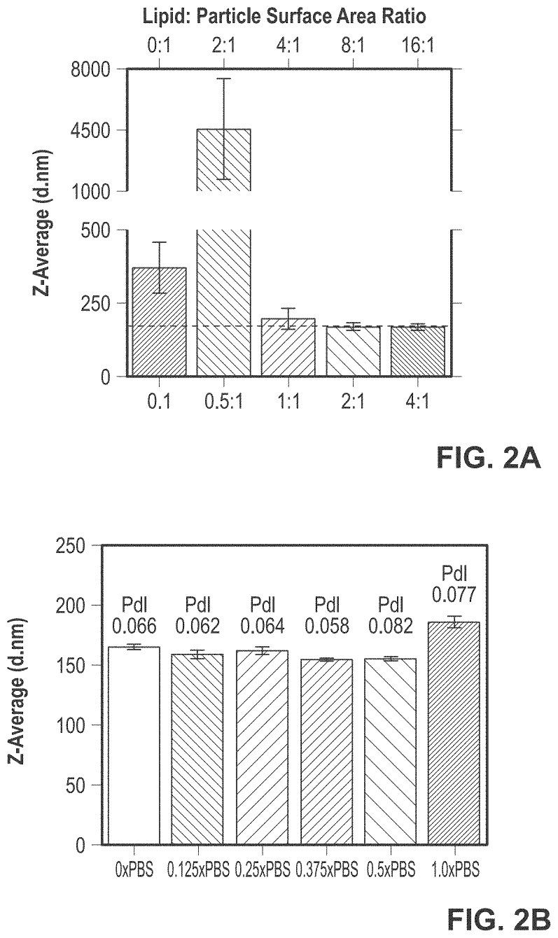

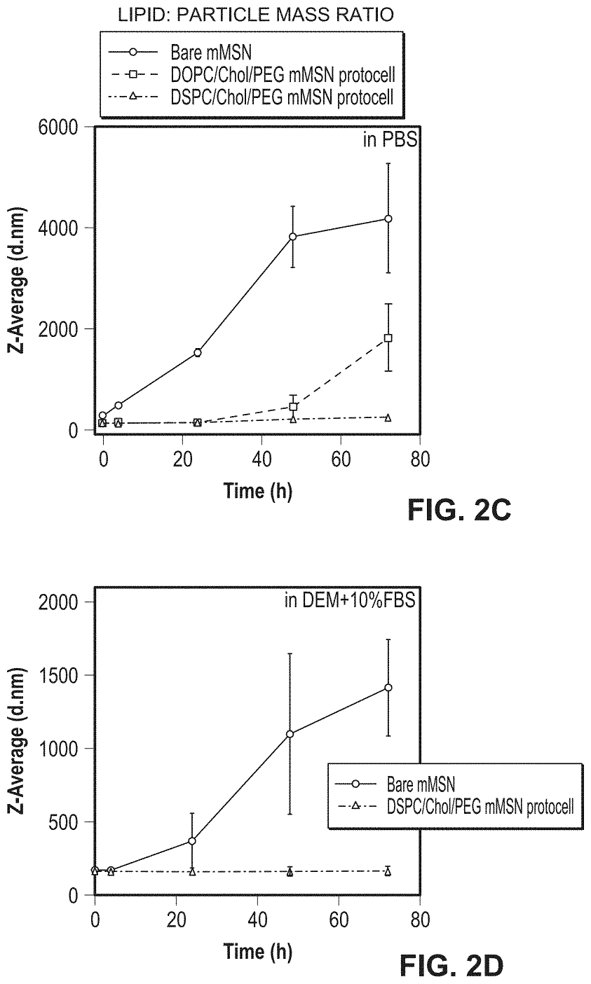

The protocells particle size distribution, depending on the application, may be monodisperse or polydisperse. The silica cores can be rather monodisperse (e.g., a uniform sized population varying no more than about 5% in diameter e.g., ±10-nm for a 200 nm diameter protocell especially if they are prepared using solution techniques) or rather polydisperse (e.g., a polydisperse population can vary widely from a mean or medium diameter, e.g., up to ±200-nm or more if prepared by aerosol. See FIG. 1, attached. Polydisperse populations can be sized into monodisperse populations. All of these are suitable for protocell formation. In one embodiment, protocells may be no more than about 500 nm in diameter, e.g., no more than about 200 nm in diameter, in order to afford delivery to a patient or subject and produce an intended therapeutic effect.

In certain embodiments, protocells generally range in size from greater than about 8-10 nm to about 5 μm in diameter, about 20-nm-3 μm in diameter, about 10 nm to about 500 nm, or about 20-200-nm (including about 150 nm, which may be a mean or median diameter). As discussed above, the protocell population may be considered monodisperse or polydisperse based upon the mean or median diameter of the population of protocells. Size for therapeutic and diagnostic aspects include particles smaller than about 8-nm diameter are excreted through kidneys, and those particles larger than about 200 nm are trapped by the liver and spleen. Thus, an embodiment of focuses in smaller sized protocells for drug delivery and diagnostics in the patient or subject.

In certain embodiments, protocells on are characterized by containing mesopores, e.g., pores which are found in the nanostructure material. These pores (at least one, but often a large plurality) may be found intersecting the surface of the nanoparticle (by having one or both ends of the pore appearing on the surface of the nanoparticle) or internal to the nanostructure with at least one or more mesopore interconnecting with the surface mesopores of the nanoparticle. Interconnecting pores of smaller size are often found internal to the surface mesopores. The overall range of pore size of the mesopores can be 0.03-50-nm in diameter. In one embodiment, pore sizes of mesopores range from about 2-30 nm; they can be monosized or bimodal or graded—they can be ordered or disordered (essentially randomly disposed or worm-like).

Mesopores (IUPAC definition 2-50-nm in diameter) are ‘molded’ by templating agents including surfactants, block copolymers, molecules, macromolecules, emulsions, latex beads, or nanoparticles. In addition, processes could also lead to micropores (IUPAC definition less than 2 nm in diameter) all the way down to about 0.03-nm e.g. If a templating moiety in the aerosol process is not used. They could also be enlarged to macropores, e.g., 50 nm in diameter.

Pore surface chemistry of the nanoparticle material can be very diverse—all organosilanes yielding cationic, anionic, hydrophilic, hydrophobic, reactive groups—pore surface chemistry, especially charge and hydrophobicity, affect loading capacity. Attractive electrostatic interactions or hydrophobic interactions control/enhance loading capacity and control release rates. Higher surface areas can lead to higher loadings of drugs/cargos through these attractive interactions. See below.

In certain embodiments, the surface area of nanoparticles, as measured by the N2 BET method, ranges from about 100 m2/g to >about 1200 m2/g. In general, the larger the pore size, the smaller the surface area. The surface area theoretically could be reduced to essentially zero, if one does not remove the templating agent or if the pores are sub-0.5-nm and therefore not measurable by N2 sorption at 77K due to kinetic effects. However, in this case, they could be measured by CO₂ or water sorption, but would probably be considered non-porous. This would apply if biomolecules are encapsulated directly in the silica cores prepared without templates, in which case particles (internal cargo) would be released by dissolution of the silica matrix after delivery to the cell.

Typically the protocells are loaded with cargo to a capacity up to over 100 weight %: defined as (cargo weight/weight of protocell)×100. The optimal loading of cargo is often about 0.01 to 30% but this depends on the drug or drug combination which is incorporated as cargo into the protocell. This is generally expressed in μM per 10¹⁰ particles where we have values ranging from 2000-100 μM per 10¹⁰ particles. In one embodiment, protocells exhibit release of cargo at pH about 5.5, which is that of the endosome, but are stable at physicological pH of 7 or higher (7.4).

The surface area of the internal space for loading is the pore volume whose optimal value ranges from about 1.1 to 0.5 cubic centimeters per gram (cc/g). Note that in certain protocells, the surface area is mainly internal as opposed to the external geometric surface area of the nanoparticle.

The lipid bilayer supported on the porous particle according to one embodiment has a lower melting transition temperature, i.e. is more fluid than a lipid bilayer supported on a non-porous support or the lipid bilayer in a liposome. This is sometimes important in achieving high affinity binding of targeting ligands at low peptide densities, as it is the bilayer fluidity that allows lateral diffusion and recruitment of peptides by target cell surface receptors. One embodiment provides for peptides to cluster, which facilitates binding to a complementary target.

In one embodiment, the lipid bilayer may vary significantly in composition. Ordinarily, any lipid or polymer which is may be used in liposomes may also be used in protocells. In one embodiment, lipid bilayers for use in protocells comprise a mixtures of lipids (as otherwise described herein) at a weight ratio of 5% DOPE, 5% PEG, 30% cholesterol, 60% DOPC or DPPC (by weight).

The charge of the mesoporous silica NP core as measured by the Zeta potential may be varied monotonically from −50 to +50 mV by modification with the amine silane, 2-(aminoethyl) propyltrimethoxy-silane (AEPTMS) or other organosilanes. This charge modification, in turn, varies the loading of the drug within the cargo of the protocell. Generally, after fusion of the supported lipid bilayer, the zeta-potential is reduced to between about −10 mV and +5 mV, which is important for maximizing circulation time in the blood and avoiding non-specific interactions.

Depending on how the surfactant template is removed, e.g. calcination at high temperature (500° C.) versus extraction in acidic ethanol, and on the amount of AEPTMS incorporated in the silica framework, the silica dissolution rates can be varied widely. This in turn controls the release rate of the internal cargo. This occurs because molecules that are strongly attracted to the internal surface area of the pores diffuse slowly out of the particle cores, so dissolution of the particle cores controls in part the release rate.

Further characteristics of protocells are that they are stable at pH 7, i.e. they don't leak their cargo, but at pH 5.5, which is that of the endosome lipid or polymer coating becomes destabilized initiating cargo release. This pH-triggered release is important for maintaining stability of the protocell up until the point that it is internalized in the cell by endocytosis, whereupon several pH triggered events cause release into the endosome and consequently, the cytosol of the cell. The protocell core particle and surface can also be modified to provide non-specific release of cargo over a specified, prolonged period of time, as well as be reformulated to release cargo upon other biophysical changes, such as the increased presence of reactive oxygen species and other factors in locally inflamed areas. Quantitative experimental evidence has shown that targeted protocells illicit only a weak immune response, because they do not support T-Cell help required for higher affinity IgG, a favorable result.

Various embodiments provide nanostructures which are constructed from nanoparticles which support a lipid bilayer(s). In some embodiments, the nanostructures include, for example, a core-shell structure including a porous particle core surrounded by a shell of lipid bilayer(s). The nanostructure, e.g., a porous silica nanostructure as described above, supports the lipid bilayer membrane structure.

In some embodiments, the lipid bilayer of the protocells can provide biocompatibility and can be modified to possess targeting species including, for example, targeting peptides, fusogenic peptides, antibodies, aptamers, and PEG (polyethylene glycol) to allow, for example, further stability of the protocells and/or a targeted delivery into a bioactive cell, in particular a cancer cell. PEG, when included in lipid bilayers, can vary widely in molecular weight (although PEG ranging from about 10 to about 100 units of ethylene glycol, about 15 to about 50 units, about 15 to about 20 units, about 15 to about 25 units, about 16 to about 18 units, etc, may be used and the PEG component which is generally conjugated to phospholipid through an amine group comprises about 1% to about 20 about 5% to about 15%, or about 10% by weight of the lipids which are included in the lipid bilayer.

Numerous lipids which are used in liposome delivery systems may be used to form the lipid bilayer on nanoparticles to provide protocells. Virtually any lipid or polymer which is used to form a liposome or polymersome may be used in the lipid bilayer which surrounds the nanoparticles to form protocells according to an embodiment. In one embodiment, lipids include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol, not technically a lipid, but presented as a lipid for purposes of an embodiment given the fact that cholesterol may be an important component of the lipid bilayer of protocells according to an embodiment. Often cholesterol is incorporated into lipid bilayers of protocells in order to enhance structural integrity of the bilayer. These lipids are all readily available commercially from Avanti Polar Lipids, Inc. (Alabaster. Ala., USA). DOPE and DPPE are particularly useful for conjugating (through an appropriate crosslinker) peptides, polypeptides, including antibodies, RNA and DNA through the amine group on the lipid.

Pegylated phospholipids include for example, pegylated 1,2-distearoyl-sn-glycero-3-phosphoethanolamine (PEG-DSPE), pegylated 1,2-dioleoyl-sn-glycero-3-phosphoethanolamine (PEG-DOPE), pegylated 1,2-dipalmitoyl-sn-glycero-3-phosphoethanolamine (PEG-DPPE), and pegylated 1,2-dimyristoyl-sn-glycero-3-phosphoethanolamine (PEG-DMPE), among others, including a pegylated ceramide (e.g. N-octanoyl-sphingosine-1-succinylmethoxy-PEG or N-palmitoyl-sphingosine-1-succinylmethoxy-PEG, among others). The PEG generally ranges in size (average molecular weight for the PEG group) from about 350-7500, about 350-5000, about 500-2500, about 1000-2000. Pegylated phospholipids may comprise the entire phospholipid monolayer of hybrid phospholipid protocells, or alternatively they may comprise a minor component of the lipid monolayer or be absent. Accordingly, the percent by weight of a pegylated phospholipid in phospholipid monolayers ranges from 0% to 100% or 0.01% to 99%, e.g., about 5%, 10%, 15%, 20%, 25%, 30%, 35%, 40%, 50%, 55%, 60% and the remaining portion of the phospholipid monolayer comprising at least one additional lipid (such as cholesterol, usually in amounts less than about 50% by weight), including a phospholipid.

In certain embodiments, the porous nanoparticulates can also be biodegradable polymer nanoparticulates comprising one or more compositions selected from the group consisting of aliphatic polyesters, poly (lactic acid) (PLA), poly (glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), poly(lactide-co-caprolactone), alginate and other polysaccharides, collagen, and chemical derivatives thereof, albumin a hydrophilic protein, zein, a prolamine, a hydrophobic protein, and copolymers and mixtures thereof.

In still other embodiments, the porous nanoparticles each comprise a core having a core surface that is essentially free of silica, and a shell attached to the core surface, wherein the core comprises a transition metal compound selected from the group consisting of oxides, carbides, sulfides, nitrides, phosphides, borides, halides, selenides, tellurides, tantalum oxide, iron oxide or combinations thereof.

The silica nanoparticles can be, for example, mesoporous silica nanoparticles and core-shell nanoparticles. The nanoparticles may incorporate an absorbing molecule, e.g. an absorbing dye. Under appropriate conditions, the nanoparticles emit electromagnetic radiation resulting from chemiluminescence. Additional contrast agents may be included to facilitate contrast in MRI, CT, PET, and/or ultrasound imaging.

Mesoporous silica nanoparticles can be, e.g., from around 5 nm to around 500 nm in size, including all integers and ranges there between. The size is measured as the longest axis of the particle. In various embodiments, the particles are from around nm to around 500 nm and from around 10 nm to around 100 nm in size. The mesoporous silica nanoparticles have a porous structure. The pores can be from around 1 to around 20 nm in diameter, including all integers and ranges there between. In one embodiment, the pores are from around 1 to around 10 nm in diameter. In one embodiment, around 90% of the pores are from around 1 to around 20 nm in diameter. In another embodiment, around 95% of the pores are around 1 to around 20 nm in diameter.

The mesoporous nanoparticles can be synthesized according to methods known in the art. In one embodiment, the nanoparticles are synthesized using sol-gel methodology where a silica precursor or silica precursors and a silica precursor or silica precursors conjugated (i.e., covalently bound) to absorber molecules are hydrolyzed in the presence of templates in the form of micelles. The templates are formed using a surfactant such as, for example, hexadecyltrimethylammonium bromide (CTAB). It is expected that any surfactant which can form micelles can be used.

The core-shell nanoparticles comprise a core and shell. The core comprises silica and an absorber molecule. The absorber molecule is incorporated in to the silica network via a covalent bond or bonds between the molecule and silica network. The shell comprises silica.

In one embodiment, the core is independently synthesized using known sol-gel chemistry, e.g., by hydrolysis of a silica precursor or precursors. The silica precursors are present as a mixture of a silica precursor and a silica precursor conjugated, e.g., linked by a covalent bond, to an absorber molecule (referred to herein as a “conjugated silica precursor”). Hydrolysis can be carried out under alkaline (basic) conditions to form a silica core and/or silica shell. For example, the hydrolysis can be carried out by addition of ammonium hydroxide to the mixture comprising silica precursor(s) and conjugated silica precursor(s).

Silica precursors are compounds which under hydrolysis conditions can form silica. Examples of silica precursors include, but are not limited to, organosilanes such as, for example, tetraethoxysilane (TEOS), tetramethoxysilane (TMOS) and the like.

The silica precursor used to form the conjugated silica precursor has a functional group or groups which can react with the absorbing molecule or molecules to form a covalent bond or bonds. Examples of such silica precursors include, but is not limited to, isocyanatopropyltriethoxysilane (ICPTS), aminopropyltrimethoxysilane (APTS), mercaptopropyltrimethoxysilane (MPTS), and the like.

In one embodiment, an organosilane (conjugatable silica precursor) used for forming the core has the general formula R₄ₙSiXₙ, where X is a hydrolyzable group such as ethoxy, methoxy, or 2-methoxy-ethoxy; R can be a monovalent organic group of from 1 to 12 carbon atoms which can optionally contain, but is not limited to, a functional organic group such as mercapto, epoxy, acrylyl, methacrylyl, or amino; and n is an integer of from 0 to 4. The conjugatable silica precursor is conjugated to an absorber molecule and subsequently co-condensed for forming the core with silica precursors such as, for example, TEOS and TMOS. A silane used for forming the silica shell has n equal to 4. The use of functional mono-, bis- and tris-alkoxysilanes for coupling and modification of co-reactive functional groups or hydroxy-functional surfaces, including glass surfaces, is also known (see Kirk-Othmer, Encyclopedia of Chemical Technology, Vol. 20, 3rd Ed., J. Wiley, N.Y.; see also E. Pluedemann, Silane Coupling Agents, Plenum Press. N.Y. 1982). The organo-silane can cause gels, so it may be desirable to employ an alcohol or other known stabilizers. Processes to synthesize core-shell nanoparticles using modified Stoeber processes can be found in U.S. patent application Ser. Nos. 10/306,614 and 10/536,569, the disclosure of such processes therein are incorporated herein by reference.

In certain embodiments of a protocell, the lipid bilayer is comprised of one or more lipids selected from the group consisting of phosphatidyl-cholines (PCs) and cholesterol.

In certain embodiments, the lipid bilayer is comprised of one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), egg PC, and a lipid mixture comprising between about 50% to about 70%, or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-cholines, DMPC [14:0] having a carbon length of 14 and no unsaturated bonds, 1,2-dipalmitoyl-sn-glycero-3-phosphocholine (DPPC) [16:0], 1,2-distearoyl-sn-glycero-3-phosphocholine (DSPC) [18:0], 1,2-dioleoyl-sn-glycero-3-phosphocholine (DOPC) [18:1 (Δ9-Cis)], POPC [16:0-18:1], and DOTAP [18:1]. In other embodiments: (a) the lipid bilayer is comprised of a mixture of (1) egg PC, and (2) one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), a lipid mixture comprising between about 50% to about 70% or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-choline, DMPC [14:0] having a carbon length of 14 and no unsaturated bonds, 1,2-dipalmitoyl-sn-glycero-3-phosphocholine (DPPC) [16:0], 1,2-distearoyl-sn-glycero-3-phosphocholine (DSPC) [18:0], 1,2-dioleoyl-sn-glycero-3-phosphocholine (DOPC) [18:1 (Δ9-Cis)], POPC [16:0-18:1] and DOTAP [18:1]; and wherein (b) the molar concentration of egg PC in the mixture is between about 10% to about 50% or about 11% to about 49%, or about 12% to about 48%, or about 13% to about 47%, or about 14% to about 48%, or about 15% to about 45%, or about 16% to about 44%, or about 17% to about 43%, or about 18% to about 42%, or about 19% to about 41%, or about 20% to about 40%, or about 21% to about 39%, or about 22% to about 38%, or about 23% to about 37%, or about 24% to about 36%, or about 25% to about 35%, or about 26% to about 34%, or about 27% to about 33%, or about 28% to about 32%, or about 29% to about 31%, or about 30%.

In certain embodiments, the lipid bilayer is comprised of one or more compositions selected from the group consisting of a phospholipid, a phosphatidyl-choline, a phosphatidyl-serine, a phosphatidyl-diethanolamine, a phosphatidylinosite, a sphingolipid, and an ethoxylated sterol, or mixtures thereof. In illustrative examples of such embodiments, the phospholipid can be a lecithin; the phosphatidylinosite can be derived from soy, rape, cotton seed, egg and mixtures thereof: the sphingolipid can be ceramide, a cerebroside, a sphingosine, and a sphingomyelin, and a mixture thereof; the ethoxylated sterol can be phytosterol, PEG-(polyethyleneglykol)-5-soy bean sterol, and PEG-(polyethyleneglykol)-5 rapeseed sterol. In certain embodiments, the phytosterol comprises a mixture of at least two of the following compositions: sistosterol, camposterol and stigmasterol.

In still other illustrative embodiments, the lipid bilayer is comprised of one or more phosphatidyl groups selected from the group consisting of phosphatidyl choline, phosphatidyl-ethanolamine, phosphatidyl-serine, phosphatidyl-inositol, lyso-phosphatidyl-choline, lyso-phosphatidyl-ethanolamine, lyso-phosphatidyl-inostol and lyso-phosphatidyl-inostol.

In still other illustrative embodiments, the lipid bilayer is comprised of phospholipid selected from a monoacyl or diacylphosphoglyceride.

In still other illustrative embodiments, the lipid bilayer is comprised of one or more phosphoinositides selected from the group consisting of phosphatidyl-inositol-3-phosphate (PI-3-P), phosphatidyl-inositol-4-phosphate (PI-4-P), phosphatidyl-inostol-5-phosphate (PI-5-P), phosphatidyl-inostol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inostol-5-phosphate (LPI-5-P), lysophosphatidyl-inostol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidic-inositol-3,4,5-triphosphate (LPI-3,4,5-P3), and phosphatidyl-inositol (PI), and lysophosphatidyl-inositol (LPI).

In still other illustrative embodiments, the lipid bilayer is comprised of one or more phospholipids selected from the group consisting of PEG-poly(ethylene glycol)-derivatized distearoylphosphatidylethanolamine (PEG-DSPE), poly(ethylene glycol)-derivatized ceramides (PEG-CER), hydrogenated soy phosphatidylcholine (HSPC), egg phosphatidylcholine (EPC), phosphatidyl ethanolamine (PE), phosphatidyl glycerol (PG), phosphatidyl insitol (PI), monosialoganglioside, spingomyelin (SPM), distearoylphosphatidylcholine (DSPC), dimyristoylphosphatidylcholine (DMPC), and dimyristoylphosphatidylglycerol (DMPG).

Protocells can comprise a wide variety of pharmaceutically-active ingredients. The term “hydrophobic drug” or “hydrophobic active agent” is used to describe an active agent which is lipophilic/hydrophobic in nature. Exemplary lipophilic/hydrophobic drugs which are useful include, for example, analgesics and anti-inflammatory agents, such as aloxiprin, auranofin, azapropazone, benorylate, diflunisal, etodolac, fenbufen, fenoprofen calcim, flurbiprofen, ibuprofen, indomethacin, ketoprofen, meclofenamic acid, mefenamic acid, nabumetone, naproxen, oxyphenbutazone, phenylbutazone, piroxicam, sulindac; Anthelmintics, such as albendazole, bephenium hydroxynaphthoate, cambendazole, dichlorophen, Ivermectin, mebendazole, oxamniquine, oxfendazole, oxantel embonate, praziquantel, pyrantel embonate, thiabendazole; Anti-arrhythmic agents such as amiodarone HCl, disopyramide, flecainide acetate, quinidine sulphate; Anti-bacterial agents such as benethamine penicillin, cinoxacin, ciprofloxacin HCl, clarthromycin, clofazimine, cloxacillin, demeclocycline, doxycycline, erythromycin, ethionamide, imipenem, nalidixic acid, nitrofurantoin, rifampicin, spiramycin, sulphabenzamide, sulphadoxine, sulphamerazine, sulphacetamide, sulphadiazine, sulphafurazole, sulphamethoxazole, sulphapyridine, tetracycline, trimethoprim; Anti-coagulants such as dicoumarol, dipyridamole, nicoumalone, phenindione; Anti-depressants such as amoxapine, maprotiline HCl, mianserin HCL, nortriptyline HCl, trazodone HCL, trimipramine maleate; Anti-diabetics such as acetohexamide, chlorpropamide, glibenclamide, gliclazide, glipizide, tolazamide, tolbutamide; Anti-epileptics such as beclamide, carbamazepine, clonazepam, ethotoin, methoin, methsuximide, methylphenobarbitone, oxcarbazepine, paramethadione, phenacemide, phenobarbitone, phenytoin, phensuximide, primidone, sulthiame, valproic acid; Anti-fungal agents such as amphotericin, butoconazole nitrate, clotrimazole, econazole nitrate, fluconazole, flucytosine, griseofulvin, Itraconazole, ketoconazole, miconazole, natamycin, nystatin, sulconazole nitrate, terbinafine HCl, terconazole, tioconazole, undecenoic acid; Anti-gout agents such as allopurinol, probenecid, sulphin-pyrazone; Anti-hypertensive agents such as amlodipine, benidipine, darodipine, dilitazem HCl, diazoxide, felodipine, guanabenz acetate, isradipine, minoxidil, nicardipine HCl, nifedipine, nimodipine, phenoxybenzamine HCl, prazosin HCL, reserpine, terazosin HCL: Anti-malarials such as amodiaquine, chloroquine, chlorproguanil HCl, halofantrine HCl, mefloquine HCl, proguanil HCl, pyrimethamine, quinine sulphate; Anti-migraine agents such as dihydroergotamine mesylate, ergotamine tartrate, methysergide maleate, pizotifen maleate, sumatriptan succinate; Anti-muscarinic agents such as atropine, benzhexol HCl, biperiden, ethopropazine HCl, hyoscyamine, mepenzolate bromide, oxyphencylcimine HCl, tropicamide; Anti-neoplastic agents and Immunosuppressants such as aminoglutethimide, amsacrine, azathioprine, busulphan, chlorambucil, cyclosporin, dacarbazine, estramustine, etoposide, lomustine, melphalan, mercaptopurine, methotrexate, mitomycin, mitotane, mitozantrone, procarbazine HCl, tamoxifen citrate, testolactone: Anti-protozoal agents such as benznidazole, cloquinol, decoquinate, diiodohydroxyquinoline, diloxanide furoate, dinitolmide, furzolidone, metronidazole, nimorazole, nitrofurazone, ornidazole, tinidazole; Anti-thyroid agents such as carbimazole, propylthiouracil; Anxiolytic, sedatives, hypnotics and neuroleptics such as alprazolam, amylobarbitone, barbitone, bentazepam, bromazepam, bromperidol, brotizolam, butobarbitone, carbromal, chlordiazepoxide, chlormethiazole, chlorpromazine, clobazam, clotiazepam, clozapine, diazepam, droperidol, ethinamate, flunanisone, flunitrazepam, fluopromazine, flupenthixol decanoate, fluphenazine decanoate, flurazepam, haloperidol, lorazepam, lormetazepam, medazepam, meprobamate, methaqualone, midazolam, nitrazepam, oxazepam, pentobarbitone, perphenazine pimozide, prochlorperazine, sulpiride, temazepam, thioridazine, triazolam, zopiclone; β-Blockers such as acebutolol, alprenolol, atenolol, labetalol, metoprolol, nadolol, oxprenolol, pindolol, propranolol; Cardiac Inotropic agents such as amrinone, digitoxin, digoxin, enoximone, lanatoside C, medigoxin; Corticosteroids such as beclomethasone, betamethasone, budesonide, cortisone acetate, desoxymethasone, dexamethasone, fludrocortisone acetate, flunisolide, flucortolone, fluticasone propionate, hydrocortisone, methylprednisolone, prednisolone, prednisone, triamcinolone; Diuretics such as acetazolamide, amiloride, bendrofluazide, bumetanide, chlorothiazide, chlorthalidone, ethacrynic acid, frusemide, metolazone, spironolactone, triamterene; Anti-parkinsonian agents such as bromocriptine mesylate, lysuride maleate; Gastro-intestinal agents such as bisacodyl, cimetidine, cisapride, diphenoxylate HCl, domperidone, famotidine, loperamide, mesalazine, nizatidine, omeprazole, ondansetron HCL, ranitidine HCl, sulphasalazine; Histamine H,-Receptor Antagonists such as acrivastine, astemizole, cinnarizine, cyclizine, cyproheptadine HCl, dimenhydrinate, flunarizine HCl, loratadine, meclozine HCl, oxatomide, terfenadine: Lipid regulating agents such as bezafibrate, clofibrate, fenofibrate, gemfibrozil, probucol; Nitrates and other anti-anginal agents such as amyl nitrate, glyceryl trinitrate, isosorbide dinitrate, isosorbide mononitrate, pentaerythritol tetranitrate; Nutritional agents such as betacarotene, vitamin A, vitamin B₂, vitamin D, vitamin E, vitamin K; Opioid analgesics such as codeine, dextropropyoxyphene, diamorphine, dihydrocodeine, meptazinol, methadone, morphine, nalbuphine, pentazocine; Sex hormones such as clomiphene citrate, danazol, ethinyl estradiol, medroxyprogesterone acetate, mestranol, methyltestosterone, norethisterone, norgestrel, estradiol, conjugated oestrogens, progesterone, stanozolol, stibestrol, testosterone, tibolone: and Stimulants such as amphetamine, dexamphetamine, dexfenfluramine, fenfluramine, mazindol, among others. Other hydrophobic drugs include rapamycin, docetaxel, paclitaxel, carbazitaxel, thiazolidinediones (e.g. rosiglitazone, pioglitazone, lobeglitazone, troglitazone, netoglitazone, riboglitazone and ciglitazone) and curcumin, among others.

Exemplary MET binding peptides can be used as targeting peptides on protocells of certain embodiments of the present invention, or in pharmaceutical compositions for their benefit in binding MET protein in a variety of cancer cells, including hepatocellular, cervical and ovarian cells, among numerous other cells in cancerous tissue. In one embodiment, the invention may use one or more of five (5) different 7 mer peptides which show activity as novel binding peptides for MET receptor (a.k.a. hepatocyte growth factor receptor, expressed by gene c-MET). These five (5) 7 mer peptides are as follows:

SEQ ID NO: 7
ASVHFPP (Ala-Ser-Val-His-Phe-Pro-Pro)
SEQ ID NO: 8
TATFWFQ (Thr-Ala-Thr-Phe-Trp-Phe-Gln)
SEQ ID NO: 9
TSPVALL (Thr-Ser-Pro-Val-Ala-Leu-Leu)
SEQ ID NO: 10
IPLKVHP (Ile-Pro-Leu-Lys-Val-His-Pro)
SEQ ID NO: 11
WPRLTNM (Trp-Pro-Arg-Leu-Thr-Asn-Met)

Other targeting peptides are known in the art. Targeting peptides may be complexed or preferably, covalently linked to the lipid bilayer through use of a crosslinking agent as otherwise described herein.

In order to covalently link any of the fusogenic peptides or endosomolytic peptides to components of the lipid bilayer, various approaches, well known in the art may be used. For example, the peptides listed above could have a C-terminal poly-His tag, which would be amenable to Ni-NTA conjugation (lipids commercially available from Avanti). In addition, these peptides could be terminated with a C-terminal cysteine for which heterobifunctional crosslinker chemistry (EDC, SMPH, and the like) to link to aminated lipids would be useful. Another approach is to modify lipid constituents with thiol or carboxylic acid to use the same crosslinking strategy. All known crosslinking approaches to crosslinking peptides to lipids or other components of a lipid layer could be used. In addition click chemistry may be used to modify the peptides with azide or alkyne for cu-catalyzed crosslinking, and we could also use a cu-free click chemistry reaction.

Exemplary crosslinking agents include, for example, 1-Ethyl-3-[3-dimethylaminopropyl]carbodiimide hydrochloride (EDC), succinimidyl 4-[N-maleimidomethyl]cyclohexane-1-carboxylate (SMCC), Succinimidyl 6-[ß-Maleimidopropionamido]hexanoate (SMPH), N-[β-Maleimidopropionic acid] hydrazide (BMPH), NHS-(PEG)ₙ-maleimide, succinimidyl-[(N-maleimidopropionamido)-tetracosaethyleneglycol] ester (SM(PEG)₂₄), and succinimidyl 6-[3′-(2-pyridyldithio)-propionamido] hexanoate (LC-SPDP), among others.

As discussed in detail above, the porous nanoparticle core can include porous nanoparticles having at least one dimension, for example, a width or a diameter of about 3000 nm or less, about 1000 nm or less, about 500 nm or less, about 200 nm or less. In one embodiment, the nanoparticle core is spherical with a diameter of about 500 nm or less, or about 5-10 nm to about 200 nm. In embodiments, the porous particle core can have various cross-sectional shapes including a circular, rectangular, square, or any other shape. In certain embodiments, the porous particle core can have pores with a mean pore size ranging from about 2 nm to about 30 nm, although the mean pore size and other properties (e.g., porosity of the porous particle core) are not limited in accordance with various embodiments of the present teachings.

In general, protocells are biocompatible. Drugs and other cargo components are often loaded by adsorption and/or capillary filling of the pores of the particle core up to approximately 50% by weight of the final protocell (containing all components). In certain embodiments, the loaded cargo can be released from the porous surface of the particle core (mesopores), wherein the release profile can be determined or adjusted by, for example, the pore size, the surface chemistry of the porous particle core, the pH value of the system, and/or the interaction of the porous particle core with the surrounding lipid bilayer(s) as generally described herein.

In one embodiment, the porous nanoparticle core used to prepare the protocells can be tuned in to be hydrophilic or progressively more hydrophobic as otherwise described herein and can be further treated to provide a more hydrophilic surface. For example, mesoporous silica particles can be further treated with ammonium hydroxide and hydrogen peroxide to provide a higher hydrophilicity. In certain aspects, the lipid bilayer is fused onto the porous particle core to form the protocell. Protocells can include various lipids in various weight ratios, including 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycero (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. In one embodiment, the lipid monolayer includes a PEGylated lipid.

The lipid bilayer which is used to prepare protocells can be prepared, for example, by extrusion of hydrated lipid films through a filter with pore size of, for example, about 100 nm, using standard protocols known in the art or as otherwise described herein. The filtered lipid bilayer films can then be fused with the porous particle cores, for example, by pipette mixing. In certain embodiments, excess amount of lipid bilayer or lipid bilayer films can be used to form the protocell in order to improve the protocell colloidal stability.

In certain diagnostic embodiments, various dyes or fluorescent (reporter) molecules can be included in the protocell cargo (as expressed by as plasmid DNA) or attached to the porous particle core and/or the lipid bilayer for diagnostic purposes. For example, the porous particle core can be a silica core or the lipid bilayer and can be covalently labeled with FITC (green fluorescence), while the lipid bilayer or the particle core can be covalently labeled with FITC Texas red (red fluorescence). The porous particle core, the lipid bilayer and the formed protocell can then be observed by, for example, confocal fluorescence for use in diagnostic applications. In addition, as discussed herein, plasmid DNA can be used as cargo in protocells such that the plasmid may express one or more fluorescent proteins such as fluorescent green protein or fluorescent red protein which may be used in diagnostic applications.

In various embodiments, the protocell may be used in a synergistic system where the lipid bilayer fusion or liposome fusion (i.e., on the porous particle core) is loaded and sealed with various cargo components with the pores (mesopores) of the particle core, thus lipid bilayer or through dissolution of the porous nanoparticle, if applicable. In certain embodiments, in addition to fusing a single lipid (e.g., phospholipids) bilayer, multiple bilayers with opposite charges can be successively fused onto the porous particle core to further influence cargo loading and/or sealing as well as the release characteristics of the final protocell

A fusion and synergistic loading mechanism can be included for cargo delivery. For example, cargo can be loaded, encapsulated, or sealed, synergistically through liposome fusion on the porous particles. The cargo can include, for example, small molecule drugs (e.g., especially including anti-cancer drugs and/or antiviral drugs such as anti-HBV or anti-HCV drugs) and other hydrophobic cargo such as fluorescent dyes.

In other embodiments, the cargo can be loaded into the pores (mesopores) of the porous particle cores to form the loaded protocell. In various embodiments, any conventional technology that is developed for liposome-based drug delivery, for example, targeted delivery using PEGylation, can be transferred and applied to the protocells.

As discussed above, electrostatics and pore size can play a role in cargo loading. For example, porous silica nanoparticles can carry a negative charge and the pore size can be tunable from about 2 nm to about 10 nm or more. Negatively charged nanoparticles can have a natural tendency to adsorb positively charged molecules and positively charged nanoparticles can have a natural tendency to adsorb negatively charged molecules. In various embodiments, other properties such as surface wettability (e.g., hydrophobicity) can also affect loading cargo with different hydrophobicity.

In various embodiments, the cargo loading can be a synergistic lipid-assisted loading by tuning the lipid composition. For example, if the cargo component is a negatively charged molecule, the cargo loading into a negatively charged silica can be achieved by the lipid-assisted loading. In certain embodiments, for example, a negatively species can be loaded as cargo into the pores of a negatively charged silica particle when the lipid bilayer is fused onto the silica surface showing a fusion and synergistic loading mechanism. In this manner, fusion of a non-negatively charged (i.e., positively charged or neutral) lipid bilayer or liposome on a negatively charged mesoporous particle can serve to load the particle core with negatively charged cargo components. The negatively charged cargo components can be concentrated in the loaded protocell having a concentration exceed about 100 times as compared with the charged cargo components in a solution. In other embodiments, by varying the charge of the mesoporous particle and the lipid bilayer, positively charged cargo components can be readily loaded into protocells.

Once produced, the loaded protocells can have a cellular uptake for cargo delivery into a desirable site after administration. For example, the cargo-loaded protocells can be administered to a patient or subject and the protocell comprising a targeting peptide can bind to a target cell and be internalized or uptaken by the target cell, for example, a cancer cell in a subject or patient. Due to the internalization of the cargo-loaded protocells in the target cell, cargo components can then be delivered into the target cells. In certain embodiments the cargo is a small molecule, which can be delivered directly into the target cell for therapy.

### Exemplary Embodiments

In one embodiment, a population of protocells is provided comprising a population nanoparticles surrounded by a lipid bi-layer, wherein the population of protocells exhibits a polydispersity index of less than about 0.2. In one embodiment, a population of protocells comprising a population of nanoparticles surrounded by a lipid bi-layer is formed by agitating said nanoparticles with liposomes in solution and separating said nanoparticles from said solution, wherein said liposomes are present in said solution at a weight ratio of at least twice that of said nanoparticles, said population of protocells exhibits a polydispersity index of less than about 0.2. In one embodiment, the nanoparticles comprise silica. In one embodiment, the nanoparticles are mesoporous. In one embodiment, the lipid bi-layer is a supported lipid bi-layer. In one embodiment, the nanoparticles are monosized. In one embodiment, the liposomes are monosized. In one embodiment, the solution comprises buffered saline. In one embodiment, the population of protocells has a polydispersity index of less than about 0.1. In one embodiment, said nanoparticles are spheroidal, ellipsoidal, triangular, rectangular polygonal or hexagonal prisms. In one embodiment, said liposomes are unilamellar. In one embodiment, said liposomes are a mixture of unilamellar and multilamellar. In one embodiment, said liposomes have an internal surface area larger than an external surface area of said nanoparticles. In one embodiment, said lipid bi-layer has a lipid transition temperature (Tₘ) which is greater than the temperature at which said population of protocells will be stored or used. In one embodiment, said lipid bi-layer comprises more than about 50 mole percent an anionic, cationic or zwitterionic phospholipid.

In one embodiment, said lipid bi-layer comprises lipids selected from the group consisting of 1,2-dioleoyl-s-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000](16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), and mixtures thereof. The population of protocells according to any one of claims 1-16 wherein said lipid bi-layer comprises 1,2-distearoyl-sn-glycero-3-phosphocholine (DSPC), 1,2-dioleoyl-sn-glycero-3-phosphoethanolamine (DOPE), or a mixture thereof. The population of protocells according to any one of claims 1-17 wherein said lipid bi-layer comprises cholesterol. The population of protocells according to any one of claims 1-18 wherein said lipid bi-layer comprises about 0.1 mole percent to about 25 mole percent of at least one lipid comprising a functional group to which a functional moiety may be covalently attached. The population of protocells according to claim 19 wherein said lipid comprising a function group is a PEG-containing lipid.

In one embodiment, said PEG-containing lipid is selected from the group consisting of 1,2-dioleoyl-sn-glycero-3-phosphoethanolamine-N-[methoxy(polyethylene glycol)] (ammonium salt) (DOPE-PEG), 1,2-distearoyl-sn-glycero-3-phosphoethanolamine-N-[methoxy(polyethylene glycol)] (ammonium salt) (DSPE-PEG), 1,2-distearoyl-sn-glycero-3-phosphoethanolamine-N-[amino(polyethylene glycol)](DSPE-PEG-NH₂), or a mixture thereof.

In one embodiment, said protocells comprise at least one component selected from the group consisting of: a cell targeting species; a fusogenic peptide; and a cargo, wherein said cargo is optionally conjugated to a nuclear localization sequence. In one embodiment, said protocells comprise a cell targeting species. In one embodiment, said cell targeting species is a peptide, an antibody, an affibody or a small molecule moiety which binds to a cell. In one embodiment, said protocells comprise a fusogenic peptide. In one embodiment, said fusogenic peptide is H5WYG peptide, 8 mer polyarginine, RALA peptide, KALA peptide, GALA peptide, INF7 peptide, or a mixture thereof. In one embodiment, said protocells comprise a cargo. In one embodiment, said cargo is an anti-cancer agent, anti-viral agent, an antibiotic, an antifungal agent, a polynucleotide, a peptide, a protein, an imaging agent, or a mixture thereof. In one embodiment, said polynucleotide comprises encapsulated DNA, double stranded linear DNA, a plasmid DNA, small interfering RNA, small hairpin RNA, microRNA, or mixtures thereof. A storage stable composition comprising a population of protocells, in one embodiment, in an aqueous solution. In one embodiment, said aqueous solution comprises a saline solution. A pharmaceutical composition comprising a population of protocells, in one embodiment, and a pharmaceutically acceptable excipient.

A method of making protocells is provided. In one embodiment, the method include agitating a population of monosized nanoparticles with a population of monosized liposomes in solution, wherein the weight percent of liposomes to nanoparticles in solution is at least 200%, and separating said protocells from said solution. In one embodiment, said solution is an aqueous buffered solution. In one embodiment, said mMSNPs and said liposomes are agitated by sonication. In one embodiment, said protocells are separated from said solution by centrifugation. In one embodiment, said liposomes have an internal surface area which is greater than the external surface area of said nanoparticles. A method of treating a disease comprising administering to a patient an effective amount of the composition to said patient.

In one embodiment, a population of protocells comprising a population nanoparticles surrounded by a lipid bi-layer, wherein the population of protocells exhibits a polydispersity index of less than about 0.2. In one embodiment, the nanoparticles comprise silica. In one embodiment, the nanoparticles are mesoporous. In one embodiment, the lipid bi-layer is a supported lipid bi-layer. In one embodiment, the nanoparticles are monosized. In one embodiment, the population of protocells has a polydisperisty index of less than about 0.1. In one embodiment, said lipid bi-layer has a lipid transition temperature (Tₘ) which is greater than the temperature at which said population of protocells will be stored or used. In one embodiment, said lipid bi-layer comprises more than about 50 mole percent an anionic, cationic or zwitterionic phospholipid or said lipid bi-layer comprises lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycero (DOPG), 1,2-dioleoyl-sn-glycero-3-phosphoethanolamine (DOPE), 1,2-dipalmitoyl-s-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), and mixtures thereof; or wherein said lipid bi-layer comprises 1,2-distearoyl-sn-glycero-3-phosphocholine (DSPC), 1,2-dioleoyl-sn-glycero-3-phosphoethanolamine (DOPE), or a mixture thereof: or wherein said lipid bi-layer comprises cholesterol. In one embodiment, said lipid bi-layer comprises about 0.1 mole percent to about 25 mole percent of at least one lipid comprising a functional group to which a functional moiety may be covalently attached.

In one embodiment, said lipid comprising a function group is a PEG-containing lipid, optionally wherein said PEG-containing lipid is selected from the group consisting of 1,2-dioleoyl-sn-glycero-3-phosphoethanolamine-N-[methoxy(polyethylene glycol)](ammonium salt) (DOPE-PEG), 1,2-distearoyl-sn-glycero-3-phosphoethanolamine-N-[methoxy(polyethylene glycol)] (ammonium salt) (DSPE-PEG), 1,2-distearoyl-sn-glycero-3-phosphoethanolamine-N-[amino(polyethylene glycol)] (DSPE-PEG-NH₂), or a mixture thereof. In one embodiment, said protocells comprise at least one component selected from the group consisting of: a cell targeting species; a fusogenic peptide: and a cargo, wherein said cargo is optionally conjugated to a nuclear localization sequence. In one embodiment, said cell targeting species is a peptide, an antibody, an affibody or a small molecule moiety which binds to a cell. In one embodiment, said protocells comprise a fusogenic peptide, and optionally wherein said fusogenic peptide is H5WYG peptide, 8 mer polyarginine, RALA peptide, KALA peptide, GALA peptide, INF7 peptide, or a mixture thereof, said cargo is an anti-cancer agent, anti-viral agent, an antibiotic, an antifungal agent, a polynucleotide, a peptide, a protein, an imaging agent, or a mixture thereof. In one embodiment, said polynucleotide comprises encapsulated DNA, double stranded linear DNA, a plasmid DNA, small interfering RNA, small hairpin RNA, microRNA, or mixtures thereof.

A storage stable composition comprising a population of protocells in an aqueous solution is provided as well as a pharmaceutical composition comprising a population of protocells and a pharmaceutically acceptable excipient.

In one method, a method to prepare a population of protocells comprising a population of nanoparticles surrounded by a lipid bi-layer is provided, comprising agitating said nanoparticles with liposomes in solution and separating said nanoparticles from said solution, wherein said liposomes are present in said solution at a weight ratio of at least twice that of said nanoparticles, said population of protocells exhibits a polydispersity index of less than about 0.2. In one embodiment, the liposomes are monosized. In one embodiment, the solution comprises buffered saline. In one embodiment, said liposomes are unilamellar. In one embodiment, said liposomes are a mixture of unilamellar and multilamellar. In one embodiment, said liposomes have an internal surface area larger than an external surface area of said nanoparticles. In one embodiment, said agitating is by sonication.

A multilamellar protocell is also provided. The multilamellar provided a nanoporous silica or metal oxide core and a multilamellar lipid bi-layer coating said core, the multilamellar lipid bi-layer comprising at least an inner lipid bi-layer and an outer lipid bi-layer and optionally an inner aqueous layer and/or an outer aqueous layer, said inner aqueous layer separating said core from said inner lipid bi-layer and said outer aqueous layer separating said inner lipid bi-layer from said outer lipid bi-layer said outer lipid bi-layer comprising: at least one Toll-like receptor (TLR) agonist; a fusogenic peptide; and optionally at least one cell targeting species which selectively binds to a target on antigen presenting cells (APCs); said inner lipid bi-layer comprising an endosomolytic peptide.

Further provided is a unilamellar protocell comprising: a nanoporous silica or metal oxide core and a lipid bi-layer coating said core and an optional aqueous layer separating said core from said lipid i-layer, said lipid bi-layer comprising: at least one Toll-like receptor (TLR) agonist: a fusogenic peptide; optionally at least one cell targeting species which selectively binds to a target on antigen presenting cells (APCs); and an endosomolytic peptide. In one embodiment, said Toll-like receptor (TLR) agonist comprises Pam3Cys, HMGB1, Porins, HSP, GLP, BCG-CWS, HP-NAP, Zymosan, MALP2, PSK, dsRNA, Poly AU, Poly ICLC, Poly I:C, LPS, EDA, HSP, Fibrinogen, Monophosphoryl Lipid A (MPLA), Flagellin, Imiquimod, ssRNA, PolyG10, CpG, and mixtures thereof. In one embodiment, said toll-like receptor (TLR) agonist is effective to initiate an immunological signaling cascade. In one embodiment, the fusogenic peptide comprises octa-arginine (R8) peptide. In one embodiment, the fusogenic peptide induces cellular uptake of the protocell. In one embodiment, the cell targeting species selectively binds to a target on antigen presenting cells (APCs). In one embodiment, the endosomolytic peptide comprises H5WYG peptide (H2N-GLFHAIAHFIHGGWHGLIHGWYGGC-COOH, SEQ ID NO: 2), RALA peptide (NH₂-WEARLARALARALARHLARALARALRAGEA-COOH, SEQ ID NO: 18), KALA peptide (NH₂-WEAKLAKALAKALAKHLAKALAKALKAGEA-COOH, SEQ ID NO:19), GALA (NH₂-WEAALAEALAEALAEHLAEALAEALEALAA-COOH, SEQ ID NO:20) or INF7 (NH₂-GLFEAIEGFIENGWEGMIDGWYG-COOH, SEQ ID NO:21). In one embodiment, the endosomolytic peptide enhances endosomal escape. In one embodiment, said outer lipid bi-layer, said inner lipid bi-layer, and/or at least one aqueous layer comprises at least one viral antigen. In one embodiment, said core is loaded with a viral antigen. In one embodiment, the viral antigen is ubiquitinated. In one embodiment, the core is loaded with a plasmid DNA. In one embodiment, the plasmid DNA encodes a viral antigen. In one embodiment, the viral antigen is fused to ubiquitin. In one embodiment, said protocell is loaded with a DNA plasmid in the core and optionally contains a viral antigen. In one embodiment, said viral antigen is a full length viral protein, a viral protein fragment, or a mixture thereof. In one embodiment, the protocell further comprising a bioactive agent. In one embodiment, the protocell further comprising a reporter. In one embodiment, said bioactive agent is loaded into the core of said protocell. In one embodiment, said bioactive agent is a drug or an adjuvant. In one embodiment, said drug is an immunostimulant. In one embodiment, the antigen presenting cell is a professional antigen presenting cell. In one embodiment, the antigen presenting cell is a non-professional antigen presenting cell.

A pharmaceutical composition comprising a population of the protocells in combination with a pharmaceutically acceptable carrier, additive or excipient is also provided. In one embodiment, the composition further comprises a drug, reporter or adjuvant in combination with said population of protocells. A vaccine comprising the composition optionally in combination with an adjuvant, is further provided. A method of inducing an immunogenic response in a subject is provided, wherein a subject is administered an effective amount of the composition. A method inducing immunity to a microbial infection in a subject is also provided comprising administering at least once, an effective amount of the composition to a subject. In one embodiment, said composition is administered as a booster subsequent to a first administration of said composition.

In one embodiment, a multilamellar protocell is provided comprising: a nanoporous silica or metal oxide core and a multilamellar lipid bi-layer coating said core, the multilamellar lipid bi-layer comprising at least an inner lipid bi-layer and an outer lipid bi-layer and optionally an inner aqueous layer and/or an outer aqueous layer, said inner aqueous layer separating said core from said inner lipid bi-layer and said outer aqueous layer separating said inner lipid bi-layer from said outer lipid bi-layer said outer lipid bi-layer; comprising: at least one Toll-like receptor (TLR) agonist; a fusogenic peptide; and optionally at least one cell targeting species which selectively binds to a target on antigen presenting cells (APCs): said inner lipid bi-layer comprising an endosomolytic peptide.

In one embodiment, a unilamellar protocell comprising: a nanoporous silica or metal oxide core and a lipid bi-layer coating said core and an optional aqueous layer separating said core from said lipid bi-layer, said lipid bi-layer comprising: at least one Toll-like receptor (TLR) agonist; a fusogenic peptide; optionally at least one cell targeting species which selectively binds to a target on antigen presenting cells (APCs); and an endosomolytic peptide. In one embodiment, said Toll-like receptor (TLR) agonist comprises Pam3Cys, HMGB1, Porins, HSP, GLP, BCG-CWS, HP-NAP, Zymosan, MALP2, PSK, dsRNA, Poly AU, Poly ICLC, Poly I:C, LPS, EDA, HSP, Fibrinogen. Monophosphoryl Lipid A (MPLA), Flagellin, Imiquimod, ssRNA, PolyG10. CpG, and mixtures thereof. In one embodiment, said toll-like receptor (TLR) agonist is effective to initiate an immunological signaling cascade. In one embodiment, the fusogenic peptide comprises octa-arginine (R8) peptide. In one embodiment, the fusogenic peptide induces cellular uptake of the protocell. In one embodiment, the cell targeting species selectively binds to a target on antigen presenting cells (APCs). In one embodiment, the endosomolytic peptide comprises H5WYG peptide (H2N-GLFHAIAHFIHGGWHGLIHGWYGGC-COOH, SEQ ID NO: 2), RALA peptide (NH₂-WEARLARALARALARHLARALARALRAGEA-COOH, SEQ ID NO: 18), KALA peptide (NH₂-WEAKLAKALAKALAKHLAKALAKALKAGEA-COOH, SEQ ID NO:19), GALA (NH₂-WEAALAEALAEALAEHLAEALAEALEALAA-COOH, SEQ ID NO:20) or INF7 (NH₂-GLFEAIEGFIENGWEGMIDGWYG-COOH, SEQ ID NO:21). In one embodiment, the endosomolytic peptide enhances endosomal escape. In one embodiment, said outer lipid bl-layer, said inner lipid bi-layer, and/or at least one aqueous layer comprises at least one viral antigen. In one embodiment, said core is loaded with a viral antigen. In one embodiment, the core is loaded with a plasmid DNA which optionally encodes a viral antigen. In one embodiment, the viral antigen is fused to ubiquitin. In one embodiment, said protocell is loaded with a DNA plasmid in the core and optionally contains a viral antigen. In one embodiment, the protocell further comprises a bioactive agent. In one embodiment, said bioactive agent is loaded into the core of said protocell. In one embodiment, the antigen presenting cell is a professional antigen presenting cell. In one embodiment, the antigen presenting cell is a non-professional antigen presenting cell.

A pharmaceutical composition comprising a population of protocells in combination with a pharmaceutically acceptable carrier, additive or excipient is also provided, e.g., one, further comprising a drug, reporter or adjuvant in combination with said population of protocells. Further provided is a vaccine comprising the composition, optionally in combination with an adjuvant, and methods, e.g., inducing an immunogenic response in a subject comprising administering to said subject an effective amount of the composition, or, a method inducing immunity to a microbial infection in a subject comprising administering at least once, an effective amount of a composition.

The invention will be described by the following non-limiting examples.

### Example 1

### Materials

All chemicals and reagents were used as received. Ammonium hydroxide (NH₄₀H, 28-30%), 3-aminopropyltriethoxysilane (98%, APTES), ammonium nitrate (NH₄NO₃), benzyldimethylhexadecylammonium chloride (BDHAC), n-cetyltrimethylammonium bromide (CTAB), N,N-dimethyl formamide (DMF), dimethyl sulfoxide (DMSO), rhodamine B isothiocyanate (RITC), tetraethyl orthosilicate (TEOS), and Triton X-100 were purchased from Sigma-Aldrich (St. Louis, Mo.). Hydrochloric acid (36.5-38%, HC) was purchased from EMD Chemicals (Gibbstown, N.J.). Absolute (99.5%) and 95% ethanol were obtained from PHARMCO-AAPER (Brookfield, Conn.). 1,2-dioleoyl-sn-glycero-3-phosphocholine (DOPC), 1,2-distearoyl-sn-glycero-3-phosphocholine (DSPC), 1,2-dioleoyl-sn-glycero-3-phosphoethanolamine-N-[methoxy(polyethylene glycol)-2000] (ammonium salt) (DOPE-PEG₂₀₀₀), 1,2-distearoyl-sn-glycero-3-phosphoethanolamine-N-[methoxy(polyethylene glycol)-2000] (ammonium salt) (DSPE-PEG₂₀₀₀), 1,2-distearoyl-sn-glycero-3-phosphoethanolamine-N-[amino(polyethylene glycol)-2000] (DSPE-PEG₂₀₀₀-NH₂) phospholipids and cholesterol (Chol, ovine wool, >98%) were purchased from Avanti Polar Lipids (Birmingham, Ala.). Hoechst 33342, Traut's reagent, and maleimide-activated NeutrAvidin protein were obtained from Thermo Scientific (Rockford, Ill.). Alexa Fluor® 488 phalloidin and CellTracker™ green CMFDA dye were purchased from Life Technologies (Eugene, Oreg.). Heat inactivated fetal bovine serum (FBS), 10× phosphate buffered saline (PBS), 1× trypsin-EDTA solution, and penicillin streptomycin (PS) were purchased from Gibco (Logan, Utah). Dulbecco's Modification of Eagle's Medium with 4.5 g/L glucose, L-glutamine and sodium pyruvate (DMEM) and RPMI-1640 medium were obtained from CORNING cellgro (Manassas, Va.). Doxorubicin was purchased from LC Laboratories (Woburn, Mass.). Anti-EGFR antibody [EGFR1] (Biotin) (ab24293) was purchased from Abcam (Cambridge, Mass.).

Synthesis of mMSNs Composed of Hexagonally Arranged Cylindrical Pores (2.8 nm Pore Size).

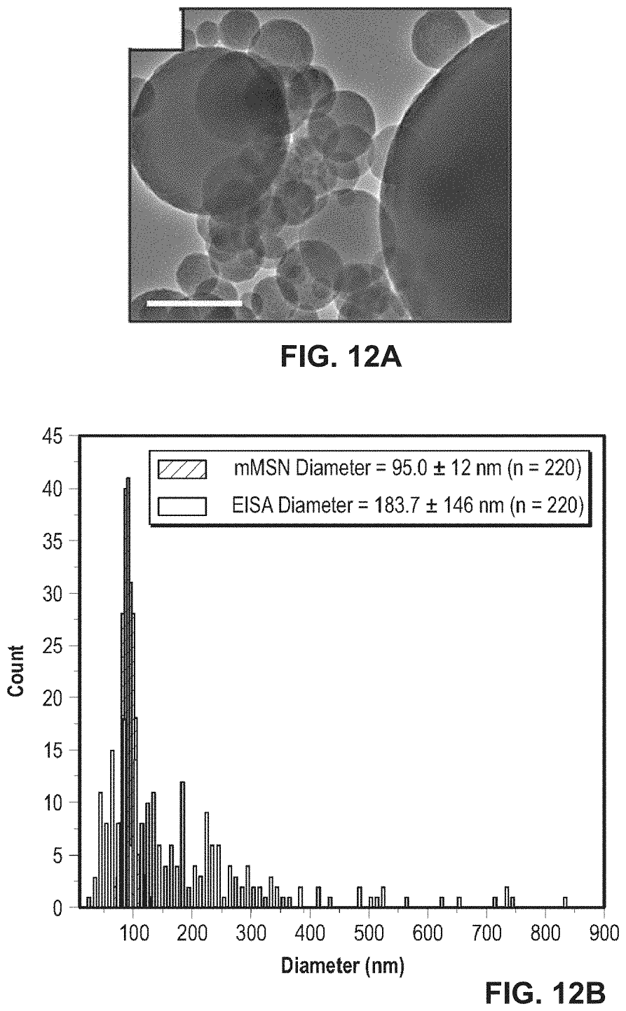

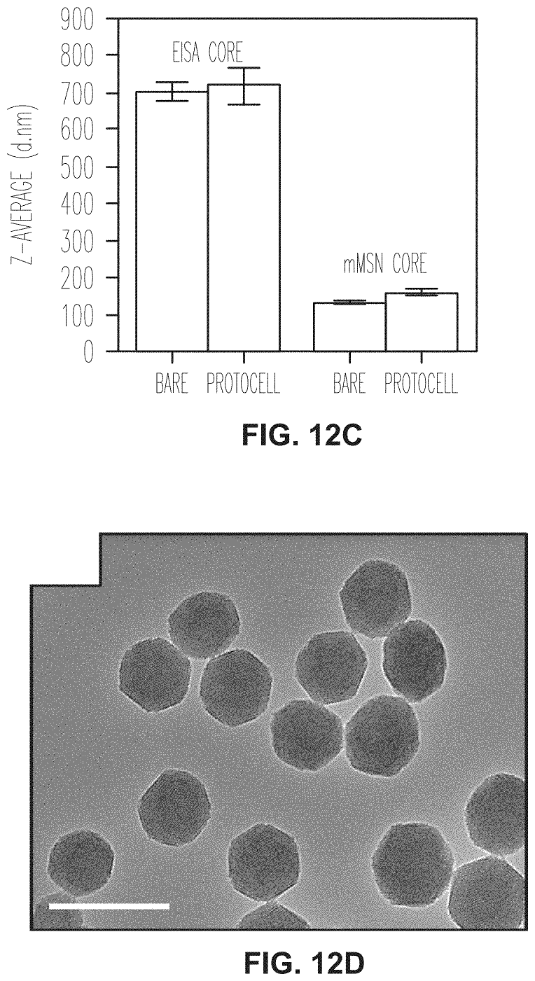

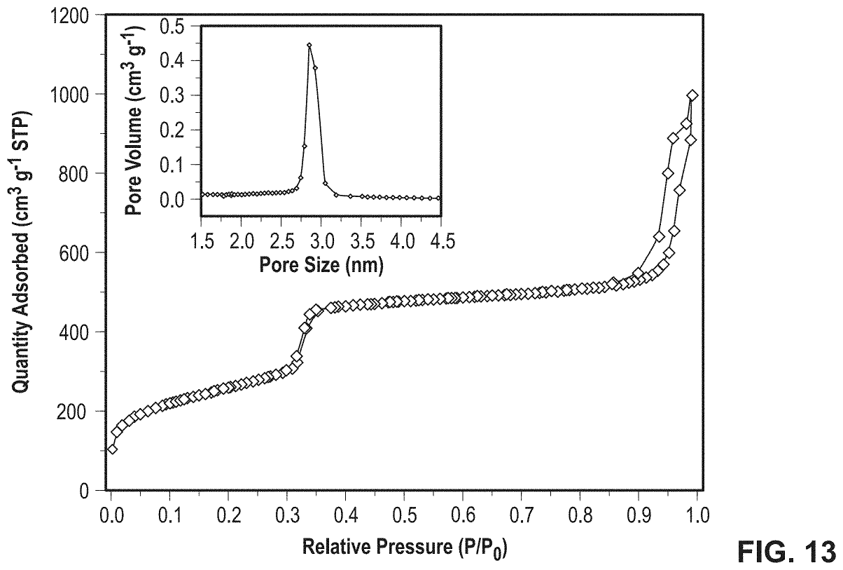

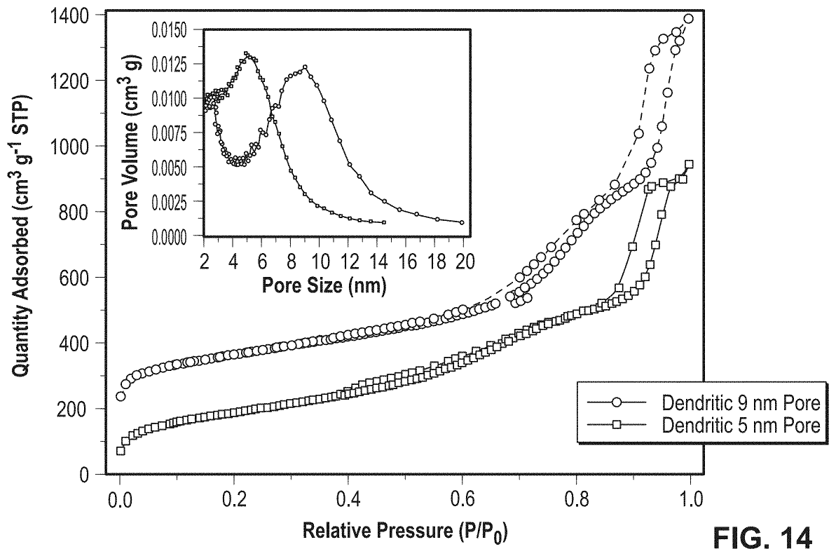

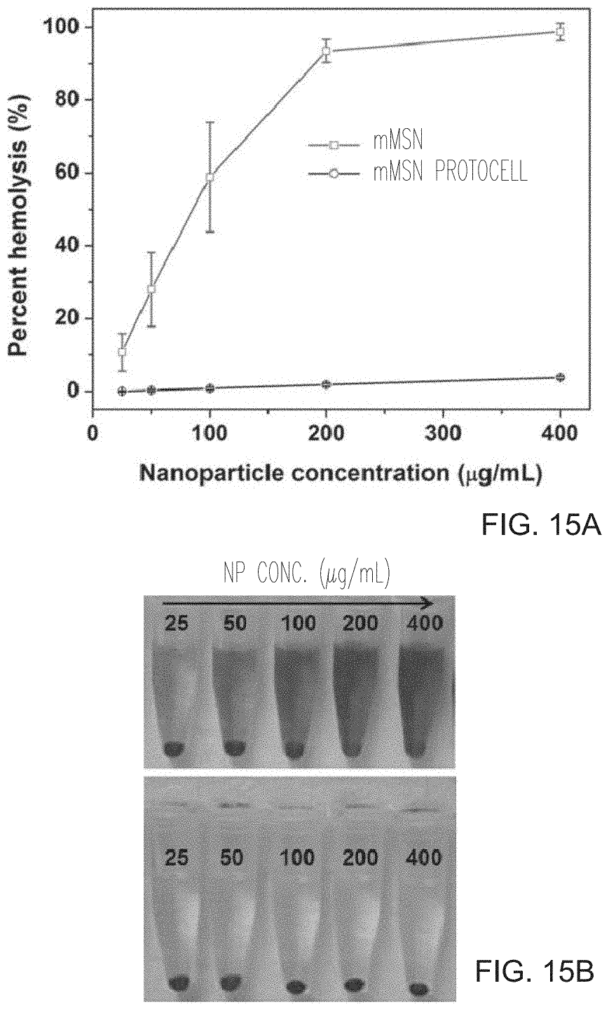

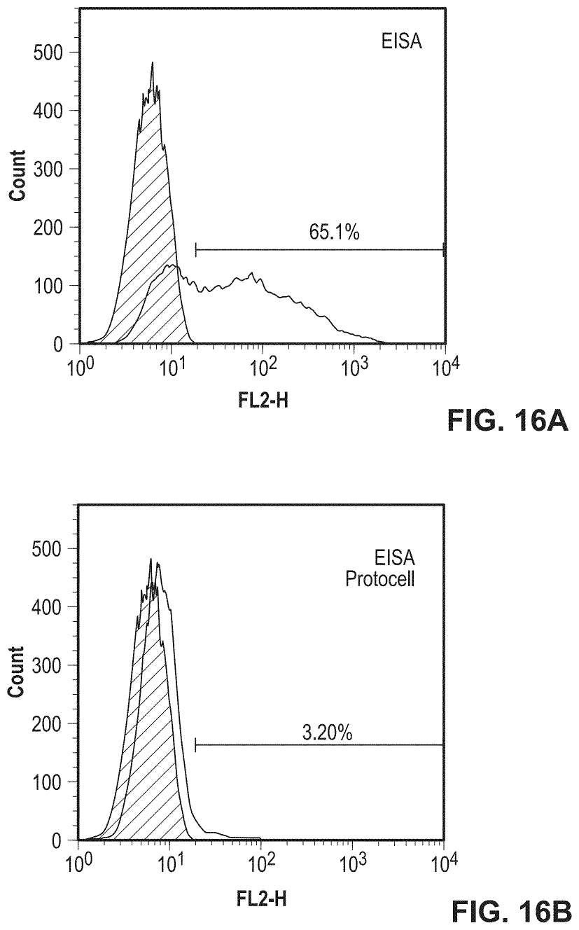

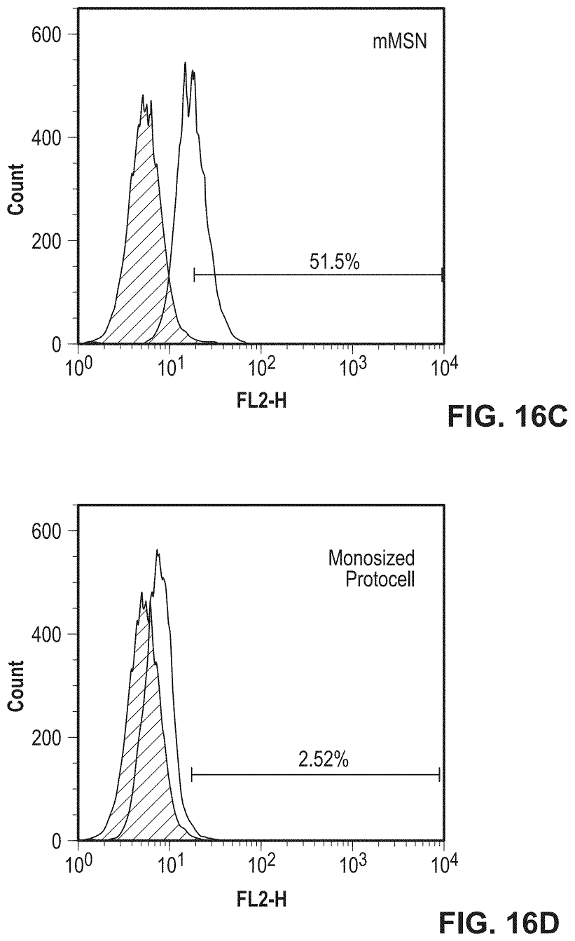

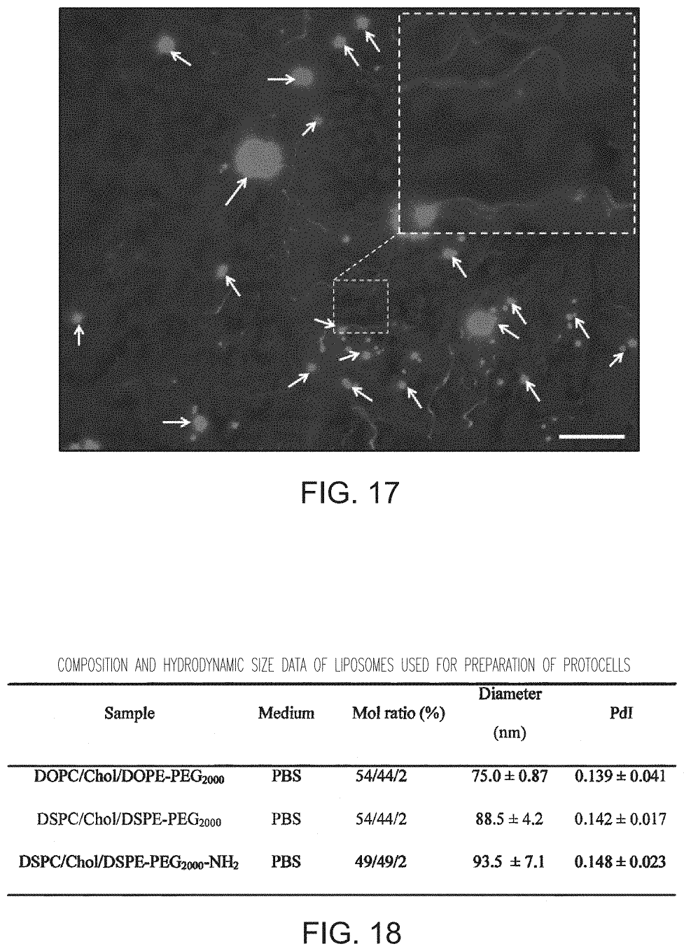

To prepare monosized dye-labeled mMSNs (about 95 nm in diameter, FIG. 12, about 130 nm in hydrodynamic size in D.I. water), 3 mg of RITC was dissolved in 2 mL of DMF followed by addition of 1.5 μL APTES Townson et al., 2013). The synthesis conditions of mMSNs are based on reported literature (Lin and Haynes, 2011). The RITC-APTES solution was incubated at room temperature for at least 1 hour. Next, 290 mg of CTAB was dissolved in 150 mL of 0.51 M ammonium hydroxide solution in a 250 mL beaker, sealed with parafilm (Neenah, Wis.), and placed in a mineral oil bath at 50° C. After continuously stirring for 1 hour, 3 mL of 0.88 M TEOS solution (prepared in ethano) and 1 mL of RITC-APTES solution were combined and added immediately to the surfactant solution. After another 1 hour of continuous stirring, the particle solution was stored at 50° C. for about 18 hours under static conditions. Next, solution was passed through a 1.0 μm Acrodisc 25 mm syringe filter (PALL Life Sciences, Ann Arbor, Mich.) followed by a hydrothermal treatment at 70° C. for 24 hours. Followed procedure for CTAB removal was as described in literature (Lin et al., 2011). Briefly, mMSNs were transferred to 75 mM ammonium nitrate solution (prepared in ethanol) then placed in an oil bath at 60° C. for 1 hour with reflux and stirring. The mMSNs were then washed in 95% ethanol and transferred to 12 mM HCl ethanolic solution and heated at 60° C. for 2 hours with reflux and stirring. Lastly, mMSNs were washed in 95% ethanol, then 99.5% ethanol, and stored in 99.5% ethanol.

Synthesis of Spherical mMSNs with Isotropic Pores (2.5 nm Pore Size).

To prepare monosized spherical mMSNs composed of isotropic mesopores, the same procedure described above for synthesis of mMSNs with hexagonally arranged pore structure was used. However, cationic surfactant BDHAC was substituted for CTAB as the template. The 3-dimensional isotropic pore arrangement is due to a larger micelle packing parameter of BDHAC, compared to CTAB surfactant (Chen et al., 2013).

Synthesis of Dendrimer-Like mMSNs Composed of Large Pores (5 nm and 9 nm Core Size).

The large pore mMSNs were synthesized by a published biphase method (Bayu et al., 2009; Wang et al., 2012; Shen et al., 2014). Syntheses of 5 nm and 9 nm pore mMSNs are based on a modified condition reported by Zhao et al. (2014). For preparation of dendritic 5 nm pore mMSNs, 0.18 g of TEA was dissolved in 38 mL of DI water and 24 mL of 25 w % CTAC in a 100 mL round bottom flask. The surfactant solution was stirred at 150 rpm and heated at 50° C. in an oil bath. After 1 hour, 20 mL of v/v % TEOS (in cyclohexane) was added to the CTAC-TEA aqueous solution. After 12 hours, the particle solution was washed with DI water twice by centrifugation. Further surfactant removal achieved by following the previously described conditions used in preparation of small pore mMSNs. For synthesis of 9 nm pore mMSNs, we adjusted the stirring rate and organic phase concentration to 300 rpm and 10 v/v % TEOS, respectively. All other steps were identical.

Synthesis of Rod-Shaped mMSNs with Hexagonally Arranged Cylindrical Cores (2.8 nm Pore Size).

The shape of mMSNs can be simply tuned to rod-like morphology by altering the CTAB concentration, stirring rate, and ammonia concentration (Huang et al., 2011; Uy et al., 2011). Briefly, 0.5 g CTAB was dissolved in 150 mL of 0.22 M ammonium hydroxide solution at 25° C. under continuous stirring (300 rpm). Next, of 1 mL TEOS was added (drop wise) to the surfactant solution with stirring. After 1 hour, the particle solution was aged under static conditions for 24 hours, then subsequently transferred to a sealed container and heated to 70° C. for 24 hours. The removal of surfactant was followed the same procedures described previously.

Liposome Preparation.

Lipids and cholesterol ordered from Avanti Polar Lipids were presolubilized in chloroform at 25 mg/mL and were stored at −20° C. To prepare liposomes, lipids were mixed at different mol % ratios including (54/44/2) for DOPC/Chol/DOPE-PEG₂₀₀₀ and DSPC/Chol/DSPE-PEG₂₀₀₀, and (49/49/2) for DSPC/Chol/DSPE-PEG₂₀₀₀-NH₂. Lipid films were prepared by drying lipid mixtures (in chloroform) under high vacuum to remove the organic solvent. Then the lipid film was hydrated in 0.5×PBS and bath sonicated for 30 minutes to obtain a liposome solution. Finally, the liposome solution was further passed through a 0.05 μm polycarbonate filter membrane (minimum 21 passes) using a mini-extruder to produce uniform and unilamellar vesicles with hydrodynamic diameters less than 100 nm.

Protocell Preparation.

To form protocells, mMSNs are transferred to D.I. water at 1 mg/mL concentration by centrifugation (15,000 g, 10 minutes) and added to liposome solution in 0.5×PBS (1:1 v/v and 1:2 w/w ratios). The mixture was bath sonicated about 10 seconds and non-fused liposomes were removed by centrifugation (15,000 g, 10 minutes). Pelleted protocells were redispersed in 1×PBS via bath sonication, this step is repeated twice.

Anti-EGFR Protocell Preparation.

First, DSPC/Cho/DSPE-PEG-NH₂ liposomes were prepared according to the method described previously. Next, a ratio (2:1, w:w) of DSPC/Chol/DSPE-PEG₂₀₀₀-NH₂ liposomes to bare RITC labeled mMSN were combined in a conical tube at room temperature for 30 minutes. The excess liposomes were removed by centrifugation (15,000 g, 10 minutes). The pelleted protocells were redispersed in 1 mL of PBS with bath sonication. To convert the surface —NH₂ to —SH groups, 50 μL of freshly prepared Traut's reagent (250 mM in PBS) was added to the protocells. After 1 hour, the particles were centrifuged, and the supernatant was removed. The particles were again redispersed in 1 mL of PBS. Then, 0.15 mg of maleimide-activated NeutrAvidin protein was added to 0.25 mL of thiolated protocells and incubated at room temperature for 12 hours. The NeutrAvidin conjugated protocells were washed with PBS via centrifugation and suspended in 0.25 mL of PBS. Then, 50 μL of biotinylated EGFR antibody (0.1 mg/mL) was mixed with 50 μL of NeutrAvidin conjugated protocells for at least 30 minutes. Finally, the antibody conjugated protocells were pelleted and redispersed in 100 μL PBS for in vivo targeting experiments.

In Vivo Red Blood Cell Compatibility.

Whole human blood was acquired from healthy donors with informed consent and stabilized in K₂EDTA tubes (BD Biosciences). hRBCs were purified following reported procedure (Liao et al., 2010), then incubated with either bare mMSNs or protocells (25, 50, 100, 200, and 400 μg/mL) at 37° C. After 3 hours of exposure, samples were centrifuged at 300 g for 3 minutes, then 100 μL of supernatant from each sample was transferred to a 96-well plate. Hemoglobin absorbance was measured using a BioTek microplate reader (Winooski, Vt.) at 541 nm. The percent hemolysis of each sample was quantified using a reported equation (Liao et al., 2011).

Cell Culture and Nanoparticle Nonspecific Binding/Uptake.

Human endothelial cells, EA.hy926 (CRL-2922) were purchased from American Type Culture Center (ATCC, Manassas, Va.). We seeded 5×10⁵ EA.hy926 cells in 6-well plates with 2 mL of DMEM+10% FBS and 1% PS at 37° C. in 5% CO₂ humidified atmosphere. After 24 hours, the media was removed and replaced with 2 mL of fresh complete media supplemented with 20 μg/mL of bare mMSNs or protocells for 4 hours at 37° C. under 5% CO₂. After nanoparticle incubation, the media was removed and the cells were gently washed twice with PBS. For imaging purposes, the nanoparticle treated cells were fixed in 3.7% formaldehyde (in PBS) at room temperature for 10 minutes, washed with PBS, then treated with 0.1% Triton X-100 for another 10 minutes. The fixed cells were washed with PBS and stored in 1 mL of PBS. The cell nuclei and F-actin were stained with 1 mL of Hoechst 33342 (3.2 μM in PBS) and 0.5 mL of Alexa Fluor® 488 phalloidin (20 nM in PBS) for 20 minutes, respectively. After staining, the cells were washed with PBS twice and stored in PBS prior to fluorescence microscope imaging. For preparation of flow cytometry samples, the control and nanoparticle treated cells were removed from plate bottom using Trypsin-EDTA (0.25%). The suspended cells were centrifuged, washed with PBS, and suspended in PBS for flow cytometry measurements.

Cell-Nanoparticle Interactions in Ex Ovo Avian Embryos.

Ex ovo avian embryos were handled according to published methods (Leong et al., 2010), with all experiments conducted following an institutional approval protocol (11-100652-T-HSC). This method included incubation of fertilized eggs (purchased from East Mountain Hatchery-Edgewood, N. Mex.) in a GQF 1500 Digital Professional egg incubator (Savannah, Ga.) for 3-4 days. Following initial in ovo incubation, embryos were removed from shells by cracking into 100 mL polystyrene weigh boats (VWR, Radnor, Pa.). Ex ovo embryos were then covered and incubated (about 39° C.) with constant humidity (about 70%). For nanoparticle injections, about 50 μg (at 1 mg/mL) of bare mMSNs or protocells in PBS were injected into secondary or tertiary veins of the CAM via pulled glass capillary needles. CAM vasculature and fluorescent nanoparticles were imaged using a customized avian embryo chamber (humidified) and a Zeiss AxioExaminer upright microscope modified with a heated stage. High speed videos were acquired on the same microscope using a Hamamatsu Orca Flash 4.0 camera.

Post-Circulation Size and Stability Analyses.

All animal care and experimental protocols were in accordance with the National INSTITUTES of Health and University of New Mexico School of Medicine guidelines. Ten- to twelve-week-old female BALB/c mice (Charles River Laboratories, Wilmington, Mass.) were administered dose of RITC-labeled protocells (10 mg/mL) in 150 μL PBS via tall vein injection. After 10 minutes of circulation, mice were euthanized and blood was drawn by cardiac puncture. Whole blood was stabilized in K₂EDTA microtainers (BD Biosciences) prior to analysis. Ex ovo avian embryos were administered dose of RITC-labeled protocells (1 mg/mL) in 50 μL PBS via secondary or tertiary veins of the CAM. After 10 minutes of circulation, blood was drawn via pulled glass capillary needles and analyzed immediately. Whole blood cells and protocell fluorescence in both mouse and avian samples were imaged on a glass slide with Zeiss AxioExaminer fixed stage microscope (Gottingen, Germany). To separate protocells from whole blood, samples were centrifuged at low speed to remove blood cells, supernatant fraction was transferred to a fresh tube then centrifuged at 15,000 g for 10 minutes. The pellets were washed (15,000 g for 10 min) twice in PBS, then protocell size was analyzed on Malvern Zetasizer Nano-ZS equipment.

In Vitro Targeting.

The pro-B-lymphocyte cell lines, Ba/F3 and Ba/F3+EGFR (Li et al., 1995) were a kind gift from Professor David F. Stem, Yale University. The Ba/F3 and Ba/F3+EGFR cells were suspended in RPMI 1640 supplemented with 10% FBS media at a concentration of about 1×10⁸ cells/mL. Then one mL of cells was incubated with anti-EGFR protocells at 5 μg/mL for 1 hour at 37° C. under 5% CO₂. The cell nuclei and membrane were stained by 1 μL of Hoechst 33342 (1.6 mM in DI) and 2 μL of CellTracker™ green CMFDA dye (2.7 mM in DMSO) for 10 minutes. The nanoparticle-treated cells were pelleted using a benchtop centrifuge, washed with PBS twice, and dispersed in PBS. The live cells were imaged on a glass slide using the Zeiss AxioExaminer upright microscope. To further examine the specificity of targeted protocells, the binding of particles was determined by a fluorescence shift measured by a Becton-Dickinson FACScalibur flow cytometer.

In Vivo Single Cell Targeting in Ex Ovo Chicken Embryos.

First, about 1×10⁶ of BAF+EGFR cells were suspended in 1 mL PBS and incubated with 2 μL of CellTracker™ green CMFDA dye for 10 minutes at 37° C. The stained cells were centrifuged, washed, and suspended in 500 μL of PBS. Next, 50 μL of cell solution was administered to ex ovo avian embryos via the previously described procedure. After 30 minutes cell circulation, the anti-EGFR protocells (100 μL, 0.2 mg/mL) were injected into embryos intravenously. The binding and internalization of targeted protocells to cancer cells was imaged at different time points using the Zeiss AxioExaminer upright microscope.

Characterization.

TEM images were acquired on a JEOL 2010 (Tokyo, Japan) equipped with a Gatan Orius digital camera system (Warrendale, Pa.) under a 200 kV voltage. The cryo-TEM samples were prepared using an FEI Vitrobot Mark IV (Eindhoven, Netherlands) on Quantifoil® R1.2/1.3 holey carbon grids (sample volume of 4 μL, a blot force of 1, and blot and drain times of 4 and 0.5 seconds, respectively). Imaging was taken with a JEOL 2010 TEM at 200 kV using a Gatan model 626 cryo stage. Nitrogen adsorption-desorption isotherms of mMSNs were obtained from on a Micromeritics ASAP 2020 (Norcross, Ga.) at 77 K. Samples were degassed at 120° C. for 12 hours before measurements. The surface area and pore size was calculated following the Brunauer-Emmet-Teller (BET) equation in the range of P/Pₒ from 0.05 to 0.1 and standard Barrett-Joyer-Halenda (BJH) method. Flow cytometry data were performed on a Becton-Dickinson FACScalibur flow cytometer (Sunnyvale, Calif.). The raw data obtained from the flow cytometer was processed with FlowJo software (Tree Star, Inc. Ashland, Oreg.). Hydrodynamic size and zeta potential data were acquired on a Malvern Zetasizer Nano-ZS equipped with a He—Ne laser (633 nm) and Non-Invasive Backscatter optics (NIBS). All samples for DLS measurements were suspended in various media (DI, PBS, and DMEM+10% FBS) at 1 mg/mL. Measurements were acquired at 25° C. or 37° C. DLS measurements for each sample were obtained in triplicate. The Z-average diameter was used for all reported hydrodynamic size measurements. The zeta potential of each sample was measured in 1×PBS using monomodal analysis. All reported values correspond to the average of at least three independent samples. The fluorescence images were captured with a Zeiss AxioExaminer fixed stage microscope (Gottingen, Germany).

### Additional Information—Calculation for Examples

Calculations to Identify Optimal Liposome to mMSN Surface Area Ratio.

To estimate the number of particles in solution (n), a spherical model was employed to calculate mMSN exterior surface area (SA) and volume (V_{mMSN}) from diameter (D) obtained from Z-average DLS measurements, pore volume (Vₚₒᵣₑ) measurements from nitrogen adsorption-desorption isotherms (0.73 cm³/g), and a mesoporous silica density (ρ) of 2 g/cm³.

The equations below were used to estimate the number of particles in solution per unit concentration.

$${SA}_{mMSN} = 4 π * {( D / 2 )}^{2}$$

$${V}_{mMSN} = 4 / 3 * π * {( {D}_{mMSN} / 2 )}^{3}$$

$${n}_{mMSN} = \frac{( m / ρ ) + ( m * {V}_{pore} )}{{V}_{mMSN }}$$

Next we determined the theoretical inner and outer surface areas (SAᵢₙₙₑᵣ and SAₒᵤₜₑᵣ) of an individual liposome using the diameter (D) obtained from Z-average DLS measurements of mMSNs and assuming lipid bi-layer thickness (d) of 5.1 nm

SAᵢₙₙₑᵣ=4π*(D/2)²

SAₒᵤₜₑᵣ=4π*[(D/2)+d]²

SAₗᵢₚₒₛₒₘₑ=SAᵢₙₙₑᵣ+SAₒᵤₜₑᵣ

To find the number of component molecules needed to occupy the total theoretical liposome surface area we use the mass used (m) and assume 0.7 nm² to represent single lipid head group area^{[1]} and 0.38 nm² for cholesterol group area.^{[2]}

$${Moles}_{component} = \frac{{m}_{component}}{M {W}_{component}}$$

$${Moles}_{liposome} = {\sum }_{i = 1}^{n} {Moles}_{{i}_{component}}$$

$${SA}_{average component} = \frac{( 0.7 * {\sum }_{i = 1}^{n} {Moles}_{{i}_{component}} + 0.38 * {Moles}_{cholesterol} )}{{Moles}_{liposome}}$$

$${Molecules}_{needed} = \frac{( {n}_{nMSN} * {SA}_{Liposome} )}{{SA}_{average component}}$$

To find the optimal mass of lipid to a fixed mMSN amount, we use the total mass of the liposome components and convert the molecules needed to mass needed.

$$M {W}_{average liposomes} = \frac{{\sum }_{i = 1}^{n} [ {Moles}_{{i}_{component}} * M {W}_{i} ]}{{Moles}_{liposome}}$$

$${m}_{liposomes} = {\sum }_{i = 1}^{n} {Concentration}_{{i}_{component}} * {V}_{{i}_{component}}$$

$${Moles}_{needed} = {Molecules}_{needed} / {N}_{A}$$

$${m}_{needed} = {Moles}_{needed} * M {W}_{average liposomes}$$

The calculated mass of fluorescent liposome (DSPC/Chol/DSPE-PEG₂₀₀₀/NBD-Chol-54/432/1 mol %) to mMSN (118.7 nm Z-average diameter) is 0.263 to 1. The experimental quantification of mass of fluorescent labeled liposome to mMSN is 0.276 to 1, as measured from fluorescence intensity of unbound liposomes in the supernatant following centrifugation of the protocells compared to a standard curve generated from known fluorescent liposome concentration. The calculated and experimental values are within 4.7% of each other, which is supportive of our method of surface area ratio calculations.

### Results

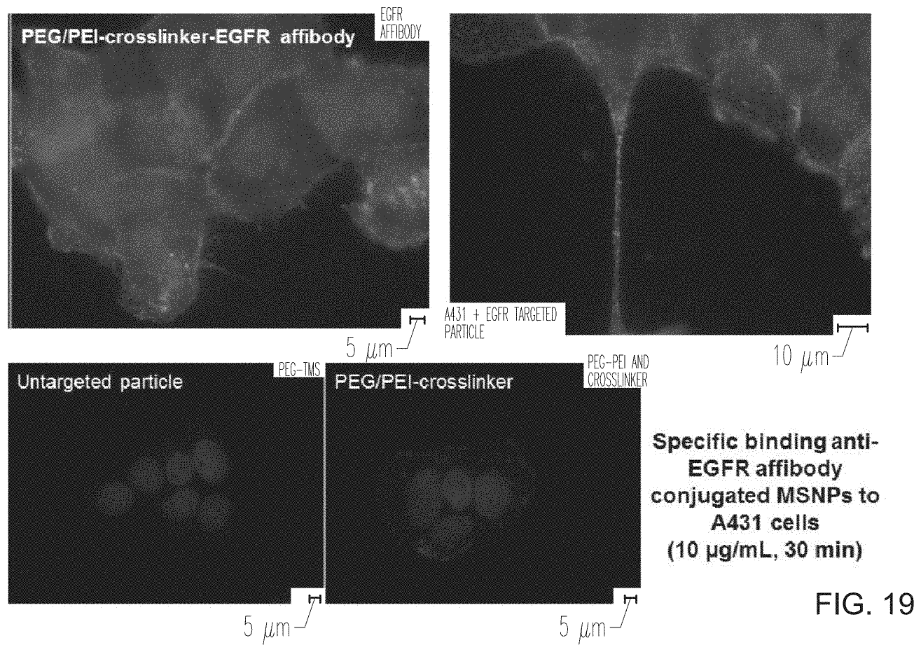

In one approach, a targeting strategy using affibody ligands attached to MSNPs was used to demonstrate crosslinking chemistry. This affibody conjugation chemistry is compatible with amine functionalized lipid head groups, for example—DSPE-PEG-Amine, DPPE-PEG-Amine, DOPE-PEG-Amine, DMPE-PEG-Amine, DSPE, DPPE, DMPE, DOPE, and any other lipid head group with a primary amine group. MSNPs and nuclei stained with DAPI are shown. FIG. 19 shows the in vitro targeting of anti-EGFR affibody MSNPs.

In another approach, a targeting strategy uses peptide ligands attached to MSNPs to demonstrate crosslinking chemistry. This peptide conjugation chemistry is compatible with amine functionalized lipid head groups, for Example—DSPE-PEG-Amine, DPPE-PEG-Amine, DOPE-PEG-Amine, DMPE-PEG-Amine, DSPE, DPPE, DMPE, DOPE, and any other lipid head group with a primary amine group. MSNPs, cytoskeleton stained with phalloidin actin stain, and nuclei stained with DAPI are shown.

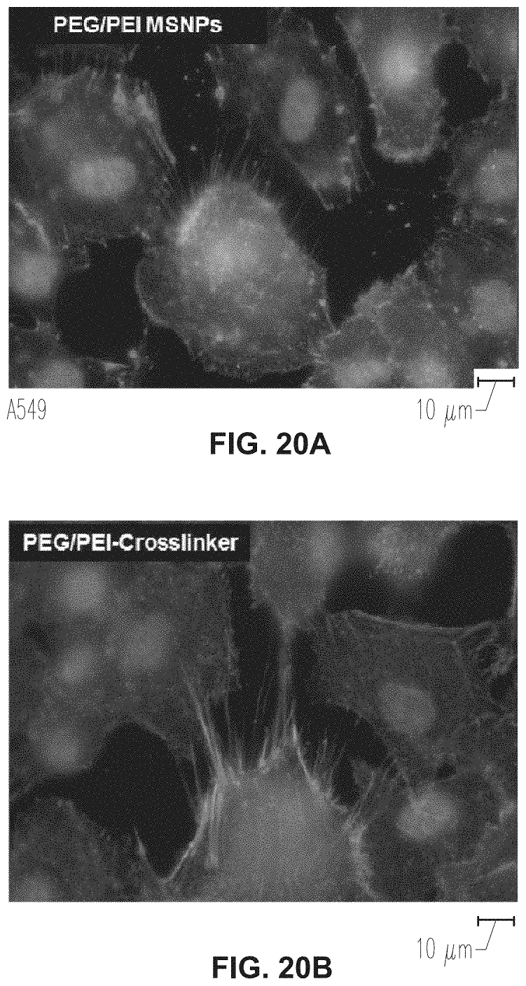

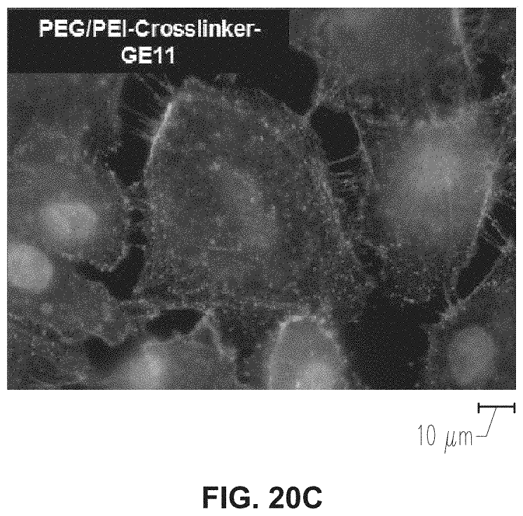

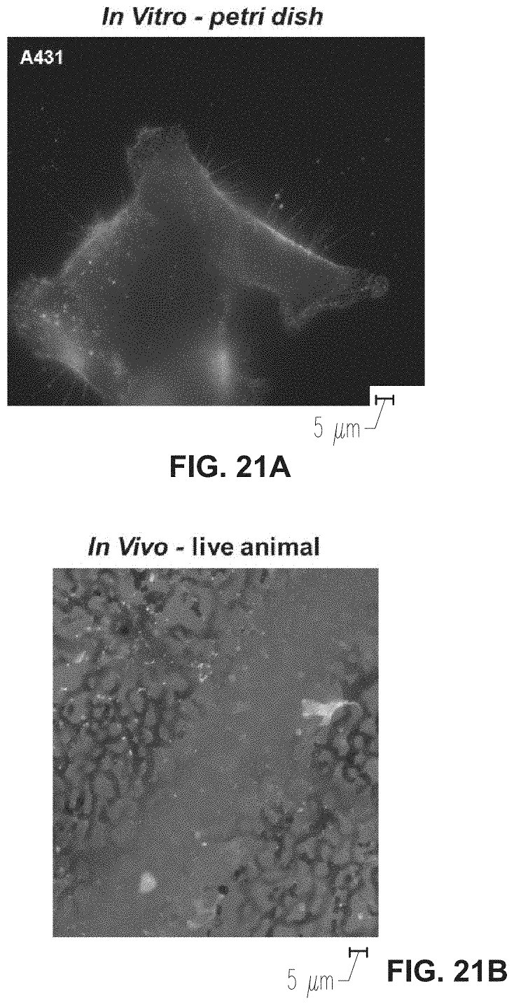

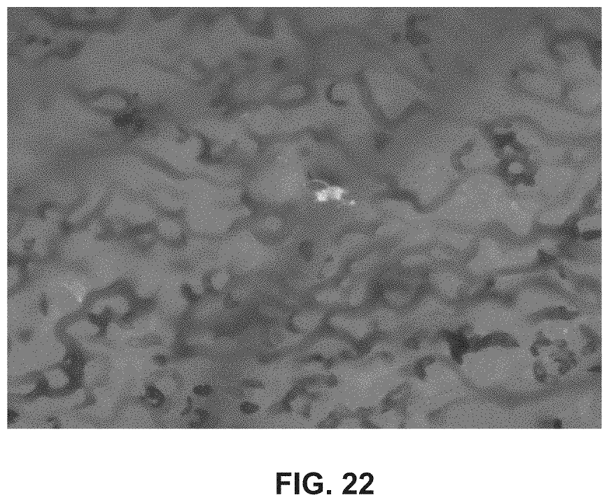

FIG. 20 shows the in vitro targeting of GE11 conjugated MSNPs. FIG. 21 shows evidence of affibody binding both in vitro and in vivo. Left=nanoparticles, with nuclei, right=extravascular space, including nanoparticles, and target A431 cells. Evidence of peptide crosslinked nanoparticles binding to target Hep3B cells ex ovo is shown in FIG. 22. The extravascular space, nanoparticles, and target Hep3B cells are shown.

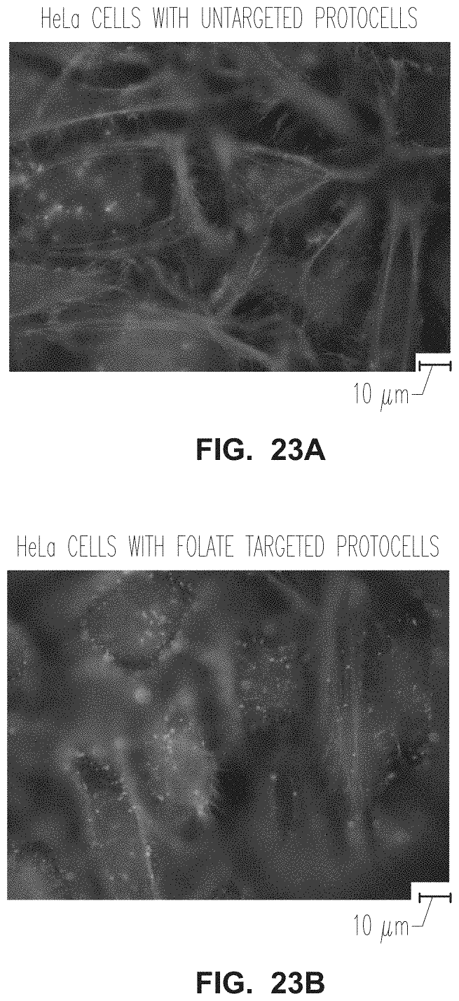

In another example, evidence of successful molecular folate targeting strategy with folate was used to bind to target HeLa cells in vitro. In FIG. 23, the top image shows untargeted protocells do not bind to cells, but with folate conjugated to the SLB a high degree of specific binding is observed (bottom image). This targeting strategy can be achieved using heterobifunctional crosslinker chemistry, copper free click chemistry, copper based click chemistry, homobifunctional crosslinker chemistry, commercially available DSPE-PEG-folate can also be incorporated into standard SLB formulations.

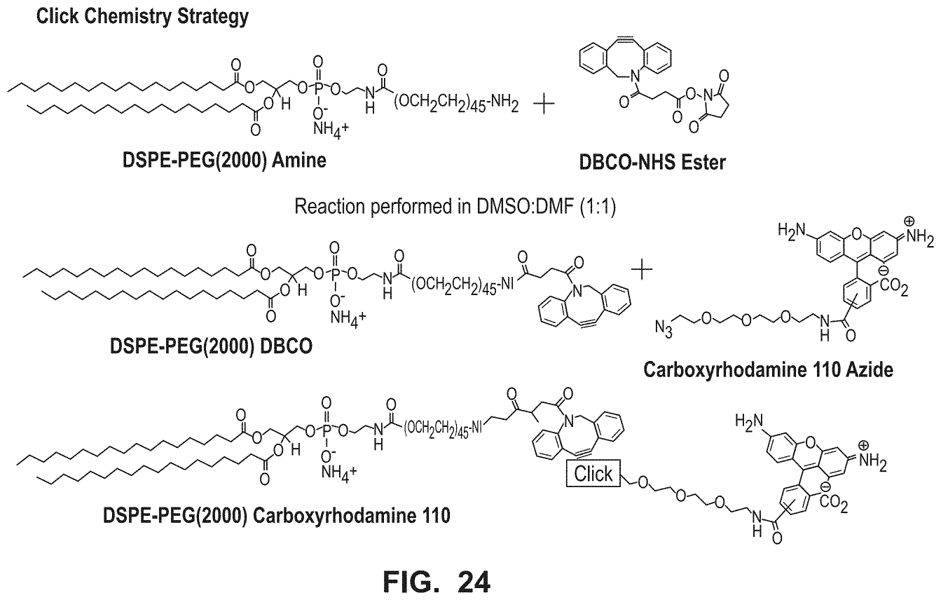

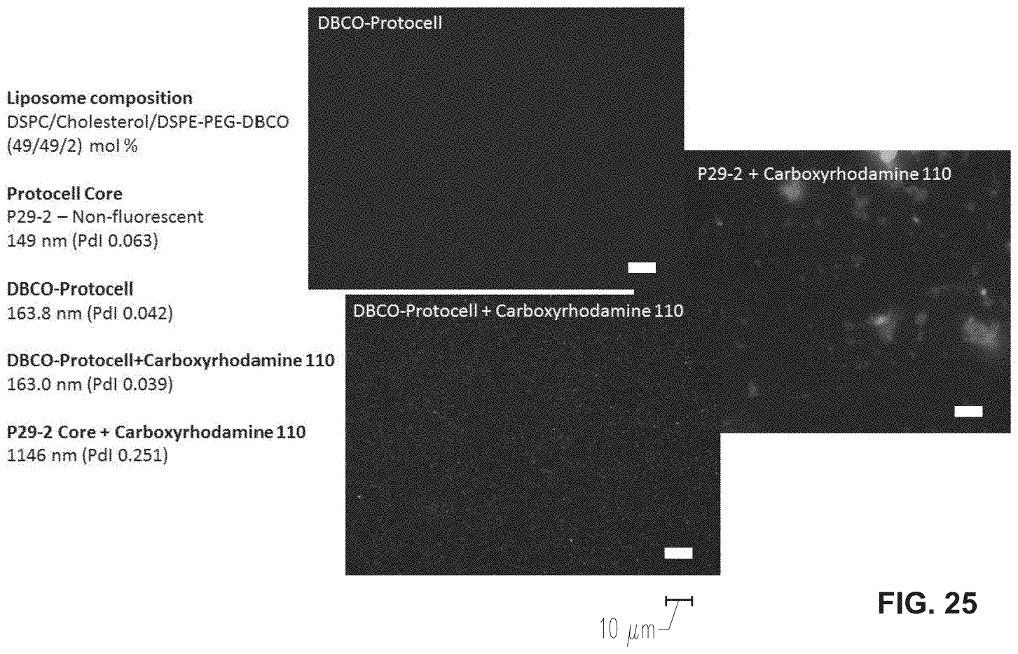

The schematic set forth in FIG. 24 shows how amine terminated lipid head groups can be modified with copper free click moiety (DBCO) which is then capable of bonding to azide (N3) functional groups on molecules, peptides, antibodies, affibodies, single chain variable fragments (scFvs). DSPE-PEG-DBCO is also commercially available and will incorporated in the standard SLB formulations. Lipids can be modified before or after liposome preparation, and or fusion to MSNP support. FIG. 25 shows the measure of size and stability of protocells modified with copper free click lipid head groups (DPSE-PEG-DBCO). The figure shows protocells fluorescence due to successful click reaction to the SLB surface using Carboxyrhodamine 110. The top image shows no fluorescence because it only contains clickable lipid group, middle image shows major aggregation in the absence of SLB, and the bottom image shows disperse population of green labelled protocells in solution. Data on left show that this targeting strategy does not destabilize the protocell because the hydrodynamic size is slightly larger than the MSNP core and the PdI<0.1.

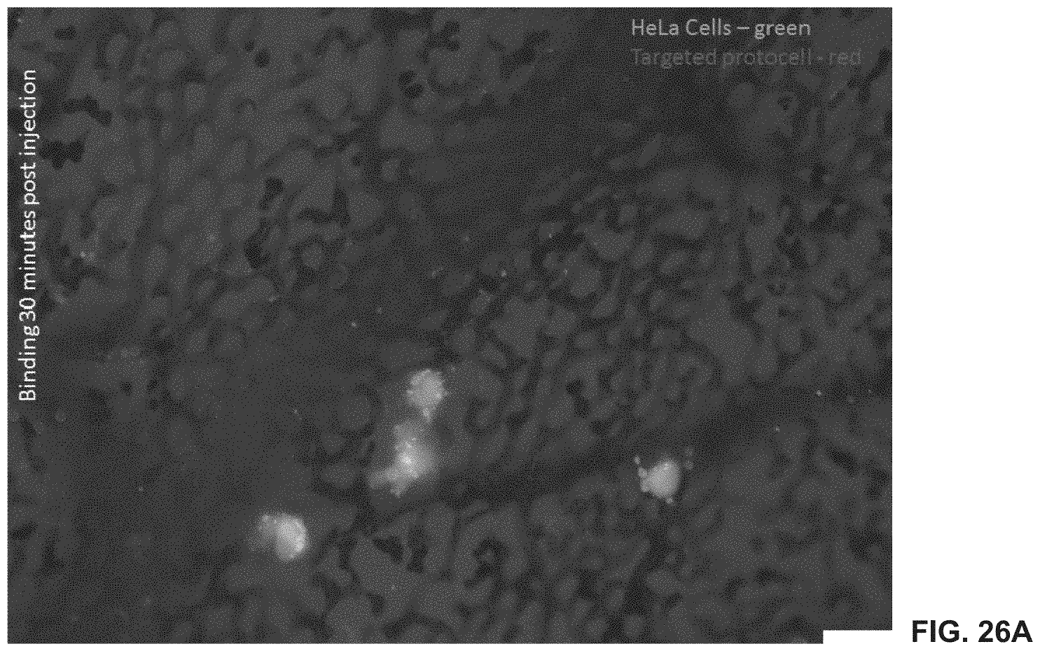

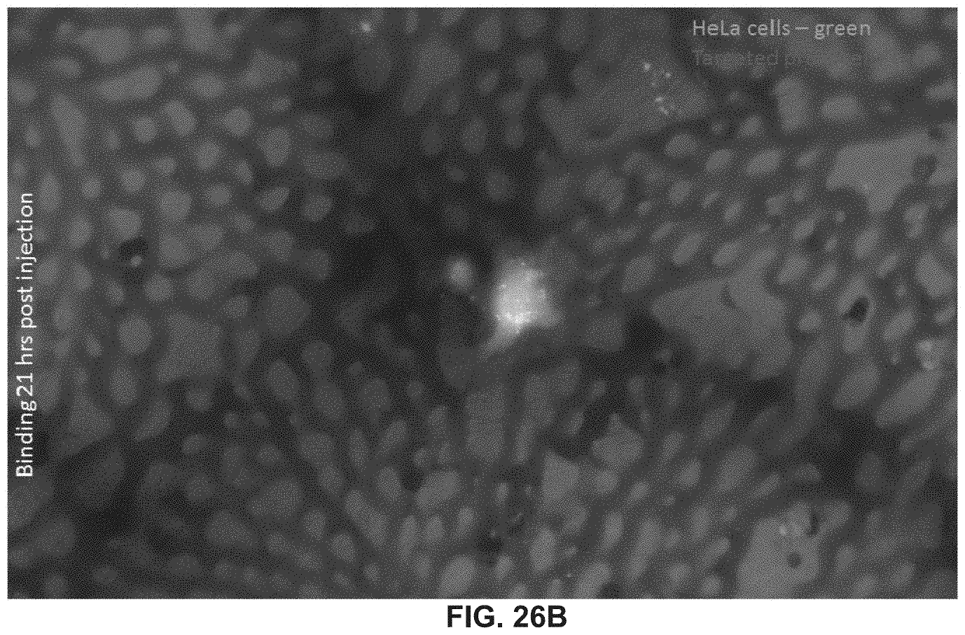

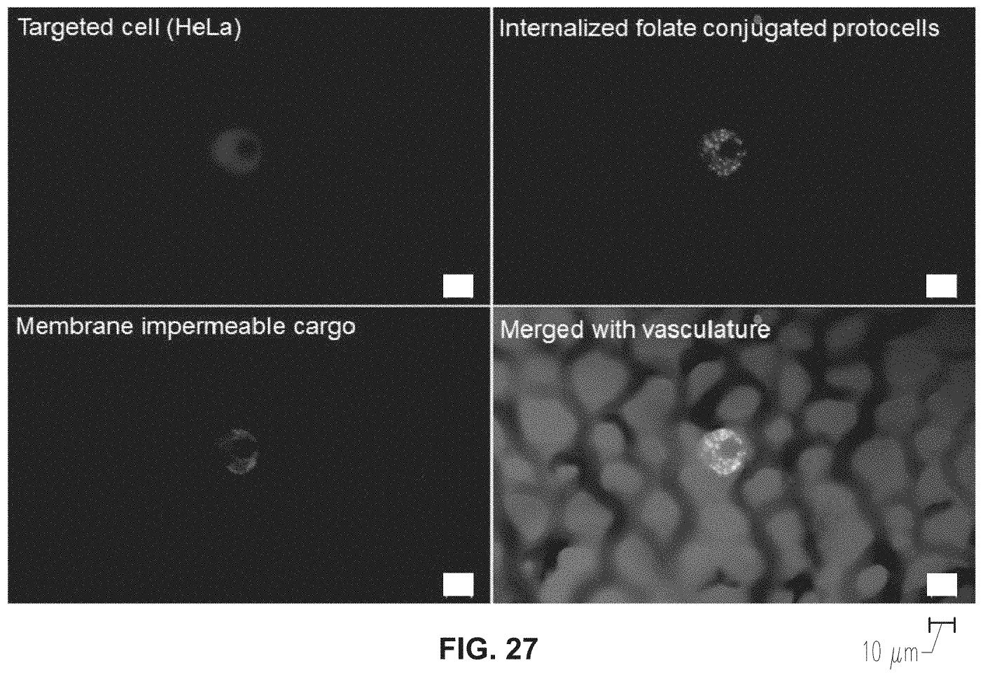

Monosized protocell targeting can be achieved in complex biological systems. FIG. 26 shows highly specific protocell binding observed 30 minutes post injection using intravital imaging technique, demonstrating that monosized protocell targeting can be achieved in complex biological systems. FIG. 27 shows protocell binding with high affinity and or internalization is observed 21 hours post injection using intravital imaging technique, demonstrating that monosized protocell targeting can be achieved longer term in complex biological systems.

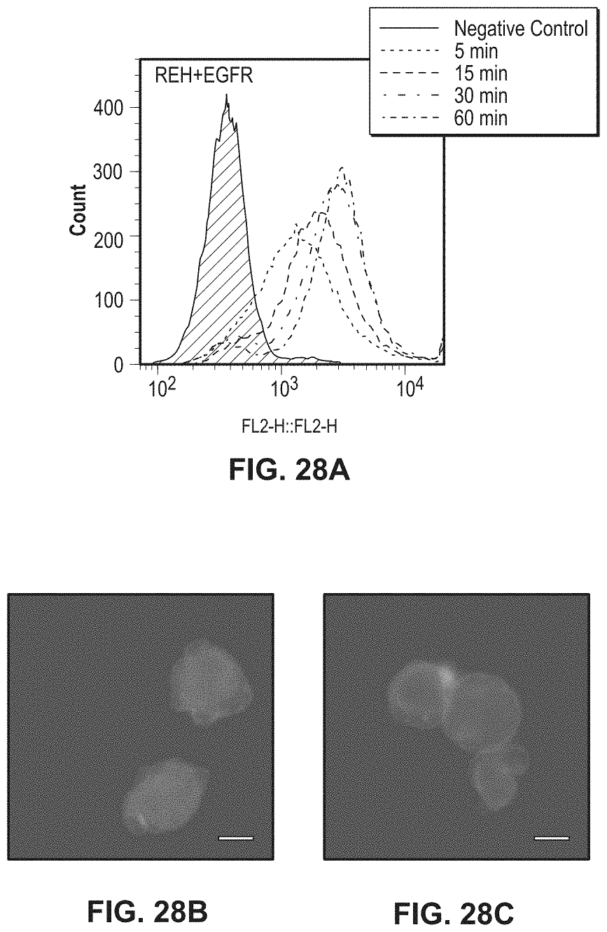

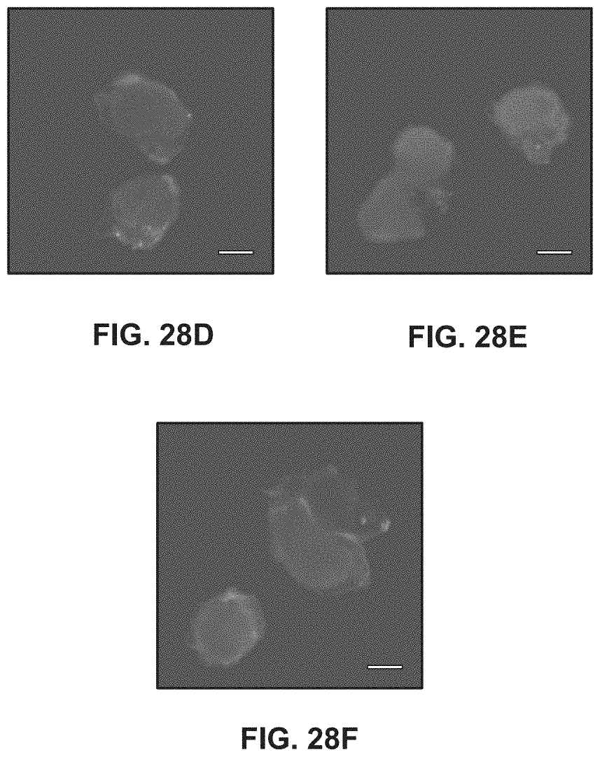

The targeted protocells exhibit specific binding and internalizing, and release of cargo within target cell within a living complex animal system. FIG. 28 shows membrane impermeable cargo was loaded into MSNP core then sealed inside with a supported lipid bi-layer with folate targeting ligand. Target cells were injected into CAM followed by injection of loaded/folate targeted protocells. Protocells bound to cells and became internalized as evidenced by fluorescent cargo release within the cell. This dye would be incapable of entering the cell without the protocell carrier.

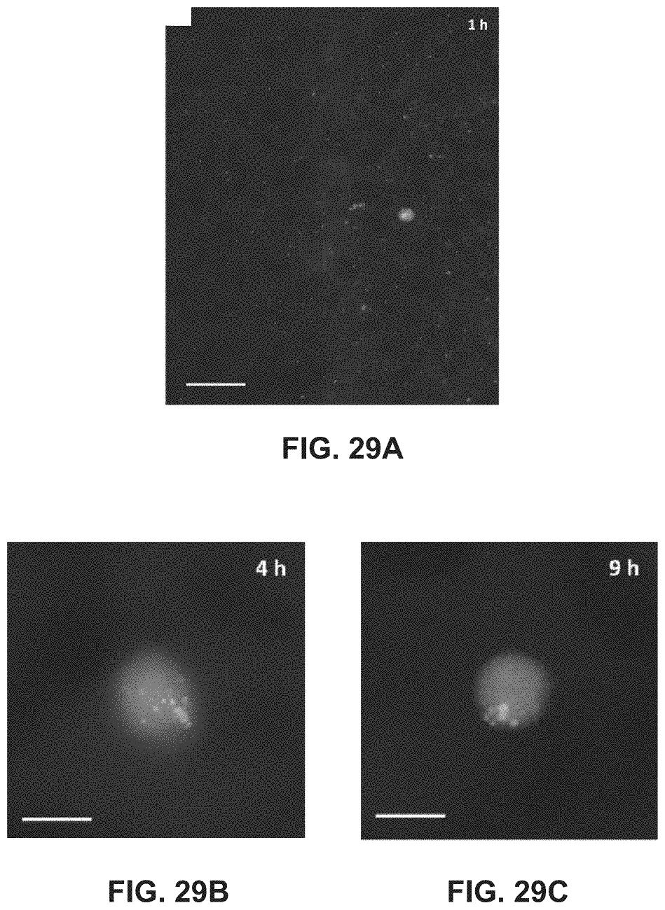

FIG. 29 shows flow cytometry analysis of REH+EGFR cells incubated with red fluorescent EGFR targeted protocells at multiple time points. Corresponding fluorescent microscopy analysis of REH+EGFR cells fixed and stained (nuclei, cytoskeleton, protocells) at (b) untreated, (c) 5 minutes, (d) 15 minutes, (e) 30 minutes, and (f) 60 minutes incubation times. These data illustrate rapid in vitro protocell binding in as little as 5 minutes in complete medium, and maximal protocell accumulation after 30 minutes. Scale bar=5 μm.

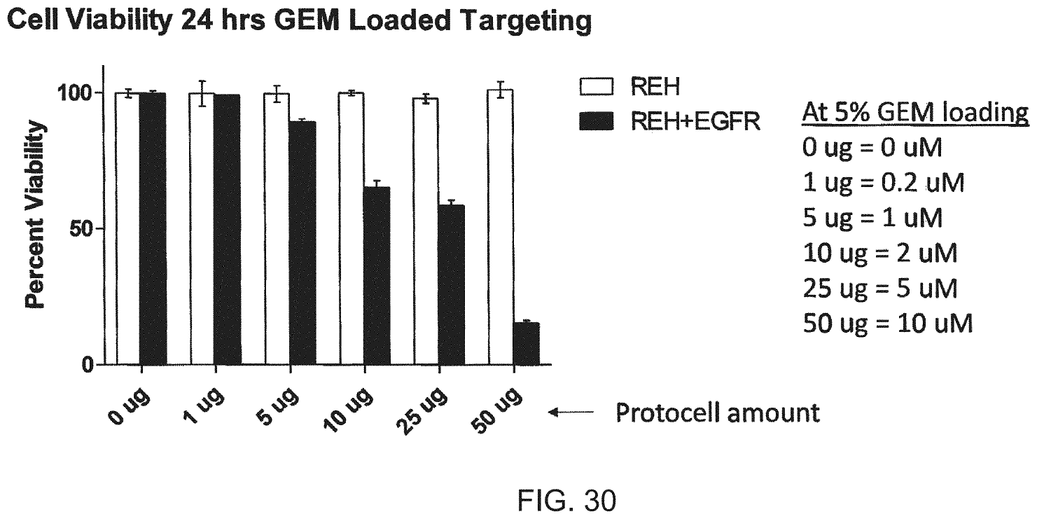

FIG. 30 shows the decrease in viability of REH+EGFR cells with increasing concentration of GEM loaded EGFR-targeted protocells. REH+EGFR cells incubated with protocells from 0 to 50 ug/ml for 1 h, then washed to remove unbound protocells. Viability was assessed at 24 hours. Viability data highlights target specific delivery of cytotoxic cargo using monosized protocell platform. Data represents mean±SD, n=3.

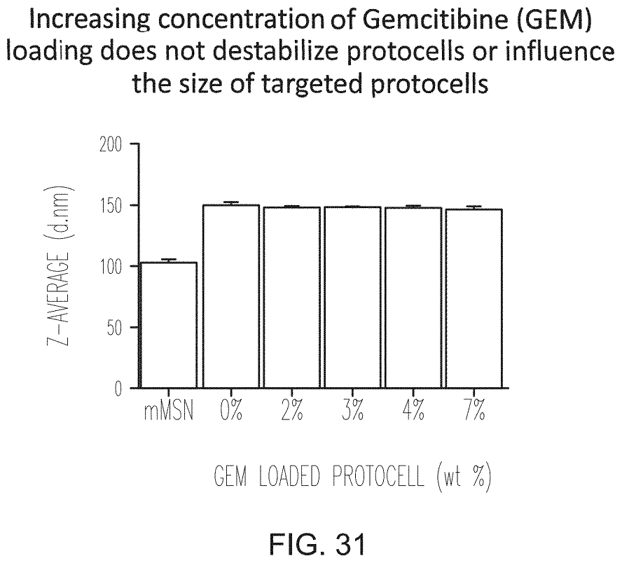

The presently claimed monosized protocells can increase the loading of cargo. FIG. 31 shows that increasing the concentration of Gemcitabine (GEM) loading does not destabilize the protocells or influence the size of targeted protocells.

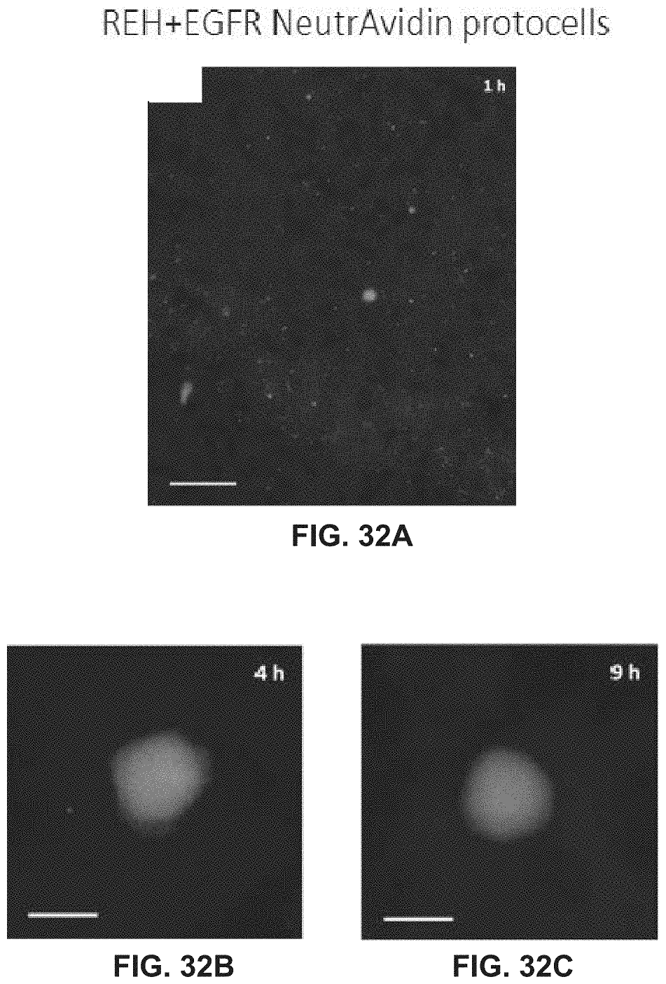

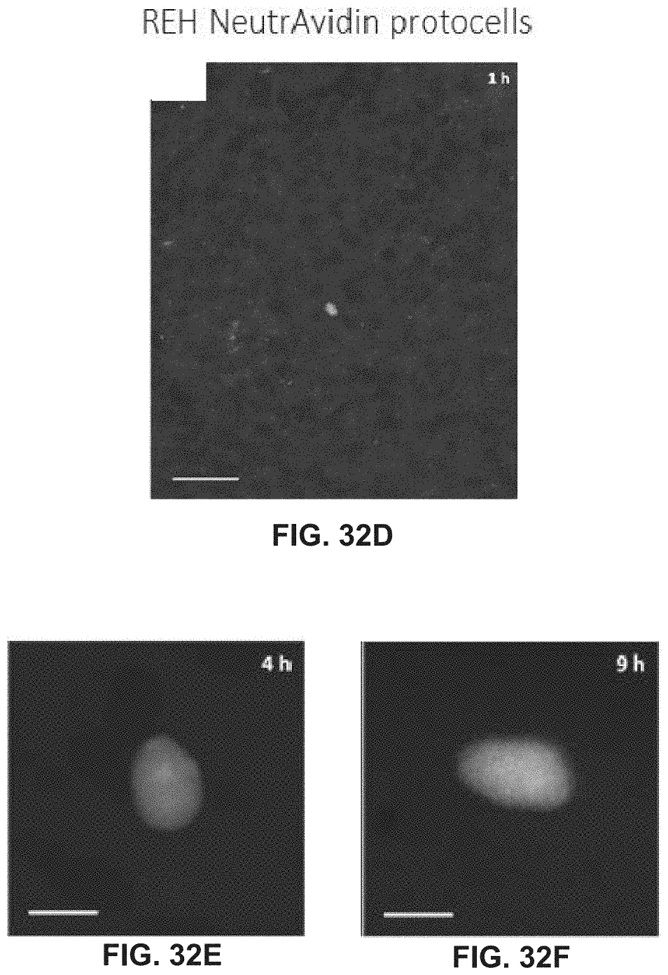

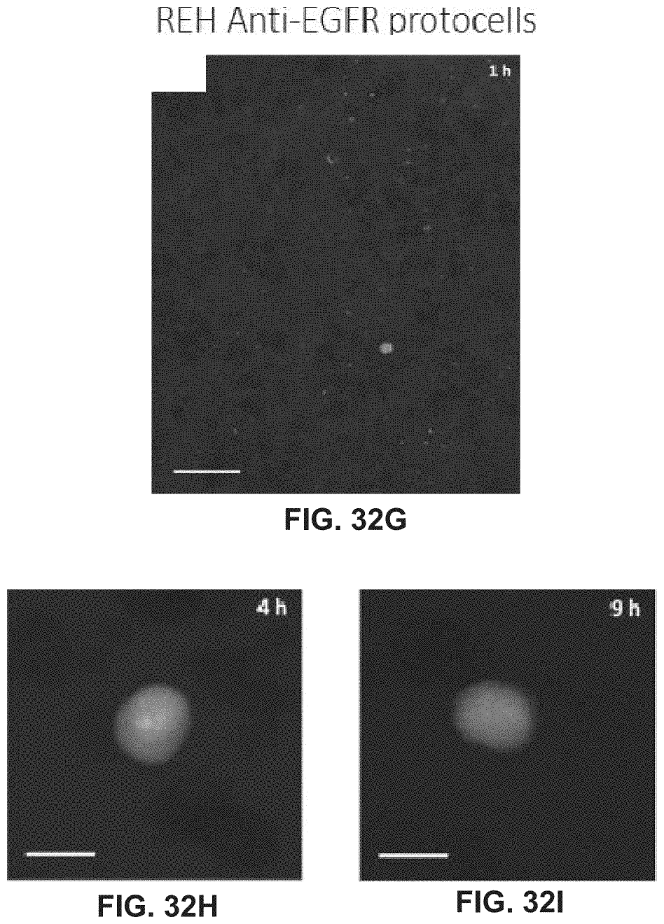

FIG. 32 shows that intravital fluorescent microscopy images acquired ex ovo in the CAM model reveal stable circulation of non-targeted protocells but no association with (a) REH+EGFR cells and (b) parental REH cells in circulation at 1 hour (left), 4 hours (top right), and 9 hours (bottom right) time points. Similarly, EGFR targeted protocells circulate but do not associate with parental REH cell in circulation (c). Scale bar (left)=50 μm, Scale bars (right)=10 μm.

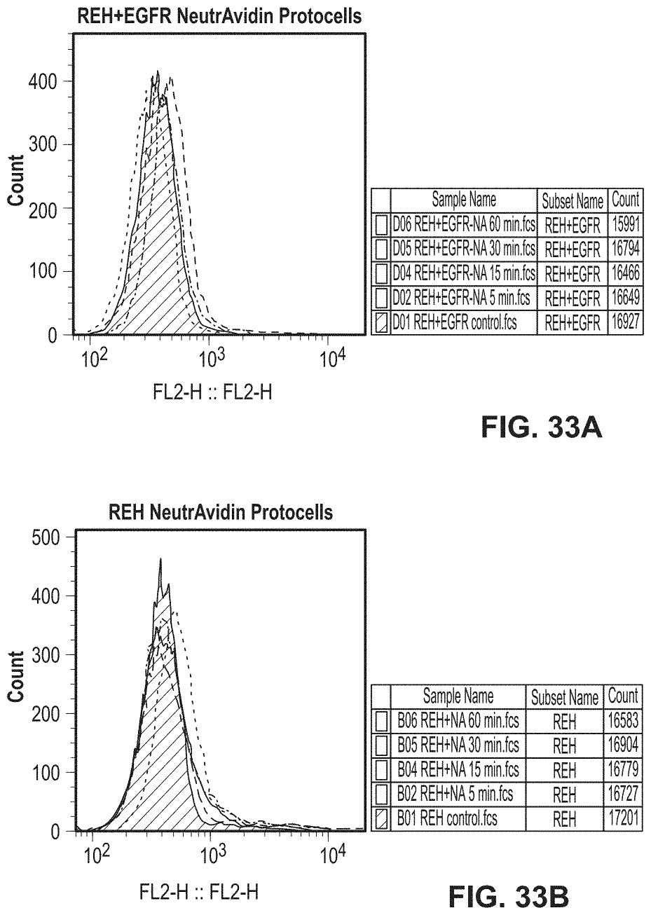

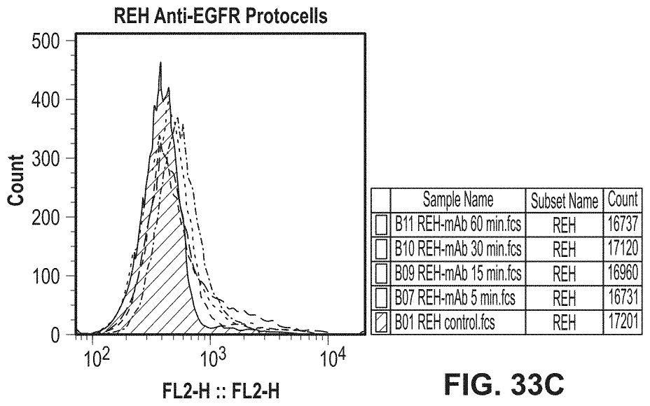

The present protocells demonstrates a high degree of specificity with the targeting strategy. FIG. 33 shows flow cytometry analysis of red fluorescent non-targeted protocells incubated with (a) REH+EGFR cells and (b) parental REH cells at multiple time points. Flow cytometry data confirm components used with our targeting strategy do not contribute to non-specific binding in vitro. In addition, red fluorescent EGFR-targeted protocells incubated with (c) parental REH cells at multiple time points do not bind, demonstrating a high degree of specificity with the targeting strategy.

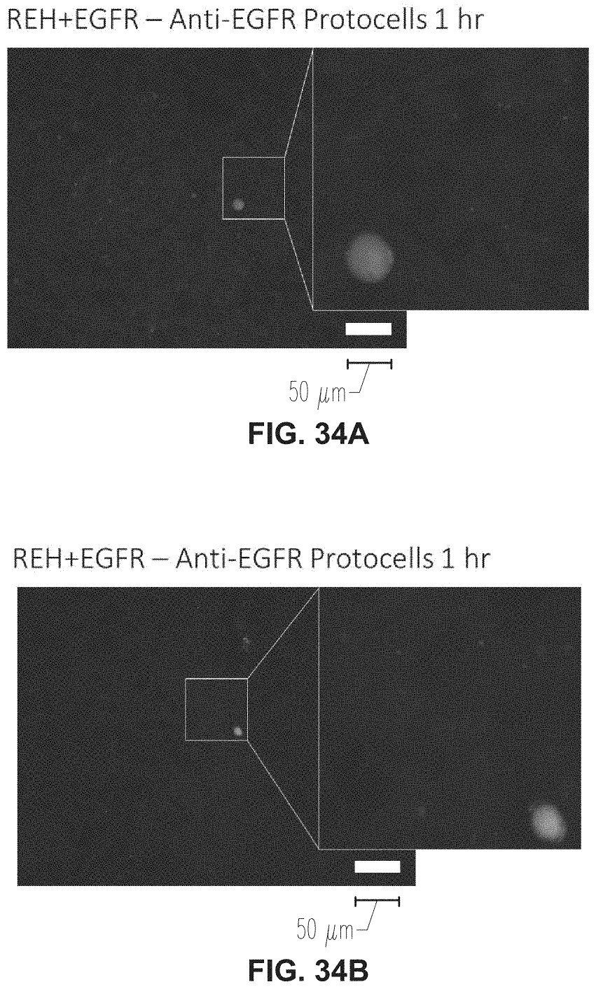

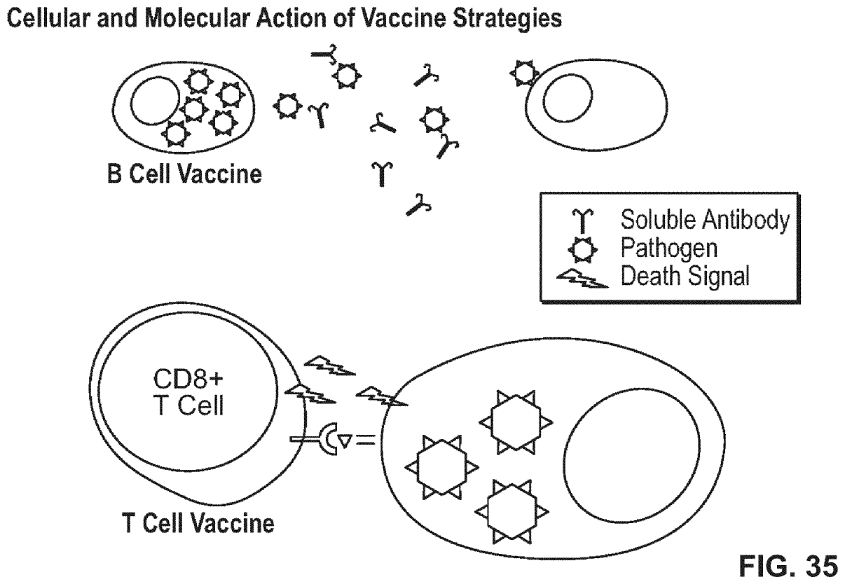

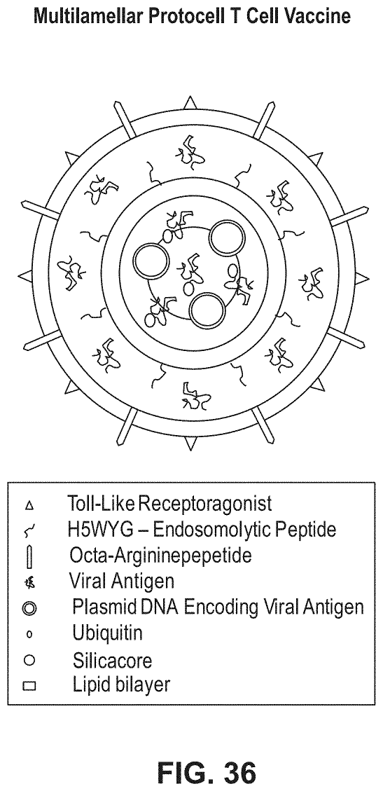

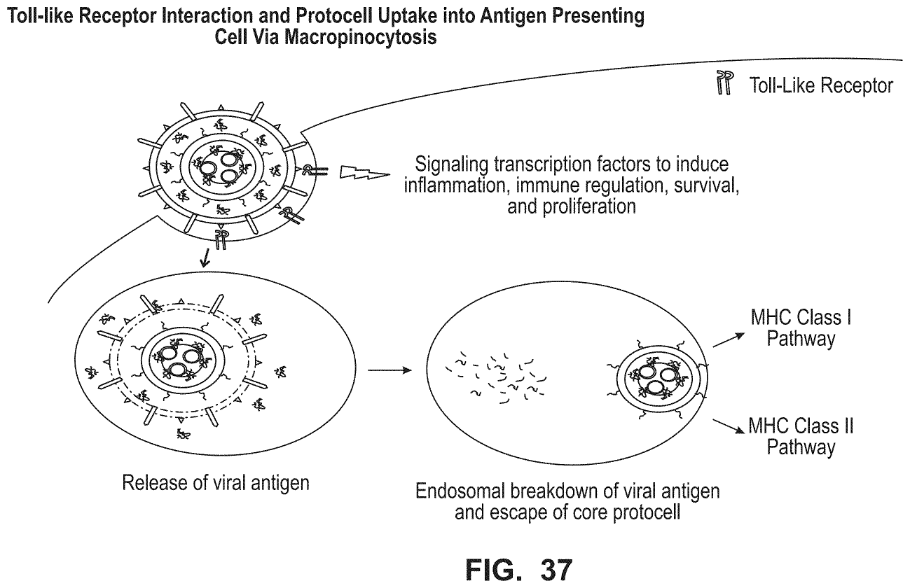

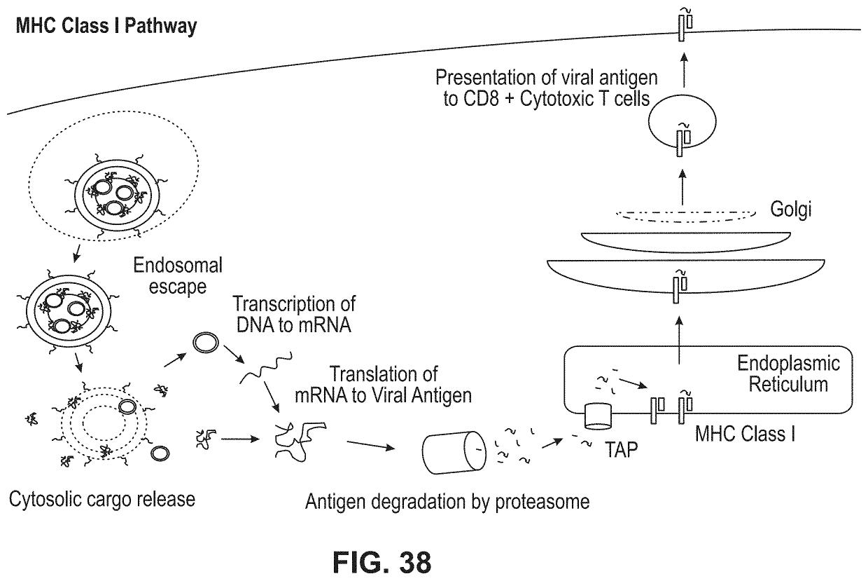

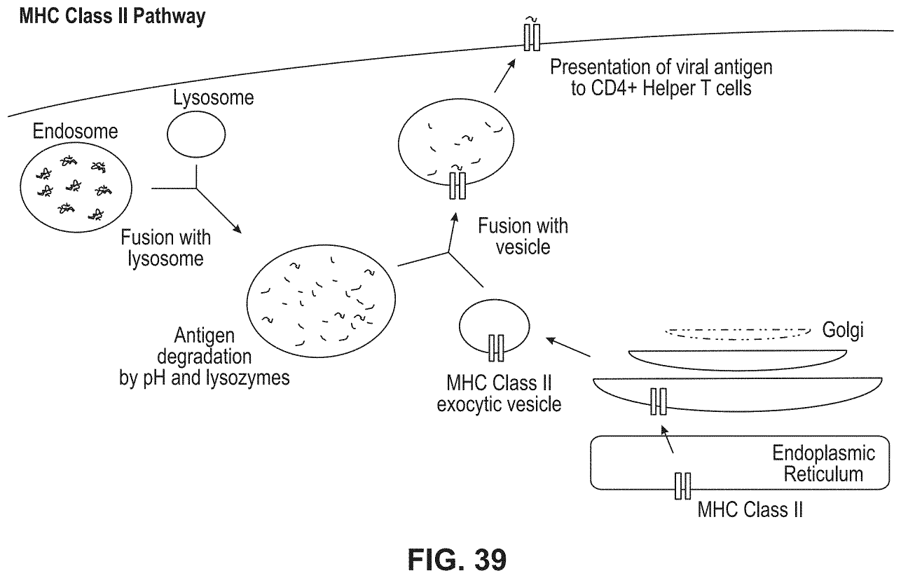

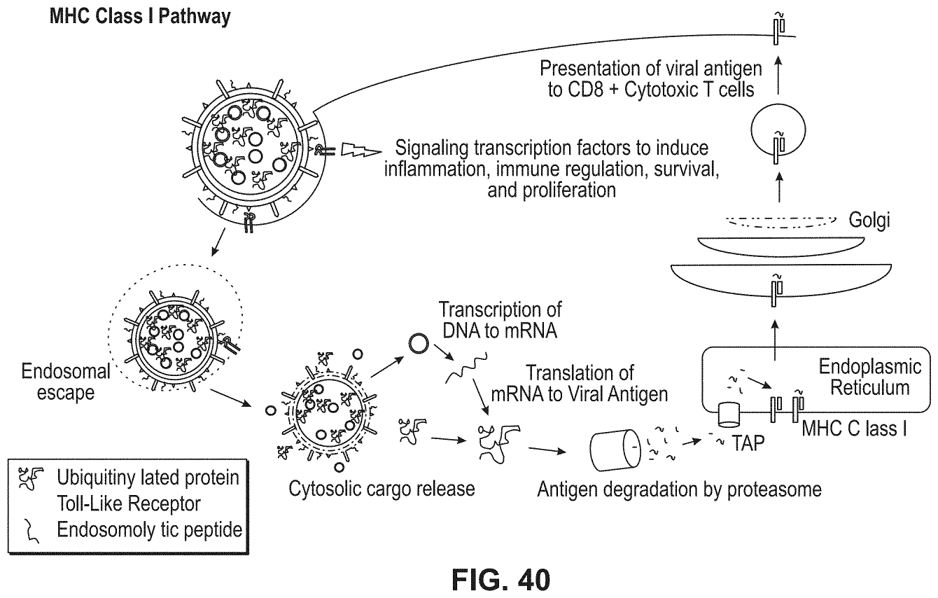

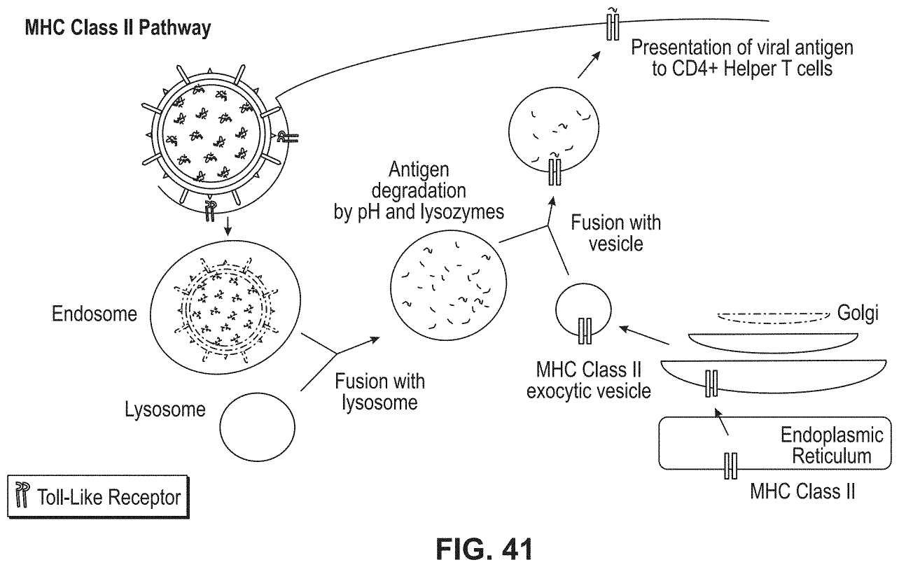

In a further example, Green fluorescent EGFR expressing cells injected into chorioallantoic member (CAM) and allowed to circulate and arrest in the capillary bed for 30 minutes. After 30 minutes, monosized anti EGFR targeted protocells were injected and allowed to circulate for 1 hour. In FIG. 34, intravital imaging reveals significant targeted protocell binding with target cells. In addition, flow patterns observed in red fluorescent lines indicate that targeted protocells maintain colloidal stability while circulating in a live animal system.

### Example 2

Preliminary experiments were performed in vitro, to optimize protocells for APC uptake and TLR-mediated stimulation. In addition, localization of the protocell in the endosome and confirmation of escape into the cytoplasm are measured through confocal fluorescence microscopy. The plasmid and viral protein cargo are fluorescently tagged to monitor release and cellular localization. In addition, toxicology studies will be performed to assess the degree of oxidative stress induced in APCs by protocells.

In vivo experiments are performed to determine the ability of protocells to activate an effective T cell-mediated immune response against virus. Animals are inoculated with protocells; blood will be extracted and analyzed for increased activated T cell population and soluble antibody production. To assess prophylactic potential, animals will be immunized with protocell T cell vaccine and challenged with live virus (BSL-4). Finally, to examine the therapeutic potential, animals will be observed after infection with live virus followed by treatment with protocells.

Nipah Virus, a highly contagious member of the genus Henipavirus in the family Paramyxoviridae, is responsible for several fatal outbreaks across Southeast Asia. The incubation time in humans is rapid and symptoms range from flu-like symptoms to fatal encephalitis. Currently no treatment or vaccine is available, and the virus is classified as a biosafety level 4 (BSL4) pathogen. Nipah Virus is extremely important from an engineered biological weapon standpoint, since an outbreak could cause high human fatality rates, significant fear and social disruption, as well as substantial economic loss from infected livestock. From a national security perspective, there is a critical need for the development and production of safe and effective vaccine and treatment options to combat and control Nipah Virus infection.

4 goal of these examples is the development of nanocarriers that simultaneously address the multiple requirements of targeted delivery, such as specificity, stability, cargo capacity, multicomponent delivery, biocompatibility, and innate immune activation. For example, the examples will demonstrate selective targeting and delivery of Nipah virus-specific protein and plasmid cargo to antigen presenting cells (APCs) to elicit both a cytotoxic and helper T cell response.

### Design, Synthesis, and Characterization of Nanocarrier Silica-Supported Multilamellar Lipid Bi-Laver (Protocells).

Protocells are composed of a nanoporous nanoparticle core that supports a lipid bi-layer, which is further conjugated with targeting peptides and polyethylene glycol (PEG). Through engineering the pore size and surface chemistry, as well as the degree of condensation of the nanoporous particle core (which serves as a reservoir for arbitrary multicomponent cargos), the cargo loading and release characteristics we tailored to achieve optimized pharmacokinetics and biodistribution of therapeutic agents via in vitro and in vivo studies. The biophysical and biochemical properties of the supported lipid bi-layer, such as fluidity and peptide types and concentrations, are refined through iterative studies to maximize binding to and internalization within target cells. The outer protocell surfaces are functionalized with octa-arginine (R8) peptide, to induce cellular uptake of the protocell through macropinocytosis. In addition, Toll-like receptor (TLR) agonists including Monophosphoryl lipid A (MPLA), a derivative of the lipopolysaccharide layer of Salmonella minnesota recognized by TLR-4, and Flagellin, a protein monomer that contains highly conserved regions recognized by TLR-5, among numerous others as described hereinabove. The innermost lipid bi-layer win be functionalized with H5WYG, an endosomolytic peptide that promotes endosomal escape to allow for delivery of cargo components to the cytoplasm of the target cell.

### Cell Culture Studies of Targeted Protocell Selectivity and Fluorescently Labeled Cargo Delivery.

Flow cytometry is employed to determine the specific affinity of protocells modified with various densities of TLR agonists to cultured peripheral blood mononuclear cell (PBMC) derived dendritic cells. The full length viral proteins incorporated into the protocell will be fluorescently labeled. In addition, the proteins encapsulated in the core will be ubiquitinylated to facilitate rapid proteasome degradation. The degree of R8/TLR induced protocell internalization and the intracellular fate of internalized cargo will be assessed using fluorescence confocal microscopy. As described above, the fluidity of the protocells is modified and the degree of PEG present on the nanocarrier surfaces altered to modulate targeting efficacy, maximize the ratio of internalized versus surface-bound nanoparticles, and increase colloidal stability in the presence of serum proteins and physiological salt concentrations. In vitro toxicology studies are performed by assessing the degree of oxidative stress induced in target and control cells by protocells.

### Targeting of APCs to Initiate Adaptive Immune Response to Nipah Virus-Specific Proteins in an Animal Model.

Animals are inoculated intramuscularly with multiple Protocell variations and compared to Nipah viral proteins alone. The animals are immunized two times at two-week intervals, and blood will be collected from animals two weeks after each inoculation via intraocular bleed. Activated T cells are isolated from whole blood and total T cell population will be compared to negative control to determine whether protocells effectively stimulate T cell proliferation. In addition, titers of the resulting anti-Nipah viral protein antibodies elicited are assayed by indirect ELISA. Following immunization, animals are challenged with live Nipah virus (BSL-4 in Texas). Animals are sacrificed day X post infection, and tissue including brain, lung, mediastinal lymph nodes, spleen, and kidney will be harvested for immunohistochemistry analysis using antisera to Nipah virus. Lastly, to examine the therapeutic potential of protocells, animals are infected with Nipah virus and at different time points after exposure, will be inoculated with Protocells. Blood will be collected from the animals at multiple time points and viral load will be assessed by indirect ELISA.

### Example 3

Many nanocarrier cancer therapeutics currently under development, as well as those used in the clinic, rely upon the enhanced permeability and retention (EPR) effect to passively accumulate in the tumor microenvironment and kill cancer cells. For leukemia treatment, where circulating cancer cells make up the bulk of the disease profile, the EPR effect is largely inoperative. In this case it is necessary to target and bind to individual cells—a moving target. Here, the synthesis conditions and lipid bi-layer composition needed to achieve highly monodisperse mesoporous silica nanoparticle (MSN)-supported lipid bi-layers (protocells) were established the protocells that remain stable in complex media as assessed in vitro by dynamic light scattering and cryo-electron microscopy and ex ovo by direct imaging within a chick chorioallantoic membrane (CAM) model. For vesicle fusion conditions where the lipid surface area exceeds the external surface area of the MSN and the ionic strength exceeds 20 mM, monosized protocells (polydispersity index<0.1) on MSN cores were formed with varying size, shape, and pore size whose conformal zwitterionic supported lipid bi-layer confers excellent stability as judged by circulation in the CAM and minimal opsonization in vivo in a mouse model. Having established protocell formulations that are stable colloids, they were further modified with anti-EGFR antibodies and their monodispersity and stability re-verified. Then using intravital imaging in the CAM we directly observed in real time the progression of selective targeting of individual REH leukemia cells and delivery of a model cargo were directly observed in real-time. Thus, the effectiveness of the protocell platform for individual cell targeting and delivery needed for leukemia and other disseminated disease was established.

It is now widely recognized that nanoparticle based drug delivery provides a new ability to package poorly soluble and/or highly toxic drugs, and protect drugs and molecular cargos from enzymatic degradation, and enhance their circulation and biodistribution compared to free drug. Furthermore ‘passive’ or ‘active’ targeted delivery promises precise administration of therapeutic cargos to specific cells and tissues, while sparing collateral damage to healthy cells/tissues and potentially overcoming multiple drug resistance mechanisms (Bertrand et al., 2014; Sun et al., 2014; Tarn et al., 2013). So-called passive targeting occurs through the enhanced permeability and retention (EPR) effect resulting from 200-2000 nm fenestrations in the tumor vasculature that are permeable to blood components including nanoparticles (Bertrand et al., 2014). Nanoparticles are retained because the lymphatic function of the tumor may be defective and does not support convective flow back into the interstitial fluid (Padera et al., 2004), and because diffusion of nanoparticles may be highly limited due to their dimensions (Chauhan et al., 2012). Arguably all nanoparticle therapeutics smaller than several micrometers could accumulate in tumor microenvironments according to the EPR effect; but their efficiency is strongly dependent on physicochemical factors such as size, shape, surface charge, and hydrophobicity, which control colloidal stability, and accordingly circulation time, non-specific binding, opsonization, and uptake by the mononuclear phagocyte system (-MPS) (Bertrand et al., 2014; Blanco et al., 2015). Active targeting relies on modifying the nanocarrier with ligands that bind to receptors that are over expressed or uniquely expressed on the targeted cancer cells versus normal cell (Peer et al., 2007). Typically active targeting also relies upon the EPR effect, and its efficiency is governed by the same physicochemical factors as those for passive targeting (Bartlett et al., 2007). The difference is that targeting ligands can enhance binding and, therefore, retention by the targeted cell and can often promote internalization via receptor-mediated endocytotic pathways (Bertrand et al., 2014; Barlett et al., 2007). Targeting ligands, however, increase size, complexity, and cost and potentially alter the same physicochemical parameters that govern the EPR effect, requiring reoptimization of the surface chemistry (Bertrand et al., 2014). For this reason the benefits of active targeting are often not clear-cut, and consequently considerably fewer actively targeted nanoparticle therapeutics are used clinically (Lammers et al., 2012; Shi et al., 2011). A major exception is targeted delivery to individual or small groups of cells or circulating cells, where by definition the EPR effect is likely inoperative. Here, nanoparticle delivery to leukemias is an important case in point. Because conventional anti-cancer drugs used for leukemia therapy are systemic and non-targeted, they may result in significant acute and long term side effects to normal tissue for leukemia patients. There is a need to increase the efficacy and reduce toxicity of therapeutic interventions by direct targeting of specific sites or cells (Iyer et al., 2013; Markman et al., 2013). Individual cell targeting, however, remains a significant challenge in cancer nanomedicine and has yet to be thoroughly demonstrated (Adamson et al., 2015). In the case of leukemia therapeutics, active targeting is required to allow specific delivery to leukemic cells in circulation and those in organ reservoirs such as bone marrow and spleen. It should be emphasized that targeting cannot be achieved at the expense of colloidal stability because the EPR effect cannot be relied upon and increased circulation half-life has been shown to increase delivery to bone marrow, spleen, and liver disease sites where leukemia cells may frequently home (Adamson et al., 2015).

Given the unique challenge of nanoparticle-based delivery to leukemia cells, it is worthwhile to consider the optimal drug delivery platform. An effectively targeted nanocarrier for leukemia treatment would ideally possess the following combined characteristics: 1) uniform and controllable particle size and shape; 2) high colloidal stability under physiological and storage conditions; 3) minimal non-specific binding interactions, uptake by the MPS, or removal by excretory systems, allowing extended circulation time; 4) high specificity to diseased cells or tissues; 5) high capacity for and precise release of diverse therapeutic cargos; and 6) low cytotoxicity. Liposomes are one of the most successful classes of nanocarriers for achieving both passive and active targeted delivery, and numerous Food and Drug Administration (FDA) approved formulations exist (Allen et al., 2004; Iwamoto, 2013; Egusquiaguirre et al., 2012; Pattni et al., 2015). Of candidate nanocarriers, liposomes exhibit many advantageous properties, including ease of synthesis, high biocompatibility, flexible formulation, targetability, and increased circulation times compared to free drugs (Peer et al., 2007; Davis et al., 2008; Deshpande et a, 2013; Farokhzad and Langer, 2009; Torchilin, 2005). However, it has proven difficult to identify stable lipid formulations that allow drug encapsulation but prevent leakage (Ca\check{g}das et al., 2014; Reynolds et al., 2012). Polymeric based therapeutic nanocarriers, have also been developed, and several formulations are currently being tested in clinical trials (Egusquiaguirre et al., 2012). Similar to liposomes, many polymer based nanocarriers are biocompatible and easy to manufacture, however they also suffer from limited stability in vivo and dose dependent toxicity (Elsabahy et al., 2012; Draz et al., 2014; Williford et al., 2014). Furthermore, both liposomes and polymer based nanoparticles suffer the issues of invariant size and shape, uncontrollable, often burst release profiles, and highly interdependent properties, whereby changing one property, such as loading efficiency, affect numerous other properties, such as size, charge, and stability (Peer et al., 2007: Davis et al., 2008; Farokhzad and Langer 2009; Torchilin, 2005). By comparison, mesoporous silica nanoparticles (MSN) have controlled size and shape and are composed of high surface area (500 to >1000 m²/g) networks of uniformly sized pores whose size and surface chemistry can be varied widely to accommodate high payloads of disparate cargos (Li et al., 2012; Vivero-Escoto et al., 2010). Furthermore, colloidal mesoporous silica is biodegradable and generally recognized as safe (GRAS) by the FDA (Butler et al., 2016). The drawbacks of MSN are that often coatings are required to contain the cargo and shield surface silanols (≡Si—OH) and deprotonated silanols (≡Si—O⁻) that are highly lipophilic and known to promote non-specific binding and MPS uptake (Zhang et al., 2012; Meng et al., 2011; Brinker and Scherer, 2013). In this context, MSN-supported lipid bi-layers (protocells), a rapidly emerging class of nanocarriers, unique attributes. Protocells are formed by the encapsulation of the MSN core within a supported lipid bi-layer (SLB) followed optionally by conjugation of polymers, such as PEG, and targeting and/or trafficking ligands to the surface of the SLB ((Wang et al., 2010; Ashley et al., 2012; Epler et al., 2012; Cauda et al., 2010; Meng et al., 2015; Wang et al., 2013; Zhang et al., 2014; Ashley et al., 2011; Liu et al., 2016; Huang et al., 2016; Mackowiak et al., 2013; Porotto et al., 2011; Han et al., 2015; Liu et al., 2009; Liu et al., 2009). Protocells synergistically combine the advantages of liposomes, viz. low inherent toxicity and immunogenicity, and long circulation times, with the advantages of MSNs, viz. size and shape control and an enormous capacity for multiple cargos and disparate cargo combinations. Moreover, many studies have revealed that protocells and related MSN supported bi-layer nanocarriers are stable at neutral pH but exhibit pH triggered cargo release under endosomal conditions (Ashley et al., 2012; Epler et al., 2012; Cauda et al., 2010; Meng et al., 2015; Wang et al., 2013; Zhang et al., 2014; Ashley et al., 2011; Han et al., 2015).

To date, protocell based nanocarriers have shown to be effective for the delivery of multiple classes of cargos and cargo combinations to various cell types (Butler at al., 2016). The majority of studies conducted have reported efficacy in vitro (Ashley et al., 2012; Epler et al., 2012; Ashley et al., 2011), but numerous recent reports also show excellent in vivo results, where passive and active targeting to solid tumors via the EPR effect have been demonstrated (Meng et al., 2015; Wang at al., 2013; Zhang et al., 2014; Liu et al., 2009). However, the targeting of individual cells in vivo or in living systems has yet to be reported, and there have been no direct observations/determinations of in vivo colloidal stability. Here, in vivo colloidal stability is paramount to achieving synthetic factors (e.g., the lipid/silica ratio and ionic strength during SLB formation) and variation of modular protocell components (e.g., MSN size, shape, and pore size, lipid bi-layer fluidity, extent of PEGylation, and surface display of targeting ligands) on the influence colloidal stability was explored as judged in vitro and in vivo by particle size stability and polydispersity and by direct observation ex ovo in a chick chorioallantoic membrane (CAM) model. Processing conditions were established for particle size monodispersity and size stability for protocells with differing size, shape, and pore morphology. Using optimized processing conditions, long circulation times were demonstrated, and avoidance of non-specific binding and minimal opsonization ex ovo and in vivo. Having achieved in vivo colloidal stability, targeted binding and cargo delivery to individual leukemia cells in vitro and ex ovo by direct observation was shown in the CAM model.

### Experimental Section

Materials.

All chemicals and reagents were used as received. Ammonium hydroxide (NH₄OH, 28-30%), 3-aminopropyltriethoxysilane (98%, APTES), ammonium nitrate (NH₄NO₃), benzyldimethylhexadecylammonium chloride (BDHAC), n-cetyltrimethylammonium bromide (CTAB), N,N-dimethyl formamide (DMF), dimethyl sulfoxide (DMSO), rhodamine B Isothiocyanate (RITC), tetraethyl orthosilicate (TEOS), Triton X-100, and Buffer solution pH 5.0 (citrate buffer) were purchased from Sigma-Aldrich (St. Louis, Mo.). Hydrochloric acid (36.5-38%, HCl) was purchased from EMD Chemicals (Gibbstown, N.J.). Absolute (99.5%) and 95% ethanol were obtained from PHARMCO-AAPER (Brookfield, Conn.). 1,2-dioleoyl-sn-glycero-3-phosphocholine (DOPC), 1,2-distearoyl-sn-glycero-3-phosphocholine (DSPC), 1,2-dioleoyl-sn-glycero-3-phosphoethanolamine-N-[methoxy(polyethylene glycol)-2000] (ammonium salt) (DOPE-PEG₂₀₀₀), 1,2-distearoyl-sn-glycero-3-phosphoethanolamine-N-[methoxy(polyethylene glycol)-2000] (ammonium salt) (DSPE-PEG₂₀₀₀), 1,2-distearoyl-sn-glycero-3-phosphoethanolamine-N-[amino(polyethylene glycol)-2000] (DSPE-PEG₂₀₀₀-NH₂) phospholipids and cholesterol (chol, ovine wool, >98%) were purchased from Avanti Polar Lipids (Birmingham, Ala.). Hoechst 33342, Traut's reagent, YO-PRO®-1, and maleimide-activated NeutrAvidin protein were obtained from Thermo Scientific (Rockford, Ill.). Alexa Fluor® 4488 phalloidin, CellTracker™ Blue CMAC dye, and CellTracker™ green CMFDA dye were purchased from Life Technologies (Eugene, Oreg.). Heat inactivated fetal bovine serum (FBS), 10× phosphate buffered saline (PBS), 1× trypsin-EDTA solution, and penicillin streptomycin (PS) were purchased from Gibco (Logan, Utah). Dulbecco's Modification of Eagle's Medium with 4.5 g/L glucose, L-glutamine and sodium pyruvate (DMEM) and RPMI-1640 medium were obtained from CORNING cellgro (Manassas, Va.). Gemcitabine (GEM) was purchased from LC Laboratories (Woburn, Mass.). Anti-EGFR antibody [EGFR1] (Biotin) (ab24293) was purchased from Abcam (Cambridge, Mass.). CellTiter-Glo® 2.0 Assay was purchased from Promega (Madison, Wis.). DyLight 649 Lens Culinaris Agglutinin was purchased from Vector Laboratories (Burlingame. Calif.). Spectra-Por® Float-A-Lyzer® G2 Dialysis Device MWCO: 3.5-5 kD purchased from Spectrum Laboratories Inc. (Rancho Dominguez, Calif.).

Synthesis of mMSNs Composed of Hexagonally Arranged Cylindrical Pores (2.8 nm Pore Size), Hexagonal mMSN.

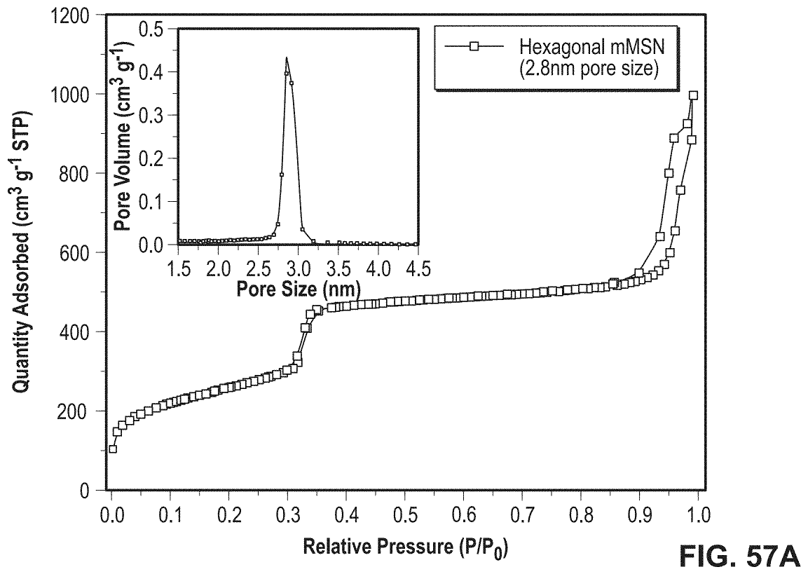

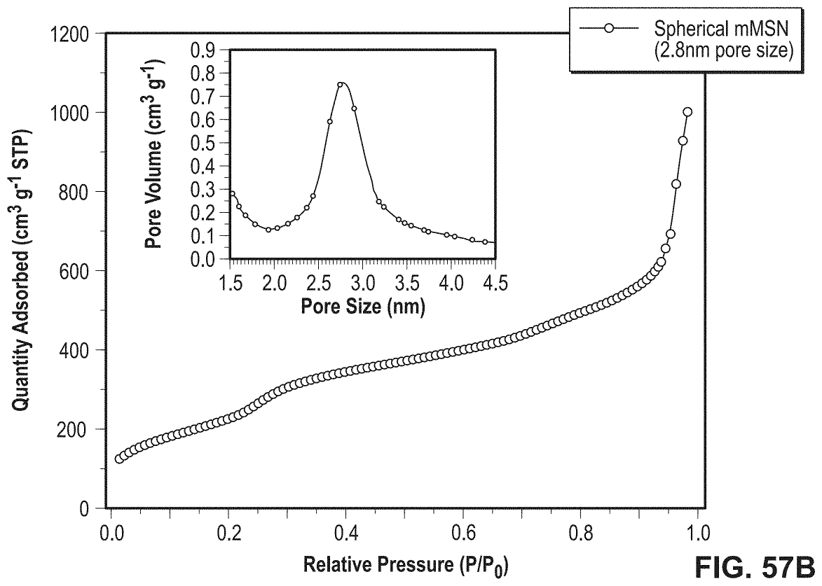

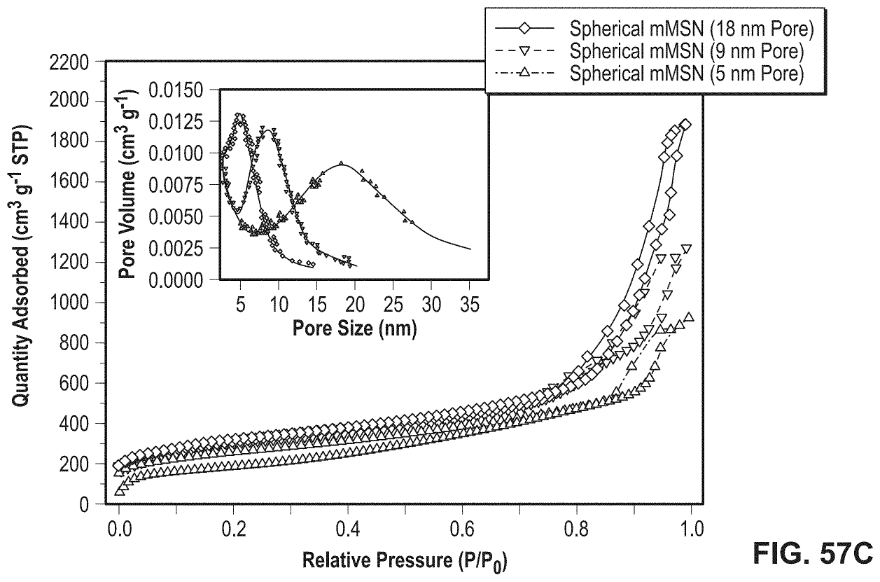

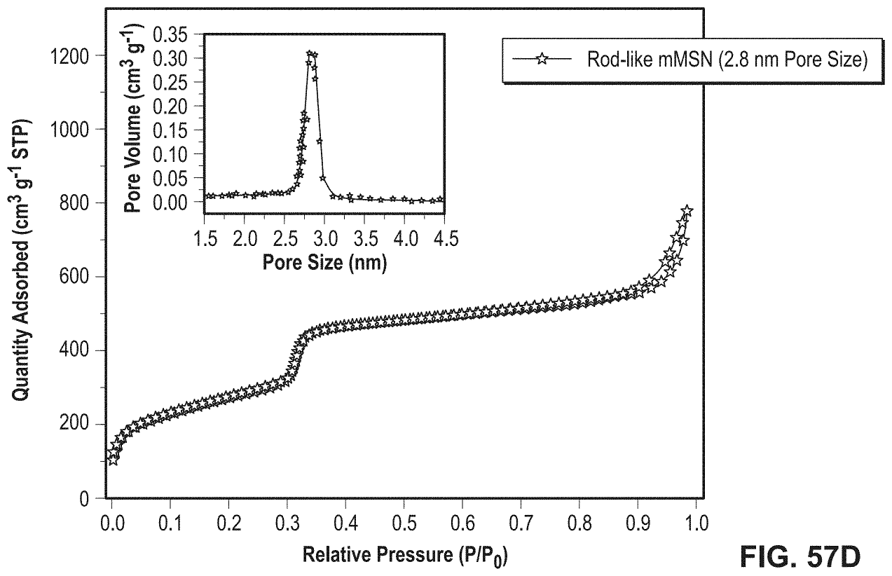

To prepare monosized dye-labeled mMSNs (about 95 nm in diameter, FIG. 57, about 130 nm in hydrodynamic size in D.I. water), 3 mg of RITC was dissolved in 2 mL of DMF followed by addition of 1.5 μL APTES (Townson et al., 2013). The synthesis conditions of Hexagonal mMSNs is based on reported literature (Buranda et al., 2003). The RITC-APTES solution was incubated at room temperature for at least 1 hour. Next, 290 mg of CTAB was dissolved in 150 mL of 0.51 M ammonium hydroxide solution in a 250 mL beaker, sealed with parafilm (Neenah, Wis.), and placed in a mineral oil bath at 50° C. After continuously stirring for 1 hour, 3 mL of 0.88 M TEOS solution (prepared in ethanol) and 1 mL of RITC-APTES solution were combined and added immediately to the surfactant solution. After another 1 hour of continuous stirring, the particle solution was stored at 50° C. for about 18 hours under static conditions. Next, solution was passed through a 1.0 μm Acrodisc 25 mm syringe filter (PALL Life Sciences. Ann Arbor, Mich.) followed by a hydrothermal treatment at 70° C. for 24 hours. Followed procedure for CTAB removal was as described in literature (Lin and Haynes, 2010). Briefly, mMSNs were transferred to 75 mM ammonium nitrate solution (prepared in ethanol) then placed in an oil bath at 60° C. for 1 hour with reflux and stirring. The mMSNs were then washed in 95% ethanol and transferred to 12 mM HCl ethanolic solution and heated at 60° C. for 2 hours with reflux and stirring. Lastly, Hexagonal mMSNs were washed in 95% ethanol, then 99.5% ethanol, and stored in 99.5% ethanol.

Synthesis of Spherical mMSNs with Isotropic Pores (2.8 nm Pore Size).

To prepare monosized spherical mMSNs composed of isotropic mesopores, the same procedure described above was used for synthesis of mMSNs with hexagonally arranged pore structure. However, we substituted cationic surfactant BDHAC for CTAB as the template. The 3-dimensional isotropic pore arrangement is due to a larger micelle packing parameter of BDHAC, compared to CTAB surfactant.

Synthesis of Spherical mMSNs Composed of Dendritic Large Pores (5 nm, 9 nm, and 18 nm Pore Size).

The large pore spherical mMSNs were synthesized by a published biphase method (Nandiyanto et al., 2009; Wang et al., 2012; Shen et al., 2014). Syntheses of 5 nm, 9 nm, and 18 nm pore mMSNs are based on a modified condition reported by Zhao et al. (2014). For preparation of 5 nm dendritic pore mMSNs, 0.18 g of TEA was dissolved in 36 mL of DI water and 24 mL of 25 w % CTAC In a 100 mL round bottom flask. The surfactant solution was stirred at 150 rpm and heated at 50° C. in an oil bath. After 1 hour, 20 mL of 20 v/v % TEOS (in cyclohexane) was added to the CTAC-TEA aqueous solution. After 12 hours, the particle solution was washed with DI water twice by centrifugation. Further surfactant removal achieved by following the previously described conditions used in preparation of small pore mMSNs. For synthesis of 9 nm dendritic pore mMSNs, the stirring rate and organic phase concentration were adjusted to 300 rpm and 10 v/v % TEOS, respectively. For synthesis of 18 nm dendritic pore mMSNs, the TEOS concentration in the organic phase was changed to 5 v/v %. All other steps were identical.

Synthesis of Rod-Shaped mMSNs with Hexagonally Arranged Cylindrical Pores (2.8 nm Pore Size).

The shape of mMSNs can be simply tuned to rod-like morphology by altering the CTAB concentration, stirring rate, and ammonia concentration (Huang et al., 2011; Yu et al., 2011). Briefly, 0.5 g CTAB was dissolved in 150 mL of 0.22 M ammonium hydroxide solution at 25° C. under continuous stirring (300 rpm). Next, of 1 mL TEOS was added (drop wise) to the surfactant solution with stirring. After 1 hour, the particle solution was aged under static conditions for 24 hours, then subsequently transferred to a sealed container and heated to 70° C. for 24 hours. The removal of surfactant was followed the same procedures described previously.

Liposome Preparation.

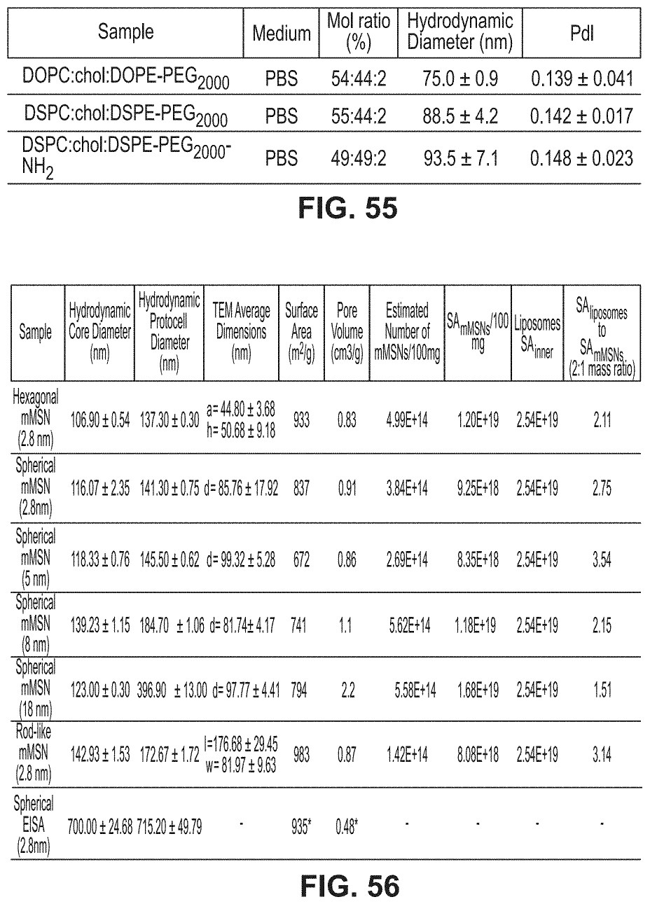

Lipids and chol ordered from Avanti Polar Lipids were presolubilized in chloroform at 25 mg/mL and were stored at −20° C. To prepare liposomes, lipids were mixed at different mol % ratios including (54/44/2) for DOPC/chol/DOPE-PEG₂₀₀₀ and DSPC/chol/DSPE-PEG₂₀₀₀, and (49/49/2) for DSPC/chol/DSPE-PEG₂₀₀₀-NH₂ (FIG. 55). Lipid films were prepared by drying lipid mixtures (in chloroform) under high vacuum to remove the organic solvent. Then the lipid film was hydrated in 0.5×PBS and bath sonicated for 30 minutes to obtain a liposome solution. Finally, the liposome solution was further passed through a 0.05 μm polycarbonate filter membrane (minimum 21 passes) using a mini-extruder to produce uniform and unilamellar vesicles with hydrodynamic diameters less than 100 nm.

Protocell Assembly.

To form protocells, mMSNs are transferred to D.I. water at 1 mg/mL concentration by centrifugation (15,000 g, 10 minutes) and added to liposome solution (2 mg/mL) in 0.5×PBS (1:1 v/v and 1:2 w/w ratios). The mixture was bath sonicated about 10 seconds and non-fused liposomes were removed by centrifugation (15,000 g, 10 minutes). Pelleted protocells were redispersed in 1×PBS via bath sonication, this step is repeated twice.

Anti-EGFR protocell preparation. First, DSPC/chol/DSPE-PEG-NH₂ liposomes were prepared according to the method described previously. Next, a ratio (2:1, w:w) of DSPC/chol/DSPE-PEG₂₀₀₀-NH₂ liposomes to bare fluorescent-labeled Hexagonal mMSN were combined in a conical tube at room temperature for 30 minutes. The excess liposomes were removed by centrifugation (15,000 g, 10 minutes). The pelleted protocells were redispersed in 1 mL of PBS with bath sonication. To convert the surface —NH₂ to —SH groups, 50 μL of freshly prepared Traut's reagent (250 mM in PBS) was added to the protocells. After 1 hour, the particles were centrifuged, and the supernatant was removed. The particles were again redispersed in 1 mL of PBS. Then, 0.15 mg of maleimide-activated NeutrAvidin protein was added to 0.25 mL of thiolated protocells and incubated at room temperature for 12 hours. The NeutrAvidin conjugated protocells were washed with PBS via centrifugation and suspended in 0.25 mL of PBS. Then, 50 μL of biotinylated EGFR antibody (0.1 mg/mL) was mixed with 50 μL of NeutrAvidin conjugated protocells for at least 30 minutes. Finally, the antibody conjugated protocells were pelleted and redispersed in 100 μL PBS for in vitro targeting experiments.

Protocell Biocompatibility Assessment.

Whole human blood was acquired from healthy donors with informed consent and stabilized in K₂EDTA tubes (BD Biosciences). hRBCs were purified following reported procedure (Liao et al., 2011), then incubated with either bare mMSNs or protocells (25, 50, 100, 200, and 400 μg/mL) at 37° C. After 3 hours of exposure, samples were centrifuged at 300 g for 3 minutes, then 100 μL of supernatant from each sample was transferred to a 96-well plate. Hemoglobin absorbance was measured using a BioTek microplate reader (Winooski, Vt.) at 541 nm. The percent hemolysis of each sample was quantified using a reported equation (Lao et al., 2011). In addition, we examined the biocompatibility of anti-EGFR targeted protocells in vitro. We incubated about 1.5×10⁵ cells/mL of REH and REH+EGFR cell lines with either 12.5, 25, 50, 100, and 200 μg/mL of anti-EGFR targeted protocells in complete medium for 1 hour at 37° C. Cells were washed twice in complete media and transferred to a 96-well plate for 24 hours at 37° C. Cell viability was assessed by CellTiter-Glo 2.0 Assay as measured by BioTek microplate reader. The cell viability was calculated as a percentage of non-protocell treated sample.

Cell Culture and Nanoparticle Nonspecific Binding/Uptake.

Human endothelial cells, EA.hy926 (CRL-2922) were purchased from American Type Culture Center (ATCC, Manassas, Va.). We seeded 5×10⁵ EA.hy926 cells in 6-well plates with 2 mL of DMEM+10% FBS and 1% PS at 37° C. in 5% CO₂ humidified atmosphere. After 24 hours, the media was removed and replaced with 2 mL of fresh complete media supplemented with 20 μg/mL of bare mMSNs or protocells for 4 hours at 37° C. under 5% CO₂. After nanoparticle incubation, the media was removed and the cells were gently washed twice with PBS. For imaging purposes, the nanoparticle treated cells were fixed in 3.7% formaldehyde (in PBS) at room temperature for 10 minutes, washed with PBS, then treated with 0.1% Triton X-100 for another 10 minutes. The fixed cells were washed with PBS and stored in 1 mL of PBS. The cell nuclei and F-actin were stained with 1 mL of Hoechst 33342 (3.2 μM in PBS) and 0.5 mL of Alexa Fluor® 488 phalloidin (20 nM in PBS) for 20 minutes, respectively. After staining, the cells were washed with PBS twice and stored in PBS prior to fluorescence microscope imaging. For preparation of flow cytometry samples, the control and nanoparticle treated cells were removed from plate bottom using Trypsin-EDTA (0.25%). The suspended cells were centrifuged, washed with PBS, and suspended in PBS for flow cytometry measurements.

Cell-Nanoparticle Interactions in Ex Ovo Avian Embryos.

Ex ovo avian embryos were handled according to published methods (Leong et al., 2010), with all experiments conducted following an institutional approval protocol (11-100652-T-HSC). This method included incubation of fertilized eggs (purchased from East Mountain Hatchery-Edgewood, N. Mex.) in a GQF 1500 Digital Professional egg incubator (Savannah, Ga.) for 3-4 days. Following initial in ovo incubation, embryos were removed from shells by cracking into 100 mL polystyrene weigh boats (VWR, Radnor, Pa.). Ex ovo embryos were then covered and incubated (about 39° C.) with constant humidity (about 70%). For nanoparticle injections, about 50 μg (at 1 mg/mL) of bare mMSNs or protocells in PBS were injected into secondary or tertiary veins of the CAM via pulled glass capillary needles. CAM vasculature and fluorescent protocells were imaged using a customized avian embryo chamber (humidified) and a Zeiss AxioExaminer upright microscope modified with a heated stage. High speed videos were acquired on the same microscope using a Hamamatsu Orca Flash 4.0 camera.

Post-Circulation Size and Stability Analyses.

All animal care and experimental protocols were in accordance with the National Institutes of Health and University of New Mexico School of Medicine guidelines. Ten- to twelve-week-old female BALB/c mice (Charles River Laboratories, Wilmington, Mass.) were administered dose of fluorescent protocells (10 mg/mL) in 150 μL PBS via tall vein injection. After 10 minutes of circulation, mice were euthanized and blood was drawn by cardiac puncture. Whole blood was stabilized in K₂EDTA microtainers (BD Biosciences) prior to analysis. Ex ovo avian embryos were administered dose of fluorescent protocells (1 mg/mL) in 50 μL PBS via secondary or tertiary veins of the CAM. After 10 minutes of circulation, blood was drawn via pulled glass capillary needles and analyzed immediately. Whole blood cells and protocell fluorescence in both mouse and avian samples were imaged on a glass slide with Zeiss AxioExaminer fixed stage microscope (Gottingen, Germany). To separate protocells from whole blood, samples were centrifuged at low speed to remove blood cells, supernatant fraction was transferred to a fresh tube then centrifuged at 15,000 g for 10 minutes. The pellets were washed (15,000 g for 10 minutes) twice in PBS, then protocell size was analyzed on Malvern Zetasizer Nano-ZS equipment.

In Vitro Targeting Comparison of REH and REH+EGFR Cell Lines.

The human leukemia cell lines, REH and REH+EGFR (Riese et al., 1995) were a kind gift from Professor David F. Stern, Yale University. The REH and REH+EGFR cells were suspended in RPMI 1840 supplemented with 10% FBS media at a concentration of about 5×10⁵ cells/mL. Then one mL of cells was incubated with either NeutrAvidin terminated protocells or anti-EGFR protocells at 10 μg/mL for 5, 15, 30, and 60 minutes respectively at 37° C. under 5% CO₂. The nanoparticle-treated cells were pelleted using a benchtop centrifuge, washed with PBS twice. Cells were fixed in 4% paraformaldehyde for 5 minutes, then washed in PBS, then permeabilized with 0.1% Triton X100 for 5 minutes. The cell cytoskeleton and nuclei were stained by 0.1 mM of Alexa Fluor® 488 phalloidin in PBS for 15 minutes, then washed in PBS, followed by 1.6 μM Hoechst 33342 in PBS for 10 minutes, followed by a final wash in PBS. Stained cells were imaged on a glass slide using the Zeiss AxioExaminer upright microscope. Binding quantification of targeted protocells was determined by a fluorescence shift measured by a BD Accuri™ C6 flow cytometer.

Single Cell Targeting and Model Drug Delivery in Chicken Embryos.

First, about 1×10⁷ of either REH or REH+EGFR cells were suspended in 1 ml PBS and incubated with 2 μL of CellTracker™ green CMFDA dye (2.7 mM in DMSO) for 10 minutes at 37° C. The stained cells were centrifuged, washed, and suspended in 500 μL of PBS. Next, 50 μL of cell solution was administered to ex ovo avian embryos via the previously described procedure. After 30 minutes cell circulation, the anti-EGFR protocells (100 μL, 0.2 mg/mL) were injected into embryos intravenously. Binding of targeted protocells was assessed by fluorescence microscopy at 1, 4, and 9 hours using the Zeiss AxioExaminer upright microscope. To assess internalization and cargo delivery, REH+EGFR cells were stained with CellTracker™ Blue CMAC dye and injected as described above, followed by injection of YO-PRO®-1 loaded RITC labelled protocells (50 μL, 1 mg/mL). Prior to imaging of we injected with DyLight 645 Len Culinaris Agglutin lectin stain to visualize the vasculature, we then imaged the binding, Internalization, and cargo release by fluorescence microscopy at 4 and 16 hours using the Zeiss AxioExaminer upright microscope.

Characterization.

TEM Images were acquired on a JEOL 2010 (Tokyo, Japan) equipped with a Gatan Orius digital camera system (Warrendale, Pa.) under a 200 kV voltage. The Cryo-TEM samples were prepared using an FEI Vitrobot Mark IV (Eindhoven, Netherlands) on Quantifoil® R1.2/1.3 holey carbon grids (sample volume of 4 μL, a blot force of 1, and blot and drain times of 4 and 0.5 seconds, respectively). Imaging was taken with a JEOL 2010 TEM at 200 kV using a Gatan model 626 cryo stage. Nitrogen adsorption-desorption isotherms of mMSNs were obtained from on a Micromeritics ASAP 2020 (Norcross, Ga.) at 77 K. Samples were degassed at 120° C. for 12 hours before measurements. The surface area and pore size was calculated following the Brunauer-Emmet-Teller (BET) equation in the range of P/Po from 0.05 to 0.1 and standard Barrett-Joyer-Halenda (BJH) method. Flow cytometry data were performed on a Becton-Dickinson FACScalibur flow cytometer (Sunnyvale, Calif.). The raw data obtained from the flow cytometer was processed with FlowJo software (Tree Star, Inc. Ashland, Oreg.). Hydrodynamic size and zeta potential data were acquired on a Malvern Zetasizer Nano-ZS equipped with a He—Ne laser (633 nm) and Non-Invasive Backscatter optics (NIBS). All samples for DLS measurements were suspended in various media (DI, PBS, and DMEM+10% FBS) at 1 mg/mL. Measurements were acquired at 25° C. or 37° C. DLS measurements for each sample were obtained in triplicate. The Z-average diameter was used for all reported hydrodynamic size measurements. The zeta potential of each sample was measured in 1×PBS using monomodal analysis. All reported values correspond to the average of at least three independent samples. The fluorescence images were captured with a Zeiss AxioExaminer fixed stage microscope (Gottingen, Germany).

In Vitro Targeting Comparison of Ba/F3 and Ba/F3+EGFR Cell Lines.

The pro-B-lymphocyte cell lines, Ba/F3 and Ba/F3+EGFR (Riese et al., 1995) were a kind gift from Professor David F. Stern. Yale University. The Ba/F3 and Ba/F3+EGFR cells were suspended in RPMI 1640 supplemented with 10% FBS media at a concentration of about 1×10⁶ cells/mL. Then one mL of cells was incubated with anti-EGFR protocells at 5 μg/mL for 1 hour at 37° C. under 5% CO₂. The cell nuclei and membrane were stained by 1 μL of Hoechst 33342 (1.6 mM in DI) and 2 μL of CellTracker™ green CMFDA dye (2.7 mM in DMSO) for 10 minutes. The nanoparticle-treated cells were pelleted using a benchtop centrifuge, washed with PBS twice, and dispersed in PBS. The live cells were imaged on a glass slide using the Zeiss AxioExaminer upright microscope. To further examine the specificity of targeted protocells, the binding of particles was determined by a fluorescence shift measured by a Becton-Dickinson FACScalibur flow cytometer.

Cargo Loading and Release Kinetics.

Model drug loading was achieved by adding 1% volume YO-PRO®-1 (1 mM in DMSO) to mMSNs (1 mg/mL in H₂O) and stored for 12 hours at 4° C. After loading, targeted protocells were prepared using method described earlier in Anti-EGFR targeted protocell preparation. We observed a color change in the pelleted YO-PRO®-1 loaded protocells and did not observe any color in the supernatant during protocell assembly. The interaction between YO-PRO®-1 and mMSNs may largely be driven by electrostatics, since YO-PRO®-1 carries a positive charge. Moreover, YO-PRO®-1 is membrane impermeable, therefore, it should remain encapsulated by the SLB of the protocell until it is broken down in the intracellular environment. To quantify YO-PRO®-1 loading, protocells were pelleted by centrifugation and resuspended in DMSO with bath sonication, this step was repeated twice. Supernatants were pooled and concentration was determined using a microplate reader fluorescence measurement at 480/510 nm. A mean 25% loading efficiency of YO-PRO®-1 was calculated for protocells used in the model drug delivery experiments in vitro and ex ovo. To load and quantify gemcitabine (GEM), 0.5 mg of Hexagonal mMSNs (m_{mMSN}) were suspended in 50 μL of GEM dissolved n DI water at 10 mg/mL (m_{GEM}=0.5 mg) and stored for 12 hours at 4° C. After drug loading, targeted protocells were prepared using method described earlier in Anti-EGFR targeted protocell preparation. At each step, supernatant was collected, pooled (v₁=2.55 mL), and GEM loading was determined using a microplate reader absorbance measurement at 265 nm. A standard curve generated from a serial dilution of GEM in PBS (n=3) was used to calculate the concentration of GEM in the supernatant. To account for absorbance signal from non-GEM components in the supernatant, unloaded protocells were prepared simultaneously under identical conditions and measured at 285 nm. This absorbance value (Abs_{control}) was subtracted from the value obtained from supernatant containing GEM (Abs_{GEM}) prior to calculation of GEM concentration based on standard curve [c₁=(Abs−0.0507)/7.7115]. For example, we used (m_{mMSN}=0.5 mg), and (m_{GEM}=0.5 mg) and we obtained (Abs_{GEM}=2.51) and (Abs_{control}=1.18). To solve for the amount loaded [Abs_{GEM}−Abs_{control}]=1.33, then GEM amount in the supernatant can be calculated by [c, =(1.33−0.0507)/7.7115]=0.17 mg/mL. The total volume of the pooled supernatant is used to calculate the amount of GEM In the supernatant (m₁=c₁*v₁) or (m₁=0.17 mg/ml*2.55 mL)=0.43 mg. The supernatant amount (m₁) was then subtracted from the starting GEM amount (m_{GEM}) to estimate the total amount loaded into protocells [m_{loaded}=m₀−m₁] or (0.5 mg-0.43 mg)=0.07 mg. To estimate the loading capacity as a percentage of weight we use the formula [(m_{loaded}/m_{mMSN})*100%] or (0.07 mg/0.5 mg)*100%=14% (w/w). This experiment was repeated 4 times with different Hexagonal mMSN preparations and we determined the average GEM loading capacity of protocell=15.25%±1.6% (mean±SD). While the loading percentage of our protocells is lower than what was reported by Dr. Nel's group, the present loading conditions contain half the amount of GEM that was described by the Meng et al. (2015). Since GEM is neutral at physiological pH, and mMSNs are negatively charged, we do not suspect an electrostatic interaction to play a significant role in loading, instead suspect the GEM and mMSNs will reach an equilibrium state where the small molecule drug will occupy the high internal space of the pores and will then be encapsulated with the addition of the lipid bi-layer in protocell assembly. A 3.5-5 kD MWCO Float-A-Lyzer was used to evaluate GEM release kinetics in either PBS (pH 7.4) or citrate buffer (pH 5.0). GEM was encapsulated into protocells as described above, then protocells were loaded into Float-A-lyzers and sealed in 50 mL conical tubes containing either PBS or citrate buffer, and stored at 37° C. while stirring. 0.5 mL of dialysate was removed for 265 nm absorbance analysis on BioTek microplate reader at multiple time points, then added 0.5 mL of fresh dialysate solution to the conical tube. To assess protocell size at 24 and 72 hours a sample removed from the Float-a-Lyzer, and the hydrodynamic size measured on Malvern Zetasizer Nano ZS, then it was placed back inside the Float-a-Lyzer and stored at 37° C. while stirring. Consistent with findings reported by Meng et al. (2015), there was no evidence of drug precipitation and the effective release of GEM was determined by cell viability analysis. In addition, the loaded and targeted protocells maintained monodispersity.

Targeted Protocell GEM Delivery and Cytotoxicity Assessment.

About 1.5×10⁵ cells/mL of REH and REH+EGFR cell lines were incubated with either 0, 1, 5, 10, 25, or 50 μg/mL of GEM loaded (about 15% w/w) anti-EGFR targeted protocells in complete medium for 1 hour at 37° C. Cells were centrifuged (500 g, 3 minutes) and washed twice in complete media and transferred to a white 96-well plate for 24 hours at 37° C. In comparison, about 1.5×10⁵ cells/mL of REH and REH+EGFR cell lines were incubated with either 0, 0.6, 3, 6, 15, or 30 μM of free GEM, the equivalent doses based on 15% (w/w) GEM loading into protocells, under identical experimental conditions. Cell viability was assessed by CellTiter-Glo® 2.0 Assay as measured by BioTek microplate reader. The cell viability was calculated as a percentage of non-protocell treated sample.

In Vitro Internalization and Cargo Release Assay.

REH+EGFR cells were suspended in RPMI 1640 supplemented with 10% FBS media at a concentration of 5×10⁵ cells/mL. Then one mL of cells was incubated with YO-PRO®-1 loaded, RITC-labelled anti-EGFR protocells at 10 μg/mL for 60 minutes at 37° C., washed twice in media to remove unbound protocells, and incubated for 1, 8, 16, and 24 hours respectively at 37° C. under 5% CO₂. The protocell-treated cells were pelleted using a benchtop centrifuge, at each time point, and resuspended in an acid wash solution (0.2 M acetic acid, 0.5 M NaCl, pH 2.8) and incubated on ice for 5 minutes. Cells were then washed twice with PBS by centrifugation and protocell internalization was assessed by a red fluorescence shift and cargo release was assessed by a green fluorescence shift as measured by a BD Accuri™ C6 flow cytometer. Additionally, live cells were imaged on a glass slide using the Leica DMI3000 B inverted microscope.

Calculations to Identify Optimal Liposome to mMSN Surface Area Ratio.

To estimate the number of particles in solution (n), a shape applicable model was employed to calculate mMSN exterior surface area (SA) and volume (V_{mMSN}) from dimensional measurements obtained from TEM image analysis (n=50), pore volume (Vₚₒᵣₑ) measurements from nitrogen adsorption-desorption isotherms, a mesoporous silica density (φ of 2 g/cm³, and a sample mass (m). The equations below were used to estimate the number of particles in solution per unit concentration (mg/mL) and the external particle surface areas (nm²) used in determination of the lipid silica surface area ratio.

Hexagonal mMSN Calculations

$${SA}_{mMSN} = 6 ah + 3 \sqrt{3} * {a}^{2}$$

$${V}_{mMSN} = \frac{3 \sqrt{3}}{2} {a}^{2} h$$

$${n}_{mMSN} = \frac{( m / ρ ) + ( m * {V}_{pore} )}{{V}_{mMSN }}$$

For example—a=44.80 nm, h=50.68 nm, m=0.1 g, ρ=2 g/cm³, Vₚₒᵣₑ=0.83 cm³/g SA_{mMSN}=2.41*10⁴ nm², V_{mMSN}=2.64*10⁵ nm³, n_{mMSN}=4.9*10⁴ mMSNs
Spherical mMSN Calculations

$${SA}_{mMSN} = 4 {π ( D / 2 )}^{2}$$

$${V}_{mMSN} = \frac{4}{3} {π ( {D}_{mMSN} / 2 )}^{3}$$

$${n}_{mMSN} = \frac{( m / ρ ) + ( m * {V}_{pore} )}{{V}_{mMSN }}$$

For example (5 nm pore mMSN)—d=99.32 nm, m=0.1 g, ρ=2 g/cm³, Vₚₒᵣₑ=0.86 cm³/g
SA_{mMSN}=3.11*10⁴ nm², V_{mMSN}=5.17*10⁵ nm³, n_{mMSN}=2.69*10¹⁴ mMSNs
Rod-Like mMSN Calculations

$${SA}_{mMSN} = 2 π ( w / 2 ) ( l ) + 2 {π ( w / 2 )}^{2}$$

$${V}_{mMSN} = {π ( w )}^{2} l$$

$${n}_{mMSN} = \frac{( m / ρ ) + ( m * {V}_{pore} )}{{V}_{mMSN }}$$

For example—w=81.97 nm, l=176.68 nm, m=0.1 g, ρ=2 g/cm³, Vₚₒᵣₑ=0.87 cm³/g SA_{mMSN}=5.69*10⁴ nm², V_{mMSN}=9.77*10⁵ nm³, n_{mMSN}=1.42*10¹⁴ mMSNs

Next the surface area (SA) of liposomes was estimated by calculating the number of lipid molecules per unit mass (m) and assumed 0.59 nm² to represent the area of a single lipid head group. It was also assumed that cholesterol area does not contribute to the external surface area of liposomes. Finally, it was assumed that the internal surface area (SAᵢₙₙₑᵣ) is equal to half the total SA of the liposomes per unit mass.

$${Moles}_{component} = \frac{{m}_{component}}{M {W}_{component}} {N}_{A}$$

$${SA}_{inner} = ( 0.59 * {\sum }_{i = 1}^{n} {Moles}_{{i}_{component}} ) / 2$$

For example—DSPC:chol:DSPE-PEG₂₀₀₀ liposomes—mol ratio (49:49:2)
DSPC MW=790.145 g/mol, DSPE-PEG₂₀₀₀ MW=2805.497 g/mol, m=0.2 g
SAᵢₙₙₑᵣ=2.54*10¹⁹ nm²

To estimate the interior liposome surface area to total exterior mMSN surface area, the SA_{mMSN} was multiplied by the number of mMSNs (n) per unit mass, then liposomes interior SA was divided by mMSNs surface area per unit mass at the 2:1 mass ratio experimentally determined as below.

SA_{total mMSNs}=n_{mMSN}*SA_{mMSN}

SA ratio=SA_{liposome inner}/SA_{total mMSNs}

For example—Hexagonal mMSNs (calculated above) m=0.1 g, SA_{mMSN}=2.41*10⁴ nm², n_{mMSN}=4.99*10¹⁴ mMSNs, Liposomes (calculated above) SAᵢₙₙₑᵣ=2.54*10¹⁹ nm²
SA ratio=2.11:1

The calculated mass of fluorescent liposome (DSPC:chol:DSPE-PEG₂₀₀₀:NBD-Chol—54:43:2:1 mol %) to mMSN (118.7 nm) is 0.283 to 1. The experimental quantification of mass of fluorescent labeled liposome to mMSN is 0.276 to 1, as measured from fluorescence intensity of unbound liposomes in the supernatant following centrifugation of the protocells compared to a standard curve generated from known fluorescent liposome concentration. The calculated and experimental values are within 4.7% of each other, which is supportive of our method of surface area ratio calculations.

### Results and Discussion

### Synthesis Criteria for Monosized Protocells

Protocells were formed by fusion of zwitterionic lipid-based vesicles on monosized MSN (mMSN) cores synthesized with varying size, shape, and pore morphologies (See Experimental Section for detailed synthesis procedures). Vesicle fusion on silica glass substrates to form planar supported lipid bi-layers has been extensively studied using atomic force microscopy, quartz crystal microbalance, deuterium nuclear magnetic resonance, surface plasmon resonance, fluorescence microscopy and ellipsometry (Bayer and Bloom, 1990; Johnson et al., 1991; Keller et al., 2000; Reviakine et al., 2000: Johnson et al., 2002; Richter and Brisson, 2005), where the fusion process has been shown to involve vesicle adsorption followed (in some cases at a specific surface coverage) by vesicle rupture and desorption of excess lipid to form a bi-layer separated from the glass surface by an intervening 1-2 nm thick water layer. Generally, the process of phospholipid vesicle fusion with smooth glass supports is governed by the same Derjaguin-Landau-Verwey-Overbeek (DLVO) forces that are responsible for colloid aggregation; hence, both vesicle-substrate and vesicle-vesicle interactions need to be considered. DLVO theory models the forces in such systems as consisting of an electrostatic interaction combining with a van der Waals attraction; as such, SLB fusion depends on pH, which controls the extent of deprotonation of surface silanol groups to form anionic ≡Si—O⁻ species above pH 2, and the ionic strength and cationic component of the buffer, which dictate, respectively, the Debye length (mediating electrostatic interactions) and the cation hydration diameter (Cremer and Boxer, 1999). Cremer and Boxer studied fusion of positively charged, neutral and negatively charged vesicles onto glass as a function of pH (3-12) and ionic strength (0-90 mM). They found neutral and positively charged vesicles fuse under all conditions, whereas negatively charged vesicles fuse only above a certain ionic strength, which increased with pH (negative charge of silica surface). This is in keeping with expectations of DLVO theory as increasing ionic strength reduces electrostatic repulsion between vesicles and the glass surface (Cremer and Boxer, 1999).

Although considerably fewer studies have been performed on vesicle fusion on silica nanoparticles, the mechanism and governing forces are expected to be comparable but further influenced by the nanoparticle curvature. Using differential scanning calorimetry (DSC) in combination with dynamic light scattering (DLS), Savarala et al. (2011) studied the fusion of the zwitterionic 1,2-dimyristoyl-sn-glycero-3-phosphocholine (DMPC) vesicles on silica beads with diameters ranging from 100 to 4-6 nm at neutral pH and ionic strengths ranging from 0 to 0.75 mM NaCl. For a specific ratio of lipid surface area to silica surface area of 1 (SA_{lipid}:SA_{silica}=1), they found no (or very slow) vesicle fusion to occur in pure water and that higher ionic strengths achieved fusion on successively smaller particles (100-20 nm) (Savarala et al., 2010). 4-6 nm silica beads did not form supported lipid bi-layers; rather, these beads appeared to associate with the exterior surfaces of the vesicles (Savarala et al., 2010). These results differ somewhat from flat surfaces and, in keeping with DLVO theory, suggest that, for progressively smaller particles, possible repulsive electrostatic interactions must be reduced by increasing ionic strength and/or attractive electrostatic interactions promoted by cation association with phosphocholine to compensate for increased membrane curvature (assuming conformal SLBs). This result is consistent with a study by Garcia-Manyes et al. that showed the surface charge of zwitterionic DMPC liposomes at neutral pH is negative at <100 mM NaCl solution and positive at higher ionic strength. Excess lipid, i.e., SA_{lipid}:SA_{silica}>1 appears to promote SLB formation on silica nanoparticles (Garcia-Manyes et al., 2005). Mornet et al. studied the fusion of 30-50 nm diameter negatively charged 1,2-dioleoyl-sn-glycero-3-phosphocholine (DOPC)/1,2-dioleoyl-sn-glycero-3-phospho-L-serine (DOPS) vesicles on about 110 nm diameter spherical silica colloids by direct cryogenic transmission electron microscopy (Cryo-TEM). For SA_{lipid}:SA_{silica}=15 and a buffer ionic strength of 152 mM, they observed conformal about 5 nm thick SLBs to form by a process involving conformal vesicle adsorption followed by rupture to form SLB patches (Mornet et al., 2005). Multiple adsorption and fusion events resulted in complete SLBs that conformed to the moderate surface roughness/microporosity of the Stöber silica nanoparticle surface (Mornet et al., 2005).

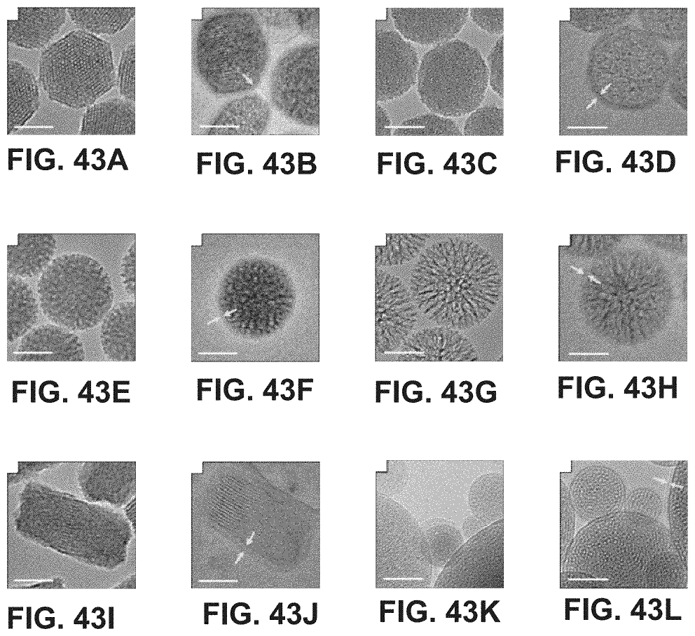

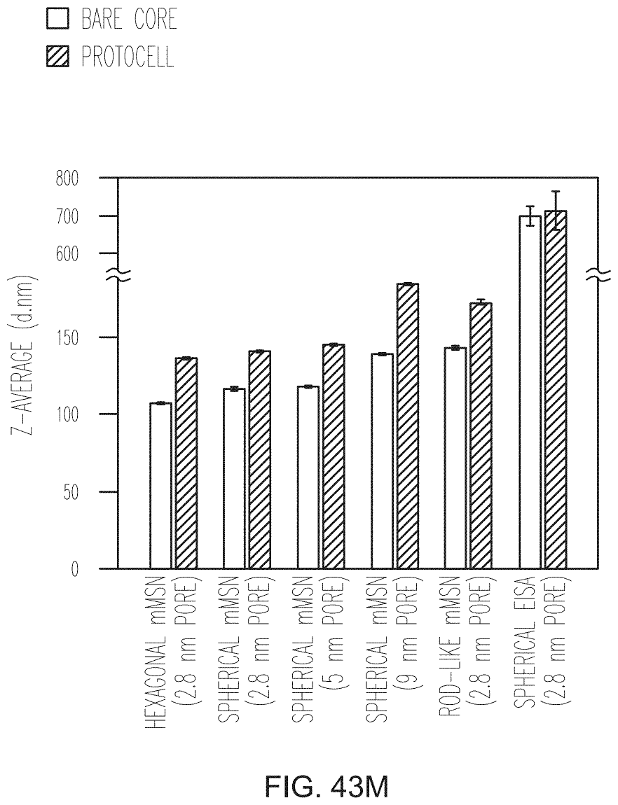

Numerous researchers have studied vesicle fusion on mesoporous silica macroparticles and nanoparticles as a means to form cell-like biomimetic materials (Buranda et al., 2003) and lipid bi-layer encapsulated nanoparticles for drug delivery (Ashley et al., 2012; Epler et al., 2012; Cauda et al., 2010; Meng et al., 2015; Wang et al., 2013; Zhang et al., 2014; Ashley et al., 2011; Liu et al., 2016; Han et al., 2015). To date, nanoparticle studies have employed primarily spherical cetyltrimethylammonium bromide (CTAB)-templated MSN formed by aerosol-assisted evaporation-induced self-assembly (EISA) (Ashley et al., 2012; Epler et al., 2012; Ashley et al., 2011; Liu et al., 2009a; Liu et al., 2009b; Dengler et al., 2013) or colloidal processing and characterized by worm-like or isotropic mesopores with diameters of about 2-3 nm (Meng et al., 2015; Wang et al., 2013; Zhang et al., 2014; Liu et al., 2016; Han et al., 2015). Direct Cryo-TEM observations of protocells have shown the bi-layer thickness to range from about 4-7 nm (Meng et al., 2015; Ashley et al., 2011; Liu et al., 2009; Dengler et al., 2013), corresponding to that measured for solid silica nanoparticle SLBs (Mornet et al., 2005) or planar SLBs (Johnson et al., 2002). SLBs span the surface mesopores and remain conformal to the MSN surface, as we, and others, have shown by Cryo-TEM imaging (see, for example, FIG. 43). With respect to SLB formation, surface porosity decreases the areal fraction of silica at the nanoparticle surface and, assuming spanning lipid bi-layers, reduces accordingly the possible magnitude of both van der Waals and electrostatic interactions that drive vesicle fusion. The fact that the modular MSN features of size, shape, pore size, pore volume, and pore morphology are important for their ultimate use as nanocarriers prompts us to ask how MSN physicochemical characteristics along with processing conditions influence vesicle fusion to form MSN-supported lipid bi-layers aka ‘protocells’ for use as nanocarriers—where key criteria are size monodispersity, preservation of shape, and stability within physiologically relevant complex biological media.

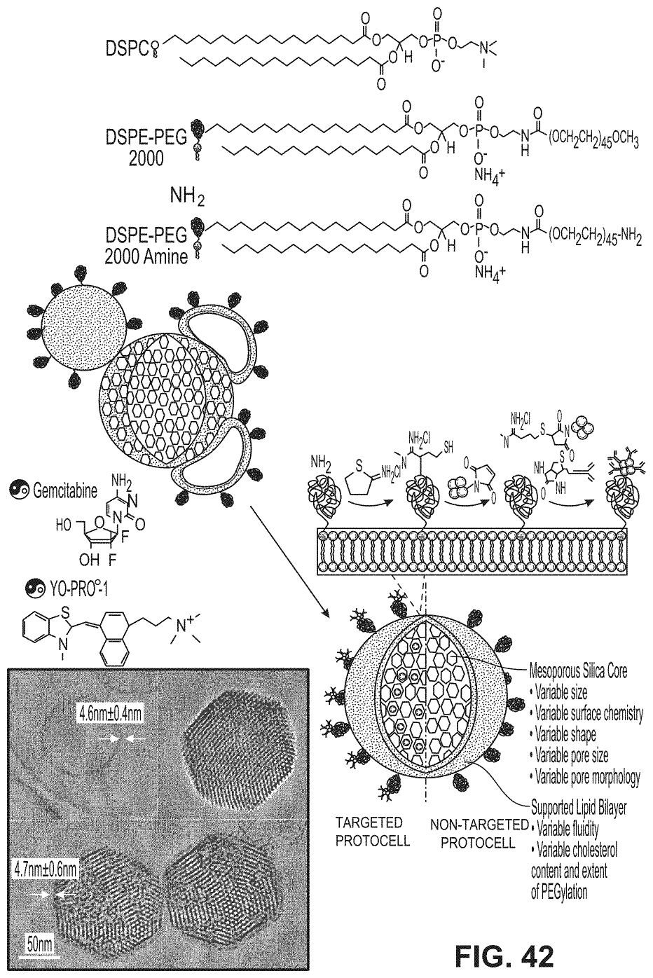

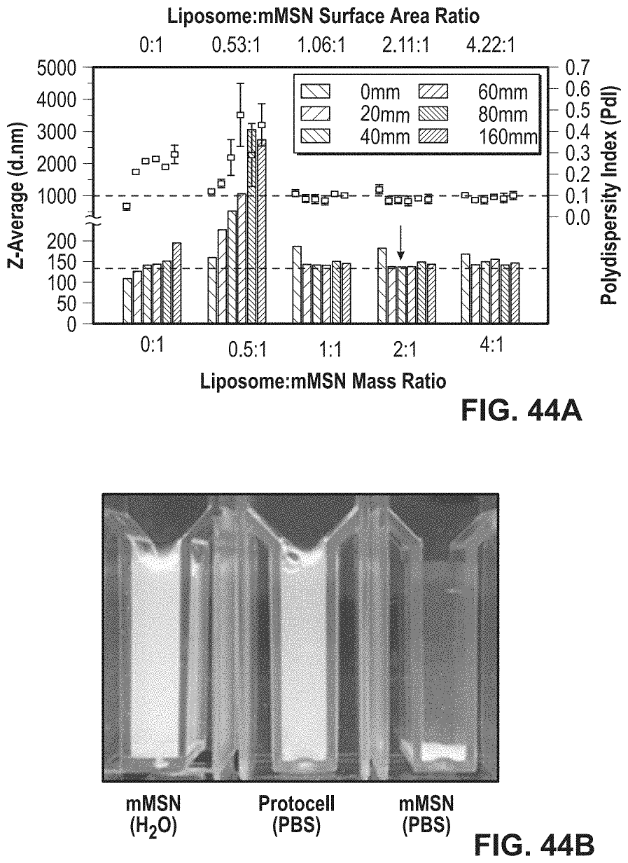

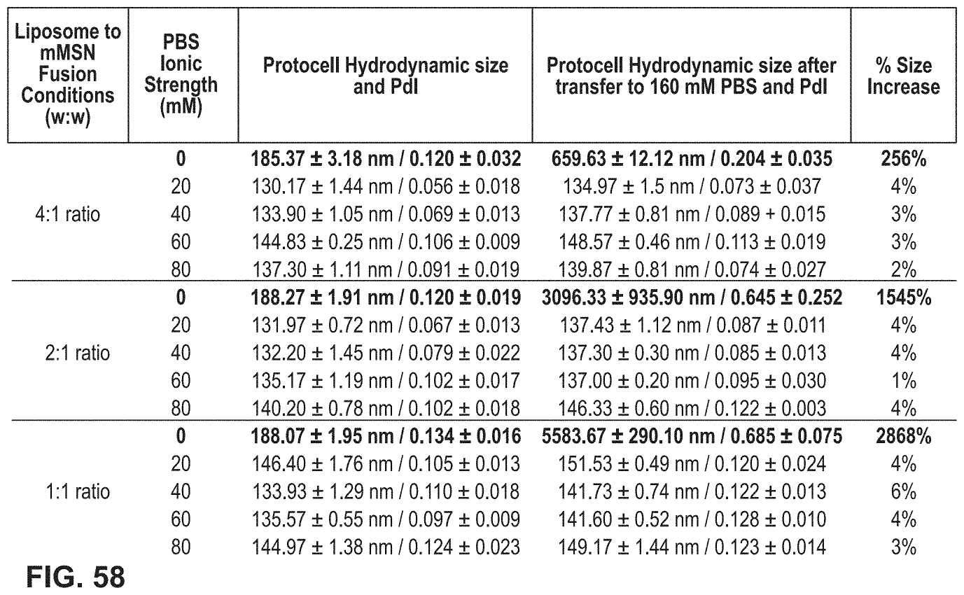

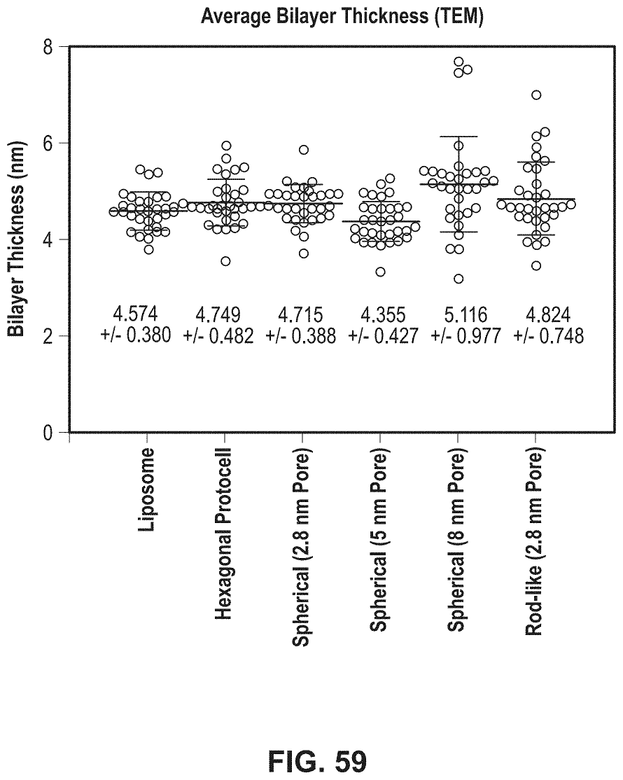

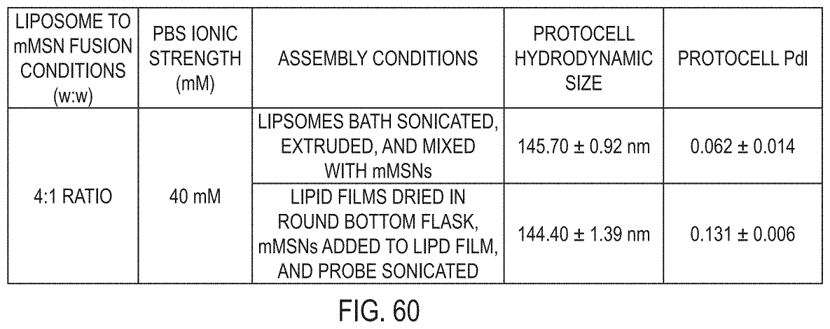

To address this question, monosized, about 107 nm (hydrodynamic diameter measured by DLS), single-crystal-like mMSN composed of close-packed cylindrical pores confined within a hexagonally shaped nanoparticle that is disc-shaped in cross-section were studied (FIG. 42 and FIG. 43). This highly asymmetric mMSN (referred to as Hexagonal mMSN) has opposing porous surfaces adjoined by grooved silica facets, thereby providing two distinct surfaces for vesicle fusion. To understand the roles of SA_{lipid}:SA_{silica} and ionic strength on vesicle fusion, we assembled protocells by mixing Hexagonal mMSNs with about 90 nm hydrodynamic diameter liposomes (composition=1,2-distearoyl-sn-glycero-3-phosphocholine (DSPC), cholesterol (-chol), and 1,2-distearoyl-sn-glycero-3-phosphoethanolamine-N-[methoxy(polyethylene glycol)-2000] (DSPE-PEG₂₀₀₀)—where the molar ratio of DSPC:chol:DSPE-PEG₂₀₀₀ equaled 54:44:2, FIG. 55). Liposomes were prepared by extrusion in a series of solutions consisting of 0 mM, 40 mM, 80 mM, 120 mM, 160 mM, and 320 mM ionic strength phosphate buffered saline (PBS). To complete the assembly process, the protocells were washed twice by centrifugation and resuspended in the final buffer solution with bath sonication and pipetting. Through variation of the lipid:silica ratio (wt:wt) and PBS concentration, we were able to adjust the SA_{lipid}:SA_{silica} from 0 (mMSN alone used as a control) to 4.22:1 and the ionic strength of the fusion conditions from 0 (water) to 160 mM spanning physiologically relevant ranges needed for in vivo applications (vide infra). A shape applicable model was used to calculate the external SA_{silica} from dimensional measurements of mMSNs obtained from TEM images (FIG. 58), using the pore volume obtained from nitrogen sorption data (FIGS. 58 and 57), and assuming 2.0 g/cm³ as the silica framework density (Brinker and Scherer, 2013); SA_{lipid} was calculated assuming 0.59 nm² as the phospholipid head group area (Marsh, 2013); and that cholesterol does not contribute to SA_{lipid}. Using a Malvern Zetasizer Nano ZS, the hydrodynamic diameter, polydispersity index (PdI), and zeta-potential (ζ) of protocells was measured. FIG. 44A plots hydrodynamic diameter and PdI as a function of SA_{lipid}:SA_{silica} and ionic strength. Consistent with our expectations from DLVO theory, without lipid, mMSNs (ζ=−28.1 mV) aggregate with increasing ionic strength due to the reduced Debye length and concomitant reduction in the range of electrostatic repulsion. For samples prepared with SA_{lipid}:SA_{silica}<1, the ratio to cover the external surface of the mMSN with a single phospholipid bi-layer, we observed severe aggregation that increases with ionic strength indicative of aggregation of exposed silica surfaces accompanied by liposome adsorption and possible bridging. For samples prepared with SA_{lipid}:SA_{silica}>1, it was observed much more uniformly sized particles (PdI<0.1) with hydrodynamic diameters ca 30 nm larger than the parent mMSN and zeta-potentials in the range (ζ=−3.3 mV) consistent with the formation of a PEGylated zwitterionic SLB that shields the mMSN charge and provides a repulsive hydration barrier that stabilizes the protocells within biologically relevant media (vide infra). The exception are samples prepared in pure water (ionic strength=0 mM) where for all SA_{lipid}:SA_{silica} we observed diameters 50 to 80 nm greater than the parent mMSN along with a trend of increasing PdI (FIG. 58). Samples prepared in pure water have a zeta potential comparable to the parent mMSN (ζ=−41.0 mV) and aggregate when transferred to 180 mM PBS (ζ=−28.1 mV). These ionic strength effects indicate fusion to be inhibited in pure water and are consistent with those obtained by Savarala et al. for fusion of single component 1,2-dipalmitoyl-sn-glycero-3-phosphocholine (DPPC) zwitterionic vesicles on solid 100 nm silica beads at SA_{lipid}:SA_{silica}=1, where ionic strengths≥0.05 mM NaCl were needed for fusion as assessed by DSC (Savarala et al., 2010). Direct Cryo-TEM observation of Hexagonal mMSNs fused with DSPC-based liposomes at SA_{lipid}:SA_{silica}=2.11:1 and ionic strength 40 mM show a conformal SLB with thickness 4.7±0.5 nm (FIG. 42 and FIG. 59) observed both on the porous and grooved surfaces (FIG. 43B). The increased diameter of about 10 nm determined by TEM is inconsistent with the about 25 nm increase measured by DLS. Such discrepancies are often reported in the literature (Meng et al., 2015; Lin et al., 2011). Considering that the SA_{lipid} of a 90 nm liposome is less than that of a Hexagonal mMSN, multiple liposome fusion events may be needed to create a complete SLB (FIG. 42). In time-dependent Cryo-TEM. Mornet et al. showed liposome fusion on 100 nm colloidal silica nanoparticles to occur by a ‘two-step’ process involving adsorption followed by deformation and rupture (Mornet et al., 2005). Although a time-dependent Cryo-TEM study wasn't conducted, evidence of deformed vesicles that conform to the mMSNs was observed, which likely subsequently rupture to form SLBs in a similar ‘two-step’ process. Although it has been suggested that SLB formation on spherical, isotropic MSNs via probe sonication of dried lipid films in saline solution may proceed through a pathway other than vesicle fusion, Implementing the identical probe sonication technique (Meng et al., 2015; Liu et al., 2016) for Hexagonal mMSNs results in protocells indistinguishable (i.e., nearly identical hydrodynamic diameter and PdI) from those formed by fusion with DSPC-based liposomes at SA_{lipid}:SA_{silica}=4.22:1 and ionic strength 40 mM (FIG. 60). Finally, to help avoid any accompanying aggregation from occurring at the ionic strengths needed for vesicle fusion (and ultimately for ex ovo and in vivo applications, vide infra), conditions of excess of lipid and a low but sufficient ionic strength may serve to increase the relative rate of vesicle fusion with respect to aggregation thus allowing the formation of monosized protocells with a low PdI (FIG. 44A).

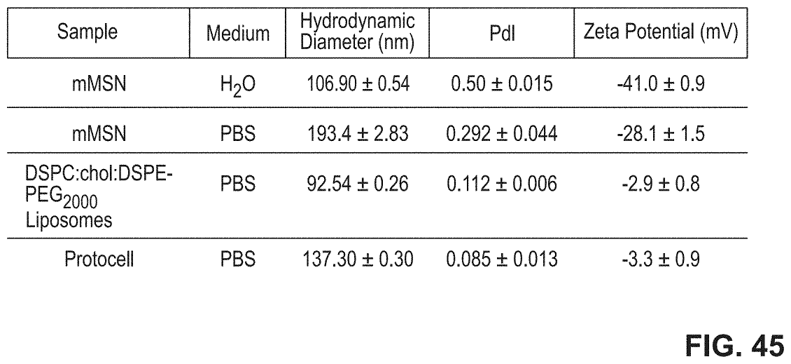

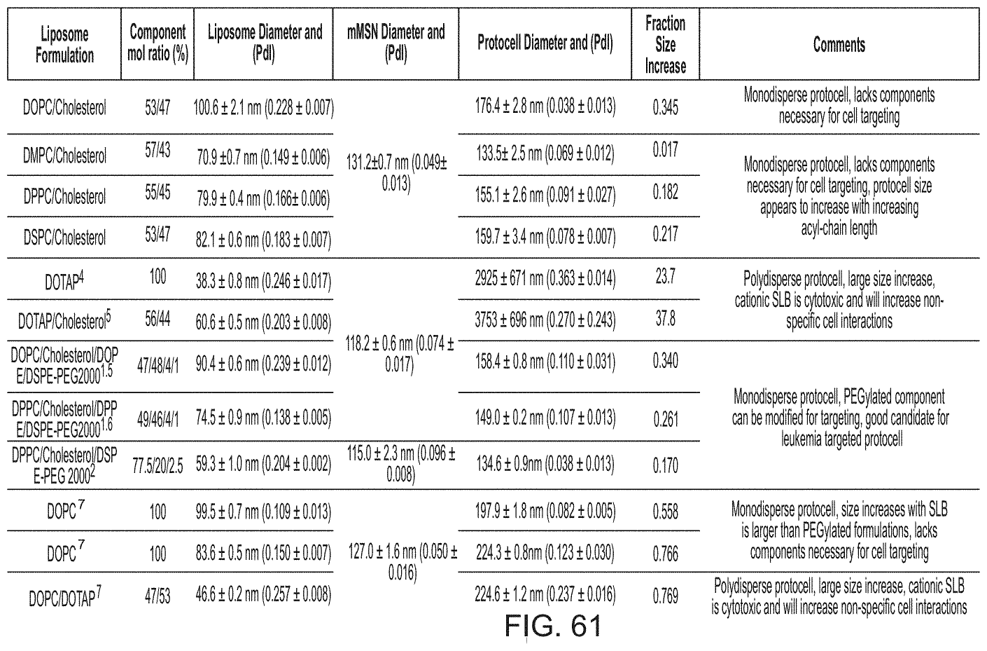

The results on vesicle fusion on Hexagonal mMSN established a wide processing window in which to synthesize rather monosized protocells. As noted above, a SA_{lipid}:SA_{silica}≈2:1 and ionic strength 40 mM appeared to represent an optimal fusion condition resulting in the smallest combination of hydrodynamic diameter and PdI (highlighted by a green arrow in FIG. 44A). To test how this condition depended on bi-layer fluidity or charge, vesicles were prepared containing unsaturated or saturated phosphatidylcholine (e.g., DOPC or DSPC) or the cationic lipid 1,2-dioleoyl-3-trimethylammonium-propane (DOTAP) based on liposomal formulations reported in the literature (FIG. 61). In general, that these conditions resulted in monosized protocells for zwitterionic lipid based formulations, whereas DOTAP resulted in aggregate formation. To further understand the influence of MSN physicochemical properties on protocell formation, the optimized fusion conditions were tested on a ‘library’ of MSNs with differing shapes (e.g., spherical or rod-like), particle size distributions (mMSN or EISA MSN), pore diameters (2.8 to 18 nm), and pore morphologies (aligned cylindrical, isotropic worm-like, and dendritic) (Lin et al., 2005; Lin et al., 2010; Chen et al., 2013; Nandiyanto et al., 2009; Wang et al., 2012; Shen et al., 2014; Huang et al., 2011; Yu et al., 2011). (See FIG. 43 and FIG. 56 for a summary of the mMSN and EISA MSN physicochemical properties). As observed by direct Cryo-TEM observation, about 4 to 5 nm thick conformal SLBs formed on all of the tested particles (FIGS. 43A-L and FIG. 59), and DLS showed a consistent increase in diameter of about 25 to 40 nm (FIG. 43M). By visual examination, a well-suspended and transparent dispersion of protocells in PBS contrasted with bare mMSNs that settle under normal gravity was observed (FIG. 44B). The exception was for spherical mMSNs prepared with dendritic pore diameters of about 18 nm. In this case it was observed, by Cryo-TEM, vesicle adsorption and deformation on the mMSN surface but little evidence of complete SLB fusion (FIG. 45). It is proposed that for this highly porous particle the magnitude of possible van der Waals and electrostatic interactions (that all scale nominally with surface silica concentration) is insufficient to cause rupture/fusion to form an SLB. Moreover, the topography of the silica surface is influential in the spreading process of the SLB, where 10-30 nm deep scratches were found to arrest spreading of egg phosphatidycholine bi-layers on borosilicate glass due to unfavorable bending interactions needed to maintain conformity (Cremer and Boxer, 1999 Sackmann, 1994). It is likely that for mMSNs there is a pore size above which the highly contoured regions of the pore arrest spreading and fusion. This pore size should be sensitive to the SLB composition, which dictates the bending modulus. Using unsaturated lipids and potentially decreasing the cholesterol content to make the membrane more flexible and promote SLB formation on mMSNs with larger pore size (Henriksen et al., 2006; Sackmann, 1995). However, at the cholesterol used (44%), it is unlikely that the transition temperature (−Tₘ) of the phosphatidylcholine SLB component is a major factor in size stability. It is also conceivable that fusion might be promoted by doping the buffer with divalent ions like Ca²⁺ or Mg²⁺ that, through several possible electrostatically mediated pathways, are known to promote vesicle fusion on glass (Nollert et al., 1995; Seantier and Kasemo, 2009). Finally adsorption of drugs within the pores would increase the solid fraction of the surface and potentially promote DLVO interaction and vesicle formation.

### Factors Influencing Colloidal Stability of Monosized Protocells for Use In Vivo

Having established a generalized process by which to reliably form monosized protocells in vitro, the physicochemical properties of the SLB that influence colloidal stability in complex biological media was studied. As noted above in vivo colloidal stability allows for both passive and active targeting as any process that non-selectively removes nanoparticles from circulation reduces concomitantly the number of particles that could accumulate in the tumor microenvironment due to the EPR effect or those that are available to selectively bind to target cells or tissues. Despite its importance, few papers unambiguously establish the stability of nanocarriers, which may in part explain inconsistent and unreproducible results in the literature, as are now generally recognized (Crist et al., 2013; Lin et al., 2012; Zarschler at al., 2014). Problematic is that in vivo colloidal stability is difficult to predict from in vitro measurements. For example, cationic MSNs with identical size, shape, and surface charge (and therefore indistinguishable according to NCI NCL standards) were shown to have completely different circulation and non-specific binding behaviors as elucidated by direct observation ex ovo in a CAM model (Townson et al., 2013) and SPECT imaging in a rat model (Adolphi et al. private communication). Here, colloidal stability was evaluated by determination of hydrodynamic size and polydispersity index in complex biological media and by direct observation in the CAM.

First, it was examined how the encapsulating SLB and its fluidity affected long term stability compared to the bare mMSN surface. Liposomes were prepared with zwitterionic lipids using either unsaturated DOPC or saturated DSPC as the major liposome component. The comparison between DOPC and DSPC is ideal because these lipids possess nearly identical molar mass, have the same acyl tail length, and yet exhibit Tₘ (about 20° C. and 55° C., respectively) below and above the storage and physiological temperatures (22° C. and 37° C., respectively). Additionally, the cis-configuration double bonds present in the DOPC acyl chains (absent in DSPC) are highly susceptible to oxidation, which can lead to structural instability (Lis et al., 2011). Unsaturated DOPC-based (composition=DOPC, chol, and 1,2-dioleoyl-sn-glycero-3-phosphoethanolamine-N-[methoxy(polyethylene glycol)-2000] (DOPE-PEG₂₀₀₀)-DOPC:chol:DOPE-PEG₂₀₀₀ mol ratio of 54:44:2) and saturated DSPC-based (composition=DSPC:chol:DSPE-PEG₂₀₀₀ mol ratio of 54:44:2) were prepared. Liposome compositions and hydrodynamic diameters are summarized in FIG. 55, where all possessed a hydrodynamic diameter<100 nm and low PdI value<0.2. Liposome to mMSN fusion was achieved in 40 mM PBS as described earlier then protocells were finally redispersed in 160 mM PBS. The formation of a complete SLB surrounding the MSN cores was verified by combinated techniques. DLS measurements show the hydrodynamic diameter, to be about 30 nm compared to mMSNs while maintaining a low PdI (<0.1) (FIGS. 44 and 56). Zeta potential measurements initiated Hexagonal mMSNs and protocells to have zeta potential (about −3.3 mV) similar protocells to the corresponding zwitterionic liposomes (−2.9 mV) and much lower than the mMSN (−28.1 mV). Direct observation by Cryo-TEM shoed the presence of a uniform conformal SLB surrounding the mMSN cores (FIG. 43A).

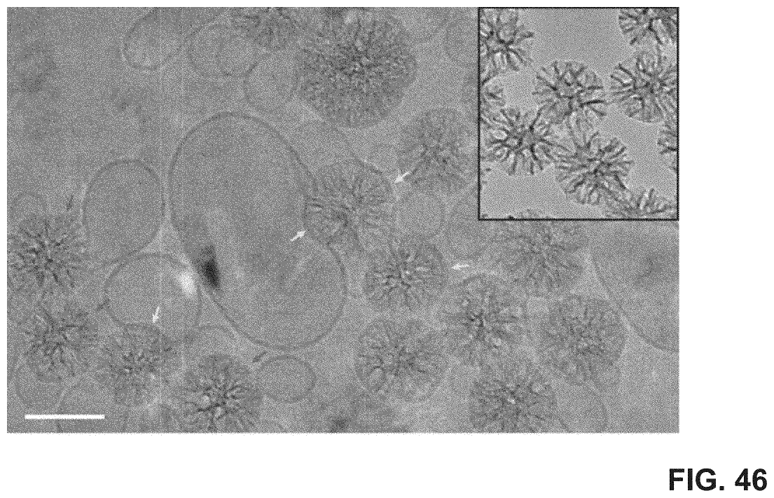

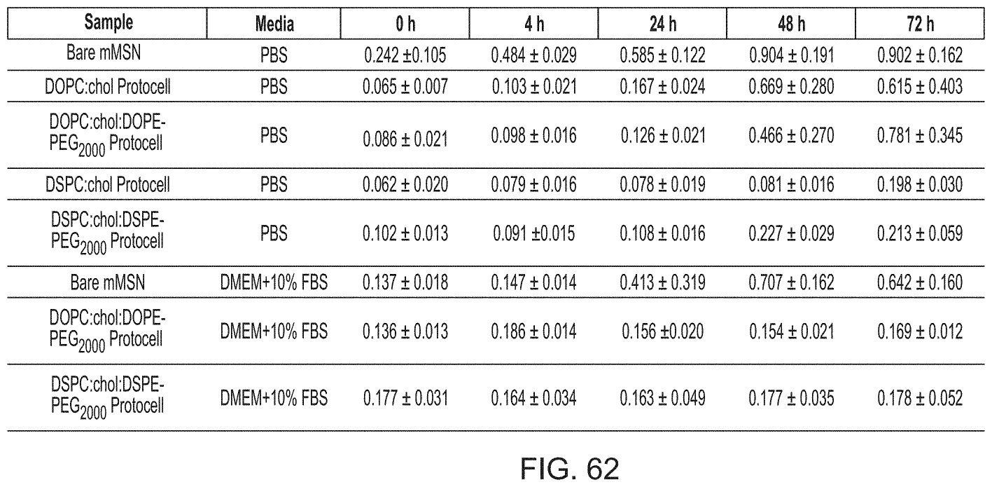

FIG. 46A shows changes in hydrodynamic size of protocells for 72 h at 37° C. compared to bare mMSN controls (see FIG. 62 for corresponding PdI). Whereas the hydrodynamic size of bare mMSNs increases within minutes of transfer to PBS at room temperature, and more rapidly at 37° C., both DOPC-based and DSPC-based protocells maintain uniform size for 24 hours. These results suggest that the colloidal stability of the protocells is primarily due to the zwitterionic SLB component rather than the PEG component, as the trends observed of DOPC and DSPC-based protocells prepared with and without PEG are nearly identical (FIG. 46A). The stabilizing effect of the zwitterionic SLB can be attributed to several factors. Zwitterionic coatings are shown to increase nanoparticle stability in high salt concentration solutions due to hydration repulsion which also minimizes non-specific protein adsorption in serum containing solutions (Estephan et al., 2010: Zhu at al., 2014; Soo Choi et al., 2007; Nag and Awasthi, 2013). In addition, the presence of both positively and negatively charged functional groups on nanoparticle surfaces has been shown to increase solubility in water over a wide pH range, limit non-specific interactions with cultured cells, and display a non-toxic profile upon interaction with cells based on cell viability assessment (Breus et al., 2009). That the protocells are encapsulated completely within a zwitterionic SLB is evidenced by the hydrodynamic size/PdI change of bare mMSNs, increasing from 106.9 nm/0.050 to 193.4 nm/0.292 in PBS after centrifugation (FIG. 45) along with their rapidly settling in PBS solution (FIG. 44B); incomplete SLB coverage would similarly result in the formation of irreversible aggregates via electrostatic destabilization and van der Waals forces, vide supra.

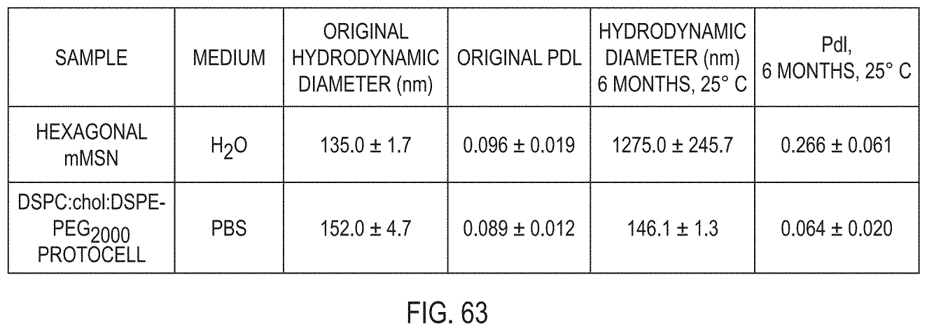

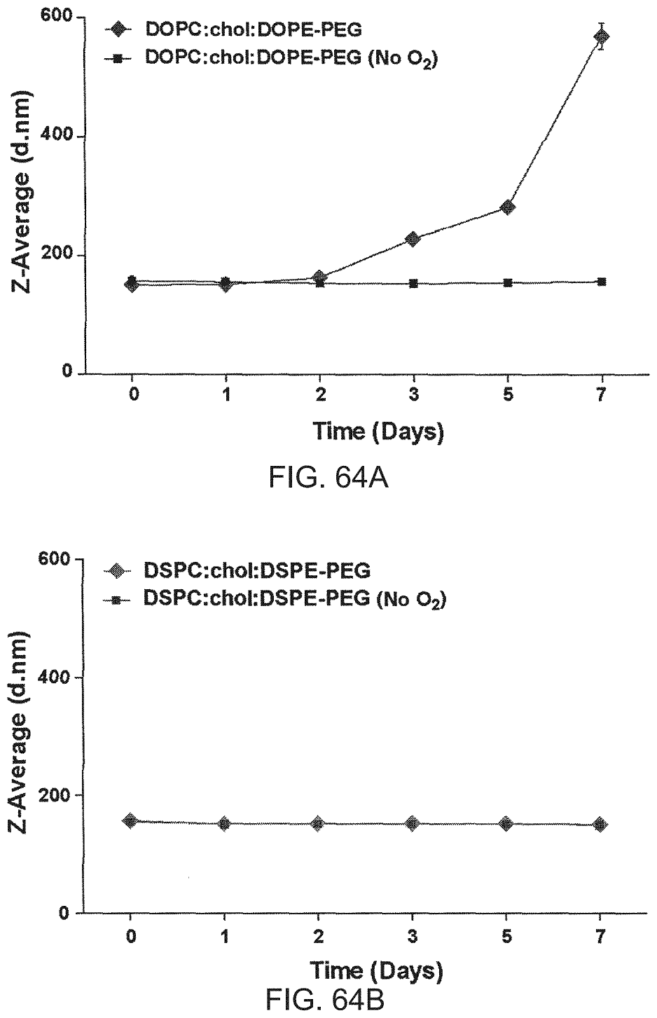

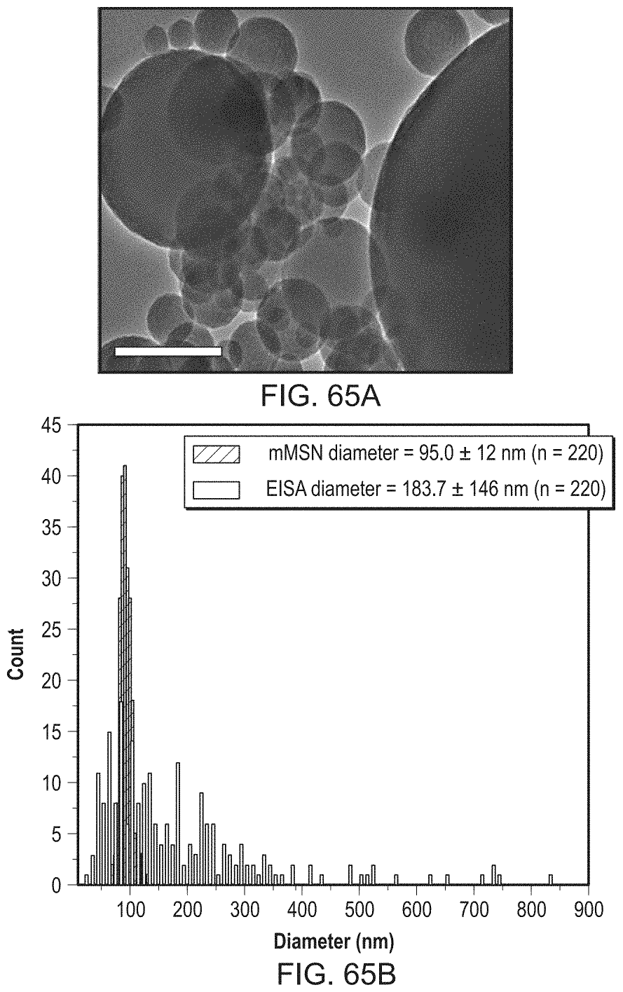

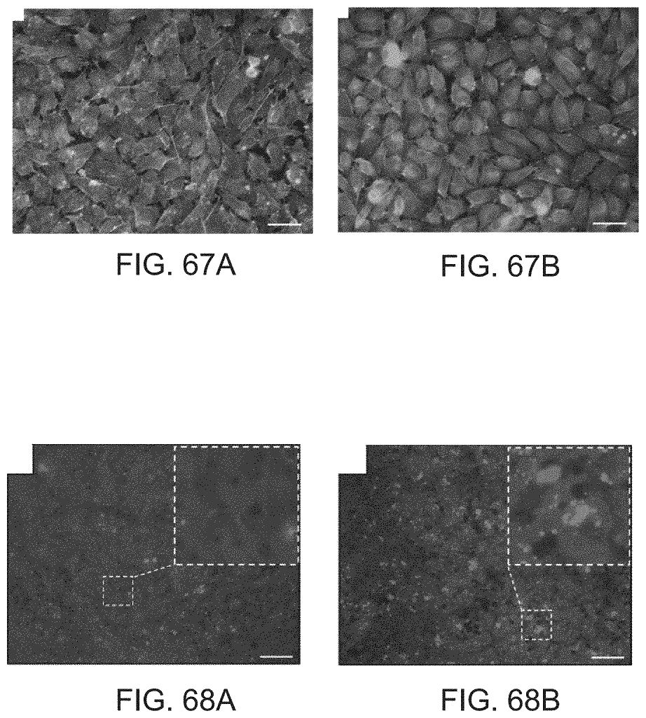

Concerning the influence of lipid bilayer composition on long-term stability, although both DOPC-based and DSPC-based protocells are stable for 24 hours, the size of both PEGylated and non-PEGylated DOPC-based protocells increases progressively from 24 to 72 hours in PBS. In comparison, DSPC-based protocells remain stable for >72 hours at 37° C. In PBS (FIG. 46A) and for over 6 months at room temperature (FIG. 67). To assess the possible role of lipid oxidation as being the cause of instability of DOPC-based protocells, protocells were prepared in deoxygenated PBS and the hydrodynamic size of protocells during storage of protocells for 7 days at 37° C. was examined. Interestingly, DOPC-based protocells were stable in an oxygen reduced buffer whereas the aggregate in standard PBS. In comparison the presence or absence of oxygen made no difference in DSPC-based protocell size stability (FIG. 63). This result indicates the oxidative state of double bonds present in the acyl chains play a significant role in the long-term stability of protocells. At the high cholesterol concentration used in our experiments, it is unlikely that the Tₘ of the phosphatidylcholine SLB component is a major factor in size stability, however, it is conceivable that there could be lipid exchange between protocells resulting in fusion or simply loss of lipid due to its finite residence time, leading to aggregation. Both of these effects should be mitigated by storage in either deoxygenated PBS or excess lipid.

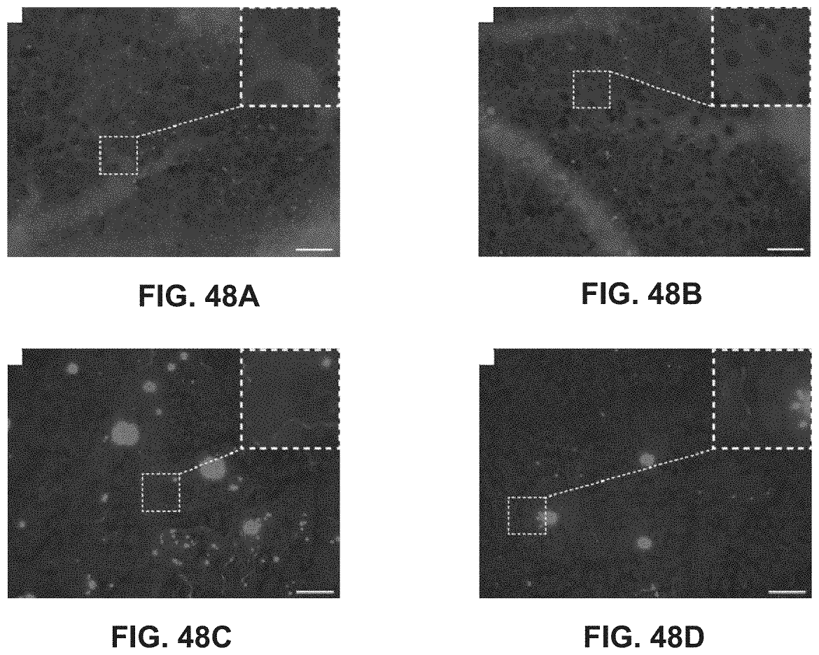

Although, colloidal stability of the protocells is primarily due to the zwitterionic SLB component, modification of nanocarriers with hydrophilic polymers has been widely shown to prolong in vivo circulations times, reduce protein adsorption, and reduce phagocytosis by immune cells (Ferrari, 2008). Therefore, only PEGylated protocells were compared to examine the influence of Tₘ in a more complex medium. Protocells were prepared in PBS and then transferred them to a cell culture medium containing fetal bovine serum. Similar to the previous experiment, DSPC-based protocells maintain size stability for >72 hours at 37° C. (FIG. 48B), Indicating minimal protein binding and destabilization of the SLB. Interestingly, we observe the identical size stability for DOPC-based protocells in complete media, suggesting that protein adsorption stabilizes the DOPC-based SLB and/or provides a steric barrier toward fusion and aggregation despite there being no measureable increase in hydrodynamic diameter.

Overall, the zwitterionic SLB confers excellent colloidal stability to the protocell in physiologically relevant media. Both unsaturated and non-fluid SLBs prepared with and without PEG have greatly enhanced stability compared to the parent mMSN. Nevertheless, the measured long-term stability of DSPC-based monosized protocells, compatibility with the majority of mMSN cores tested, and potential to incorporate functional modifications to PEGylated lipids, in particular amine terminated 1,2-distearoyl-sn-glycero-3-phosphoethanolamine-N-[amino(polyethylene glycol)-2000](DSPE-PEG₂₀₀₀-NH₂) which can be chemically modified with a functional component, prompted us to choose the DSPC-PEG-based protocell formulation for further in vitro, ex ovo, and in vivo studies.

### Influence of Protocell Size Dispersity on In Vitro and Ex Ovo Performance

For the development of therapeutic nanocarriers specifically targeted to leukemia cells, prolonged circulation times are needed to enhance the probability of delivery to distributed cells, within the blood, marrow and other tissue spaces and it is reported that particle size is an important determinant in delivery to tissue sites characteristic of this disseminated disease (Krishnan and Rajasekaran, 2014). Therefore, it is of interest to understand the effect of protocell size dispersity on in vivo performance. Potentially, a broad particle size distribution could effect/direct broad dissemination of protocells to differing tissues in addition to the peripheral vasculature and other tissues (liver, spleen, bone marrow) which may harbor leukemic cells, or, protected tissues which serve as sanctuaries for leukemic cells (testes, brain) and are frequent regions of recurrent or relapsed disease following systemic chemotherapy treatment. However, it is presently unclear as to how particle size polydispersity influences particle entrapment, non-specific binding, and circulation time. In order to assess the dependence of polydisperisty on non-specific binding and circulation, we compared monosized protocells with protocells assembled from MSN cores prepared by aerosol assisted EISA as previously reported (Lu et al., 1999). EISA cores are characterized by spherical MSNs with a power law particle size distribution ranging from ˜20 to ˜800 nm (see TEM images in FIGS. 43K, 43L, and 60) that results from the size distribution of the aerosol generator. EISA MSNs have a pore diameter of about 2.5 nm and a zeta-potential of about −31 mV (Liu et al., 2009a), comparable to those of Hexagonal mMSNs, so the comparison of their behaviors depends principally on polydisperisty (See FIG. 56 for other physicochemical parameters of the EISA MSN and protocells). Hexagonal and EISA protocells were prepared by fusion of vesicles with composition=DSPC:chol:DSPE-PEG₂₀₀₀ mol ratio of 54:44:2 according to methods described previously. The hydrodynamic diameter and PdI of EISA protocells was about 715 nm and 0.434 compared to about 137 nm and 0.085 for hexagonal protocells (FIG. 43M and FIG. 56).

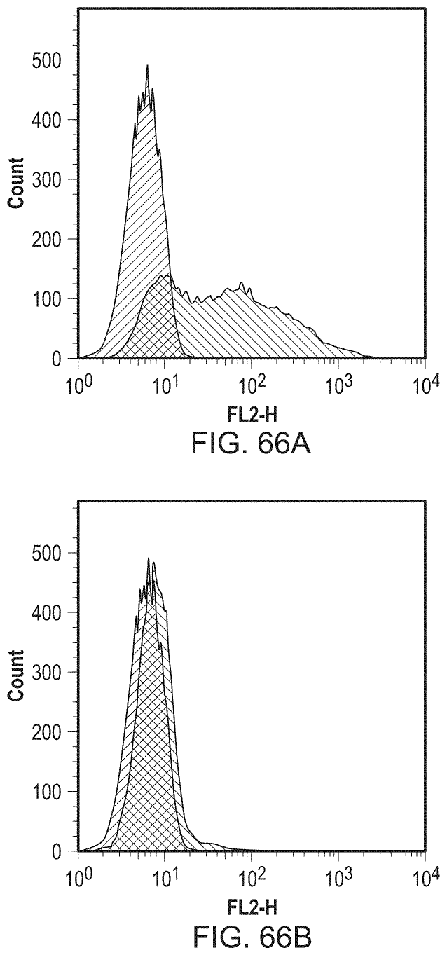

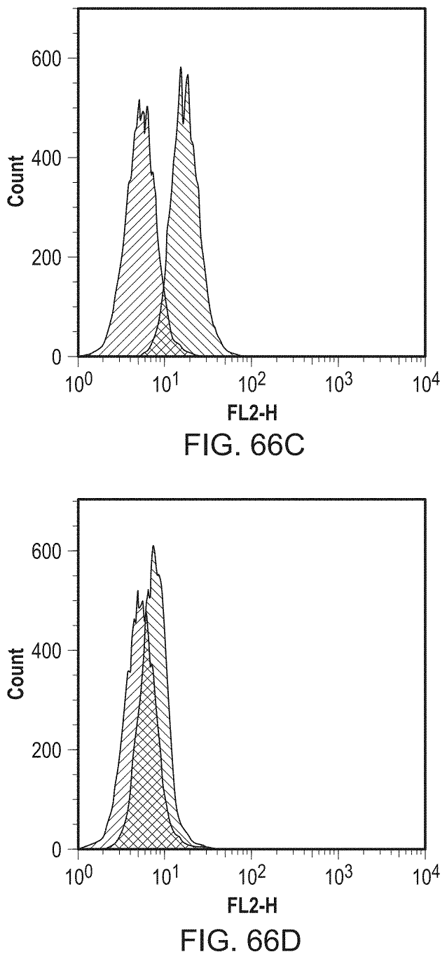

To investigate the role of polydispersity on in vitro MSN and protocell non-specific binding interactions, endothelial cells were incubated with either fluorescently labelled EISA or mMSN cores and their corresponding protocells (20 μg/mL) for 4 hours with complete medium under normal cell culturing conditions. Flow cytometry analysis showed both EISA and mMSN particles to have significant levels of non-specific binding to EA.hy926 cells (FIG. 66) where for EISA MSN the extended breadth of the FL2-H intensity curve reflected the size (and therefore) fluorescence intensity distribution of individual MSNs. Correspondingly, the fluorescence intensity binding curve for mMSNs was rather monodisperse. For both EISA and mMSN derived protocells, we observe a 20-fold decrease in non-specific binding relative to the parent core particle (FIG. 61, see also fluorescence microscopy images in FIG. 67). This indicates that the conformal and complete SLB serves to effectively shield lipophilic surface silanol groups (≡Si—OH) and anionic deprotonated silanols (≡Si—O⁻) present on the bare MSN and known to promote internalization via micropinocytosis and other non-specific endocytotic pathways (Meng et al., 2011). Our findings underscore the importance of the SLB in helping to prevent non-specific cell binding events, and support previous reports demonstrating minimal nonspecific cell binding affinity of polydisperse EISA protocells in vitro (Ashley et al., 2012; Ashley et al., 2011).

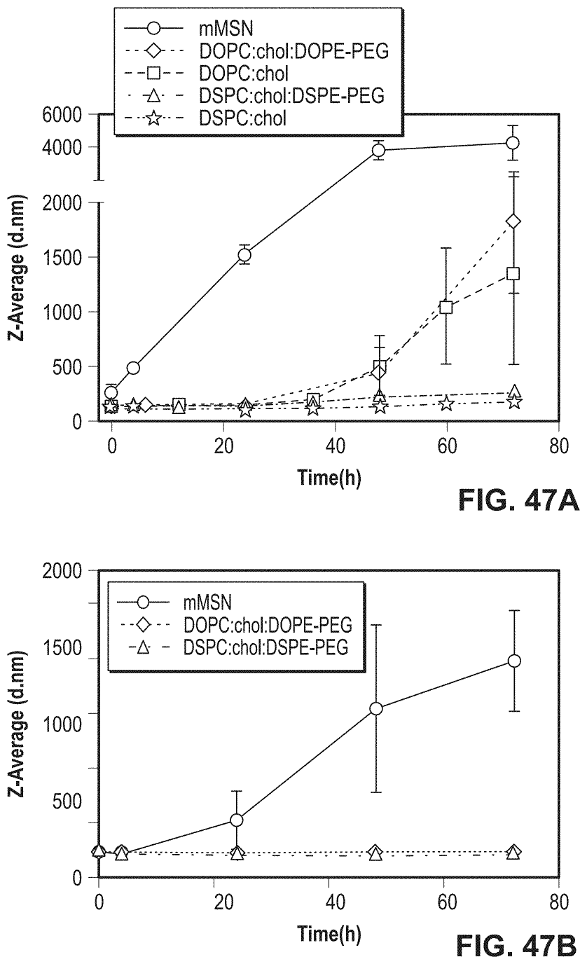

However, in vitro studies of nanoparticle behavior may be poor indicators of in vivo outcomes as they lack the complexities of in vivo conditions that present major obstacles to nanoparticle stability and target cell binding (Dobrovolskaia and McNeil, 2013). These obstacles include flow dynamics within the diverging and converging vasculature, opsonization by plasma proteins and uptake by the mononuclear phagocyte system, and the need for translocation across the capillary bed for tissue penetration. To assess MSN and protocell behavior in a more relevant model, the CAM model was employed as an in vivo (ex ovo) model of the vascular system in which to observe nanoparticle circulation, flow characteristics, non-specific interactions, and particle stability in a living system using intravital imaging (Townson et al., 2013 Vargas et al., 2007; Leong et al, 2010). Fluorescently labeled nanoparticles can be injected intravenously into the CAM vasculature and imaged over time. As investigated previously in vitro, mMSN cores as well as EISA and mMSN protocells were examined to assess the influence of the SLB and polydispersity on biodistribution in a more complex environment. The influence of the SLB on nanoparticle flow dynamics and non-specific ex ovo binding was immediately evident as bare mMSN cores bound to endothelial cells and arrested in the vessels of the CAM within 5 minutes of injection (FIG. 68A) and were largely taken up by phagocytic white blood cells after 30 minutes, reducing correspondingly the concentration of circulating mMSNs (FIG. 68B). By comparison, monosized protocells exhibited significantly lower non-specific binding and uptake by white blood cells leading to greatly improved circulation characteristics (FIGS. 47A and 47B). Striking was the contrast between mMSN and EISA protocells. Even though the in vitro outcomes were nearly identical, rapid sequestration of EISA protocells by immune cells, aggregation, and diminished circulation was noted within 5 minutes in the vascular CAM system (FIG. 47C), with a more pronounced effect after 30 minutes (FIG. 47D). The rapid uptake and reduced circulation are likely due to polydispersity leading to the majority of particles falling within a size range that either encourages immune cell uptake or advances unpredictable systemic circulation and distribution (He et al., 2011). The CAM results highlight the need for reduced size polydispersity to maintain circulation within highly vascularized networks and elucidate a major limitation of in vitro models in predicting in vivo results. In this regard, the vascularized CAM model improves greatly on in vitro models of specific and non-specific binding and more realistically assess the behavior of nanoparticles designed for in vivo use (Townson et al., 2013).

### Biocompatibility and Protocell Size Stability Ex Ovo and In Vivo

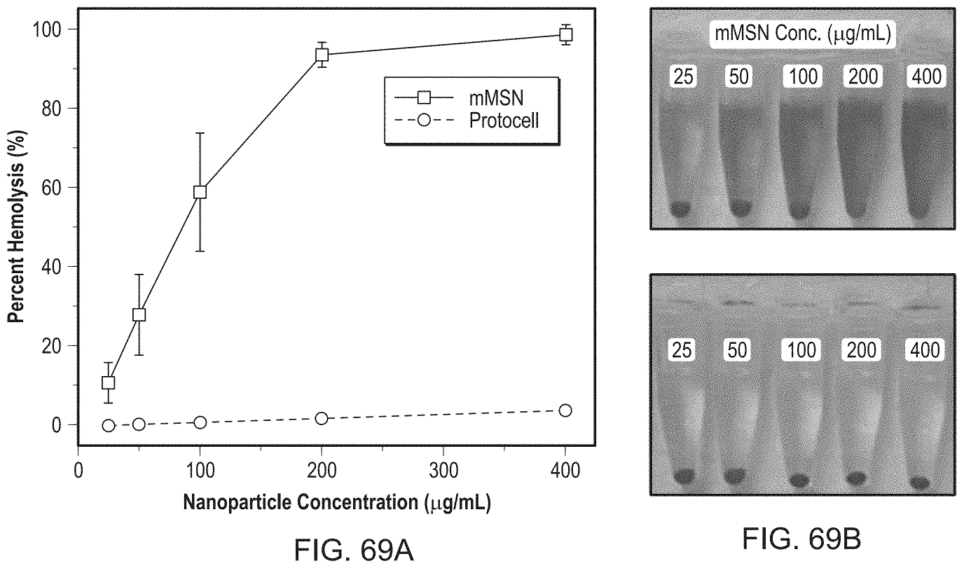

Previous studies have shown mesoporous silica to be a biocompatible material; however, the interpretation of the overall biocompatibility of MSN-based nanocarriers is complex due to several factors including methods of synthesis, physicochemical properties, size distribution, and surface modifications (Asefa and Tao, 2012). Therefore, to assess the influence of the SLB on biocompatibility and to determine the uniformity of the SLB coating, mMSNs and protocells were incubated with human red blood cells (hRBCs). The hemolytic activity and potential toxicity of bare mMSNs can be completely abolished with a SLB (FIG. 69). This result supports evidence of a complete (defect-free) lipid bi-layer coating that screens silanols (≡Si—OH) and anionic deprotonated silanols (≡Si—O⁻) Implicated in hemolysis (Zhang et al., 2012) and, thereby, provides enhanced biocompatibility of the protocells vis-à-vis mMSNs.

Earlier it was established that monosized protocells maintain long-term colloidal stability in PBS and complete cell culture media; however, we sought a more rigorous test for our platform under more dynamic conditions. Protein corona formation onto nanoparticle surfaces has been shown to occur immediately upon exposure to a live animal system (Lynch and Dawson, 2008), thus, protocell size stability after intravenous injection and circulation was examined because there apparently are no current reports that examine nanoparticle stability post injection. Fluorescent nanoparticle labeling provided useful qualitative analysis of stability within the CAM vasculature, which led to quantitative measure of protocell size after separation from blood samples extracted post-injection from both CAM and mouse models. Fluorescent protocells were detected in whole blood samples extracted from the CAM (FIG. 48A); we then separated protocells from whole blood by centrifugation and the measured hydrodynamic size. Remarkably, the average protocell size is nearly identical pre- and post-injection (FIG. 48B). In addition, we examined protocell size after circulation for multiple time points and found only a modest, time-dependent, average hydrodynamic diameter increase of 9% at 30 minutes and increasing to 23% at 240 minutes (FIG. 69). In vivo stability characteristics were further examined by intravenous tall vein injection of protocells into a BALB/c mouse. After 10 minutes of protocell circulation, blood was extracted from the mouse, fluorescent protocells imaged in whole blood (FIG. 48C), separated protocells using centrifugation, and found protocells maintain size stability in a mouse model (FIG. 48D). Thus, qualitative and quantitative confirmation of both ex ovo and in vivo protocell stability were demonstrated in unique and separate model systems. While these data indicate that the protocell platform possesses a distinctive ability to circulate and avoid aggregation in a complex living system for a short period of time, more comprehensive analysis of protocell circulation and biodistribution in animal models of disease may provide for a more complete pre-clinical understanding of in vivo protocell performance.

### Protocell Targeting Specificity In Vitro and Ex Ovo

Once the biological compatibility and in vivo stability of the monosized protocell platform was verified, receptor specific targeting was examined both in vitro and ex ovo. As a model system we chose leukemia cell lines engineered to express epidermal growth factor receptor (EGFR) and compared them to the parental EGFR-negative cell line so as to have a matched control. Targeting was accomplished using the NeutrAvidin/biotin conjugation strategy to modify an amine functionalized SLB (prepared with mol ratio DSPC:chol:DSPE-PEG₂₀₀₀-NH₂=49:49:2—FIG. 55) with anti-EGFR monoclonal antibodies as depicted in FIG. 42.

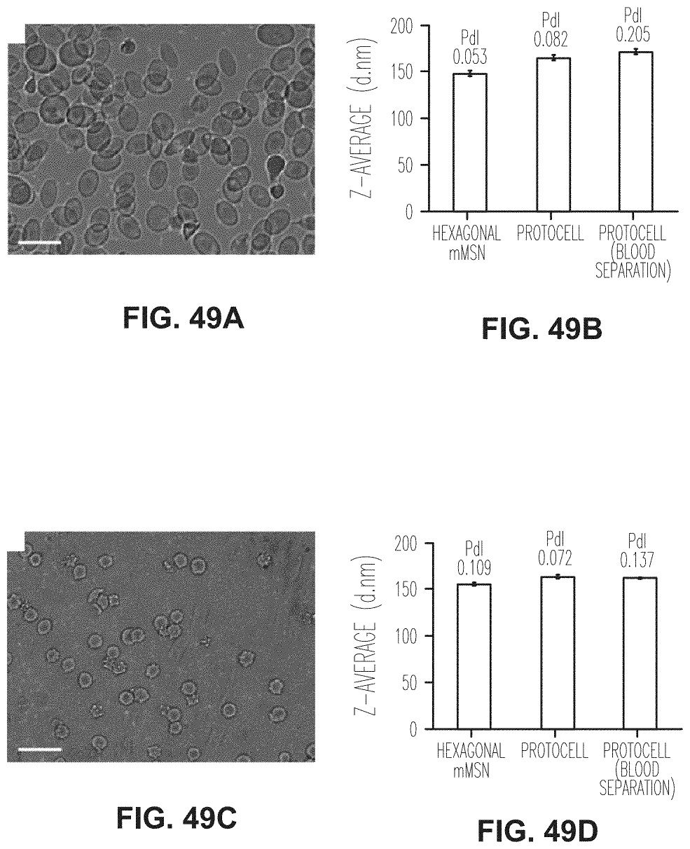

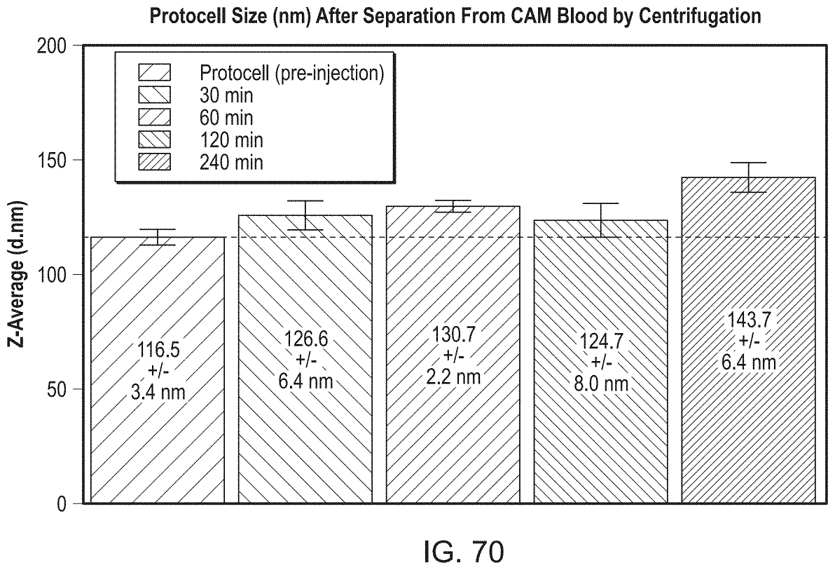

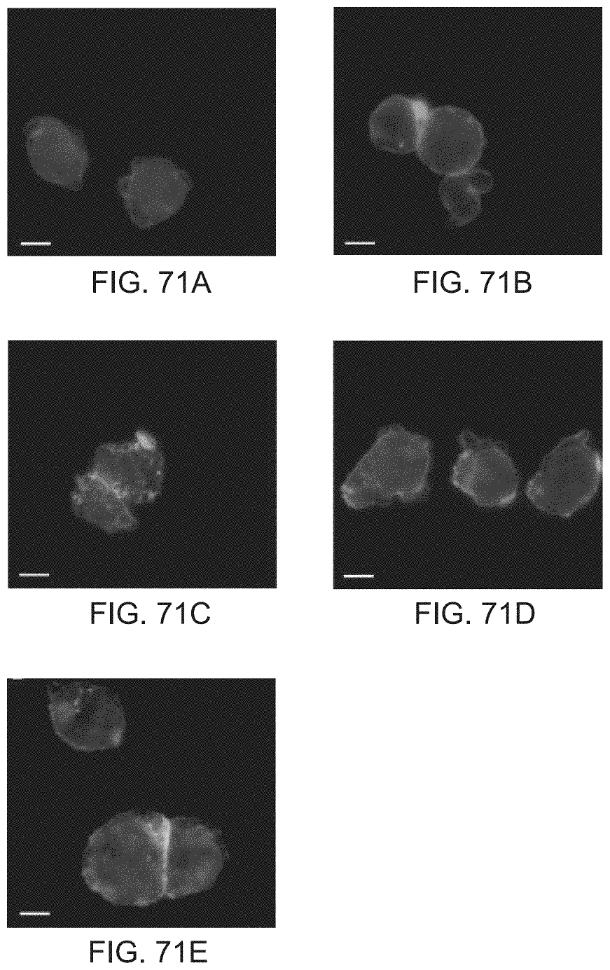

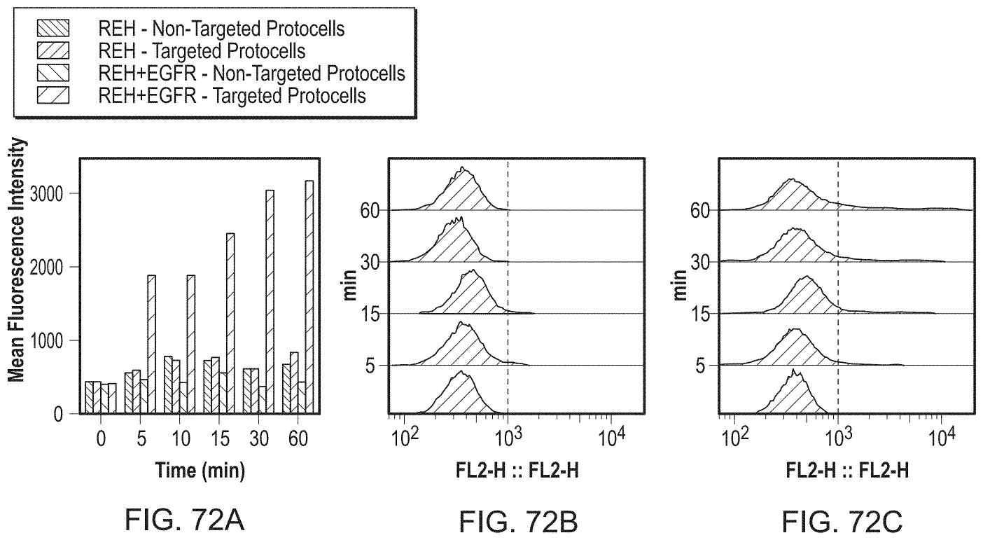

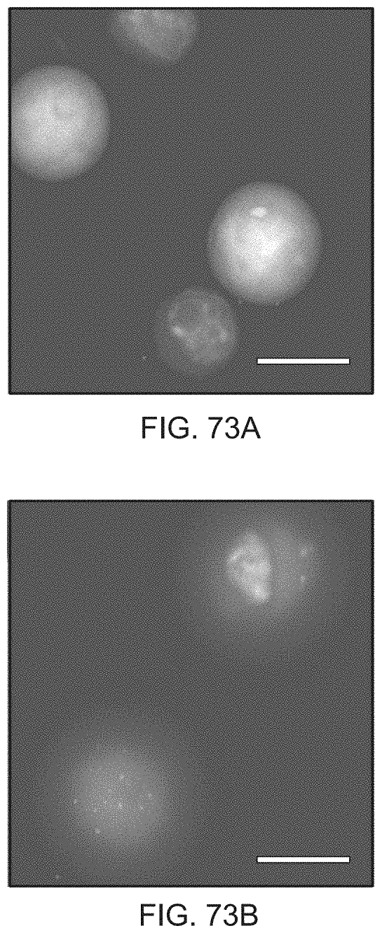

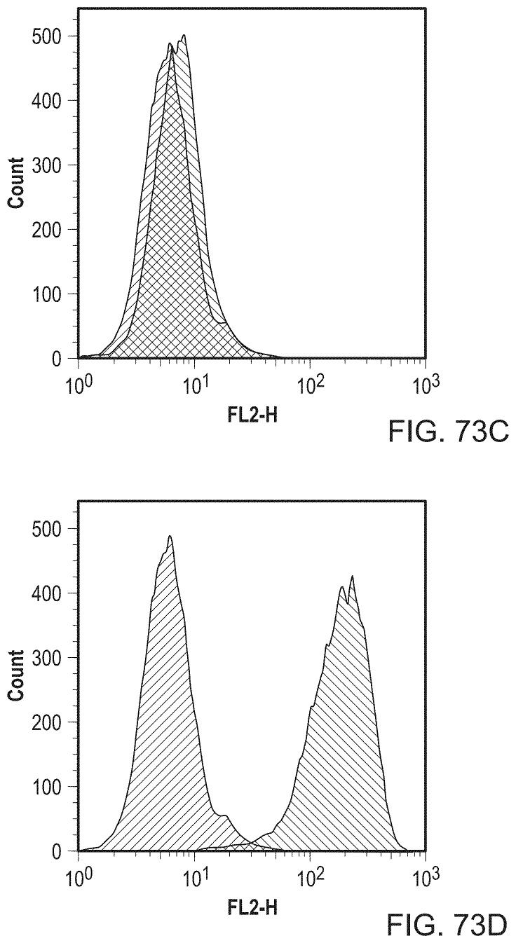

To examine targeting specificity, protocell interactions with both the human REH and also with a murine B precursor ALL line, Ba/F3 were compared. The performance of these parental, complimentary EGFR-negative control parental cell line controls, and the corresponding REH and Ba/F3 clones engineered to express ectopic human EGFR, designated REH+EGFR and Ba/F3+EGFR, respectively (Riese et al., 1995). To assess the kinetics of protocell binding, anti-EGFR antibody-labeled was incubated fluorescent protocells with REH and REH+EGFR cells for various time points in vitro. Significant binding was observed within 5 minutes and maximal binding at 30 minutes of incubation in complete media under normal cell culture conditions by both flow cytometry (FIG. 49A) and fluorescence microscopy (FIG. 71). As expected, from the absence of non-specific binding shown previously (FIGS. 68 and 67), protocell binding was not observed in the REH parent cell line (FIG. 49B), nor was non-targeted (anti-EGFR negative) protocell binding to either REH or REH+EGFR cell lines observed, as measured by flow cytometry (FIG. 72). To confirm that target specific binding is not cell line specific, anti-EGFR protocells were incubated with Ba/F3 and Ba/F3+EGFR cells for 60 minutes using previously described conditions for REH and REH+EGFR cells. Using fluorescence microscopy, we observed minimal non-specific binding of EGFR-targeted protocells to parental Ba/F3 cells; conversely we observed significant selective binding to Ba/F3+EGFR cells (FIGS. 73A and 73B). Flow cytometry analyses revealed the targeted protocells have a much greater binding affinity to Ba/F3+EGFR cells compared to the control Ba/F3 cell line in vitro (FIGS. 73C and 73D).

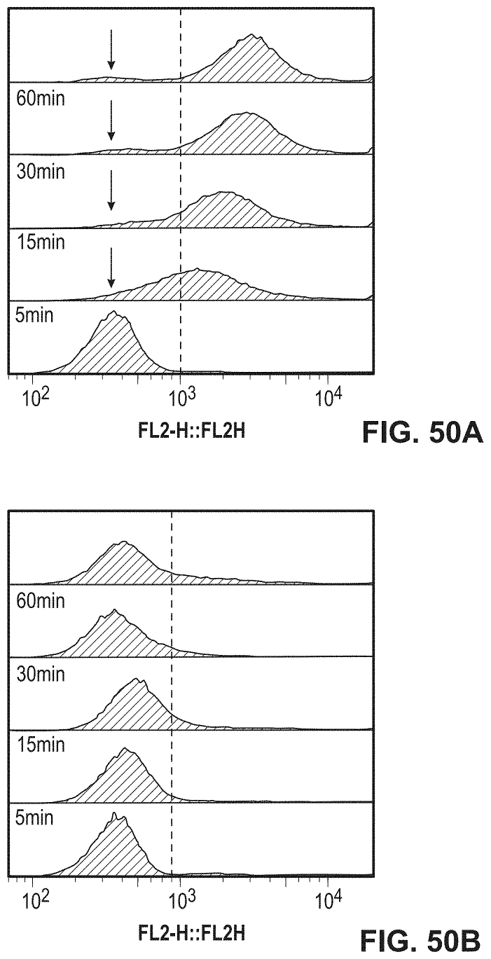

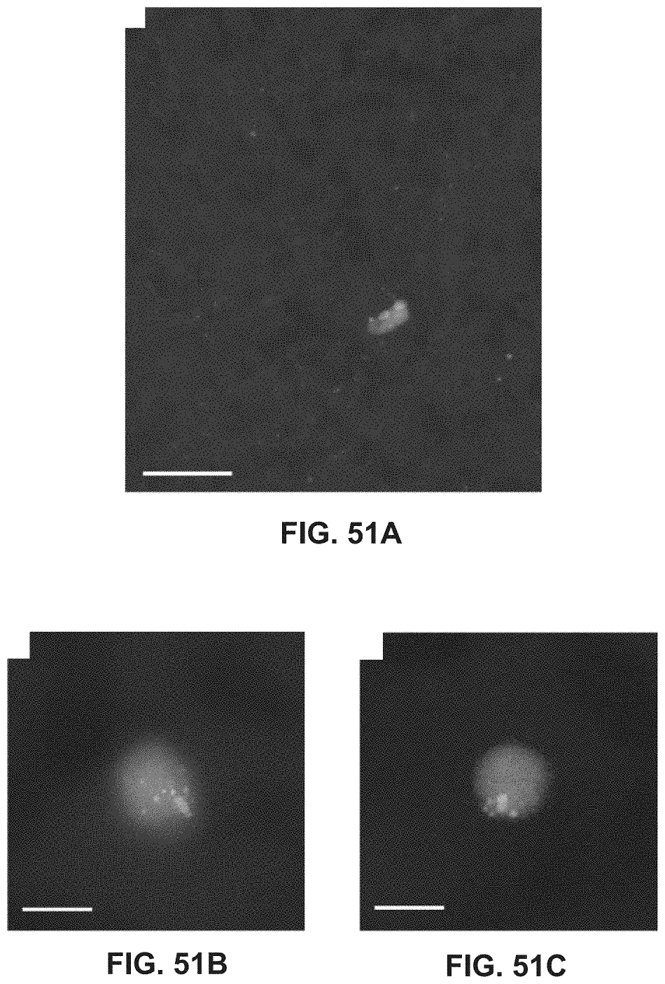

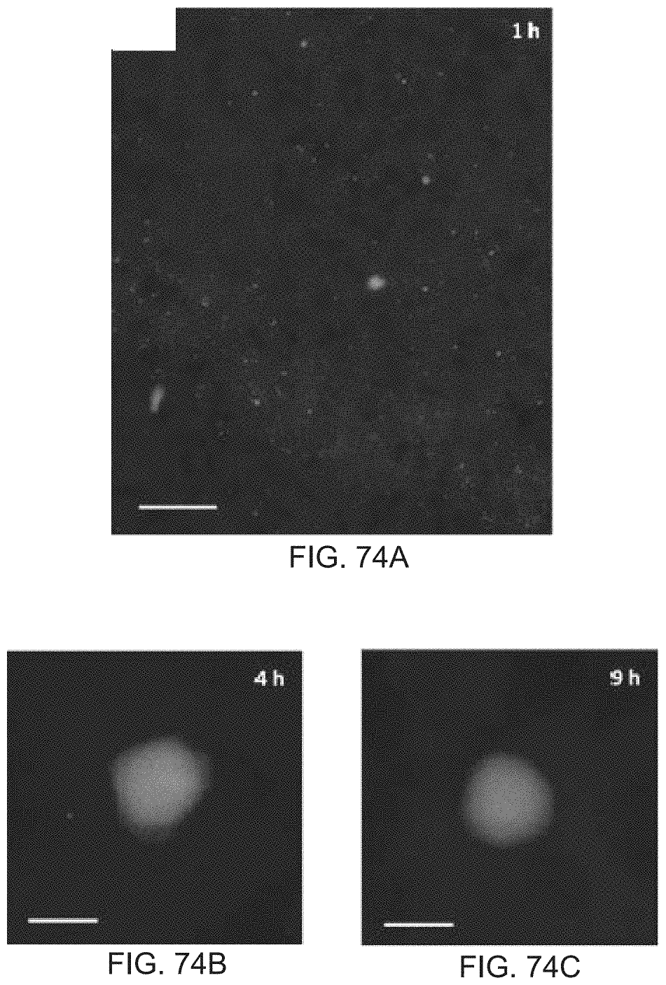

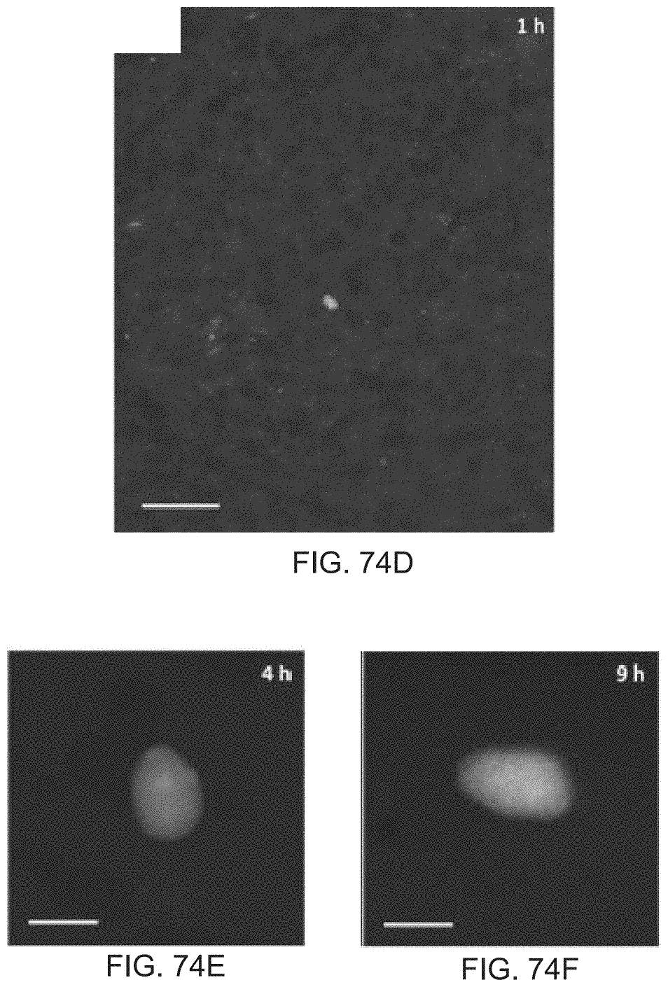

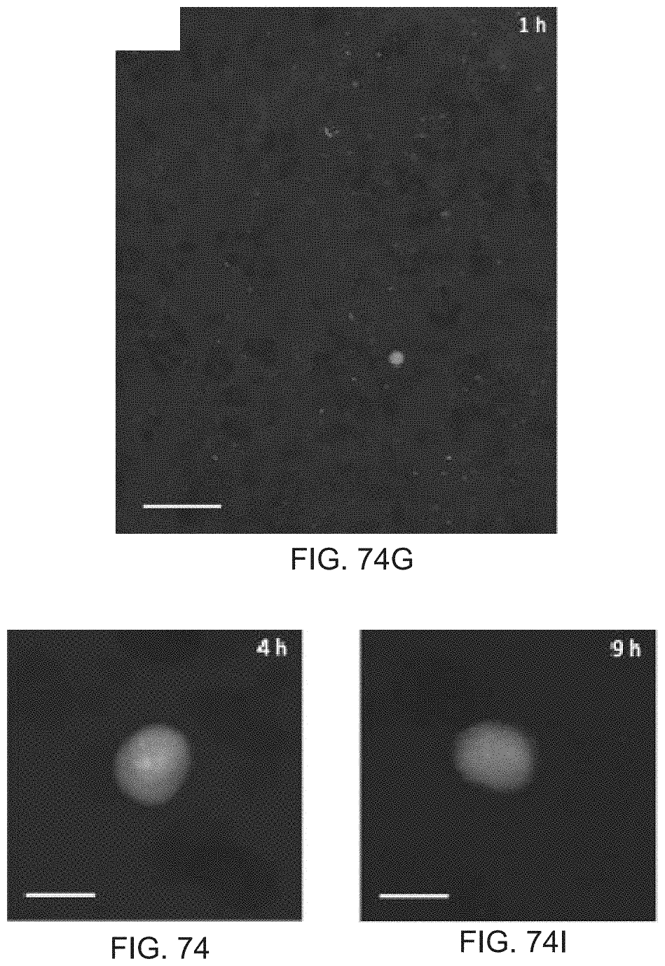

To provide an in vivo relevant assessment of targeted binding, the characteristics of the targeted protocell binding was evaluated using real-time intravital imaging in the CAM model. Green fluorescent labeled REH or REH+EFGR cells were injected into the CAM and the cells allowed to arrest in the capillary bed (about 30 minutes). Next, either anti-EGFR targeted or non-targeted red fluorescent protocells were injected into the CAM and imaged protocell flow and binding dynamics at 1, 4, and 9 hours time points. Protocells were observed flowing in the blood stream at 1 hour (FIG. 50A), as well as cell specific binding of the anti-EFGR protocells to the REH+EGFR cells. While flow had diminished at 4 and 9 hours time points, we still observed targeted protocell co-localization with the target cells (FIGS. 50B and 50C). Since it was observed a significant targeted protocell binding to REH+EGFR cells at 1 hour and our in vitro experiments showed binding within 5 minutes, we sought to capture targeted protocell binding within a vascularized system in real time; thus, intravital imaging in the CAM was performed immediately after protocell injection and several binding events on multiple cells observed (FIG. 51) within 5 to 10 minutes post protocell injection. To verify that protocell binding was indeed EGFR specific, anti-EGFR targeted protocells with REH cells and non-targeted protocells with REH and REH+EGFR cells lines were tested and similar flow patterns for the protocells were found at 1 hour time points; however, the protocells did not interact with the leukemia cells (FIG. 74) providing further support for the targeting methodology. As a final step, it was investigated whether protocell binding was influenced by the particular engineered cell line. Ba/F3+EGFR cells were injected ex ovo, followed by anti-EGFR protocell injection, and target cell specific binding observed at 10 minutes and 20 hours (FIG. 75). Based on these findings, biologically stable protocells with a high degree of specificity evaluated both in vitro and by intravital imaging in the CAM model to bind to individual target cells, have been engineered.

### Protocell Cargo Loading and Delivery to Targeted Cells

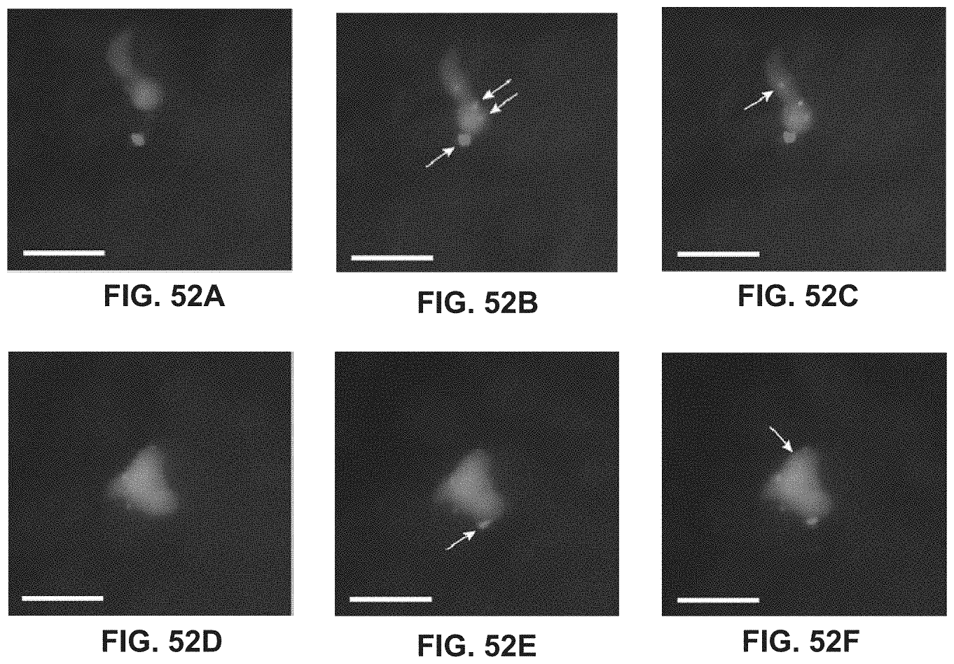

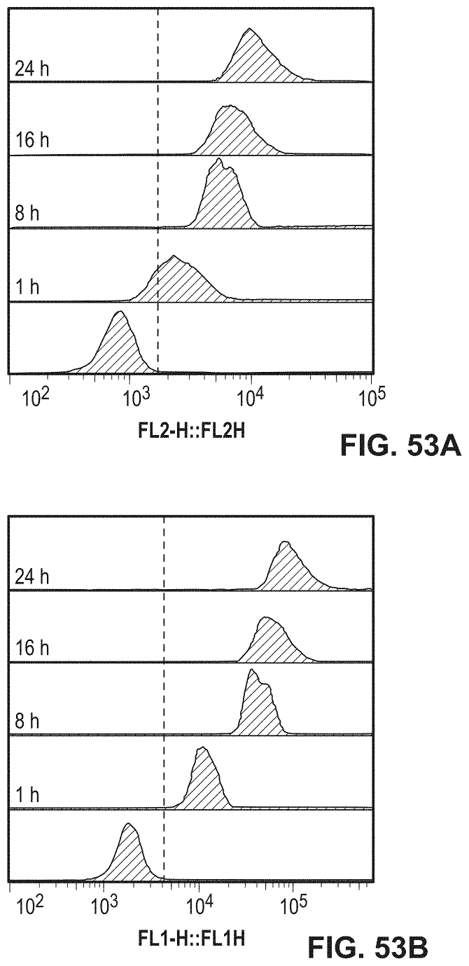

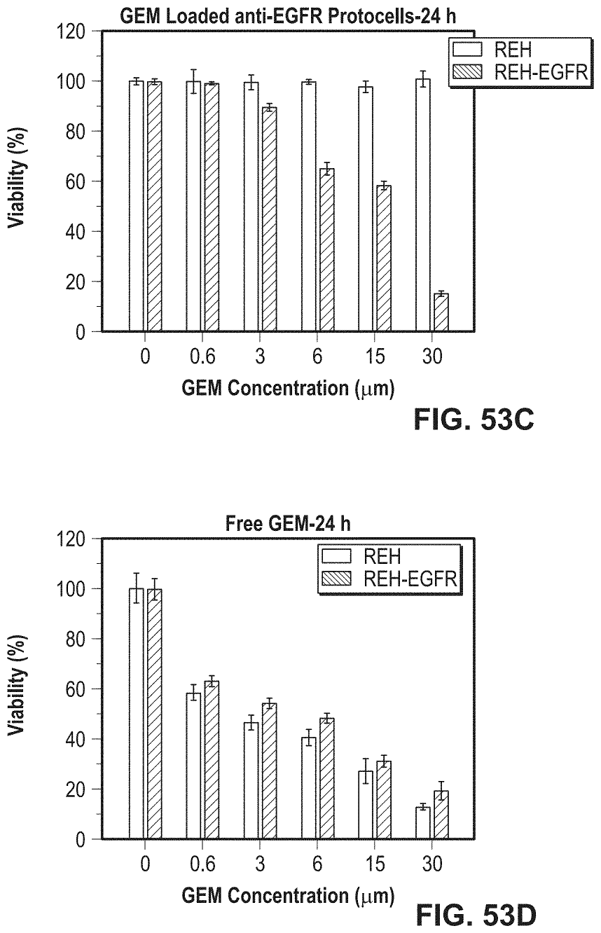

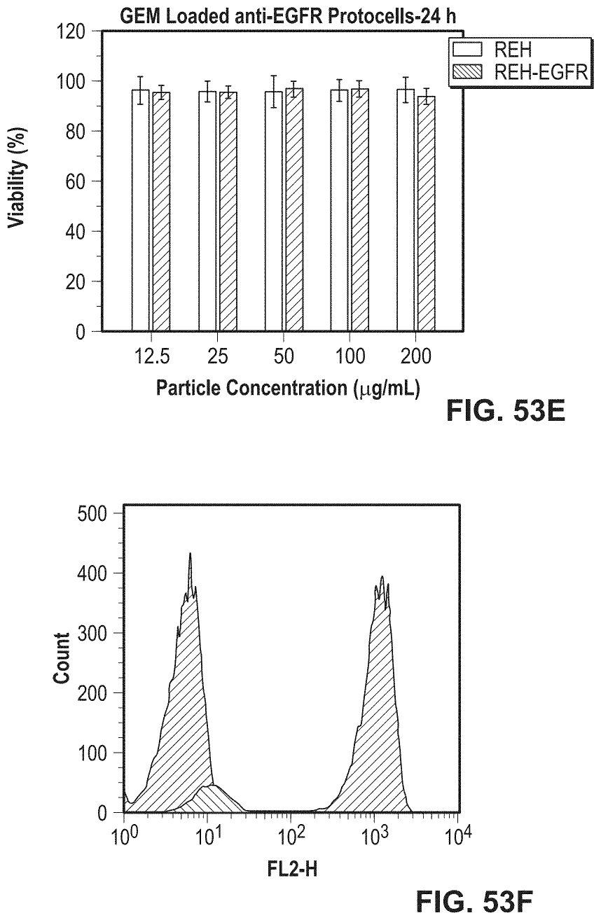

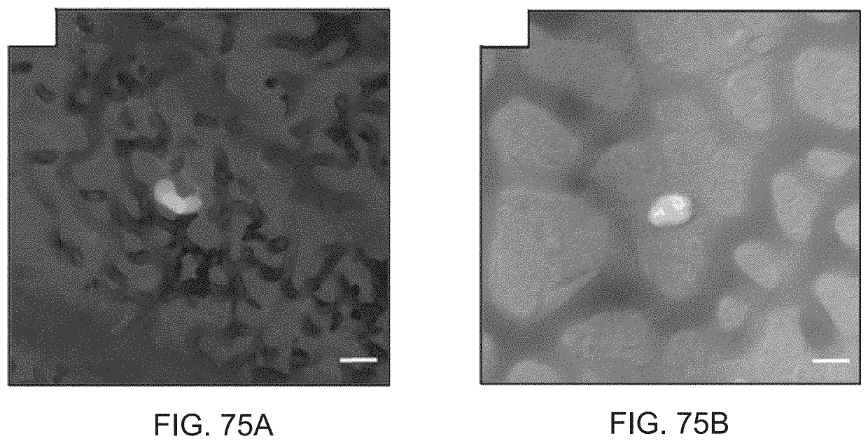

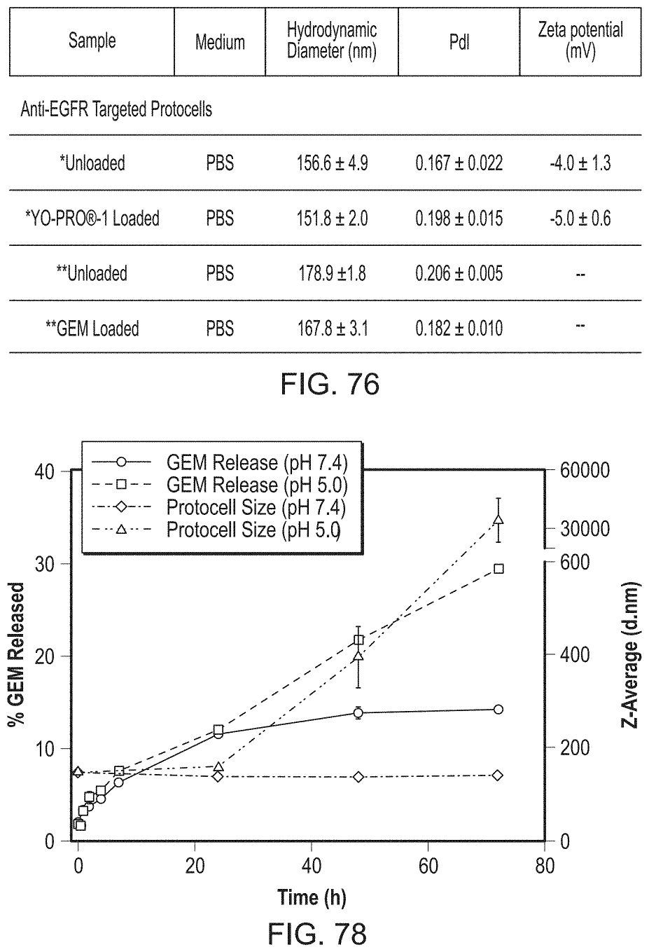

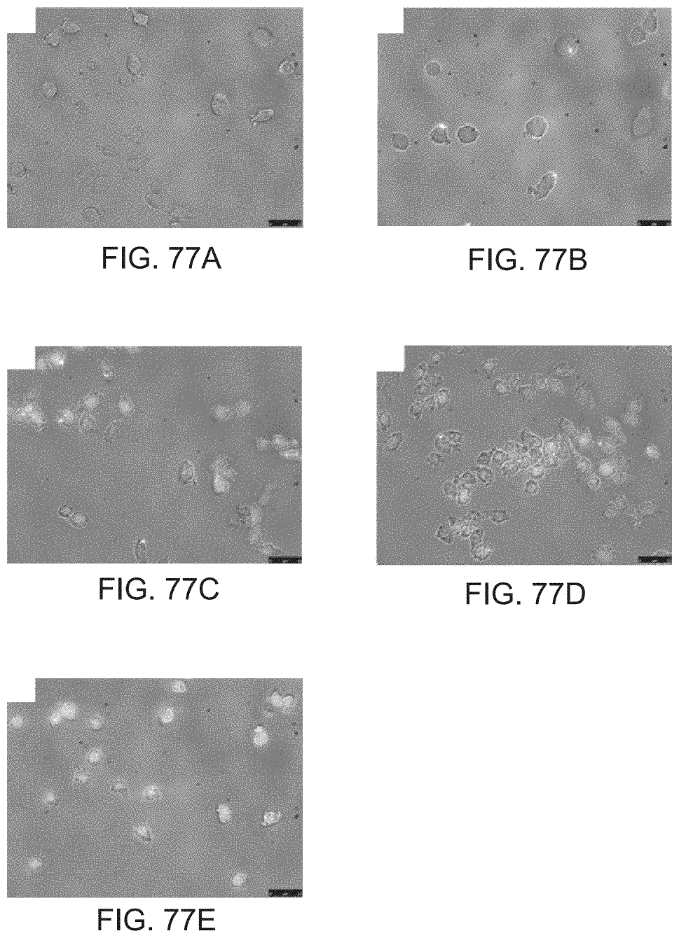

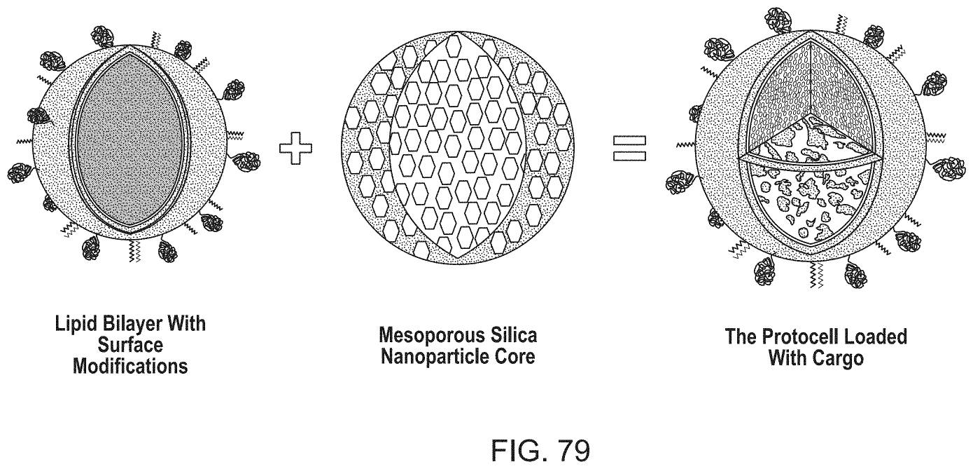

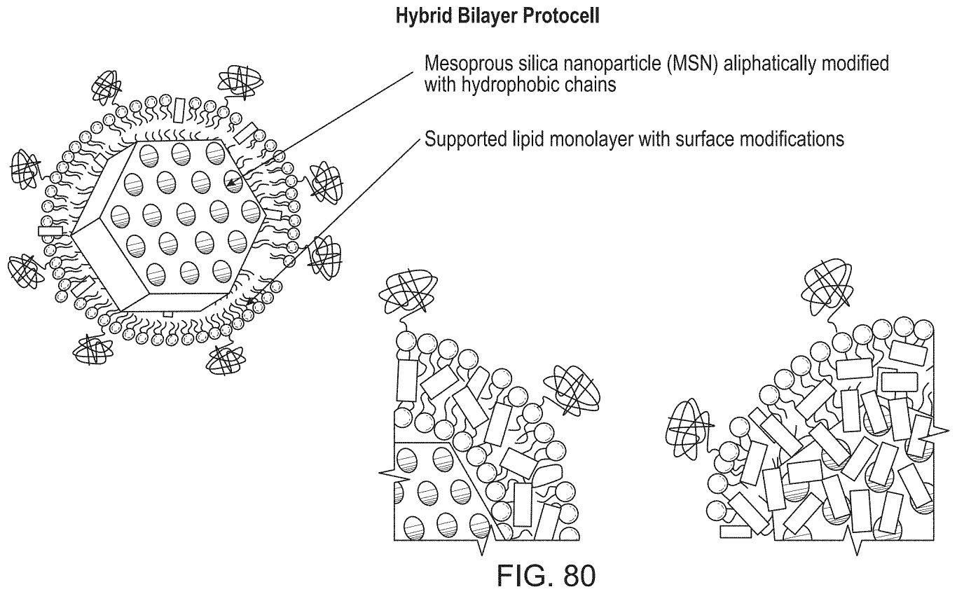

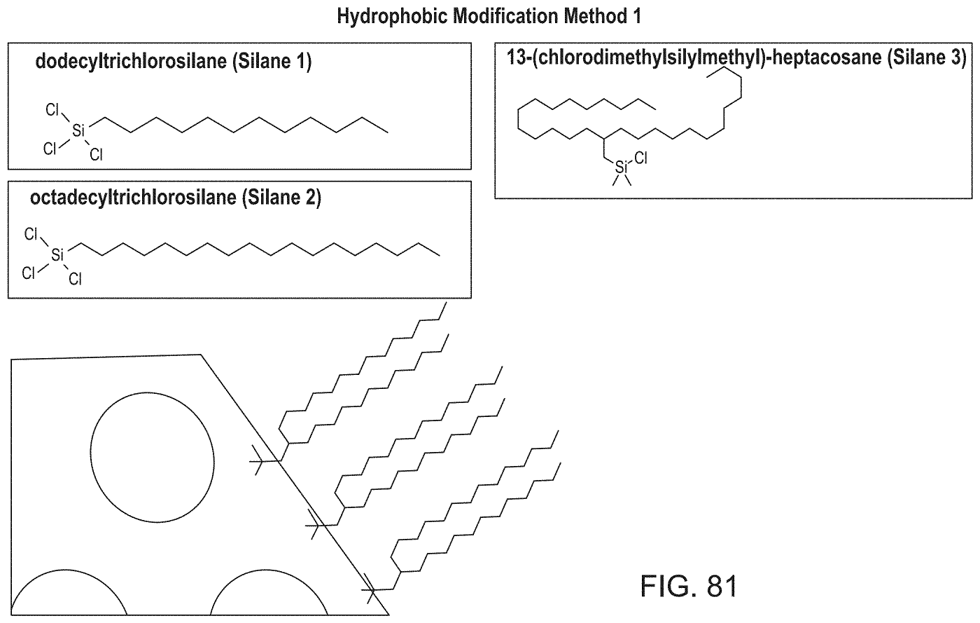

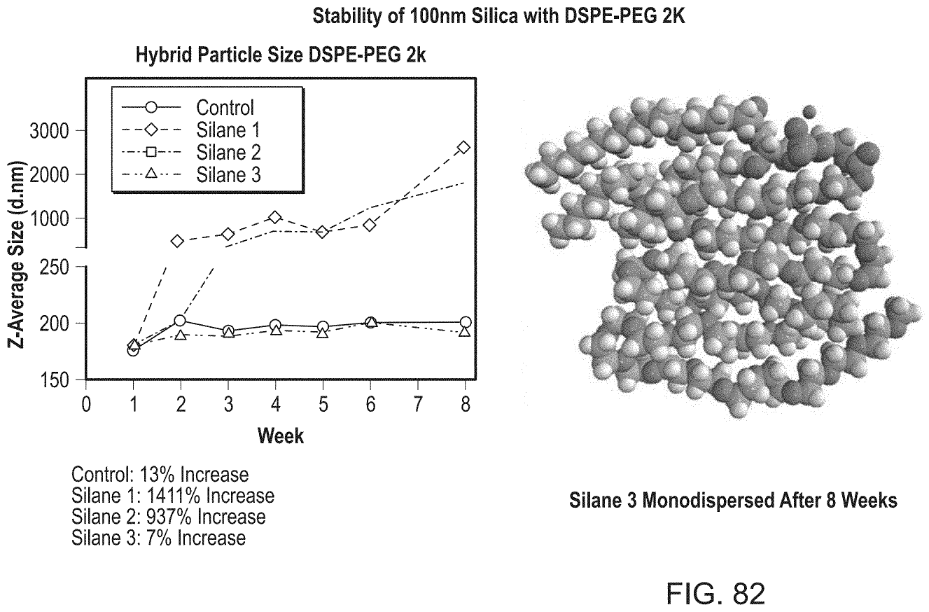

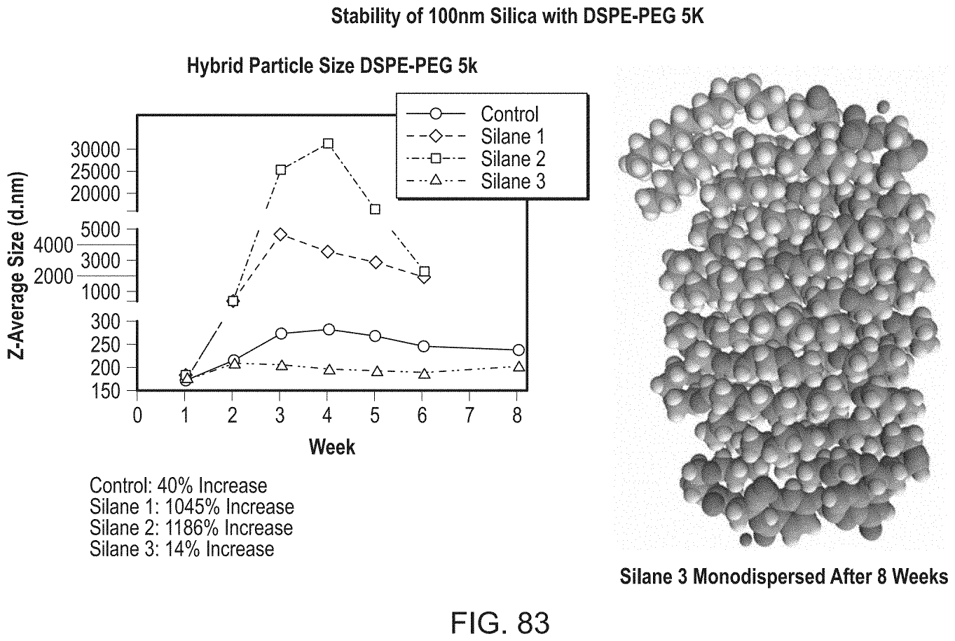

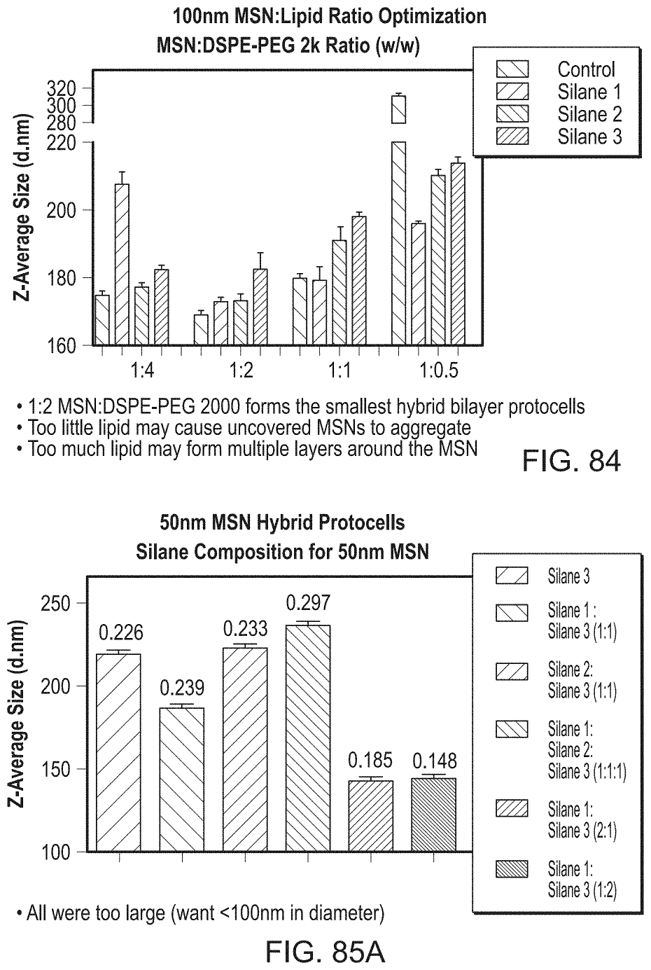

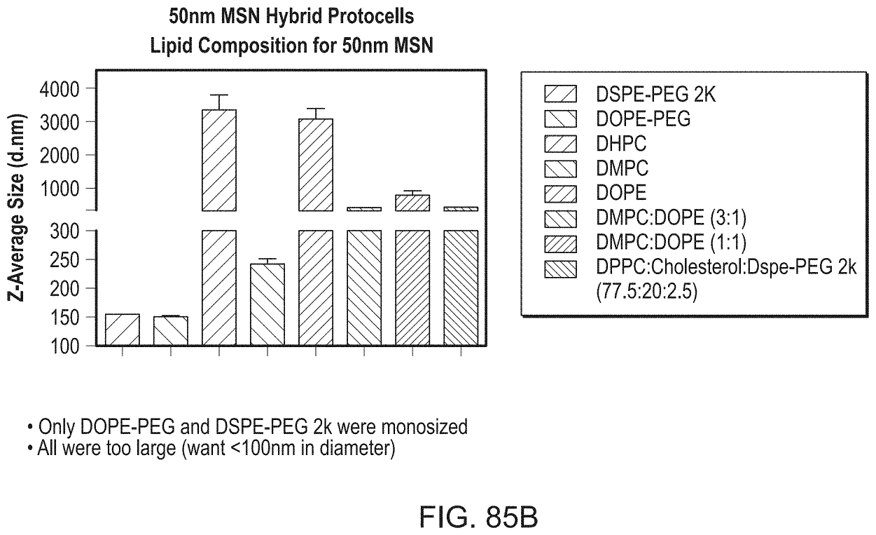

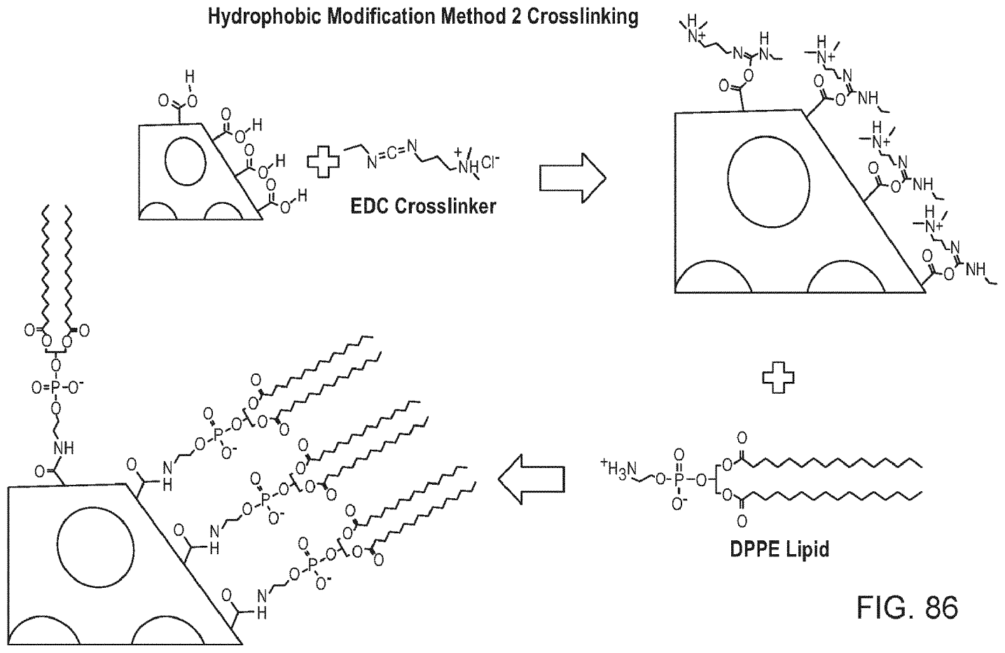

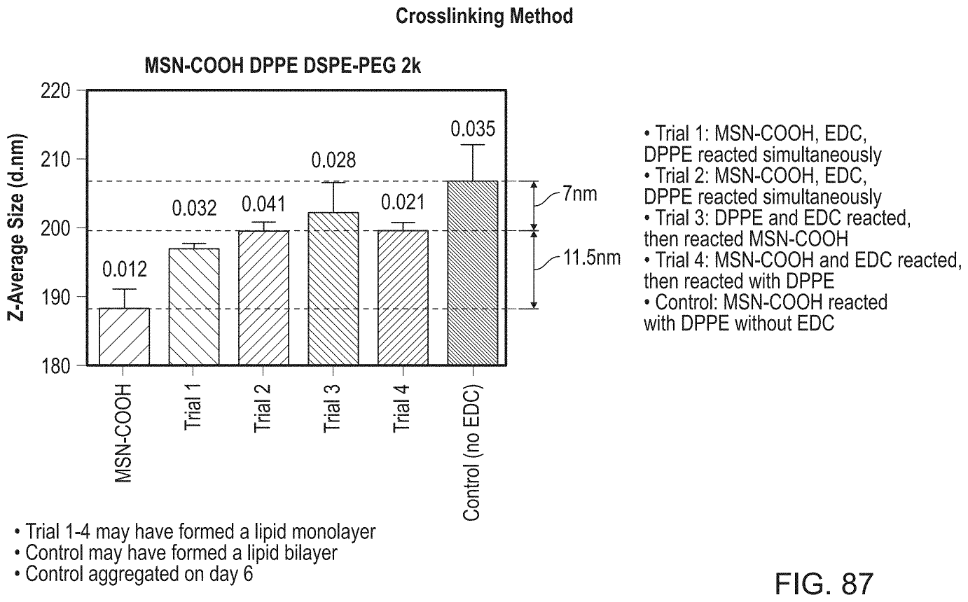

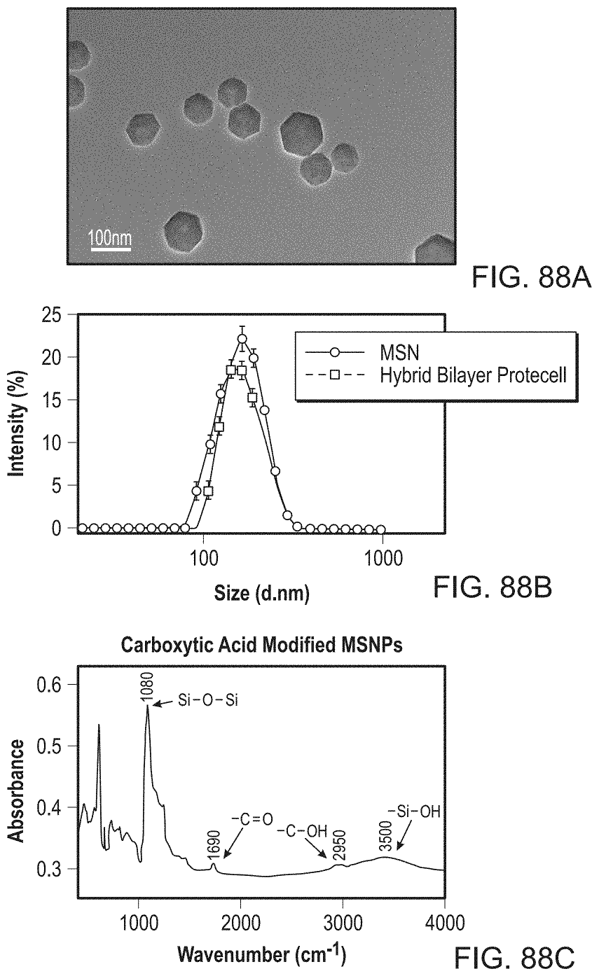

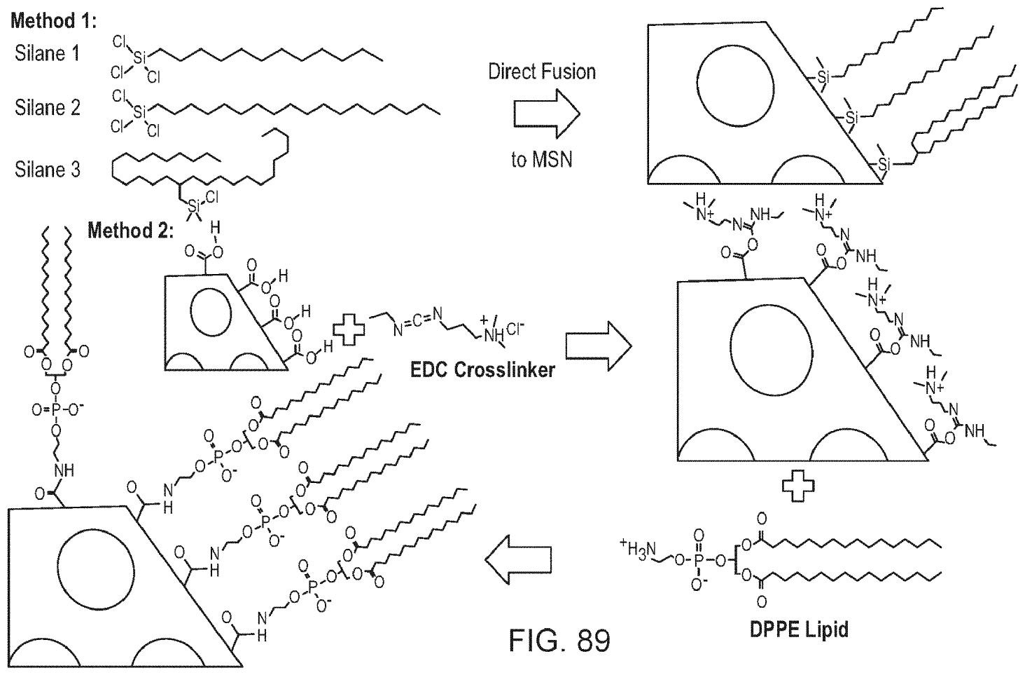

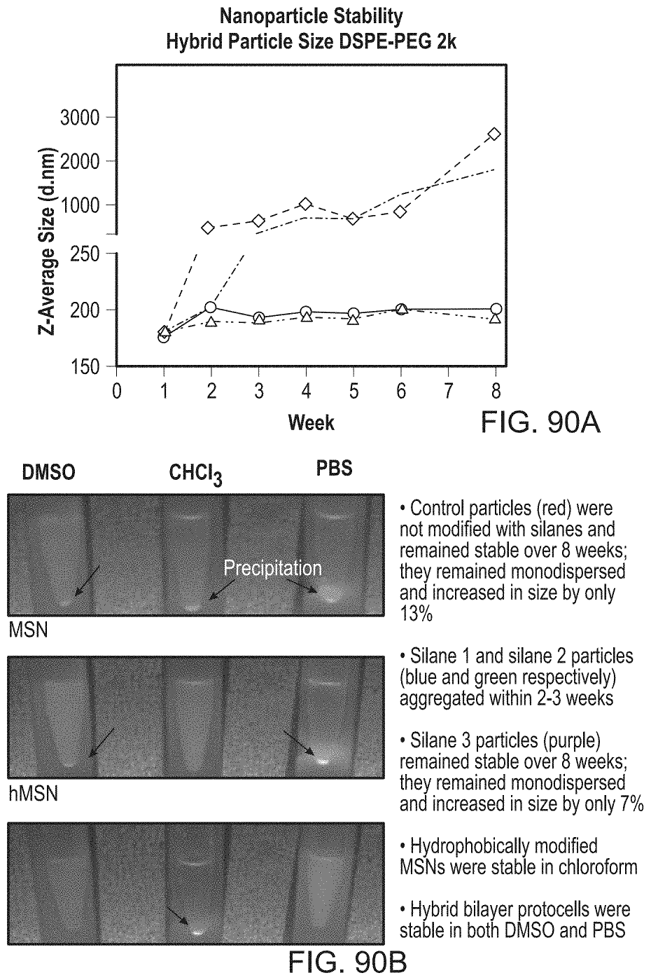

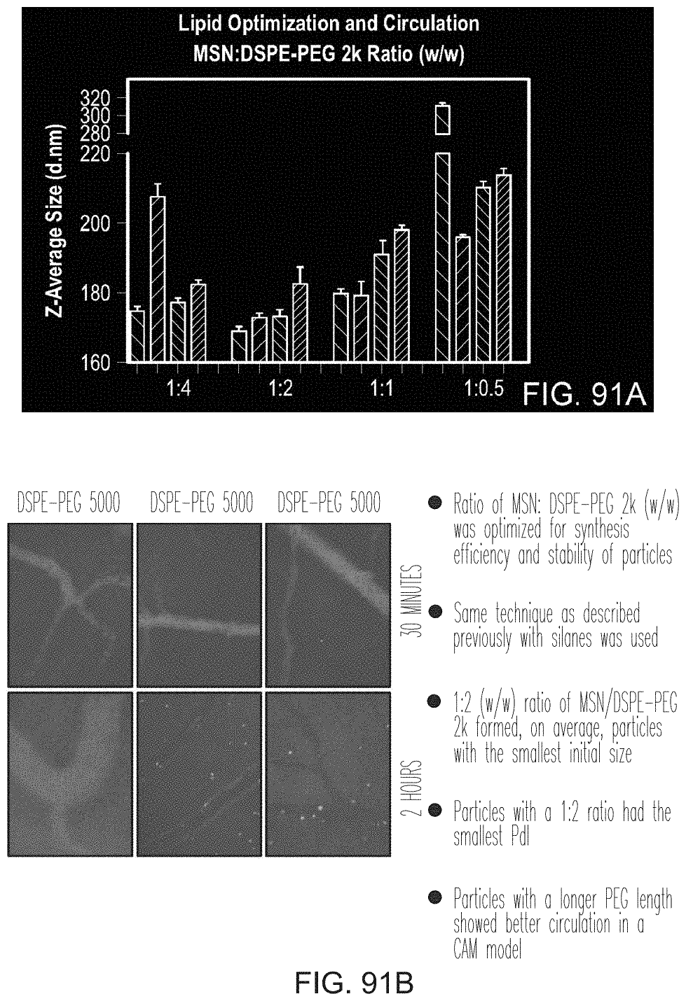

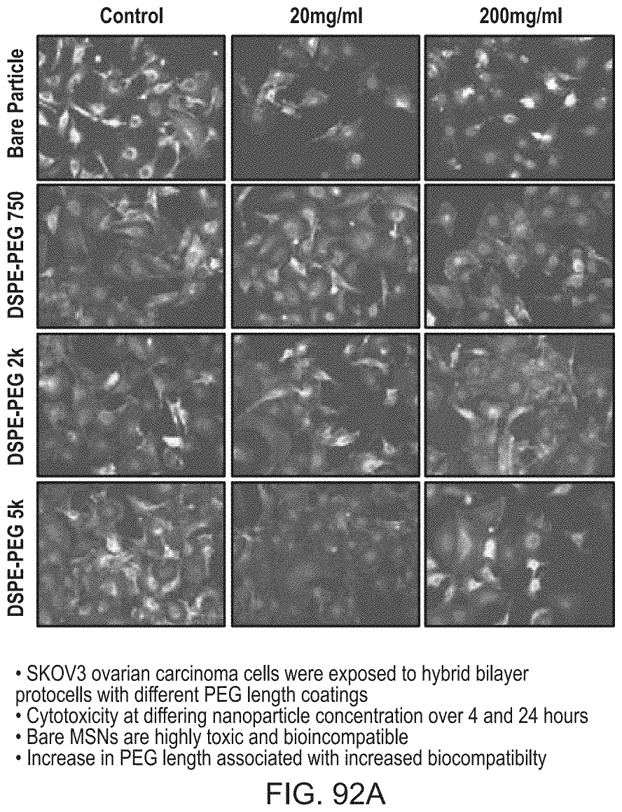

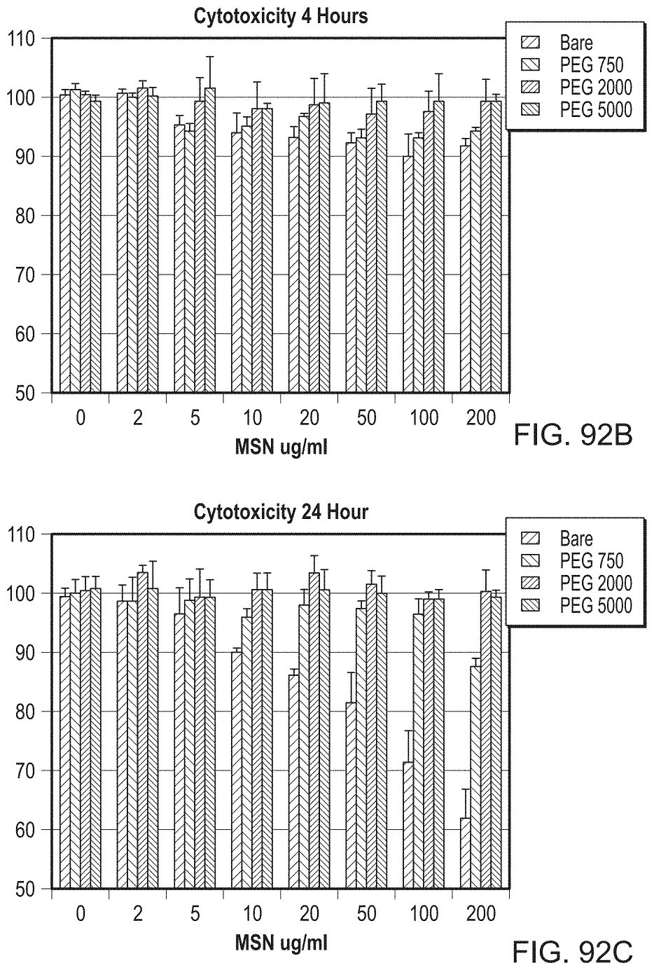

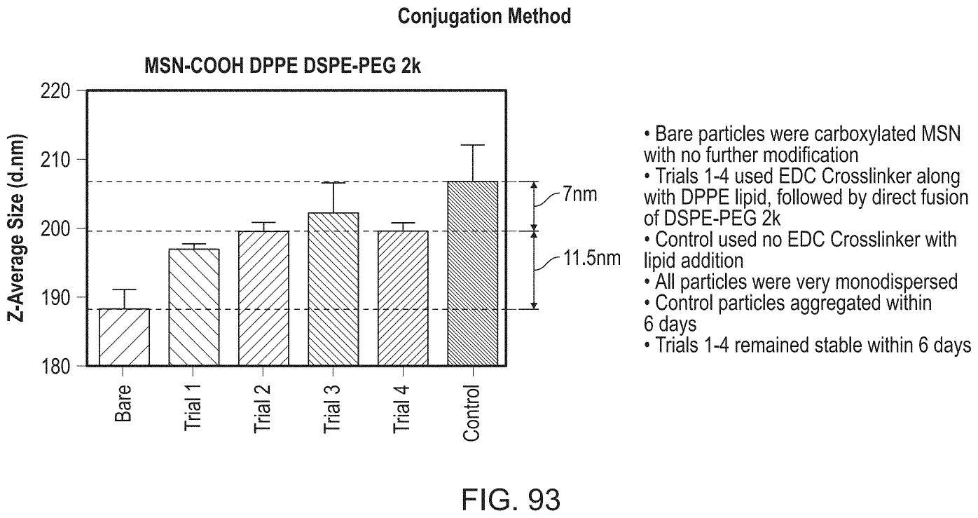

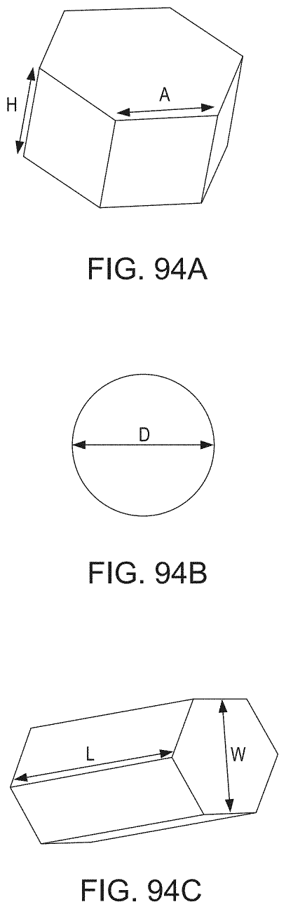

Next, the cargo loading and targeted delivery characteristics of monosized protocells were evaluated both in vitro and ex ovo. As a surrogate for a true drug, YO-PRO®-1, a green fluorescent membrane impermeable molecular cargo was selected. YO-PRO®-1 was added to red-fluorescent labelled mMSNs, fused liposomes, and conjugated anti-EGFR targeting components to the surface following the steps illustrated in FIG. 42. Anti-EGFR targeted protocells loaded with YO-PRO®-1 exhibited similar size and zeta potential characteristics to unloaded protocells assembled under identical conditions (FIG. 76). A 25% loading efficiency was calculated by disrupting the SLB of loaded protocells with a detergent and measuring the fluorescence intensity of YO-PRO®-1 extracted in DMSO (Details in the Experimental Section). Next, targeted protocell internalization was assayed as a measure of time using an acid wash technique to remove surface bound protocells at specific time points. Using flow cytometry and fluorescence microscopy, it was found that anti-EGFR targeted protocell binding and internalization occurs within 1 hour (FIGS. 53A and 77); however cargo release, as measured by intracellular green fluorescent cargo diffusion, occurred more slowly (FIGS. 52B, and 76).

To assess protocell targeted cell specific killing, in vitro, gemcitabine (GEM) was chosen as a model anti-cancer cytotoxic agent due to its low molecular weight, which allows it to access and adsorb to the high surface area mMSN mesostructure, as well as its relative membrane impermeability (Federico et al., 2012; de Sous Cavalcante et al., 2014), which allows the SLB to essentially seal the cargo in the protocells and to prevent off-target effects due to drug leakage. Moreover, GEM requires a nucleoside transporter to cross the cell membrane, and reduced expression of the nucleoside transporter is known to be associated with gemcitabine resistance (Federico et al., 2012; de Sous Cavalcante et al., 2014). Furthermore, the plasma half-life of GEM is only 8-17 minutes due to rapid conversion to an inactive form that is excreted by the kidneys (Federico et al., 2012); therefore, GEM requires frequent doses to overcome this clearance rate. Thus, encapsulation of GEM within a targeted protocell may overcome many of the challenges associated with conventional GEM-based therapy.

Cargo delivery was assessed using REH and REH+EGFR cells incubated with GEM loaded anti-EGFR protocells in vitro. To prepare GEM loaded, anti-EGFR targeted protocells, mMSNs were resuspended in a solution of GEM prepared in H₂O then assembled protocells by fusing GEM-soaked mMSNs with liposomes following the steps illustrated in FIG. 42. The supernatant from each step was collected and combined; the GEM content was determined by measuring the absorbance (265 nm) using a microplate reader. The described GEM loading strategy resulted in a calculated 15 wt. % GEM encapsulation. Cargo loading did not influence the final targeted protocell size (FIG. 77), a result consistent with GEM loading of the internal mesoporosity.

To examine the drug release profile under simulated lysosomal conditions, GEM loaded protocells were prepared in PBS, then the samples dialyzed in either PBS (pH 7.4) or 1 M citrate buffer (pH 5.0) for 72 hours at 37° C. The absorbance (265 nm) of supernatant collected at several time points was measured to determine the quantity of GEM released under these conditions. We observed a greater total drug release percentage at pH 5.0 (about 30%) compared to pH 7.4 (about 14%) after 72 hours (FIG. 78). A significant hydrodynamic size increase was observed at 48 hours in pH 5.0, correlating with the increase in drug release observed at the same time point, while protocells maintain size stability at pH 7.4 under the same experimental conditions (FIG. 78). These data suggest that drug release is increased at a lower pH primarily due to SLB destabilization as evidenced by aggregation. However, the influence of only a single variable (pH) was examined, while other conditions exist in the lysosomal pathway including degradative enzymes, for example phospholipase A2 (Schulze et al., 2009), which could affect drug release Therefore the functional release of GEM was examined as a measure of cell viability in vitro. To evaluate the target specific drug delivery, REH and REH+EGFR cells were incubated with increasing concentrations of anti-EGFR GEM-loaded protocells in complete media under normal culturing conditions. A distinct EGFR-target specific decrease in viability correlating to an increase in targeted protocell concentration was observed (FIG. 53C). Finally, the killing specificity of free-GEM, and observed decreased cell viability was observed with increasing GEM concentration in a non-specific manner (FIG. 53D). To verify that the cargo is responsible for the killing as opposed to the protocell itself, anti-EGFR targeted protocells were incubated with REH and REH+EGFR cells with increasing concentrations and observed no loss in viability for up to 200 μg/mL of protocells (FIG. 53E). Worth mentioning, a subset of REH+EGFR engineered cells appear to lose EGFR expression overtime (FIG. 52F and FIG. 49A—red arrow); therefore, the remaining viable cells in the maximum dose tested (50 μg protocells/30 μM GEM) (FIG. 53C) are likely to be EGFR negative.

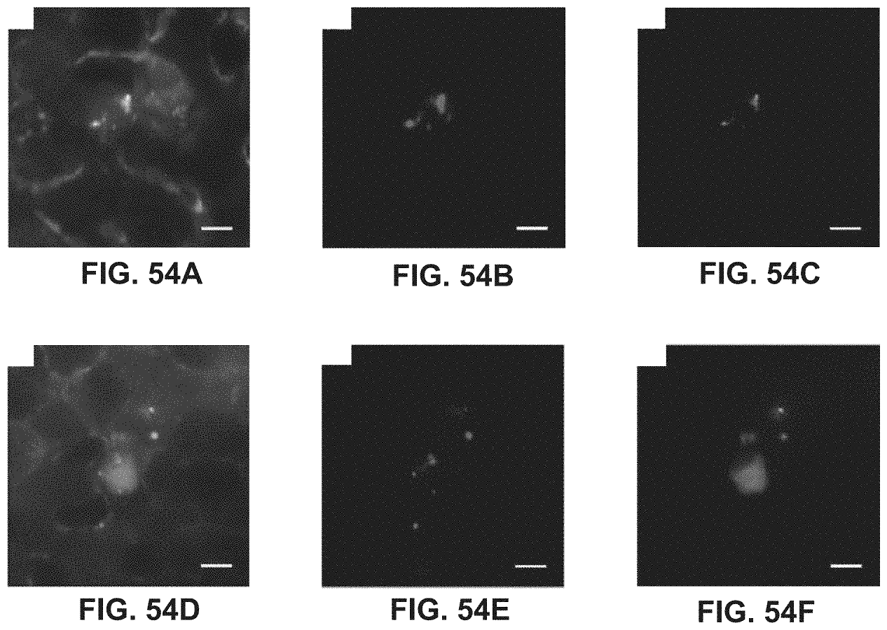

To test targeted binding and cargo delivery in a complex living system, the CAM was injected with fluorescent labelled REH+EGFR cells followed after 30 minutes by injection of YO-PRO®-1 loaded anti-EFGR protocells, prior to intravital imaging a lectin vascular stain was injected to provide contrast in the blood vessels. Intravital fluorescent imaging of the steps of binding, internalization, and cargo release was performed at 4 and 18 hours post ex ovo injection based on in vitro experiments (FIG. 77) that showed binding in as little as five minutes (FIG. 53A) but YO-PRO®-1 delivery and release to the cytosol to occur between 1 and 8 hours (FIG. 53B). FIG. 54A shows target specific binding to an individual REH+EFGR cell trapped within the CAM vasculature 4 hours post injection. There is no evidence of cargo release. FIG. 54B shows targeted binding to an individual REH+EFGR cell 16 hours post injection, where YO-PRO®-1 is dispersed throughout the cell similar to the in vitro results (FIG. 77). To better illustrate the targeted protocell binding, internalization, and cargo release at 16 hours, 0.25 μm sections of a targeted cell were imaged and the images stacked.

Further targeted delivery studies in a murine leukemia model to test protocell co-localization and disease elimination must be evaluated. Thus, highly specific targeted drug delivery in vitro combined with surrogate drug delivery ex ovo provides compelling evidence for the single-cell targeting utility of the monosized protocell therapeutic delivery platform.

### CONCLUSIONS

Here, by systematically evaluating the influence of SA_{lipid}:SA_{silica} and ionic strength on vesicle fusion to MSNs, a robust processing protocol was established to prepare colloidally stable mMSNs supported lipid bi-layers aka protocells characterized by size uniformity (PdI<0.1) and long-term stability in biologically relevant media. The protocol developed (SA_{lipid}:SA_{silica}≈2:1 and ionic strength=40 mM) using prismatic Hexagonal mMSNs was shown to be transferable to MSNs of differing size, shape, and pore morphology. Only for mMSNs prepared with the largest pores (about 18 nm) did fusion not occur—presumably due to reduced van der Waals and electrostatic interactions and/or surface roughness arrested bi-layer spreading.

Having established a robust process to prepare monosized protocells, their long-term stability was evaluated in biologically relevant media in vitro, ex ovo, and in vivo models. It was found that zwitterionic SLBs prepared with or without PEG conferred excellent stability to the protocells compared to the parent mMSN. DSPC-based SLBs were shown to have longer-term stability than DOPC-based protocells in PBS at 37° C. However, DOPC-based protocell stability was restored by the removal of soluble oxygen. Furthermore protocells prepared with both unsaturated DOPC and saturated DSPC SLBs were stable for over 72 hours in FBS enriched media suggesting that preparation and storage in deoxygenated buffer or exposure to proteins prior to use would allow either formulation to be implemented in vivo depending on the desired characteristics of the specific application. While saturated SLBs, with demonstrated stability in standard PBS are easier to prepare and store, protocells prepared with unsaturated SLBs might be used for in vivo targeting, where the fluid bi-layer could support lateral diffusion of targeting ligands, enabling high avidity binding with low targeting ligand density, as previously reported in vitro (Ashley et al., 2011).

The behavior of DSPC-PEG-based protocells was assessed ex ovo in the CAM model whose diverging and converging vasculature recapitulates features of the liver and spleen and whose immune system is replete with professional phagocytic cells including Kupffer cells and sinusoidal macrophages. High-speed intravital imaging of protocells and target cells injected into the vasculature of the CAM model allowed direct observation of circulation, non-specific binding to the endothelium, uptake by white blood cells, and binding to target cells in a complex setting, containing blood proteins and a developing immune system. While in vitro assessment is standard practice and provides important information, we contend it lacks the complexity to accurately forecast in vivo outcomes. For example, by comparing monosized protocells with highly size polydisperse protocells, size monodispersity was demonstrated to be important for avoiding arrest in the capillary bed and uptake by immune cells. Monosized DSPC-PEG-based protocells, shown to be stable within complex CAM and in vivo mouse models, were conjugated with anti-EGFR antibodies while maintaining size monodispersity.

Flow cytometry combined with fluorescence microscopy showed a high degree of binding specificity of EGFR-targeted protocells to REH-EGFR and Ba/F3-EGFR ALL cells compared to EGFR negative parental control cells. Using intravital imaging in the CAM, selective binding of EGFR-targeted protocells to individual leukemic cells followed by delivery of a membrane impermeant cargo, while non-specific binding to endothelial cells and uptake by immune cells were directly observed. Overall, it was demonstrated that zwitterionic monosized protocells prepared by vesicle fusion on mMSN cores have long-term stability in complex biological media as judged by intravital imaging in the experimentally accessible CAM model. Colloidal stability is crucial to achieving targeting to individual (leukemic) distributed cells, where the EPR effect is inoperative.

Finally, the highly specific therapeutic efficacy of targeted protocells was demonstrated by delivery of the anti-cancer cytotoxic cargo gemcitabine to an engineered EGFR-expressing leukemic cell line, while sparing EGFR-negative parental cells from off-target effects. Further, the biocompatibility of the protocell platform was confirmed. Thus, monosized protocell design has great potential for the active targeting, detection and treatment of highly disseminated metastatic cells including difficult to target circulating leukemia cells as well as combined passive and active tumor targeting employing the EPR effect.

Monosized protocells prepared from mMSNPs provide an advantageous approach to treatment of a large variety of disease states and conditions, especially where targeted drug delivery provides an advantageous approach to such treatment by increasing the therapeutic effect and/or reducing side effects associated with the use of prior art formulations and methods. In addition, in certain embodiments, protocells exhibit enhanced colloidal and/or storage stability in solution.

### Example 4

To increase the loading of hydrophobic cargo hydrophobic aliphatic chains were incorporated on the surface of a MSN to enable direct fusion of lipid moieties to its surface. The resulting construct retains many features of the original protocell, while simplifying the synthetic procedure and increasing loading space for hydrophobic cargo. Herein, the synthesis, circulation, and biodistribution of the “hybrid bilayer protocell” constructs are described. Specifically, the effect of surface coating on circulation and retention of nanoparticle constructs in the avian embryo chorioallantoic membrane (CAM) will be observed via direct, real-time fluorescent imaging.

The present example is directed to hybrid bilayer protocells which comprise a mesoporous silica nanoparticle which has been modified on its surface with a silica hydrocarbon, the nanoparticle to be coated with a phospholipid monolayer. Optionally the mesoporous silica nanoparticle is further modified to contain a carboxylic acid group to allow derivatization of the surface nanoparticle. The hybrid bilayer protocells pursuant to the present embodiment are hydrophobic in chemical character, both within the nanoparticle and at the surface of the nanoparticle which has been modified with a silica hydrocarbon. These hybrid protocells are particularly useful to accommodate lipophilic cargo, especially lipophilic drugs at high levels of loading which cannot be readily achieved using mesoporous silica nanoparticles coated with a lipid bilayer (protocells). These hybrid protocells can be used to deliver hydrophobic drugs, diagnostic agents and other cargo at high concentrations of cargo, thus facilitating therapy and diagnosis using lipophilic cargo.

### Carboxylic Acid Modification

MSNP synthesis was according to Lin et al. 2010, and Lin et al., 2011. Prior to hydrothermal treatment, 3-(triethoxysilyl)propylsuccinic anhydride (2% molar ratio to TEOS) was added and stir for 1 hour. The rest of the purification was as described.

To make large pore spherical COOH modified MSNPs, the synthesis procedure of Wang et al., 2012 and Shen et al., 2014, after 12 hour synthesis, the organic phase was removed and replaced with cyclohexane+3-(triethoxysilyl)propylsuccinic anhydride (e.g., 1% molar ratio to TEOS) and stir for 1 hour. Then hydrothermal treatment step was added for 24 hours at 70° C. Purification process was the same as described by Yu-Shen's papers.

For the COOH-silane to TEOS ratios. 0.5% to 15% molar ratio may be used.

### Hydrophobic Silane Modification

Prior to hydrothermal treatment, the MSNPs were transferred to ethanol:chloroform (1:1) and 1.3 (chlorodimethylsilyl-methyl) heptacosane (7.5% molar ratio to TEOS) added. After 12 hours, particles were purified. This process can also be done post-purification. Final product is stored in ethanol:chloroform (1:1). The same method for other hydrophobic silanes was used. This method also works for the large pore spherical MSNPs.

### Hybrid Bilayer Protocell Assembly

Hydrophobic silane modified MSNPs were mixed with DSPE-PEG-2K in organic solvent, was dried into a film using rotary evaporation, hydrated in PBS and then washed several times by centrifugation.

Carboxylic acid modified MSNPs were incubated with EDC (1-ethyl-3-(3-dimethylaminopropyl)carbodiimide hydrochloride) crosslinker in H₂O for 0-2 hours at ambient temperature. DOPE or DMPE or DPPE or DSPE (or any lipid with a primary amine modified headgroup) http://www.avantilipids.com/index.php?option=com_content&view=article&id=125&Itemid=133) was dried into a film using rotary evaporation. EDC crosslinked COOH-MSNPs (in H₂O) were added to lipid film under sonication. EDC crosslinks the COOH on the MSNP surface to the amine on the lipid headgroup to form a covalent linkage. These crosslinked (now hydrophobic) MSNPs were transferred to ethanol:chloroform (1:1) solvent, centrifuged, then transferred to pure chloroform and washed twice in chloroform (to remove any unbound lipid). Then DSPE-PEG-2k (or other PEGylated lipids of different PEG lengths and hydrophobic tall lengths including saturated and unsaturated tall groups) was mixed with lipid tethered MSNPs in chloroform and dried together into a film using rotary evaporation. The film was then hydrated in PBS and washed several times by centrifugation.

Phospholipids that can be used to form the outer portion of the protocell include all of the PEGylated lipids in the following link—http://www.avantilipids.com/index.ohp?option=com_content&view=article&id=143&Itemid=151, as well as those PEG phospholipids described above.

Also functionalized PEG lipids can be used to conjugate targeting ligands or other components of the cell to be conjugated to the Lipid surface. http://www.avantilipids.com/index.php?option=com_content&view=article&id=145&Itemid=15. For example, it s possible to mix 0.5-7.5, e.g., about 2-5% mol of functionalized PEG lipids to the 95-98% mol standard PEG lipids.

Any phosphatidylcholine lipid may make up the rest of the hybrid bilayer composition—see the enclosed or as otherwise described herein. See http://www.avantilipids.com/index.php?option=com_content&view=article&id=123&Itemid=131

A simplified approach towards lipid incorporation on MSN was shown via step-wise covalent modification of MSN cores with aliphatic moieties, followed by subsequent self-assembly of free lipid molecules on its surface through long-range hydrophobic interactions. The formation of hydrophobically modified MSNs was confirmed because they were stable in chloroform, a hydrophobic solvent. The resulting construct, termed “hybrid bilayer protocell”, formed using hydrophobic silane 3 remains stable in phosphate buffered saline over an 8 week time span showing that lipid fusion was successful. In addition, particles formed using EDC Crosslinker on carboxylated MSNs were very stable, more so than any particles formed without the crosslinker. The hybrid bilayer protocell retains the ability to circulate within CAM models and prove to be biocompatible. The process for forming these particles present a more efficient and simplified approach toward lipid fusion upon mesoporous silica cores.

### REFERENCES

Adamson, Cancer J. Clin., 5:212 (2015).
Akbarzadeh et al., Nanoscale Res. Lett., 8:102 (2013).
Allen and Cullis, Science, 33:1818 (2004).
Asefa and Tao, Chem. Res. Toxicol., 2:2265 (2012).
Ashley et Example al. ACS Nano, 6:2174 (2012).
Ashley et al., Nat. Mater., 1.0:389 (2011).
Attwood et al., Int. J. Mol. Sci., 14:3514 (2013).
Bae, J. Controlled Release, 133:2 (2009).
Bartlett et al., Proc. Natl. Acad. Sci. U.S.A 2007, 104, 15549-15554.
Bayerl and Bloom, Biophys. J., 58:357 (1990).
Bayu et al., Microporous Mesoporous Mater., 120:447 (2009).
Bertrand et al. Adv. Drug Deliv. Rev., 66:2 (2014).
Blanco et al., Nat. Biotechnol., 33:941 (2015).
Breus et al., ACS Nano, 3:2573 (2009).
Brinker and Scherer, Sol-Gel Science: The Physics and Chemistry of Sol-Gel Processing. Academic press: 2013.
Buranda et al., Lanamuir, 19:1654 (2003).
Butler et al., Protocells: Modular Mesoporous Silica Nanoparticle-Supported Lipid Bi-layers for Drug Delivery. Small 2016.
Ça\check{g}das̨ et al., S. Liposomes as Potential Drug Carrier Systems for Drug Delivery. INTECH: 2014.
Carrol et al., Lanamuir. 25:13540 (2009).
Cauda et al., Nano Lett., 10:2484 (2010).
Chauhan et al., Nat. Nanotechnol., 7:383 (2012).
Chen et al., Chem. Mater., 2:4269 (2013).
Choi et al., Nat. Blotechnol., 25:1165 (2007).
Cremer and Boxer, J. Phys. Chem. B. 103:2554 (1999).
Crist et al., Integr. Biol., 5:66 (2013).
Davis et al., Nat Rev Drug Discov., 7:771 (2008).
de Sousa Cavalcante et al., Eur. J. Pharmacol., 741:8 (2014).
Dengler et al., J. Controlled Release, 168:209 (2013).
Deshpande et al., Nanomedicine (London, U. K.), 8:10.2217 (2013).
Dobrovolskaia and McNeil, J. Controlled Release. 172:458 (2013).
Draz at al., Theranostics, 4:872 (2014).
Egusquiaguirre et al., Clin. Transl. Oncol., 14:83 (2012).
Elsabahy and Wooley, Chem. Soc. Rev., 41:2545 (2012).
Epler et al., Adv. Healthcare Mater., 1:348 (2012).
Estephan et al., Langmuir, 26:16884 (2010).
Farokhzad and Langer, ACS Nano, 3:16. (2009).
Federico et al., Int. J. Nanomed., 7:5423 (2012).
Ferrari, Nat. Nanotechnol., 3:131 (2008).
Garcia-Manyes et al., Biophys. J., 89:1812 (2005).
Han et al., ACS Appl. Mater. Interfaces, 7:3342 (2015).
He et al., Small, 7:271 (2011).
Henriksen et al., ______ (2006).
Hrkach et al., Sci. Transl. Med., 4:128ra39 (2012).
Huang et al., ACS Nano, 10:648 (2016).
Huang et al., ACS Nano. 1:5390 (2011).
Ii et al., Mol. Cell. Biol., 15:5770 (1995).
Iwamoto, Biol. Pharm. Bull., 36:715 (2013).
Iyer et al., Adv. Drug Delivery Rev., 65:1784 (2013).
Johnson et al., Biophys. J., 59:289 (1991).
Johnson et al., Biophys. J., 83:3371 (2002).
Keller et al., Phys. Rev. Lett., 84:5443 (2000).
Kirk-Othmer, Encyclopedia of Chemical Technology, Vol. 20, 3rd Ed., J. Wiley, N.Y.
Kohli et al., J. Control. Release. 190:274 (2014).
Kresge et al., Nature, 359:710 (1992).
Krishnan and Rajasekaran, Clin. Pharmacol. Ther., 95:168 (2014).
LaCasse et al., Nucl. Acids Res., 23:1647 (1995).
Lammers et al., J. Controlled Release, 161:175 (2012).
Lee et al., Acc. Chem. Res., 44:893 (2011).
Lee et al., Chem. Soc. Rev., 41:2856 (2012).
Leong et al., Nat. Protoc., 5:1406 (2010).
Li et al., Chem. Soc. Rev., 41:2590 (2012).
Liao et al., Interfaces, 3:2607 (2011).
Lin and Haynes, J. Am. Chem. Soc., 132:4834 (2010).
Lin and Haynes. J. Am. Chem. Soc., 4834 (2010)
Lin et al., Chem. Commun., 47:532 (2011).
Lin et al., Chem. Mater., 17:4570 (2005).
Lin et al., J. Am. Chem. Soc., 133:20444 (2011).
Lin et al., J. Phvs. Chem. Lett., 3:384 (2012).
Lis et al., Physical Chemistry Chemical Physics, 13:17555 (2011).
Liu et al., ACS Nano, 10:2702 (2016).
Liu et al., Chem. Comm., _:5100 (2009).
Liu et al., J. Am. Chem. Soc., 131:1354 (2009a).
Liu et al., J. Am. Chem. Soc., 131:7567 (2009b).
Lu et al., Nature. 398:223 (1999).
Lynch et al. Nano Today, 3:40 (2008).
Mackowiak et al., Nano Lett., 13:2576 (2013).
Markman et al., Adv. Drug Delivery Rev., 65:1866 (2013).
Marsh, CRC Press, (2013).
Meng et al., ACS Nano, 5:4434 (2011).
Meng et al., ACS Nano, 9:3540 (2015).
Moon et al., Nat. Mater., 10:243 (2011).
Mornet et al., Nano Lett., 5:281 (2005).
Murat Cokol, Raj Nair & Burkhard Rost, at the website ubic.bloc.columbia.edu/papers/2000 nls/paper.html#ab2.
Nag and Awasthi, Pharmaceutics, 5:542 (2013).
Nandiyanto et al., Microporous Mesoporous Mater., 120:447 (2009).
Nollert et al., Biophys. J., 69:1447 (1995).
Padera et al., Nature, 427:695 (2004).
Pattni et al., Chem. Rev. Chemical Reviews, 115:10938 (2015).
Peer et al., Nat. Nanotechnol., 2:751 (2007).
Perry et al., Nano Lett., 12:5304 (2012).
Petros and DeSimone, Nat. Rev. Drug Discov., 9:615 (2010).
Pluedemann, Silane Coupling Agents, Plenum Press, N.Y. 1982.
Porotto et al., PloS one. 6:e16874 (2011).
Reviakine and Brisson, Langmuir, 16:1806 (2000).
Reynolds et al., Toxicol. Appl. Pharmacol., 262:1 (2012).
Richter and Brisson, Biophys. J., 88:3422 (2005).
Riese et al., Mol. Cel. Biol., 15, 5770-5776.
Roggers et al., Mol. Pharm., 9:2770 (2012).
Sackmann, FEBS Lett., 346:3:16 (1994).
Sackmann, Handbook of Biological Physics, 1:213 (1995).
Savarala et al., Langmuir, 26:12081 (2010).
Schulze et al., Blochimica et Biophysica Acta (BBA)—Molecular Cell Research, 1793:674 (2009).
Seantier and Kasemo, Langmuir, 25:5767 (2009).
Shen et al., Nano Lett., 14:923 (2014).
Shi et al., Acc. Chem. Res., 44:1123 (2011).
Steichen et al., Eur. J. Pharm. Sci., 48:416 (2013).
Sun et al., Angew. Chem., Int. Ed., 53:12320 (2014).
Tam et al., Acc. Chem. Res., 46:792 (2013).
Torchilin, Nat. Rev. Drug Discovery, 4:145 (2005).
Townson et al., J. Am. Chem. Soc., 135:16030 (2013).
Vargas et al., Adv. Drug Deliv. Rev., 59:1162 (2007).
Vivero-Escoto et al., Small, 6:1952 (2010).
Wang et al., ACS Nano, 4:4371 (2010).
Wang et al., Bomaterials, 34:7662 (2013).
Wang et al., J. Colloid Interface Sci., 385:41 (2012).
Wang et al., RSC Adv., 2:11338 (2012).
Weis, TIBS, 23:185 (1998).
Weis, Trends Biochem. Sci., 2:235 (1998).
Williford et al., Annu. Rev. Biomed. Eng., 16:347 (2014).
Yeagle, CRC press: (2004).
Yu et al., ACS Nano, 5:5717 (2011).
Zarschler et al., Nanoscale, 6:6048 (2014).
Zhang et al., Adv. Fund. Mater., 24:2352 (2014).
Zhang et al., Biomaterials, 35:3650 (2014).
Zhang et al., J. Am. Chem. Soc., 134:15790 (2012).
Zhu et al., Bomacromolecules, 15:1845 (2014).

All publications, patents and patent applications are incorporated herein by reference. While in the foregoing specification, this invention has been described in relation to certain embodiments thereof, and many details have been set forth for purposes of illustration, it will be apparent to those skilled in the art that the invention is susceptible to additional embodiments and that certain of the details herein may be varied considerably without departing from the basic principles of the invention.

1-24. (canceled)

25. A multilamellar protocell comprising:
a nanoporous silica or metal oxide core and a multilamellar lipid bi-layer coating said core, the multilamellar lipid bi-layer comprising at least an inner lipid bi-layer and an outer lipid bi-layer and optionally an inner aqueous layer and/or an outer aqueous layer, said inner aqueous layer separating said core from said inner lipid bi-layer and said outer aqueous layer separating said inner lipid bi-layer from said outer lipid bi-layer
said outer lipid bi-layer comprising: at least one Toll-like receptor (TLR) agonist; a fusogenic peptide; and optionally at least one cell targeting species which selectively binds to a target on antigen presenting cells (APCs);
said inner lipid bi-layer comprising an endosomolytic peptide.

26. A unilamellar protocell comprising:
a nanoporous silica or metal oxide core and a lipid bi-layer coating said core and an optional aqueous layer separating said core from said lipid bi-layer,
said lipid bi-layer comprising: at least one Toll-like receptor (TLR) agonist; a fusogenic peptide; optionally at least one cell targeting species which selectively binds to a target on antigen presenting cells (APCs); and an endosomolytic peptide.

27. The protocell of claim 25, wherein said Toll-like receptor (TLR) agonist comprises Pam3Cys, HMGB1, Porins, HSP, GLP, BCG-CWS, HP-NAP, Zymosan, MALP2, PSK, dsRNA, Poly AU, Poly ICLC, Poly I:C, LPS, EDA, HSP, Fibrinogen, Monophosphoryl Lipid A (MPLA), Flagellin, Imiquimod, ssRNA, PolyG10, CpG, and mixtures thereof.

28. The protocell of claim 26, wherein the fusogenic peptide comprises octa-arginine (R8) peptide.

29. The protocell of claim 25, wherein the cell targeting species selectively binds to a target on antigen presenting cells (APCs).

30. The protocell of claim 25, wherein the endosomolytic peptide comprises H5WYG peptide (H₂N-GLFHAIAHFIHGGWHGLIHGWYGGC-COOH, SEQ ID NO: 2), RALA peptide (NH₂—WEARLARALARALARHLARALARALRAGEA-COOH, SEQ ID NO: 18), KALA peptide (NH₂—WEAKLAKALAKALAKHLAKALAKALKAGEA-COOH, SEQ ID NO:19), GALA (NH2-WEAALAEALAEALAEHLAEALAEALEALAA-COOH, SEQ ID NO:20) or INF7 (NH2-GLFEAIEGFIENGWEGMIDGWYG-COOH, SEQ ID NO:21).

31. The protocell of claim 25, wherein said outer lipid bi-layer, said inner lipid bi-layer, and/or at least one aqueous layer comprises at least one microbial protein which is optionally a viral antigen.

32. The protocell of claim 25, wherein said core is loaded with a microbial antigen or with a plasmid DNA which optionally encodes a microbial antigen.

33. The protocell of claim 32, wherein the microbial antigen is fused to ubiquitin.

34. The unilamellar protocell of claim 26, wherein said protocell is loaded with a DNA plasmid in the core and optionally contains a microbial antigen.

35. The protocell according to claim 25, wherein the antigen presenting cell is a professional antigen presenting cell.

36. A pharmaceutical composition comprising a population of protocells according to claim 25 in combination with a pharmaceutically acceptable carrier, additive or excipient.

37. A vaccine comprising the composition according to claim 36, optionally in combination with an adjuvant.

38. A method of inducing an immunogenic response in a subject comprising administering to said subject an effective amount of a composition according to claim 36 to said subject.

39. A method inducing immunity to a microbial infection in a subject comprising administering at least once, an effective amount of a composition according to claim 36.


SEQUENCE LISTING (25 sequences)

SEQ ID NO: 1 - protein, 8 aa, Artificial Sequence
  A synthetic peptide
RRRRRRRR

SEQ ID NO: 2 - protein, 25 aa, Artificial Sequence
  A synthetic peptide
GLFHAIAHFIHGGWHGLIHGWYGGC

SEQ ID NO: 3 - protein, 12 aa, Artificial Sequence
  A synthetic peptide
SFSIILTPILPL

SEQ ID NO: 4 - protein, 25 aa, Artificial Sequence
  A synthetic peptide
GLFHAIAHFIHGGWHGLIHGWYGGC

SEQ ID NO: 5 - protein, 8 aa, Artificial Sequence
  A synthetic peptide
RRRRRRRR

SEQ ID NO: 6 - protein, 18 aa, Artificial Sequence
  A synthetic peptide
SFSIILTPILPLEEEGGC

SEQ ID NO: 7 - protein, 7 aa, Artificial Sequence
  A synthetic peptide
ASVHFPP

SEQ ID NO: 8 - protein, 7 aa, Artificial Sequence
  A synthetic peptide
TATFWFQ

SEQ ID NO: 9 - protein, 7 aa, Artificial Sequence
  A synthetic peptide
TSPVALL

SEQ ID NO: 10 - protein, 7 aa, Artificial Sequence
  A synthetic peptide
IPLKVHP

SEQ ID NO: 11 - protein, 7 aa, Artificial Sequence
  A synthetic peptide
WPRLTNM

SEQ ID NO: 12 - protein, 25 aa, Artificial Sequence
  A synthetic peptide
GLFHAIAHFIHGGWHGLIHGWYGGC

SEQ ID NO: 13 - protein, 8 aa, Artificial Sequence
  A synthetic peptide
RRRRRRRR

SEQ ID NO: 14 - protein, 30 aa, Artificial Sequence
  A synthetic peptide
WEARLARALARALARHLARALARALRAGEA

SEQ ID NO: 15 - protein, 30 aa, Artificial Sequence
  A synthetic peptide
WEAKLAKALAKALAKHLAKALAKALKAGEA

SEQ ID NO: 16 - protein, 30 aa, Artificial Sequence
  A synthetic peptide
WEAALAEALAEALAEHLAEALAEALEALAA

SEQ ID NO: 17 - protein, 23 aa, Artificial Sequence
  A synthetic peptide
GLFEAIEGFIENGWEGMIDGWYG

SEQ ID NO: 18 - protein, 30 aa, Artificial Sequence; identical to SEQ ID NO: 14
  A synthetic peptide

SEQ ID NO: 19 - protein, 30 aa, Artificial Sequence; identical to SEQ ID NO: 15
  A synthetic peptide

SEQ ID NO: 20 - protein, 30 aa, Artificial Sequence; identical to SEQ ID NO: 16
  A synthetic peptide

SEQ ID NO: 21 - protein, 23 aa, Artificial Sequence
  A synthetic peptide
GLFEAIEGFIENGWEGMIDGWYG

SEQ ID NO: 22 - protein, 42 aa, Artificial Sequence
  A synthetic nuclear localization sequence
GNQSSNFGPMKGGNFGGRSSGPYGGGGQYFAKPRNQGGYGGC

SEQ ID NO: 23 - protein, 7 aa, Artificial Sequence
  A synthetic nuclear localization sequence
RRMKWKK

SEQ ID NO: 24 - protein, 7 aa, Artificial Sequence
  A synthetic localization sequence
PKKKRKV

SEQ ID NO: 25 - protein, 16 aa, Artificial Sequence
  A synthetic localization sequence
KRPAATKKAGQAKKKK